SEQUENCE LISTING (154 sequences)

SEQ ID NO: 1 - DNA, 696 bp, Synechococcus elongatus
atgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttga

SEQ ID NO: 2 - protein, 231 aa, Synechococcus elongatus
MPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 3 - DNA, 696 bp, Synechocystis sp.
atgcccgagcttgctgtccgcaccgaatttgactattccagcgaaatttacaaagacgcctatagccgcatcaacgccattgtcattgaaggcgaacaggaagcctacagcaactacctccagatggcggaactcttgccggaagacaaagaagagttgacccgcttggccaaaatggaaaaccgccataaaaaaggtttccaagcctgtggcaacaacctccaagtgaaccctgatatgccctatgcccaggaatttttcgccggtctccatggcaatttccagcacgcttttagcgaagggaaagttgttacctgtttattgatccaggctttgattatcgaagcttttgcgatcgccgcctataacatatatatccctgtggcggacgactttgctcggaaaatcactgagggcgtagtcaaggacgaatacacccacctcaactacggggaagaatggctaaaggccaactttgccaccgctaaggaagaactggagcaggccaacaaagaaaacctacccttagtgtggaaaatgctcaaccaagtgcagggggacgccaaggtattgggcatggaaaaagaagccctagtggaagattttatgatcagctacggcgaagccctcagtaacatcggcttcagcaccagggaaattatgcgtatgtcttcctacggtttggccggagtctag

SEQ ID NO: 4 - protein, 231 aa, Synechocystis sp.
MPELAVRTEFDYSSEIYKDAYSRINAIVIEGEQEAYSNYLQMAELLPEDKEELTRLAKMENRHKKGFQACGNNLQVNPDMPYAQEFFAGLHGNFQHAFSEGKVVTCLLIQALIIEAFAIAAYNIYIPVADDFARKITEGVVKDEYTHLNYGEEWLKANFATAKEELEQANKENLPLVWKMLNQVQGDAKVLGMEKEALVEDFMISYGEALSNIGFSTREIMRMSSYGLAGV

SEQ ID NO: 5 - DNA, 699 bp, Nostoc punctiforme
atgcagcagcttacagaccaatctaaagaattagatttcaagagcgaaacatacaaagatgcttatagccggattaatgcgatcgtgattgaaggggaacaagaagcccatgaaaattacatcacactagcccaactgctgccagaatctcatgatgaattgattcgcctatccaagatggaaagccgccataagaaaggatttgaagcttgtgggcgcaatttagctgttaccccagatttgcaatttgccaaagagtttttctccggcctacaccaaaattttcaaacagctgccgcagaagggaaagtggttacttgtctgttgattcagtctttaattattgaatgttttgcgatcgcagcatataacatttacatccccgttgccgacgatttcgcccgtaaaattactgaaggagtagttaaagaagaatacagccacctcaattttggagaagtttggttgaaagaacactttgcagaatccaaagctgaacttgaacttgcaaatcgccagaacctacccatcgtctggaaaatgctcaaccaagtagaaggtgatgcccacacaatggcaatggaaaaagatgctttggtagaagacttcatgattcagtatggtgaagcattgagtaacattggtttttcgactcgcgatattatgcgcttgtcagcctacggactcataggtgcttaa

SEQ ID NO: 6 - protein, 232 aa, Nostoc punctiforme
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 7 - DNA, 696 bp, Nostoc sp.
atgcagcaggttgcagccgatttagaaattgatttcaagagcgaaaaatataaagatgcctatagtcgcataaatgcgatcgtgattgaaggggaacaagaagcatacgagaattacattcaactatcccaactgctgccagacgataaagaagacctaattcgcctctcgaaaatggaaagccgtcacaaaaaaggatttgaagcttgtggacggaacctacaagtatcaccagatatggagtttgccaaagaattctttgctggactacacggtaacttccaaaaagcggcggctgaaggtaaaatcgttacctgtctattgattcagtccctgattattgaatgttttgcgatcgccgcatacaatatctacattcccgttgctgacgattttgctcgtaaaatcactgagggtgtagtcaaagatgaatacagccacctcaacttcggcgaagtttggttacagaaaaattttgcccaatccaaagcagaattagaagaagctaatcgtcataatcttcccatagtttggaaaatgctcaatcaagtcgcggatgatgccgcagtcttagctatggaaaaagaagccctagtcgaagattttatgattcagtacggcgaagcgttaagtaatattggcttcacaaccagagatattatgcggatgtcagcctacggacttacagcagcttaa

SEQ ID NO: 8 - protein, 231 aa, Nostoc sp.
MQQVAADLEIDFKSEKYKDAYSRINAIVIEGEQEAYENYIQLSQLLPDDKEDLIRLSKMESRHKKGFEACGRNLQVSPDMEFAKEFFAGLHGNFQKAAAEGKIVTCLLIQSLIIECFAIAAYNIYIPVADDFARKITEGVVKDEYSHLNFGEVWLQKNFAQSKAELEEANRHNLPIVWKMLNQVADDAAVLAMEKEALVEDFMIQYGEALSNIGFTTRDIMRMSAYGLTAA

SEQ ID NO: 9 - DNA, 696 bp, Acaryochloris marina
atgccccaaactcaggctatttcagaaattgacttctatagtgacacctacaaagatgcttacagtcgtattgacggcattgtgatcgaaggtgagcaagaagcgcatgaaaactatattcgtcttggcgaaatgctgcctgagcaccaagacgactttatccgcctgtccaagatggaagcccgtcataagaaagggtttgaagcctgcggtcgcaacttaaaagtaacctgcgatctagactttgcccggcgtttcttttccgacttacacaagaattttcaagatgctgcagctgaggataaagtgccaacttgcttagtgattcagtccttgatcattgagtgttttgcgatcgcagcttacaacatctatatccccgtcgctgatgactttgcccgtaagattacagagtctgtggttaaggatgagtatcaacacctcaattatggtgaagagtggcttaaagctcacttcgatgatgtgaaagcagaaatccaagaagctaatcgcaaaaacctccccatcgtttggagaatgctgaacgaagtggacaaggatgcggccgttttaggaatggaaaaagaagccctggttgaagacttcatgatccagtatggtgaagcccttagcaatattggtttctctacaggcgaaattatgcggatgtctgcctatggtcttgtggctgcgtaa

SEQ ID NO: 10 - protein, 231 aa, Acaryochloris marina
MPQTQAISEIDFYSDTYKDAYSRIDGIVIEGEQEAHENYIRLGEMLPEHQDDFIRLSKMEARHKKGFEACGRNLKVTCDLDFARRFFSDLHKNFQDAAAEDKVPTCLVIQSLIIECFAIAAYNIYIPVADDFARKITESVVKDEYQHLNYGEEWLKAHFDDVKAEIQEANRKNLPIVWRMLNEVDKDAAVLGMEKEALVEDFMIQYGEALSNIGFSTGEIMRMSAYGLVAA

SEQ ID NO: 11 - DNA, 696 bp, Thermosynechococcus elongatus
atgacaacggctaccgctacacctgttttggactaccatagcgatcgctacaaggatgcctacagccgcattaacgccattgtcattgaaggtgaacaggaagctcacgataactatatcgatttagccaagctgctgccacaacaccaagaggaactcacccgccttgccaagatggaagctcgccacaaaaaggggtttgaggcctgtggtcgcaacctgagcgtaacgccagatatggaatttgccaaagccttctttgaaaaactgcgcgctaactttcagagggctctggcggagggaaaaactgcgacttgtcttctgattcaagctttgatcatcgaatcctttgcgatcgcggcctacaacatctacatcccaatggcggatcctttcgcccgtaaaattactgagagtgttgttaaggacgaatacagccacctcaactttggcgaaatctggctcaaggaacactttgaaagcgtcaaaggagagctcgaagaagccaatcgcgccaatttacccttggtctggaaaatgctcaaccaagtggaagcagatgccaaagtgctcggcatggaaaaagatgcccttgtggaagacttcatgattcagtacagtggtgccctagaaaatatcggctttaccacccgcgaaattatgaagatgtcagtttatggcctcactggggcataa

SEQ ID NO: 12 - protein, 231 aa, Thermosynechococcus elongatus
MTTATATPVLDYHSDRYKDAYSRINAIVIEGEQEAHDNYIDLAKLLPQHQEELTRLAKMEARHKKGFEACGRNLSVTPDMEFAKAFFEKLRANFQRALAEGKTATCLLIQALIIESFAIAAYNIYIPMADPFARKITESVVKDEYSHLNFGEIWLKEHFESVKGELEEANRANLPLVWKMLNQVEADAKVLGMEKDALVEDFMIQYSGALENIGFTTREIMKMSVYGLTGA

SEQ ID NO: 13 - DNA, 732 bp, Synechococcus sp.
atggccccagcgaacgtcctgcccaacacccccccgtcccccactgatgggggcggcactgccctagactacagcagcccaaggtatcggcaggcctactcccgcatcaacggtattgttatcgaaggcgaacaagaagcccacgacaactacctcaagctggccgaaatgctgccggaagctgcagaggagctgcgcaagctggccaagatggaattgcgccacatgaaaggcttccaggcctgcggcaaaaacctgcaggtggaacccgatgtggagtttgcccgcgcctttttcgcgcccttgcgggacaatttccaaagcgccgcagcggcaggggatctggtctcctgttttgtcattcagtctttgatcatcgagtgctttgccattgccgcctacaacatctacatcccggttgccgatgactttgcccgcaagatcaccgagggggtagttaaggacgagtatctgcacctcaattttggggagcgctggctgggcgagcactttgccgaggttaaagcccagatcgaagcagccaacgcccaaaatctgcctctagttcggcagatgctgcagcaggtagaggcggatgtggaagccatttacatggatcgcgaggccattgtagaagacttcatgatcgcctacggcgaggccctggccagcatcggcttcaacacccgcgaggtaatgcgcctctcggcccagggtctgcgggccgcctga

SEQ ID NO: 14 - protein, 243 aa, Synechococcus sp.
MAPANVLPNTPPSPTDGGGTALDYSSPRYRQAYSRINGIVIEGEQEAHDNYLKLAEMLPEAAEELRKLAKMELRHMKGFQACGKNLQVEPDVEFARAFFAPLRDNFQSAAAAGDLVSCFVIQSLIIECFAIAAYNIYIPVADDFARKITEGVVKDEYLHLNFGERWLGEHFAEVKAQIEAANAQNLPLVRQMLQQVEADVEAIYMDREAIVEDFMIAYGEALASIGFNTREVMRLSAQGLRAA

SEQ ID NO: 15 - DNA, 708 bp, Gloeobacter violaceus
gtgaaccgaaccgcaccgtccagcgccgcgcttgattaccgctccgacacctaccgcgatgcgtactcccgcatcaatgccatcgtccttgaaggcgagcgggaagcccacgccaactaccttaccctcgctgagatgctgccggaccatgccgaggcgctcaaaaaactggccgcgatggaaaatcgccacttcaaaggcttccagtcctgcgcccgcaacctcgaagtcacgccggacgacccgtttgcaagggcctacttcgaacagctcgacggcaactttcagcaggcggcggcagaaggtgaccttaccacctgcatggtcatccaggcactgatcatcgagtgcttcgcaattgcggcctacaacgtctacattccggtggccgacgcgtttgcccgcaaggtgaccgagggcgtcgtcaaggacgagtacacccacctcaactttgggcagcagtggctcaaagagcgcttcgtgaccgtgcgcgagggcatcgagcgcgccaacgcccagaatctgcccatcgtctggcggatgctcaacgccgtcgaagcggacaccgaagtgctgcagatggataaagaagcgatcgtcgaagactttatgatcgcctacggtgaagccttgggcgacatcggtttttcgatgcgcgacgtgatgaagatgtccgcccgcggccttgcctctgccccccgccagtga

SEQ ID NO: 16 - protein, 235 aa, Gloeobacter violaceus
MNRTAPSSAALDYRSDTYRDAYSRINAIVLEGEREAHANYLTLAEMLPDHAEALKKLAAMENRHFKGFQSCARNLEVTPDDPFARAYFEQLDGNFQQAAAEGDLTTCMVIQALIIECFAIAAYNVYIPVADAFARKVTEGVVKDEYTHLNFGQQWLKERFVTVREGIERANAQNLPIVWRMLNAVEADTEVLQMDKEAIVEDFMIAYGEALGDIGFSMRDVMKMSARGLASAPRQ

SEQ ID NO: 17 - DNA, 732 bp, Prochlorococcus marinus
atgcctacgcttgagatgcctgtggcagctgttcttgacagcactgttggatcttcagaagccctgccagacttcacttcagatagatataaggatgcatacagcagaatcaacgcaatagtcattgagggcgaacaggaagcccatgacaattacatcgcgattggcacgctgcttcccgatcatgtcgaagagctcaagcggcttgccaagatggagatgaggcacaagaagggctttacagcttgcggcaagaaccttggcgttgaggctgacatggacttcgcaagggagttttttgctcctttgcgtgacaacttccagacagctttagggcaggggaaaacacctacatgcttgctgatccaggcgctcttgattgaagcctttgctatttcggcttatcacacctatatccctgtttctgacccctttgctcgcaagattactgaaggtgtcgtgaaggacgagtacacacacctcaattatggcgaggcttggctcaaggccaatctggagagttgccgtgaggagttgcttgaggccaatcgcgagaacctgcctctgattcgccggatgcttgatcaggtagcaggtgatgctgccgtgctgcagatggataaggaagatctgattgaggatttcttaatcgcctaccaggaatctctcactgagattggctttaacactcgtgaaattacccgtatggcagcggcagctcttgtgagctga

SEQ ID NO: 18 - protein, 243 aa, Prochlorococcus marinus
MPTLEMPVAAVLDSTVGSSEALPDFTSDRYKDAYSRINAIVIEGEQEAHDNYIAIGTLLPDHVEELKRLAKMEMRHKKGFTACGKNLGVEADMDFAREFFAPLRDNFQTALGQGKTPTCLLIQALLIEAFAISAYHTYIPVSDPFARKITEGVVKDEYTHLNYGEAWLKANLESCREELLEANRENLPLIRRMLDQVAGDAAVLQMDKEDLIEDFLIAYQESLTEIGFNTREITRMAAAALVS

SEQ ID NO: 19 - DNA, 717 bp, Prochlorococcus marinus
atgcaaacactcgaatctaataaaaaaactaatctagaaaattctattgatttacccgattttactactgattcttacaaagacgcttatagcaggataaatgcaatagttattgaaggtgaacaagaggctcatgataattacatttccttagcaacattaattcctaacgaattagaagagttaactaaattagcgaaaatggagcttaagcacaaaagaggctttactgcatgtggaagaaatctaggtgttcaagctgacatgatttttgctaaagaattcttttccaaattacatggtaattttcaggttgcgttatctaatggcaagacaactacatgcctattaatacaggcaattttaattgaagcttttgctatatccgcgtatcacgtttacataagagttgctgatcctttcgcgaaaaaaattacccaaggtgttgttaaagatgaatatcttcatttaaattatggacaagaatggctaaaagaaaatttagcgacttgtaaagatgagctaatggaagcaaataaggttaaccttccattaatcaagaagatgttagatcaagtctcggaagatgcttcagtactagctatggatagggaagaattaatggaagaattcatgattgcctatcaggacactctccttgaaataggtttagataatagagaaattgcaagaatggcaatggctgctatagtttaa

SEQ ID NO: 20 - protein, 238 aa, Prochlorococcus marinus
MQTLESNKKTNLENSIDLPDFTTDSYKDAYSRINAIVIEGEQEAHDNYISLATLIPNELEELTKLAKMELKHKRGFTACGRNLGVQADMIFAKEFFSKLHGNFQVALSNGKTTTCLLIQAILIEAFAISAYHVYIRVADPFAKKITQGVVKDEYLHLNYGQEWLKENLATCKDELMEANKVNLPLIKKMLDQVSEDASVLAMDREELMEEFMIAYQDTLLEIGLDNREIARMAMAAIV

SEQ ID NO: 21 - DNA, 726 bp, Prochlorococcus marinus
atgcaagcttttgcatccaacaatttaaccgtagaaaaagaagagctaagttctaactctcttccagatttcacctcagaatcttacaaagatgcttacagcagaatcaatgcagttgtaattgaaggggagcaagaagcttattctaattttcttgatctcgctaaattgattcctgaacatgcagatgagcttgtgaggctagggaagatggagaaaaagcatatgaatggtttttgtgcttgcgggagaaatcttgctgtaaagcctgatatgccttttgcaaagacctttttctcaaaactccataataattttttagaggctttcaaagtaggagatacgactacctgtctcctaattcaatgcatcttgattgaatcttttgcaatatccgcatatcacgtttatatacgtgttgctgatccattcgccaaaagaatcacagagggtgttgtccaagatgaatacttgcatttgaactatggtcaagaatggcttaaggccaatctagagacagttaagaaagatcttatgagggctaataaggaaaacttgcctcttataaagtccatgctcgatgaagtttcaaacgacgccgaagtccttcatatggataaagaagagttaatggaggaatttatgattgcttatcaagattcccttcttgaaataggtcttgataatagagaaattgcaagaatggctcttgcagcggtgatataa

SEQ ID NO: 22 - protein, 241 aa, Prochlorococcus marinus
MQAFASNNLTVEKEELSSNSLPDFTSESYKDAYSRINAVVIEGEQEAYSNFLDLAKLIPEHADELVRLGKMEKKHMNGFCACGRNLAVKPDMPFAKTFFSKLHNNFLEAFKVGDTTTCLLIQCILIESFAISAYHVYIRVADPFAKRITEGVVQDEYLHLNYGQEWLKANLETVKKDLMRANKENLPLIKSMLDEVSNDAEVLHMDKEELMEEFMIAYQDSLLEIGLDNREIARMALAAVI

SEQ ID NO: 23 - DNA, 732 bp, Synechococcus sp.
atgccgacccttgagacgtctgaggtcgccgttcttgaagactcgatggcttcaggctcccggctgcctgatttcaccagcgaggcttacaaggacgcctacagccgcatcaatgcgatcgtgatcgagggtgagcaggaagcgcacgacaactacatcgccctcggcacgctgatccccgagcagaaggatgagctggcccgtctcgcccgcatggagatgaagcacatgaaggggttcacctcctgtggccgcaatctcggcgtggaggcagaccttccctttgctaaggaattcttcgcccccctgcacgggaacttccaggcagctctccaggagggcaaggtggtgacctgcctgttgattcaggcgctgctgattgaagcgttcgccatttccgcctatcacatctacatcccggtggcggatcccttcgctcgcaagatcactgaaggtgtggtgaaggatgagtacacccacctcaattacggccaggaatggctgaaggccaattttgaggccagcaaggatgagctgatggaggccaacaaggccaatctgcctctgatccgctcgatgctggagcaggtggcagccgacgccgccgtgctgcagatggaaaaggaagatctgatcgaagatttcctgatcgcttaccaggaggccctctgcgagatcggtttcagctcccgtgacattgctcgcatggccgccgctgccctcgcggtctga

SEQ ID NO: 24 - protein, 243 aa, Synechococcus sp.
MPTLETSEVAVLEDSMASGSRLPDFTSEAYKDAYSRINAIVIEGEQEAHDNYIALGTLIPEQKDELARLARMEMKHMKGFTSCGRNLGVEADLPFAKEFFAPLHGNFQAALQEGKVVTCLLIQALLIEAFAISAYHIYIPVADPFARKITEGVVKDEYTHLNYGQEWLKANFEASKDELMEANKANLPLIRSMLEQVAADAAVLQMEKEDLIEDFLIAYQEALCEIGFSSRDIARMAAAALAV

SEQ ID NO: 25 - DNA, 681 bp, Synechococcus sp.
atgacccagctcgactttgccagtgcggcctaccgcgaggcctacagccggatcaacggcgttgtgattgtgggcgaaggtctcgccaatcgccatttccagatgttggcgcggcgcattcccgctgatcgcgacgagctgcagcggctcggacgcatggagggagaccatgccagcgcctttgtgggctgtggtcgcaacctcggtgtggtggccgatctgcccctggcccggcgcctgtttcagcccctccatgatctgttcaaacgccacgaccacgacggcaatcgggccgaatgcctggtgatccaggggttgatcgtggaatgtttcgccgtggcggcttaccgccactacctgccggtggccgatgcctacgcccggccgatcaccgcagcggtgatgaacgatgaatcggaacacctcgactacgctgagacctggctgcagcgccatttcgatcaggtgaaggcccgggtcagcgcggtggtggtggaggcgttgccgctcaccctggcgatgttgcaatcgcttgctgcagacatgcgacagatcggcatggatccggtggagaccctggccagcttcagtgaactgtttcgggaagcgttggaatcggtggggtttgaggctgtggaggccaggcgactgctgatgcgagcggccgcccggatggtctga

SEQ ID NO: 26 - protein, 226 aa, Synechococcus sp.
MTQLDFASAAYREAYSRINGVVIVGEGLANRHFQMLARRIPADRDELQRLGRMEGDHASAFVGCGRNLGVVADLPLARRLFQPLHDLFKRHDHDGNRAECLVIQGLIVECFAVAAYRHYLPVADAYARPITAAVMNDESEHLDYAETWLQRHFDQVKARVSAVVVEALPLTLAMLQSLAADMRQIGMDPVETLASFSELFREALESVGFEAVEARRLLMRAAARMV

SEQ ID NO: 27 - DNA, 696 bp, Cyanothece sp.
atgcaagagcttgctttacgctcagagcttgattttaacagcgaaacctataaagatgcttacagtcgcatcaatgctattgtcattgaaggggaacaagaagcctatcaaaattatcttgatatggcgcaacttctcccagaagacgaggctgagttaattcgtctctccaagatggaaaaccgtcacaaaaaaggctttcaagcctgtggcaagaatttgaatgtgaccccagatatggactacgctcaacaattttttgctgaacttcatggcaacttccaaaaggcaaaagccgaaggcaaaattgtcacttgcttattaattcaatctttgatcatcgaagcctttgcgatcgccgcttataatatttatattcctgtggcagatccctttgctcgtaaaatcaccgaaggggtagttaaggatgaatatacccacctcaattttggggaagtctggttaaaagagcattttgaagcctctaaagcagaattagaagacgcaaataaagaaaatttaccccttgtttggcaaatgctcaaccaagttgaaaaagatgccgaagtgttagggatggagaaagaagccttagtggaagatttcatgattagttatggagaagctttaagtaatattggtttctctacccgtgagatcatgaaaatgtctgcttacgggctacgggctgcttaa

SEQ ID NO: 28 - protein, 231 aa, Cyanothece sp.
MQELALRSELDFNSETYKDAYSRINAIVIEGEQEAYQNYLDMAQLLPEDEAELIRLSKMENRHKKGFQACGKNLNVTPDMDYAQQFFAELHGNFQKAKAEGKIVTCLLIQSLIIEAFAIAAYNIYIPVADPFARKITEGVVKDEYTHLNFGEVWLKEHFEASKAELEDANKENLPLVWQMLNQVEKDAEVLGMEKEALVEDFMISYGEALSNIGFSTREIMKMSAYGLRAA

SEQ ID NO: 29 - DNA, 696 bp, Cyanothece sp.
atgcctcaagtgcagtccccatcggctatagacttctacagtgagacctaccaggatgcttacagccgcattgatgcgatcgtgatcgagggagaacaggaagcccacgacaattacctgaagctgacggaactgctgccggattgtcaagaagatctggtccggctggccaaaatggaagcccgtcacaaaaaagggtttgaagcttgtggccgcaatctcaaggtcacacccgatatggagtttgctcaacagttctttgctgacctgcacaacaatttccagaaagctgctgcggccaacaaaattgccacctgtctggtgatccaggccctgattattgagtgctttgccatcgccgcttataacatctatattcctgtcgctgatgactttgcccgcaaaattaccgaaaacgtggtcaaagacgaatacacccacctcaactttggtgaagagtggctcaaagctaactttgatagccagcgggaagaagtggaagcggccaaccgggaaaacctgccgatcgtctggcggatgctcaatcaggtagagactgatgctcacgttttaggtatggaaaaagaggctttagtggaaagcttcatgatccaatatggtgaagccctggaaaatattggtttctcgacccgtgagatcatgcgcatgtccgtttacggcctctctgcggcataa

SEQ ID NO: 30 - protein, 231 aa, Cyanothece sp.
MPQVQSPSAIDFYSETYQDAYSRIDAIVIEGEQEAHDNYLKLTELLPDCQEDLVRLAKMEARHKKGFEACGRNLKVTPDMEFAQQFFADLHNNFQKAAAANKIATCLVIQALIIECFAIAAYNIYIPVADDFARKITENVVKDEYTHLNFGEEWLKANFDSQREEVEAANRENLPIVWRMLNQVETDAHVLGMEKEALVESFMIQYGEALENIGFSTREIMRMSVYGLSAA

SEQ ID NO: 31 - DNA, 702 bp, Cyanothece sp.
atgtctgattgcgccacgaacccagccctcgactattacagtgaaacctaccgcaatgcttaccggcgggtgaacggtattgtgattgaaggcgagaagcaagcctacgacaactttatccgcttagctgagctgctcccagagtatcaagcggaattaacccgtctggctaaaatggaagcccgccaccagaagagctttgttgcctgtggccaaaatctcaaggttagcccggacttagactttgcggcacagttttttgctgaactgcatcaaatttttgcatctgcagcaaatgcgggccaggtggctacctgtctggttgtgcaagccctgatcattgaatgctttgcgatcgccgcctacaatacctatttgccagtagcggatgaatttgcccgtaaagtcaccgcatccgttgttcaggacgagtacagccacctaaactttggtgaagtctggctgcagaatgcgtttgagcagtgtaaagacgaaattatcacagctaaccgtcttgctctgccgctgatctggaaaatgctcaaccaggtgacaggcgaattgcgcattctgggcatggacaaagcttctctggtagaagactttagcactcgctatggagaggccctgggccagattggtttcaaactatctgaaattctctccctgtccgttcagggtttacaggcggttacgccttag

SEQ ID NO: 32 - protein, 233 aa, Cyanothece sp.
MSDCATNPALDYYSETYRNAYRRVNGIVIEGEKQAYDNFIRLAELLPEYQAELTRLAKMEARHQKSFVACGQNLKVSPDLDFAAQFFAELHQIFASAANAGQVATCLVVQALIIECFAIAAYNTYLPVADEFARKVTASVVQDEYSHLNFGEVWLQNAFEQCKDEIITANRLALPLIWKMLNQVTGELRILGMDKASLVEDFSTRYGEALGQIGFKLSEILSLSVQGLQAVTP

SEQ ID NO: 33 - DNA, 696 bp, Anabaena variabilis
atgcagcaggttgcagccgatttagaaatcgatttcaagagcgaaaaatataaagatgcctatagtcgcataaatgcgatcgtgattgaaggggaacaagaagcatatgagaattacattcaactatcccaactgctgccagacgataaagaagacctaattcgcctctcgaaaatggaaagtcgccacaaaaaaggatttgaagcttgtggacggaacctgcaagtatccccagacatagagttcgctaaagaattctttgccgggctacacggtaatttccaaaaagcggcagctgaaggtaaagttgtcacttgcctattgattcaatccctgattattgaatgttttgcgatcgccgcatacaatatctacatccccgtggctgacgatttcgcccgtaaaatcactgagggtgtagttaaagatgaatacagtcacctcaacttcggcgaagtttggttacagaaaaatttcgctcaatcaaaagcagaactagaagaagctaatcgtcataatcttcccatagtctggaaaatgctcaatcaagttgccgatgatgcggcagtcttagctatggaaaaagaagccctagtggaagattttatgattcagtacggcgaagcactaagtaatattggcttcacaaccagagatattatgcggatgtcagcctacggactcacagcagcttaa

SEQ ID NO: 34 - protein, 231 aa, Anabaena variabilis
MQQVAADLEIDFKSEKYKDAYSRINAIVIEGEQEAYENYIQLSQLLPDDKEDLIRLSKMESRHKKGFEACGRNLQVSPDIEFAKEFFAGLHGNFQKAAAEGKVVTCLLIQSLIIECFAIAAYNIYIPVADDFARKITEGVVKDEYSHLNFGEVWLQKNFAQSKAELEEANRHNLPIVWKMLNQVADDAAVLAMEKEALVEDFMIQYGEALSNIGFTTRDIMRMSAYGLTAA

SEQ ID NO: 35 - DNA, 765 bp, Synechococcus elongatus
gtgcgtaccccctgggatccaccaaatcccacattctccctctcatccgtgtcaggagaccgcagactcatgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttga

SEQ ID NO: 36 - protein, 254 aa, Synechococcus elongatus
MRTPWDPPNPTFSLSSVSGDRRLMPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 37 - protein, 116 aa, Artificial Sequence
  Synthetic Peptide
EXXAXXXXXXXXXXXXXXXXXXXXXXXMEXXHXXXFXXXXXXXXXXXXXXXXXXXXXXXXXXXXXXXXXXXXXXXXXXQXXXXEXXAXXXYXXXXXXXXXXXXXXXXXVXXXEXXH

SEQ ID NO: 38 - protein, 19 aa, Artificial Sequence
  Synthetic Peptide
YXXAYXRXXXXVXXGEXXA

SEQ ID NO: 39 - protein, 15 aa, Artificial Sequence
  Synthetic Peptide
LXXMEXXHXXXFXXC

SEQ ID NO: 40 - protein, 17 aa, Artificial Sequence
  Synthetic Peptide
CXXXQXXXXEXFAXXAY

SEQ ID NO: 41 - protein, 19 aa, Artificial Sequence
  Synthetic Peptide
TXXVXXXEXXHXXXXXXWL

SEQ ID NO: 42 - protein, 23 aa, Artificial Sequence
  Synthetic Peptide
YXXAYXRXXXXVXXGEXXAXXXX

SEQ ID NO: 43 - protein, 21 aa, Artificial Sequence
  Synthetic Peptide
LXXMEXXHXXXFXXCXXNLXX

SEQ ID NO: 44 - protein, 21 aa, Artificial Sequence
  Synthetic Peptide
CXXXQXXXXEXFAXXAYXXYX

SEQ ID NO: 45 - protein, 26 aa, Artificial Sequence
  Synthetic Peptide
DXXAXXXTXXVXXXEXXHXXXXXXWL

SEQ ID NO: 46 - DNA, 699 bp, Nostoc punctiforme
atgcagcaactgacggatcagagcaaagaactggacttcaaaagcgaaacctacaaggacgcgtattctcgtatcaacgctatcgttatcgagggtgaacaagaagcgcacgagaattacattaccctggcgcagctgctgcctgaatcccacgatgaactgattcgtctgagcaaaatggagtcgcgtcacaaaaagggttttgaggcctgcggtcgtaacctggcggtcactccggacctgcagttcgctaaggagttcttcagcggcctgcatcaaaactttcagacggcagcggcggaaggtaaggttgtcacctgcctgctgattcaaagcctgatcattgagtgtttcgctatcgcagcctataacatttacatcccggtggcggacgattttgcacgcaagatcactgagggtgtggttaaagaagaatacagccacctgaacttcggtgaggtctggttgaaggagcactttgcggaaagcaaggcggagctggaattggcaaatcgtcaaaacctgccgatcgtgtggaaaatgctgaatcaagtggagggtgatgcacacacgatggctatggaaaaagacgctctggtggaggacttcatgatccagtacggcgaggcgctgagcaacattggctttagcacccgtgacattatgcgcctgagcgcgtatggcctgatcggtgcgtaa

SEQ ID NO: 47 - DNA, 696 bp, Acaryochloris marina
atgccgcaaacgcaagctattagcgaaattgatttctattctgacacctataaggacgcttactctcgtatcgatggtatcgtgatcgagggtgagcaagaggcgcatgagaactacattcgtctgggtgaaatgttgcctgagcatcaagacgactttatccgtttgagcaagatggaggcccgtcacaagaagggctttgaggcttgtggtcgtaacttgaaggtgacttgcgatctggacttcgcgcgtcgcttcttctcggacctgcacaagaacttccaagatgctgcggccgaggataaagttccgacctgcttggttattcagtccctgatcatcgaatgcttcgcgattgcagcgtataacatttacatcccggttgccgatgatttcgctcgtaagattaccgagagcgtcgtcaaggacgaataccagcatctgaactatggcgaggagtggctgaaggcccatttcgacgacgtgaaggccgagatccaggaagcaaatcgcaagaatctgccgatcgtttggcgtatgctgaacgaggttgacaaggacgcagcagtgctgggcatggagaaggaagcgttggttgaagacttcatgattcaatacggtgaggccctgtccaacattggcttttctaccggcgagatcatgcgtatgtctgcgtacggtctggtggcagcctaa

SEQ ID NO: 48 - DNA, 696 bp, Thermosynechococcus elongatus
atgaccaccgcgaccgcaacgccggtgctggactatcacagcgaccgctacaaggacgcatacagccgcatcaacgcgattgtcatcgaaggtgaacaagaggcccacgacaattacattgatctggctaaactgctgcctcaacaccaagaagagctgacccgtctggcgaagatggaggcccgccacaagaagggttttgaagcgtgcggtcgcaatctgtccgttaccccggatatggagttcgcgaaagcgttctttgagaagctgcgcgcgaactttcagcgtgccctggcggagggtaagaccgcaacctgtctgctgatccaggcgttgatcattgaatccttcgcaattgccgcgtacaacatttacatccctatggccgatccgtttgcgcgcaagattaccgaaagcgtcgtcaaggatgaatactctcacttgaactttggcgaaatctggttgaaggaacatttcgagagcgtcaagggcgagttggaggaagctaaccgtgcgaatctgccgctggtttggaagatgttgaatcaggtcgaggcagacgcaaaggtcctgggcatggagaaggatgctctggtggaagactttatgatccagtactccggtgcgctggagaacatcggctttaccacccgtgaaatcatgaaaatgtctgtgtatggcctgaccggcgcgtaa

SEQ ID NO: 49 - DNA, 732 bp, Synechococcus sp.
atggcgcctgcaaacgtgctgccaaatacgccgccgagcccgaccgatggtggtggtacggccctggactacagctctccgcgttaccgtcaggcgtacagccgtatcaatggcattgttatcgaaggcgagcaggaagcgcacgataactacctgaagttggcggagatgctgcctgaggctgccgaggaactgcgtaagctggcaaagatggaattgcgtcacatgaagggctttcaggcttgcggcaagaacttgcaggtggagcctgacgtcgagtttgcccgcgctttcttcgcgccgctgcgcgacaacttccaatccgcagcagcggccggtgatctggtttcctgtttcgtcatccaaagcctgatcatcgagtgttttgcgatcgctgcgtataacatttacatcccggttgcagacgacttcgcccgtaagatcacggagggcgtggttaaggacgagtatctgcatctgaatttcggcgagcgttggttgggtgaacacttcgcagaggttaaagcacagatcgaggcagccaatgcccagaacctgccgctggtgcgccaaatgctgcagcaagttgaggcggacgtcgaggcaatctatatggaccgtgaggcgatcgttgaggatttcatgattgcttatggcgaagcgctggcaagcattggcttcaacacgcgcgaagtgatgcgtctgagcgcacagggcttgcgtgcagcataa

SEQ ID NO: 50 - DNA, 732 bp, Prochlorococcus marinus
atgccgacgttggagatgccggtcgctgcggtcctggacagcacggtcggtagctctgaggcgctgccggactttaccagcgaccgctacaaagacgcttattcgcgtatcaacgcgattgtgatcgagggtgaacaagaagcccacgacaactacatcgcaattggcaccctgttgccggaccatgtggaagaactgaaacgtctggcgaaaatggaaatgcgtcacaagaaaggtttcaccgcgtgcggtaagaacttgggtgtggaagccgatatggacttcgcccgtgagttctttgccccgttgcgcgacaactttcaaaccgcgctgggtcaaggcaagacccctacgtgtctgttgatccaagcgctgctgattgaagcgttcgcgatctcggcctaccacacttacattccggttagcgatccgttcgcacgtaagatcactgaaggtgtcgttaaggacgaatacacccatctgaactacggtgaggcatggctgaaggcgaatctggagagctgccgcgaggaactgctggaagcgaaccgtgagaatctgccgctgatccgccgcatgctggatcaggtcgcgggcgacgcggcagtcctgcagatggataaggaagacctgatcgaagacttcctgattgcttaccaagagagcttgactgagatcggctttaacacgcgtgaaatcacccgtatggccgcagcggcgctggtcagctaa

SEQ ID NO: 51 - DNA, 717 bp, Prochlorococcus marinus
atgcaaaccctggagagcaacaagaaaaccaacctggaaaacagcattgacctgccagatttcacgacggacagctacaaggatgcgtattcccgtatcaatgctatcgtcattgaaggtgaacaggaagcccatgacaactatatcagcctggccaccctgatcccgaatgaactggaggaattgaccaaactggccaagatggagctgaaacacaaacgtggctttacggcatgcggtcgcaatctgggtgttcaggccgatatgatctttgcgaaagagtttttctctaagctgcacggcaacttccaagttgcgctgagcaacggtaagacgaccacctgcttgctgatccaggccatcttgattgaagccttcgcgatttccgcgtaccacgtgtacattcgtgtcgcggacccgtttgcgaaaaagattactcaaggtgtggtgaaggatgagtacctgcacctgaactatggtcaggaatggttgaaggagaatctggcaacctgtaaggacgaactgatggaagcaaacaaagttaatctgccgctgattaagaaaatgctggatcaggtgagcgaggatgcctctgtgttggctatggatcgtgaggagctgatggaggagttcatgatcgcgtatcaggacaccctgttggaaatcggtctggacaatcgtgaaattgcgcgtatggcaatggctgcgattgtgtaa

SEQ ID NO: 52 - DNA, 726 bp, Prochlorococcus marinus
atgcaggccttcgcaagcaataacctgacggtcgaaaaggaagaactgagctccaatagcctgccggatttcaccagcgagagctataaggatgcatactctcgtatcaatgccgtggttatcgaaggtgaacaagaggcttattctaactttctggacctggccaagctgatcccggagcacgccgacgagctggtgcgcttgggtaagatggaaaagaaacacatgaacggcttctgcgcgtgtggtcgtaacttggcagttaaaccagacatgccgttcgcgaagacgttctttagcaagctgcacaacaatttcctggaggcgtttaaggtgggcgatacgacgacctgtttgttgatccaatgcatcttgatcgagtcctttgccatcagcgcgtaccacgtgtacattcgcgtggcagatccgtttgccaagcgtatcacggaaggtgttgttcaagacgagtacctgcatttgaattacggtcaagagtggctgaaagcgaacctggagactgtgaagaaagacctgatgcgcgcgaacaaagagaatctgccattgattaagtctatgctggacgaagtctccaacgacgctgaagtgctgcacatggataaagaagagctgatggaagagtttatgattgcatatcaggacagcctgctggaaattggcctggacaaccgcgagatcgcacgcatggcgctggcagcggttatttaa

SEQ ID NO: 53 - DNA, 732 bp, Synechococcus sp.
atgccgaccctggaaactagcgaggtggcagttctggaagactcgatggccagcggtagccgcctgccggactttaccagcgaggcctataaggacgcgtatagccgtatcaatgcgatcgtgattgaaggcgagcaagaagcgcatgacaactacattgcactgggcacgctgatcccagaacagaaggacgagctggctcgcctggctcgtatggaaatgaaacacatgaagggctttaccagctgtggtcgtaacctgggtgtggaagcggatctgccgttcgcgaaggagttcttcgcaccgctgcatggtaactttcaggcggcgctgcaggaaggtaaggtggtgacctgtctgctgattcaggcactgctgattgaggcgttcgccattagcgcttatcacatttacattccggttgctgacccgtttgcacgcaagattaccgaaggtgttgtgaaagacgagtatacccatctgaactacggtcaagagtggttgaaggcgaatttcgaagcctccaaagacgaactgatggaagccaacaaggcgaatctgccgctgatccgttctatgctggaacaagtcgctgctgatgcggccgtgctgcaaatggagaaagaggacctgattgaagacttcctgatcgcatatcaagaagctctgtgtgagattggcttctcgtcccgtgatatcgcccgcatggcggcagccgcactggcggtttaa

SEQ ID NO: 54 - DNA, 681 bp, Synechococcus sp.
atgacccaattggactttgcatctgcggcataccgtgaggcatacagccgtatcaatggtgtcgttattgttggcgagggcctggcgaatcgtcacttccaaatgctggcgcgtcgcattccggcagaccgtgacgaattgcaacgtttgggccgcatggagggtgaccacgcaagcgcctttgttggttgcggtcgcaatctgggtgtggtcgctgatctgccgctggcacgccgcctgttccagccgctgcatgatctgttcaagcgtcacgaccacgacggtaaccgtgctgaatgcctggtgatccagggtctgattgttgagtgctttgcggttgccgcgtatcgtcattacctgccggtggcagacgcgtatgcccgtccgatcaccgctgcggttatgaatgacgagagcgaacacctggactacgcagaaacctggctgcagcgccacttcgaccaagttaaagcccgcgtgagcgctgtggttgtggaggcgctgccgctgacgctggcgatgttgcaaagcctggctgcagatatgcgccaaatcggcatggacccggtggaaacgctggcgagcttcagcgagctgtttcgtgaagcgctggaaagcgttggttttgaagcggtcgaagcgcgccgtttgctgatgcgtgctgcagctcgtatggtttaa

SEQ ID NO: 55 - protein, 28 aa, Artificial Sequence
  Synthetic Peptide
GAXGDIGSXXXXWXXXXXXXXXXXXXAR

SEQ ID NO: 56 - protein, 34 aa, Artificial Sequence
  Synthetic Peptide
ATVAXXGATGDIGSAVXRWLXXKXXXXXLXLXAR

SEQ ID NO: 57 - protein, 11 aa, Artificial Sequence
  Synthetic Peptide
XLXXXRFTTGN

SEQ ID NO: 58 - protein, 14 aa, Artificial Sequence
  Synthetic Peptide
MFGLIGHXXXXXXA

SEQ ID NO: 59 - protein, 19 aa, Artificial Sequence
  Synthetic Peptide
LXXWXXAPPXXXXXXXXXS

SEQ ID NO: 60 - protein, 21 aa, Artificial Sequence
  Synthetic Peptide
SXXGXXIXGXYXXSXFXPEML

SEQ ID NO: 61 - protein, 27 aa, Artificial Sequence
  Synthetic Peptide
KXAXRKXXXAMXXXQXXXXXIXXLGGF

SEQ ID NO: 62 - protein, 14 aa, Artificial Sequence
  Synthetic Peptide
AXXXXXXXXWVASX

SEQ ID NO: 63 - protein, 12 aa, Artificial Sequence
  Synthetic Peptide
PXXXXDGGYPKN

SEQ ID NO: 64 - protein, 25 aa, Artificial Sequence
  Synthetic Peptide
NFSWGRNXIXXXXXXXIGXXSXXHG

SEQ ID NO: 65 - protein, 9 aa, Artificial Sequence
  Synthetic Peptide
FTTGNTHTA

SEQ ID NO: 66 - DNA, 1026 bp, Synechococcus elongatus
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 67 - protein, 341 aa, Synechococcus elongatus
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 68 - DNA, 1023 bp, Synechocystis sp.
atgtttggtcttattggtcatctcacgagtttagaacacgcccaagcggttgctgaagatttaggctatcctgagtacgccaaccaaggcctggatttttggtgttcggctcctccccaagtggttgataattttcaggtgaaaagtgtgacggggcaggtgattgaaggcaaatatgtggagtcttgctttttgccggaaatgttaacccaacggcggatcaaagcggccattcgtaaaatcctcaatgctatggccctggcccaaaaggtgggcttggatattacggccctgggaggcttttcttcaatcgtatttgaagaatttaacctcaagcaaaataatcaagtccgcaatgtggaactagattttcagcggttcaccactggtaatacccacaccgcttatgtgatctgccgtcaggtcgagtctggagctaaacagttgggtattgatctaagtcaggcaacggtagcggtttgtggcgccacgggagatattggtagcgccgtatgtcgttggttagatagcaaacatcaagttaaggaattattgctaattgcccgtaaccgccaaagattggaaaatctccaagaggaattgggtcggggcaaaattatggatttggaaacagccctgccccaggcagatattattgtttgggtggctagtatgcccaagggggtagaaattgcgggggaaatgctgaaaaagccctgtttgattgtggatgggggctatcccaagaatttagacaccagggtgaaagcggatggggtgcatattctcaagggggggattgtagaacattcccttgatattacctgggaaattatgaagattgtggagatggatattccctcccggcaaatgttcgcctgttttgcggaggccattttgctagagtttgagggctggcgcactaatttttcctggggccgcaaccaaatttccgttaataaaatggaggcgattggtgaagcttctgtcaagcatggcttttgccctttagtagctctttag

SEQ ID NO: 69 - protein, 340 aa, Synechocystis sp.
MFGLIGHLTSLEHAQAVAEDLGYPEYANQGLDFWCSAPPQVVDNFQVKSVTGQVIEGKYVESCFLPEMLTQRRIKAAIRKILNAMALAQKVGLDITALGGFSSIVFEEFNLKQNNQVRNVELDFQRFTTGNTHTAYVICRQVESGAKQLGIDLSQATVAVCGATGDIGSAVCRWLDSKHQVKELLLIARNRQRLENLQEELGRGKIMDLETALPQADIIVWVASMPKGVEIAGEMLKKPCLIVDGGYPKNLDTRVKADGVHILKGGIVEHSLDITWEIMKIVEMDIPSRQMFACFAEAILLEFEGWRTNFSWGRNQISVNKMEAIGEASVKHGFCPLVAL

SEQ ID NO: 70 - DNA, 1023 bp, Cyanothece sp.
atgtttggtttaattggtcatcttacaagtttagaacacgcccactccgttgctgatgcctttggctatggcccatacgccactcagggacttgatttgtggtgttctgctccaccccaattcgtcgagcattttcatgttactagcatcacaggacaaaccatcgaaggaaagtatatagaatccgctttcttaccagaaatgctgataaagcgacggattaaagcagcaattcgcaaaatactgaatgcgatggcctttgctcagaaaaataaccttaacatcacagcattagggggcttttcttcgattatttttgaagaatttaatctcaaagagaatagacaagttcgtaatgtctctttagagtttgatcgcttcaccaccggaaacacccatactgcttatatcatttgtcgtcaagttgaacaggcatccgctaaactagggattgacttatcccaagcaacggttgctatttgcggggcaaccggagatattggcagtgcagtgtgtcgttggttagatagaaaaaccgatacccaggaactattcttaattgctcgcaataaagaacgattacaacgactgcaagatgagttgggacggggtaaaattatgggattggaggaggctttacccgaagcagatattatcgtttgggtggcgagtatgcccaaaggagtggaaattaatgccgaaactctcaaaaaaccctgtttaattatcgatggtggttatcctaagaatttagacacaaaaattaaacatcctgatgtccatatcctgaaagggggaattgtagaacattctctagatattgactggaagattatggaaactgtcaatatggatgttccttctcgtcaaatgtttgcttgttttgccgaagccattttattagagtttgaacaatggcacactaatttttcttggggacgcaatcaaattacagtgactaaaatggaacaaataggagaagcttctgtcaaacatgggttacaaccgttgttgagttggtaa

SEQ ID NO: 71 - protein, 340 aa, Cyanothece sp.
MFGLIGHLTSLEHAHSVADAFGYGPYATQGLDLWCSAPPQFVEHFHVTSITGQTIEGKYIESAFLPEMLIKRRIKAAIRKILNAMAFAQKNNLNITALGGFSSIIFEEFNLKENRQVRNVSLEFDRFTTGNTHTAYIICRQVEQASAKLGIDLSQATVAICGATGDIGSAVCRWLDRKTDTQELFLIARNKERLQRLQDELGRGKIMGLEEALPEADIIVWVASMPKGVEINAETLKKPCLIIDGGYPKNLDTKIKHPDVHILKGGIVEHSLDIDWKIMETVNMDVPSRQMFACFAEAILLEFEQWHTNFSWGRNQITVTKMEQIGEASVKHGLQPLLSW

SEQ ID NO: 72 - DNA, 1041 bp, Prochlorococcus marinus
atgtttgggcttataggtcattcaactagttttgaagatgcaaaaagaaaggcttcattattgggctttgatcatattgcggatggtgatttagatgtttggtgcacagctccacctcaactagttgaaaatgtagaggttaaaagtgctataggtatatcaattgaaggttcttatattgattcatgtttcgttcctgaaatgctttcaagatttaaaacggcaagaagaaaagtattaaatgcaatggaattagctcaaaaaaaaggtattaatattaccgctttgggggggttcacttctatcatctttgaaaattttaatctccttcaacataagcagattagaaacacttcactagagtgggaaaggtttacaactggtaatactcatactgcgtgggttatttgcaggcaattagagatgaatgctcctaaaataggtattgatcttaaaagcgcaacagttgctgtagttggtgctactggagatataggcagtgctgtttgtcgatggttaatcaataaaacaggtattggggaacttcttttggtagctaggcaaaaggaacccttggattctttgcaaaaggaattagatggtggaactatcaaaaatctagatgaagcattgcctgaagcagatattgttgtatgggtagcaagtatgccaaagacaatggaaatcgatgctaataatcttaaacaaccatgtttaatgattgatggaggttatccaaagaatctagatgaaaaatttcaaggaaataatatacatgttgtaaaaggaggtatagtaagattcttcaatgatataggttggaatatgatggaactagctgaaatgcaaaatccccagagagaaatgtttgcatgctttgcagaagcaatgattttagaatttgaaaaatgtcatacaaactttagctggggaagaaataatatatctctcgagaaaatggagtttattggagctgcttctgtaaagcatggcttctctgcaattggcctagataagcatccaaaagtactagcagtttga

SEQ ID NO: 73 - protein, 346 aa, Prochlorococcus marinus
MFGLIGHSTSFEDAKRKASLLGFDHIADGDLDVWCTAPPQLVENVEVKSAIGISIEGSYIDSCFVPEMLSRFKTARRKVLNAMELAQKKGINITALGGFTSIIFENFNLLQHKQIRNTSLEWERFTTGNTHTAWVICRQLEMNAPKIGIDLKSATVAVVGATGDIGSAVCRWLINKTGIGELLLVARQKEPLDSLQKELDGGTIKNLDEALPEADIVVWVASMPKTMEIDANNLKQPCLMIDGGYPKNLDEKFQGNNIHVVKGGIVRFFNDIGWNMMELAEMQNPQREMFACFAEAMILEFEKCHTNFSWGRNNISLEKMEFIGAASVKHGFSAIGLDKHPKVLAV

SEQ ID NO: 74 - DNA, 1053 bp, Gloeobacter violaceus
atgtttggcctgatcggacacttgaccaatctttcccatgcccagcgggtcgcccgcgacctgggctacgacgagtatgcaagccacgacctcgaattctggtgcatggcccctccccaggcggtcgatgaaatcacgatcaccagcgtcaccggtcaggtgatccacggtcagtacgtcgaatcgtgctttctgccggagatgctcgcccagggccgcttcaagaccgccatgcgcaagatcctcaatgccatggccctggtccagaagcgcggcatcgacattacggccctgggaggcttctcgtcgatcatcttcgagaatttcagcctcgataaattgctcaacgtccgcgacatcaccctcgacatccagcgcttcaccaccggcaacacccacacggcctacatcctttgtcagcaggtcgagcagggtgcggtacgctacggcatcgatccggccaaagcgaccgtggcggtagtcggggccaccggcgacatcggtagcgccgtctgccgatggctcaccgaccgcgccggcatccacgaactcttgctggtggcccgcgacgccgaaaggctcgaccggctgcagcaggaactcggcaccggtcggatcctgccggtcgaagaagcacttcccaaagccgacatcgtcgtctgggtcgcctcgatgaaccagggcatggccatcgaccccgccggcctgcgcaccccctgcctgctcatcgacggcggctaccccaagaacatggccggcaccctgcagcgcccgggcatccatatcctcgacggcggcatggtcgagcactcgctcgacatcgactggcagatcatgtcgtttctaaatgtgcccaaccccgcccgccagttcttcgcctgcttcgccgagtcgatgctgctggaattcgaagggcttcacttcaatttttcctggggccgcaaccacatcaccgtcgagaagatggcccagatcggctcgctgtctaaaaaacatggctttcgtcccctgcttgaacccagtcagcgcagcggcgaactcgtacacggataa

SEQ ID NO: 75 - protein, 350 aa, Gloeobacter violaceus
MFGLIGHLTNLSHAQRVARDLGYDEYASHDLEFWCMAPPQAVDEITITSVTGQVIHGQYVESCFLPEMLAQGRFKTAMRKILNAMALVQKRGIDITALGGFSSIIFENFSLDKLLNVRDITLDIQRFTTGNTHTAYILCQQVEQGAVRYGIDPAKATVAVVGATGDIGSAVCRWLTDRAGIHELLLVARDAERLDRLQQELGTGRILPVEEALPKADIVVWVASMNQGMAIDPAGLRTPCLLIDGGYPKNMAGTLQRPGIHILDGGMVEHSLDIDWQIMSFLNVPNPARQFFACFAESMLLEFEGLHFNFSWGRNHITVEKMAQIGSLSKKHGFRPLLEPSQRSGELVHG

SEQ ID NO: 76 - DNA, 1020 bp, Nostoc punctiforme
atgtttggtctaattggacatctgactagtttagaacacgctcaagccgtagcccaagaattgggatacccagaatatgccgatcaagggctagacttttggtgcagcgccccgccgcaaattgtcgatagtattattgtcaccagtgttactgggcaacaaattgaaggacgatatgtagaatcttgctttttgccggaaatgctagctagtcgccgcatcaaagccgcaacacggaaaatcctcaacgctatggcccatgcacagaagcacggcattaacatcacagctttaggcggattttcctcgattatttttgaaaactttaagttagagcagtttagccaagtccgaaatatcaagctagagtttgaacgcttcaccacaggaaacacgcatactgcctacattatttgtaagcaggtggaagaagcatccaaacaactgggaattaatctatcaaacgcgactgttgcggtatgtggagcaactggggatattggtagtgccgttacacgctggctagatgcgagaacagatgtccaagaactcctgctaatcgcccgcgatcaagaacgtctcaaagagttgcaaggcgaactggggcgggggaaaatcatgggtttgacagaagcactaccccaagccgatgttgtagtttgggttgctagtatgcccagaggcgtggaaattgaccccaccactttgaaacaaccctgtttgttgattgatggtggctatcctaaaaacttagcaacaaaaattcaatatcctggcgtacacgtgttaaatggtgggattgtagagcattccctggatattgactggaaaattatgaaaatagtcaatatggacgtgccagcccgtcagttgtttgcctgttttgccgaatcaatgctactggaatttgagaagttatacacgaacttttcgtggggacggaatcagattaccgtagataaaatggagcagattggccgggtgtcagtaaaacatggatttagaccgttgttggtttag

SEQ ID NO: 77 - protein, 339 aa, Nostoc punctiforme
MFGLIGHLTSLEHAQAVAQELGYPEYADQGLDFWCSAPPQIVDSIIVTSVTGQQIEGRYVESCFLPEMLASRRIKAATRKILNAMAHAQKHGINITALGGFSSIIFENFKLEQFSQVRNIKLEFERFTTGNTHTAYIICKQVEEASKQLGINLSNATVAVCGATGDIGSAVTRWLDARTDVQELLLIARDQERLKELQGELGRGKIMGLTEALPQADVVVWVASMPRGVEIDPTTLKQPCLLIDGGYPKNLATKIQYPGVHVLNGGIVEHSLDIDWKIMKIVNMDVPARQLFACFAESMLLEFEKLYTNFSWGRNQITVDKMEQIGRVSVKHGFRPLLV

SEQ ID NO: 78 - DNA, 1020 bp, Anabaena variabilis
atgtttggtctaattggacatctgacaagtttagaacacgctcaagcggtagctcaagaactgggatacccagaatacgccgaccaagggctagatttttggtgcagcgctccaccgcaaatagttgaccacattaaagttactagcattactggtgaaataattgaagggaggtatgtagaatcttgctttttaccagaaatgctagccagccgtaggattaaagccgcaacccgcaaagtcctcaatgctatggctcatgctcaaaaacatggcattgacatcaccgctttgggtggtttctcctccattatttttgaaaacttcaaattggaacagtttagccaagttcgtaatgtcacactagagtttgaacgcttcactacaggcaacactcacacagcttatatcatttgtcggcaggtagaacaagcatcacaacaactcggcattgaactctcccaagcaacagtagctatatgtggggctactggtgacattggtagtgcagttactcgctggctggatgccaaaacagacgtaaaagaattactgttaatcgcccgtaatcaagaacgtctccaagagttgcaaagcgagttgggacgcggtaaaatcatgagcctagatgaagcattgcctcaagctgatattgtagtttgggtagctagtatgcctaaaggcgtggaaattaatcctcaagttttgaaacaaccctgtttattgattgatggtggttatccgaaaaacttgggtacaaaagttcagtatcctggtgtttatgtactgaacggaggtatcgtcgaacattccctagatattgactggaaaatcatgaaaatagtcaatatggatgtacctgcacgccaattatttgcttgttttgcggaatctatgctcttggaatttgagaagttgtacacgaacttttcttgggggcgcaatcagattaccgtagacaaaatggagcagattggtcaagcatcagtgaaacatgggtttagaccactgctggtttag

SEQ ID NO: 79 - protein, 339 aa, Anabaena variabilis
MFGLIGHLTSLEHAQAVAQELGYPEYADQGLDFWCSAPPQIVDHIKVTSITGEIIEGRYVESCFLPEMLASRRIKAATRKVLNAMAHAQKHGIDITALGGFSSIIFENFKLEQFSQVRNVTLEFERFTTGNTHTAYIICRQVEQASQQLGIELSQATVAICGATGDIGSAVTRWLDAKTDVKELLLIARNQERLQELQSELGRGKIMSLDEALPQADIVVWVASMPKGVEINPQVLKQPCLLIDGGYPKNLGTKVQYPGVYVLNGGIVEHSLDIDWKIMKIVNMDVPARQLFACFAESMLLEFEKLYTNFSWGRNQITVDKMEQIGQASVKHGFRPLLV

SEQ ID NO: 80 - DNA, 1026 bp, Synechococcus elongatus; identical to SEQ ID NO: 66

SEQ ID NO: 81 - protein, 341 aa, Synechococcus elongatus; identical to SEQ ID NO: 67

SEQ ID NO: 82 - DNA, 1020 bp, Nostoc sp.
atgtttggtctaattggacatctgacaagtttagaacacgctcaagcggtagctcaagaactgggatacccagaatacgccgaccaagggctagatttttggtgtagcgctccaccgcaaatagttgaccacattaaagttactagtattactggtgaaataattgaagggaggtatgtagaatcttgctttttaccggagatgctagccagtcgtcggattaaagccgcaacccgcaaagtcctcaatgctatggctcatgctcaaaagaatggcattgatatcacagctttgggtggtttctcctccattatttttgaaaactttaaattggagcagtttagccaagttcgtaatgtgacactagagtttgaacgcttcactacaggcaacactcacacagcatatattatttgtcggcaggtagaacaagcatcacaacaactcggcattgaactctcccaagcaacagtagctatatgtggggctactggtgatattggtagtgcagttactcgctggctggatgctaaaacagacgtgaaagaattgctgttaatcgcccgtaatcaagaacgtctccaagagttgcaaagcgagctgggacgcggtaaaatcatgagccttgatgaagcactgccccaagctgatatcgtagtttgggtagccagtatgcctaaaggtgtggaaattaatcctcaagttttgaagcaaccctgtttgctgattgatgggggttatccgaaaaacttgggtacaaaagttcagtatcctggtgtttatgtactgaacggcggtatcgtcgaacattcgctggatattgactggaaaatcatgaaaatagtcaatatggatgtacctgcacgccaattatttgcttgttttgcggaatctatgctcttggaatttgagaagttgtacacgaacttttcttgggggcgcaatcagattaccgtagacaaaatggagcagattggtcaagcatcagtgaaacatgggtttagaccactgctggtttag

SEQ ID NO: 83 - protein, 339 aa, Nostoc sp.
MFGLIGHLTSLEHAQAVAQELGYPEYADQGLDFWCSAPPQIVDHIKVTSITGEIIEGRYVESCFLPEMLASRRIKAATRKVLNAMAHAQKNGIDITALGGFSSIIFENFKLEQFSQVRNVTLEFERFTTGNTHTAYIICRQVEQASQQLGIELSQATVAICGATGDIGSAVTRWLDAKTDVKELLLIARNQERLQELQSELGRGKIMSLDEALPQADIVVWVASMPKGVEINPQVLKQPCLLIDGGYPKNLGTKVQYPGVYVLNGGIVEHSLDIDWKIMKIVNMDVPARQLFACFAESMLLEFEKLYTNFSWGRNQITVDKMEQIGQASVKHGFRPLLV

SEQ ID NO: 84 - DNA, 1026 bp, Synechococcus elongatus
atgtttggtctgattggtcacctgaccagcttggaacaagcgcgtgacgtcagccgccgtatgggttatgatgaatacgctgatcaaggcctggagttttggagcagcgcgccaccgcagatcgtcgatgagatcaccgtgacctccgcaaccggtaaggtcatccacggccgctacattgagtcctgcttcctgcctgagatgctggcagctcgccgtttcaaaacggccactcgtaaggttctgaatgcgatgtcccatgcgcaaaagcatggcattgacattagcgccttgggcggttttacgtcgattatcttcgagaacttcgatctggcctctttgcgccaggtgcgtgacacgaccttggagtttgagcgttttaccacgggtaatacgcacaccgcttacgttatctgtcgccaagtcgaagcagcagccaaaaccctgggtattgatatcacccaggccaccgtcgccgtggtgggtgctaccggtgatattggttccgcggtttgccgttggctggatctgaaactgggtgttggcgatctgatcctgacggcgcgtaatcaggagcgtctggacaacctgcaagccgagttgggtcgcggtaagatcctgccgttggaggcagcgttgccggaggcagacttcatcgtctgggttgcgtctatgccgcagggtgttgttatcgacccggcgaccttgaaacagccgtgcgtgctgattgatggcggctatccgaaaaacctgggcagcaaggtccaaggcgagggtatctatgtcctgaatggcggtgtggttgagcattgcttcgacattgactggcagatcatgagcgcagcagaaatggcgcgtccggagcgccaaatgtttgcctgttttgcagaagccatgctgctggagttcgaaggctggcatacgaatttcagctggggtcgtaatcagattaccattgaaaagatggaagcgattggtgaagcaagcgtgcgtcatggttttcagccactggcgctggctatttaa

SEQ ID NO: 85 - DNA, 1041 bp, Prochlorococcus marinus
atgtttggtctgattggccacagcacgagctttgaggacgcaaagcgtaaggcgagcctgctgggctttgatcatattgctgatggcgacctggacgtctggtgcacggcacctccgcaactggttgagaatgtcgaggtgaaatcggcgattggcatttccatcgaaggctcctacatcgacagctgtttcgtgccggagatgttgagccgtttcaaaaccgcacgtcgcaaagttctgaatgcaatggagctggcacaaaagaagggcatcaacatcacggcgctgggtggtttcaccagcattatctttgagaacttcaatctgttgcagcataaacagatccgtaataccagcctggagtgggaacgctttaccacgggtaacacccacaccgcgtgggtgatctgccgccagctggagatgaatgcgccgaaaatcggtattgacctgaaaagcgcgacggtggcagttgttggcgcaactggcgacattggttcggccgtttgtcgctggctgattaacaagaccggtatcggtgaattgttgctggtcgctcgccagaaggagcctctggacagcctgcaaaaagagctggacggtggtacgatcaagaacctggatgaagcgctgccagaagcggacatcgtcgtctgggtcgcatctatgccgaaaactatggaaatcgatgccaacaatctgaaacaaccgtgcctgatgatcgatggcggctacccgaagaacttggatgagaagtttcaaggcaataacatccacgttgtgaagggtggtattgtccgtttcttcaatgatatcggttggaacatgatggaactggctgaaatgcagaacccgcaacgtgagatgttcgcttgttttgcggaggccatgattctggagttcgagaaatgccataccaatttcagctggggtcgcaacaacattagcctggagaaaatggagttcatcggcgctgcgagcgttaagcacggcttcagcgcgattggtttggataaacatccgaaggtcctggcagtttaa

SEQ ID NO: 86 - DNA, 3522 bp, Mycobacterium smegmatis strain
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctga

SEQ ID NO: 87 - protein, 1173 aa, Mycobacterium smegmatis strain
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 88 - DNA, 921 bp, Nostoc punctiforme
atgactcaagcgaaagccaaaaaagaccacggtgacgttcctgttaacacttaccgtcccaatgctccatttattggcaaggtaatatctaatgaaccattagtcaaagaaggtggtattggtattgttcaacaccttaaatttgacctatctggtggggatttgaagtatatagaaggtcaaagtattggcattattccgccaggtttagacaagaacggcaagcctgaaaaactcagactatattccatcgcctcaactcgtcatggtgatgatgtagatgataagacagtatcactgtgcgtccgccagttggagtacaagcacccagaaactggcgaaacagtctacggtgtttgctctacgcacctgtgtttcctcaagccaggggaagaggtaaaaattacagggcctgtgggtaaggaaatgttgttacccaatgaccctgatgctaatgttatcatgatggctactggaacaggtattgcgccgatgcgggcttacttgtggcgtcagtttaaagatgcggaaagagcggctaacccagaataccaatttaaaggattctcttggctaatatttggcgtacctacaactccaaaccttttatataaggaagaactggaagagattcaacaaaaatatcctgagaacttccgcctaactgctgccatcagccgcgaacagaaaaatccccaaggcggtagaatgtatattcaagaccgcgtagcagaacatgctgatgaattgtggcagttgattaaaaatgaaaaaacccacacttacatttgcggtttgcgcggtatggaagaaggtattgatgcagccttaactgctgctgctgctaaggaaggcgtaacctggagtgattaccagaagcaactcaagaaagccggtcgctggcacgtagaaacttactaa

SEQ ID NO: 89 - protein, 437 aa, Nostoc punctiforme
MYNQGAVEGAANIELGSRIFVYEVVGLRQGEETDQTNYPIRKSGSVFIRVPYNRMNQEMRRITRLGGTIVSIQPITALEPVNGKASFGNATSVVSELAKSGETANSEGNGKATPVNAHSAEEQNKDKKGNTMTQAKAKKDHGDVPVNTYRPNAPFIGKVISNEPLVKEGGIGIVQHLKFDLSGGDLKYIEGQSIGIIPPGLDKNGKPEKLRLYSIASTRHGDDVDDKTVSLCVRQLEYKHPETGETVYGVCSTHLCFLKPGEEVKITGPVGKEMLLPNDPDANVIMMATGTGIAPMRAYLWRQFKDAERAANPEYQFKGFSWLIFGVPTTPNLLYKEELEEIQQKYPENFRLTAAISREQKNPQGGRMYIQDRVAEHADELWQLIKNEKTHTYICGLRGMEEGIDAALTAAAAKEGVTWSDYQKQLKKAGRWHVETY

SEQ ID NO: 90 - DNA, 300 bp, Nostoc punctiforme
atgccaacttataaagtgacactaattaacgaggctgaagggctgaacacaacccttgatgttgaggacgatacctatattctagacgcagctgaagaagctggtattgacctgccctactcttgccgcgctggtgcttgctctacttgtgcaggtaaactcgtatcaggtaccgtcgatcaaggcgatcaatcattcttagatgacgatcaaatagaagctggatatgtactgacctgtgttgcttacccaacttctaatgtcacgatcgaaactcacaaagaagaagaactctattaa

SEQ ID NO: 91 - protein, 99 aa, Nostoc punctiforme
MPTYKVTLINEAEGLNTTLDVEDDTYILDAAEEAGIDLPYSCRAGACSTCAGKLVSGTVDQGDQSFLDDDQIEAGYVLTCVAYPTSNVTIETHKEEELY

SEQ ID NO: 92 - DNA, 369 bp, Nostoc punctiforme
atgtcccgtacatacacaattaaagttcgcgatcgcgccactggcaaaacacacaccctaaaagtgccagaagaccgttatatcctgcacactgccgaaaaacaaggtgtggaactaccgttttcctgtcgcaacggagcttgcaccgcttgtgctgtgagggtattgtcaggagaaatttatcaaccagaggcgatcggattgtcaccagatttacgtcagcaaggttatgccctgttgtgtgtgagttatccccgttctgacttggaagtagagacacaagacgaagatgaagtctacgaactccagtttgggcgctattttgctaaggggaaagttaaagcgggtttaccgttagatgaggaataa

SEQ ID NO: 93 - protein, 122 aa, Nostoc punctiforme
MSRTYTIKVRDRATGKTHTLKVPEDRYILHTAEKQGVELPFSCRNGACTACAVRVLSGEIYQPEAIGLSPDLRQQGYALLCVSYPRSDLEVETQDEDEVYELQFGRYFAKGKVKAGLPLDEE

SEQ ID NO: 94 - DNA, 321 bp, Nostoc punctiforme
atgcccaaaacttacaccgtagaaatcgatcatcaaggcaaaattcataccttgcaagttcctgaaaatgaaacgatcttatcagttgccgatgctgctggtttggaactgccgagttcttgtaatgcaggtgtttgcacaacttgcgccggtcaaataagccagggaactgtggatcaaactgatggcatgggcgttagtccagatttacaaaagcaaggttacgtattgctttgtgttgcgaaacccctttctgatttgaaacttgaaacagaaaaggaagacatagtttatcagttacaatttggcaaagacaaataa

SEQ ID NO: 95 - protein, 106 aa, Nostoc punctiforme
MPKTYTVEIDHQGKIHTLQVPENETILSVADAAGLELPSSCNAGVCTTCAGQISQGTVDQTDGMGVSPDLQKQGYVLLCVAKPLSDLKLETEKEDIVYQLQFGKDK

SEQ ID NO: 96 - protein, 117 aa, Artificial Sequence
  Synthetic Peptide
EXXXXXXXXXXXXXXXXXXXXXXXXXXXEXXHXXXXXXXXXXXXXXXXXXXXXXXXXXXXXXXXXXXXXXXXXXXXXXXXXXXEXXXXXXXXXXXXXXXXXXXXXXXXXXXXXEXXH

SEQ ID NO: 97 - DNA, 30 bp, Artificial sequence
  sll0208/9-KO1 primer
cgcggatcccttgattctactgcggcgagt

SEQ ID NO: 98 - DNA, 53 bp, Artificial sequence
  sll0208/9-KO2 primer
cacgcacctaggttcacactcccatggtataacaggggcgttggactcctgtg

SEQ ID NO: 99 - DNA, 52 bp, Artificial sequence
  sll0208/9-KO3 primer
gttataccatgggagtgtgaacctaggtgcgtggccgacaggataggcgtgt

SEQ ID NO: 100 - DNA, 30 bp, Artificial sequence
  sll0208/9-KO4 primer
cgcggatccaacgcatcctcactagtcggg

SEQ ID NO: 101 - DNA, 38 bp, Artificial sequence
  Kan-aph-F primer
catgccatggaaagccacgttgtgtctcaaaatctctg

SEQ ID NO: 102 - DNA, 32 bp, Artificial sequence
  Kan-aph-R primer
ctagtctagagcgctgaggtctgcctcgtaaa

SEQ ID NO: 103 - DNA, 45 bp, Artificial sequence
  Primer
gttttgcgatcgcagcatttaacatttacatccccgttgccgacg

SEQ ID NO: 104 - DNA, 45 bp, Artificial sequence
  Primer
gttttgcgatcgcagcatataacattttcatccccgttgccgacg

SEQ ID NO: 105 - protein, 116 aa, Artificial Sequence
  Synthetic Peptide
EXXAXXXYXXXXXXXXXXXXXXXXXXXMEXXHXXXFXXXXXXXXXXXXXXXXXXXXXXXXXXXXXXXXXXXXXXXXXXQXXXXEXXAXXXYXXXXXXXXXXXXXXXXXVXXDEXXH

SEQ ID NO: 106 - protein, 116 aa, Artificial Sequence
  Synthetic Peptide
EXXAXXXFXXXXXXXXXXXXXXXXXXXMEXXHXXXFXXXXXXXXXXXXXXXXXXXXXXXXXXXXXXXXXXXXXXXXXXQXXXXEXXAXXXYXXXXXXXXXXXXXXXXXVXXDEXXH

SEQ ID NO: 107 - protein, 116 aa, Artificial Sequence
  Synthetic Peptide
EXXAXXXFXXXXXXXXXXXXXXXXXXXMEXXHXXXFXXXXXXXXXXXXXXXXXXXXXXXXXXXXXXXXXXXXXXXXXXQXXXXEXXAXXXYXXXXXXXXXXXXXXXXXVXXEEXXH

SEQ ID NO: 108 - protein, 116 aa, Artificial Sequence
  Synthetic Peptide
EXXAXXXYXXXXXXXXXXXXXXXXXXXMEXXHXXXFXXXXXXXXXXXXXXXXXXXXXXXXXXXXXXXXXXXXXXXXXXQXXXXEXXAXXXYXXXXXXXXXXXXXXXXXVXXEEXXH

SEQ ID NO: 109 - DNA, 25 bp, Artificial Sequence
  Synthetic Primer
atgtctgacaaggggtttgacccct

SEQ ID NO: 110 - DNA, 25 bp, Artificial Sequence
  Synthetic Primer
gcacatccttatccaattgctctag

SEQ ID NO: 111 - DNA, 25 bp, Artificial Sequence
  Synthetic Primer
gatcaaacaggtgcagcagcaactt

SEQ ID NO: 112 - DNA, 25 bp, Artificial Sequence
  Synthetic Primer
attcttgacaagcgatcgcggtcac

SEQ ID NO: 113 - DNA, 25 bp, Artificial Sequence
  Synthetic Primer
attcaatacacccccctagccgatc

SEQ ID NO: 114 - DNA, 25 bp, Artificial Sequence
  Synthetic Primer
taagggtggtgggaaaaatgggcca

SEQ ID NO: 115 - protein, 116 aa, Artificial Sequence
  Synthetic Peptide
EXXXXXXXXXXXXXXXXXXXXXXXXXXXEXXHXXXXXXCXXXXXXXXXXXXXXXXXXXXXXXXXXXXXXXXXXXXXXXXXXXXEXXXXXXXXXXXXXXXXXXXXXXXXXXXXEXXH

SEQ ID NO: 116 - DNA, 20 bp, Artificial Sequence
  Synthetic Primer
gcactcgaccggaattatcg

SEQ ID NO: 117 - DNA, 20 bp, Artificial Sequence
  Synthetic Primer
tggtcgacggcgctattcag

SEQ ID NO: 118 - DNA, 20 bp, Artificial Sequence
  Synthetic Primer
cgataattccggtcgagtgc

SEQ ID NO: 119 - DNA, 20 bp, Artificial Sequence
  Synthetic Primer
ctgaatagcgccgtcgacca

SEQ ID NO: 120 - DNA, 51 bp, Artificial Sequence
  Synthetic Primer
cgatggtatcgtgatcgagggtnnscaagaggcgcatgagaactacattcg

SEQ ID NO: 121 - DNA, 22 bp, Artificial Sequence
  Synthetic Primer
accctcgatcacgataccatcg

SEQ ID NO: 122 - DNA, 49 bp, Artificial Sequence
  Synthetic Primer
gtgatcgagggtgagcaagagnnscatgagaactacattcgtctgggtg

SEQ ID NO: 123 - DNA, 21 bp, Artificial Sequence
  Synthetic Primer
ctcttgctcaccctcgatcac

SEQ ID NO: 124 - DNA, 47 bp, Artificial Sequence
  Synthetic Primer
gagcaagaggcgcatgagaacnnkattcgtctgggtgaaatgttgcc

SEQ ID NO: 125 - DNA, 21 bp, Artificial Sequence
  Synthetic Primer
gttctcatgcgcctcttgctc

SEQ ID NO: 126 - DNA, 46 bp, Artificial Sequence
  Synthetic Primer
gacgactttatccgtttgagcaagnnsgaggcccgtcacaagaagg

SEQ ID NO: 127 - DNA, 24 bp, Artificial Sequence
  Synthetic Primer
cttgctcaaacggataaagtcgtc

SEQ ID NO: 128 - DNA, 52 bp, Artificial Sequence
  Synthetic Primer
gacgactttatccgtttgagcaagatgnnsgcccgtcacaagaagggctttg

SEQ ID NO: 129 - DNA, 44 bp, Artificial Sequence
  Synthetic Primer
gagcaagatggaggcccgtnnkaagaagggctttgaggcttgtg

SEQ ID NO: 130 - DNA, 19 bp, Artificial Sequence
  Synthetic Primer
acgggcctccatcttgctc

SEQ ID NO: 131 - DNA, 45 bp, Artificial Sequence
  Synthetic Primer
gcccgtcacaagaagggcnnsgaggcttgtggtcgtaacttgaag

SEQ ID NO: 132 - DNA, 18 bp, Artificial Sequence
  Synthetic Primer
gcccttcttgtgacgggc

SEQ ID NO: 133 - DNA, 53 bp, Artificial Sequence
  Synthetic Primer
gataaagttccgacctgcttggttattnnstccctgatcatcgaatgcttcgc

SEQ ID NO: 134 - DNA, 27 bp, Artificial Sequence
  Synthetic Primer
aataaccaagcaggtcggaactttatc

SEQ ID NO: 135 - DNA, 47 bp, Artificial Sequence
  Synthetic Primer
gcttggttattcagtccctgatcatcnnstgcttcgcgattgcagcg

SEQ ID NO: 136 - DNA, 26 bp, Artificial Sequence
  Synthetic Primer
gatgatcagggactgaataaccaagc

SEQ ID NO: 137 - DNA, 54 bp, Artificial Sequence
  Synthetic Primer
cagtccctgatcatcgaatgcttcnnsattgcagcgtataacatttacatcccg

SEQ ID NO: 138 - DNA, 24 bp, Artificial Sequence
  Synthetic Primer
gaagcattcgatgatcagggactg

SEQ ID NO: 139 - DNA, 43 bp, Artificial Sequence
  Synthetic Primer
tgcttcgcgattgcagcgnnsaacatttacatcccggttgccg

SEQ ID NO: 140 - DNA, 18 bp, Artificial Sequence
  Synthetic Primer
cgctgcaatcgcgaagca

SEQ ID NO: 141 - DNA, 49 bp, Artificial Sequence
  Synthetic Primer
cgctcgtaagattaccgagagcnnkgtcaaggacgaataccagcatctg

SEQ ID NO: 142 - DNA, 22 bp, Artificial Sequence
  Synthetic Primer
gctctcggtaatcttacgagcg

SEQ ID NO: 143 - DNA, 54 bp, Artificial Sequence
  Synthetic Primer
gtaagattaccgagagcgtcgtcaagnnkgaataccagcatctgaactatggcg

SEQ ID NO: 144 - DNA, 26 bp, Artificial Sequence
  Synthetic Primer
cttgacgacgctctcggtaatcttac

SEQ ID NO: 145 - DNA, 54 bp, Artificial Sequence
  Synthetic Primer
gtaagattaccgagagcgtcgtcaaggacnnstaccagcatctgaactatggcg

SEQ ID NO: 146 - DNA, 46 bp, Artificial Sequence
  Synthetic Primer
cgtcgtcaaggacgaataccagnnsctgaactatggcgaggagtgg

SEQ ID NO: 147 - DNA, 22 bp, Artificial Sequence
  Synthetic Primer
ctggtattcgtccttgacgacg

SEQ ID NO: 148 - DNA, 48 bp, Artificial Sequence
  Synthetic Primer
cacaagaagggctttgaggctnnsggtcgtaacttgaaggtgacttgc

SEQ ID NO: 149 - DNA, 21 bp, Artificial Sequence
  Synthetic Primer
agcctcaaagcccttcttgtg

SEQ ID NO: 150 - DNA, 20 bp, Artificial Sequence
  Synthetic Primer
catatgccgcaaacgcaagc

SEQ ID NO: 151 - protein, 116 aa, Artificial Sequence; identical to SEQ ID NO: 106
  Synthetic Peptide

SEQ ID NO: 152 - protein, 116 aa, Artificial Sequence
  Synthetic Peptide
EXXAXXXYXXXXXXXXXXXXXXXXXXXIEXXHXXXFXXXXXXXXXXXXXXXXXXXXXXXXXXXXXXXXXXXXXXXXXXQXXXXEXXAXXXYXXXXXXXXXXXXXXXXXVXXDEXXH

SEQ ID NO: 153 - protein, 116 aa, Artificial Sequence
  Synthetic Peptide
EXXAXXXYXXXXXXXXXXXXXXXXXXXLEXXHXXXFXXXXXXXXXXXXXXXXXXXXXXXXXXXXXXXXXXXXXXXXXXQXXXXEXXAXXXYXXXXXXXXXXXXXXXXXVXXDEXXH

SEQ ID NO: 154 - protein, 116 aa, Artificial Sequence
  Synthetic Peptide
EXXAXXXYXXXXXXXXXXXXXXXXXXXMEXXHXXXFXXXXXXXXXXXXXXXXXXXXXXXXXXXXXXXXXXXXXXXXXXQXXXXEXXAXXXFXXXXXXXXXXXXXXXXXVXXDEXXH

(19) United States
(12) Patent Application Publication
(10) Pub. No.: US 2011/0124071 A1
(43) Pub. Date: May 26, 2011

(54) METHODS AND COMPOSITIONS FOR PRODUCING HYDROCARBONS

(75) Inventors: Andreas Schirmer, Fremont, CA (US); Mathew Rude, San Francisco, CA (US); Noah Helman, El Cerrito, CA (US)

(73) Assignee: LS9, Inc, So. San Francisco, CA (US)

(21) Appl. No.: 12/948,349

(22) Filed: Nov. 17, 2010

Related U.S. Application Data
(60) Provisional application No. 61/261,755, filed on Nov. 17, 2009.

(51) Int. Cl. C12P 5/02; C12N 9/88; C12N 1/00; C12P 5/00

(52) U.S. Cl. 435/167; 435/232; 435/243; 435/166

(57) ABSTRACT

Provided are compositions and methods for producing hydrocarbons, including aldehydes, alkanes and alkenes. The hydrocarbons can be used in biofuels.

### CROSS-REFERENCE TO RELATED APPLICATIONS

This application claims the benefit of U.S. Provisional Application No. 61/261,755, filed Nov. 17, 2009, the contents of which are hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 180 KByte ASCII (Text) file named “Sequence.TXT,” created on Nov. 17, 2010.

### BACKGROUND OF THE INVENTION

Petroleum is a limited, natural resource found in the Earth in liquid, gaseous, or solid forms. Petroleum is primarily composed of hydrocarbons, which are comprised mainly of carbon and hydrogen. It also contains significant amounts of other elements, such as, nitrogen, oxygen, or sulfur, in different forms.

Petroleum is a valuable resource, but petroleum products are developed at considerable costs, both financial and environmental. First, sources of petroleum must be discovered. Petroleum exploration is an expensive and risky venture. The cost of exploring deep water wells can exceed $100 million. Moreover, there is no guarantee that these wells will contain petroleum. It is estimated that only 40% of drilled wells lead to productive wells generating commercial hydrocarbons. In addition to the economic cost, petroleum exploration carries a high environmental cost. For example, offshore exploration disturbs the surrounding marine environments.

After a productive well is discovered, the petroleum must be extracted from the Earth at great expense. During primary recovery, the natural pressure underground is sufficient to extract about 20% of the petroleum in the well. As this natural pressure falls, secondary recovery methods are employed, if economical. Generally, secondary recovery involves increasing the well's pressure by, for example, water injection, natural gas injection, or gas lift. Using secondary recovery methods, an additional 5% to 15% of petroleum is recovered. Once secondary recovery methods are exhausted, tertiary recovery methods can be used, if economical. Tertiary methods involve reducing the viscosity of the petroleum to make it easier to extract. Using tertiary recovery methods, an additional 5% to 15% of petroleum is recovered. Hence, even under the best circumstances, only 50% of the petroleum in a well can be extracted. Petroleum extraction also carries an environmental cost. For example, petroleum extraction can result in large seepages of petroleum rising to the surface. Moreover, offshore drilling involves dredging the seabed which disrupts or destroys the surrounding marine environment.

Since petroleum deposits are not found uniformly throughout the Earth, petroleum must be transported over great distances from petroleum producing regions to petroleum consuming regions. In addition to the shipping costs, there is also the environmental risk of devastating oil spills.

In its natural form, crude petroleum extracted from the Earth has few commercial uses. It is a mixture of hydrocarbons (e.g., paraffins (or alkanes), olefins (or alkenes), alkynes, napthenes (or cylcoalkanes), aliphatic compounds, aromatic compounds, etc.) of varying length and complexity. In addition, crude petroleum contains other organic compounds (e.g., organic compounds containing nitrogen, oxygen, sulfur, etc.) and impurities (e.g., sulfur, salt, acid, metals, etc.).

Hence, crude petroleum must be refined and purified before it can be used commercially. Due to its high energy density and its easy transportability, most petroleum is refined into fuels, such as transportation fuels (e.g., gasoline, diesel, aviation fuel, etc.), heating oil, liquefied petroleum gas, etc.

Crude petroleum is also a primary source of raw materials for producing petrochemicals. The two main classes of raw materials derived from petroleum are short chain olefins (e.g., ethylene and propylene) and aromatics (e.g., benzene and xylene isomers). These raw materials are derived from longer chain hydrocarbons in crude petroleum by cracking it at considerable expense using a variety of methods, such as catalytic cracking, steam cracking, or catalytic reforming. These raw materials are used to make petrochemicals, which cannot be directly refined from crude petroleum, such as monomers, solvents, detergents, or adhesives.

One example of a raw material derived from crude petroleum is ethylene. Ethylene is used to produce petrochemicals such as, polyethylene, ethanol, ethylene oxide, ethylene glycol, polyester, glycol ether, ethoxylate, vinyl acetate, 1,2-dichloroethane, trichloroethylene, tetrachloroethylene, vinyl chloride, and polyvinyl chloride. An additional example of a raw material is propylene, which is used to produce isopropyl alcohol, acrylonitrile, polypropylene, propylene oxide, propylene glycol, glycol ethers, butylene, isobutylene, 1,3-butadiene, synthetic elastomers, polyolefins, alpha-olefins, fatty alcohols, acrylic acid, acrylic polymers, allyl chloride, epichlorohydrin, and epoxy resins.

These petrochemicals can then be used to make specialty chemicals, such as plastics, resins, fibers, elastomers, pharmaceuticals, lubricants, or gels. Particular specialty chemicals which can be produced from petrochemical raw materials are: fatty acids, hydrocarbons (e.g., long chain, branched chain, saturated, unsaturated, etc.), fatty alcohols, esters, fatty aldehydes, ketones, lubricants, etc.

Specialty chemicals have many commercial uses. Fatty acids are used commercially as surfactants, for example, in detergents and soaps. They can also be used as additives in fuels, lubricating oils, paints, lacquers, candles, salad oil, shortening, cosmetics, and emulsifiers. In addition, fatty acids are used as accelerator activators in rubber products. Fatty acids can also be used as a feedstock to produce methyl esters, amides, amines, acid chlorides, anhydrides, ketene dimers, and peroxy acids and esters.

Hydrocarbons have many commercial uses. For example, shorter chain alkanes are used as fuels. Methane and ethane are the main constituents of natural gas. Longer chain alkanes (e.g., from five to sixteen carbons) are used as transportation fuels (e.g., gasoline, diesel, or aviation fuel). Alkanes having more than sixteen carbon atoms are important components of fuel oils and lubricating oils. Even longer alkanes, which are solid at room temperature, can be used, for example, as a paraffin wax. Alkanes that contain approximately thirty-five carbons are found in bitumen, which is used for road surfacing. In addition, longer chain alkanes can be cracked to produce commercially useful shorter chain hydrocarbons.

Like short chain alkanes, short chain alkenes are used in transportation fuels. Longer chain alkenes are used in plastics, lubricants, and synthetic lubricants. In addition, alkenes are used as a feedstock to produce alcohols, esters, plasticizers, surfactants, tertiary amines, enhanced oil recovery agents, fatty acids, thiols, alkenylsuccinic anhydrides, epoxides, chlorinated alkanes, chlorinated alkenes, waxes, fuel additives, and drag flow reducers.

Fatty alcohols have many commercial uses. The shorter chain fatty alcohols are used in the cosmetic and food industries as emulsifiers, emollients, and thickeners. Due to their amphiphilic nature, fatty alcohols behave as nonionic surfactants, which are useful as detergents. In addition, fatty alcohols are used in waxes, gums, resins, pharmaceutical salves and lotions, lubricating oil additives, textile antistatic and finishing agents, plasticizers, cosmetics, industrial solvents, and solvents for fats.

Esters have many commercial uses. For example, biodiesel, an alternative fuel, is comprised of esters (e.g., fatty acid methyl ester, fatty acid ethyl esters, etc.). Some low molecular weight esters are volatile with a pleasant odor which makes them useful as fragrances or flavoring agents. In addition, esters are used as solvents for lacquers, paints, and varnishes. Furthermore, some naturally occurring substances, such as waxes, fats, and oils are comprised of esters. Esters are also used as softening agents in resins and plastics, plasticizers, flame retardants, and additives in gasoline and oil. In addition, esters can be used in the manufacture of polymers, films, textiles, dyes, and pharmaceuticals.

Aldehydes are used to produce many specialty chemicals. For example, aldehydes are used to produce polymers, resins (e.g., Bakelite), dyes, flavorings, plasticizers, perfumes, pharmaceuticals, and other chemicals. Some are used as solvents, preservatives, or disinfectants. Some natural and synthetic compounds, such as vitamins and hormones, are aldehydes. In addition, many sugars contain aldehyde groups.

Ketones are used commercially as solvents. For example, acetone is frequently used as a solvent, but it is also a raw material for making polymers. Ketones are also used in lacquers, paints, explosives, perfumes, and textile processing. In addition, ketones are used to produce alcohols, alkenes, alkanes, imines, and enamines.

In addition, crude petroleum is a source of lubricants. Lubricants derived petroleum are typically composed of olefins, particularly polyolefins and alpha-olefins. Lubricants can either be refined from crude petroleum or manufactured using raw materials refined from crude petroleum.

Obtaining these specialty chemicals from crude petroleum requires a significant financial investment as well as a great deal of energy. It is also an inefficient process because frequently the long chain hydrocarbons in crude petroleum are cracked to produce smaller monomers. These monomers are then used as the raw material to manufacture the more complex specialty chemicals.

In addition to the problems with exploring, extracting, transporting, and refining petroleum, petroleum is a limited and dwindling resource. One estimate of world petroleum consumption is 30 billion barrels per year. By some estimates, it is predicted that at current production levels, the world's petroleum reserves could be depleted before the year 2050.

Finally, the burning of petroleum based fuels releases greenhouse gases (e.g., carbon dioxide) and other forms of air pollution (e.g., carbon monoxide, sulfur dioxide, etc.). As the world's demand for fuel increases, the emission of greenhouse gases and other forms of air pollution also increases. The accumulation of greenhouse gases in the atmosphere leads to an increase global warming. Hence, in addition to damaging the environment locally (e.g., oil spills, dredging of marine environments, etc.), burning petroleum also damages the environment globally.

Due to the inherent challenges posed by petroleum, there is a need for a renewable petroleum source which does not need to be explored, extracted, transported over long distances, or substantially refined like petroleum. There is also a need for a renewable petroleum source that can be produced economically without creating the type of environmental damage produced by the petroleum industry and the burning of petroleum based fuels. For similar reasons, there is also a need for a renewable source of chemicals that are typically derived from petroleum.

### SUMMARY OF THE INVENTION

The invention is based, at least in part, on the identification of cyanobacterial genes that encode hydrocarbon biosynthetic polypeptides. Accordingly, in one aspect, the invention features a method of producing a hydrocarbon, the method comprising producing in a host cell a polypeptide comprising the amino acid sequence of SEQ ID NO: 2, 4, 6, 8, 10, 12, 14, 16, 18, 20, 22, 24, 26, 28, 30, 32, 34, or 36, or a variant thereof, and isolating the hydrocarbon from the host cell.

In some embodiments, the polypeptide comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 4, 6, 8, 10, 12, 14, 16, 18, 20, 22, 24, 26, 28, 30, 32, 34, or 36.

In some embodiments, the polypeptide comprises the amino acid sequence of SEQ ID NO: 2, 4, 6, 8, 10, 12, 14, 16, 18, 20, 22, 24, 26, 28, 30, 32, 34, or 36 with one or more amino acid substitutions, additions, insertions, or deletions. In some embodiments, the polypeptide has decarbonylase activity. In yet other embodiments, the polypeptide comprises the amino acid sequence of SEQ ID NO: 2, 4, 6, 8, 10, 12, 14, 16, 18, 20, 22, 24, 26, 28, 30, 32, 34, or 36, with one or more conservative amino acid substitutions. For example, the polypeptide comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the polypeptide has about 1, 2, 3, 4, 5, 6, 7, 8, 9, 10, 15, 20, 30, 40, 50, 60, 70, 80, 90, 100, or more amino acid substitutions, additions, insertions, or deletions. In some embodiments, the polypeptide has decarbonylase activity.

The invention is also based, at least in part, on the identification of certain structurally (or spatially) conserved motif among active hydrocarbon biosynthetic polypeptides. For example, using homology modeling techniques, referencing the known three-dimensional atomic coordinates of a hydrocarbon biosynthetic polypeptide, it has been discovered that, in their native three-dimensional (i.e., tertiary) conformation, a number of hydrocarbon biosynthetic enzymes share a core group of amino acid residues within a defined spatial distance to their putative metal centers. This is despite the fact that those hydrocarbon biosynthetic polypeptides may otherwise have modest or even low sequence homology to the known hydrocarbon biosynthetic polypeptide. Furthermore, it has been discovered that the structurally conserved amino acid residues, despite being some distance apart (i.e., not adjacent amino acid residues) in a two-dimensional amino acid sequence alignment, form a distinct and shared sequence motif across a number of hydrocarbon biosynthetic polypeptides. Accordingly, in a certain aspect, the invention features a method of producing a hydrocarbon, the method comprising producing in a host cell a polypeptide comprising a spatially conserved motif or a variant of that motif, and isolating the hydrocarbon from the host cell.

In some embodiments, the spatially conserved motif comprises the amino acid sequence SEQ ID NO: 37. In an additional embodiment, an amino acid sequence, SEQ ID NO: 115, has been discovered that may play a role in the activity levels of hydrocarbon biosynthetic polypeptides. In some embodiments, the polypeptide of SEQ ID NO: 37 can be about 60 to about 185 amino acid residues in length, for example, about 70 to about 170, about 80 to about 160, about 90 to about 150, or about 100 to about 120 amino acid residues in length. An exemplary hydrocarbon biosynthetic polypeptide comprises a 108-mer SEQ ID NO: 37. In certain embodiments, the polypeptides have decarbonylase activity.

In some embodiments, a hydrocarbon biosynthetic polypeptide of the invention comprises an amino acid sequence of SEQ ID NO: 37 or 115, with one or more amino acid substitutions, additions, insertions or deletions. In certain embodiments, the one or more amino acid substitutions, additions, insertions or deletions can be at the enumerated amino acid positions of SEQ ID NO: 37 or 115 (i.e., the non-X positions, including, for example, the positions of assigned amino acid residues, as well as the positions assigned with alternative amino acid residues). In certain other embodiments, the one or more amino acid substitutions, additions, insertions or deletions can be at the X positions. In yet other embodiments, the amino acid substitutions, additions, insertions or deletions can be present at a combination of X and non-X amino acid positions.

In yet other embodiments, a hydrocarbon biosynethtic polypeptide comprises the amino acid sequence of SEQ ID NO: 37 or 115, with one or more conserved amino acid substitutions. For example, the polypeptide comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

In some embodiments, a polypeptide of SEQ ID NO: 37 or 115 comprises about 1 or more amino acid substitutions, additions, insertions, or deletions at the non-X positions. In other embodiments, a polypeptide of SEQ ID NO: 37 or 115 comprises about 1 or more amino acid substitutions, additions, deletions, or insertions at the X positions. In certain other embodiments, a polypeptide of SEQ ID NO: 37 or 115 comprises about 1 or more amino acid substitutions, additions, insertions, or deletions at a combination of X or non-X positions.

In one aspect, 1 or more amino acid residues of a polypeptide of SEQ ID NO: 37 or 115 are bound to 1, 2, or more metal ions. Typically, the metal ions are situated at or near the active site of a hydrocarbon biosynthetic polypeptide such that the enzyme is said to comprise a “metal center.” For example, a hydrocarbon biosynthetic polypeptide comprising SEQ ID NO: 37 or 115 contains 1 or more, 2 or more, 3 or more, 4 or more, or 5 or more amino acid residues that are bound to 1 or more, 2 or more, or 3 or more metal ions. In a particular embodiment, the amino acid residues bound to (i.e., chelating) the metal ions are residues of SEQ ID NO: 37 or 115. In other embodiments, one or more of the amino acid residues bound to the metal ions are not residues of SEQ ID NO: 37 or 115.

In some embodiments, the one or more metal ions are selected from the group consisting of Zn²⁺, Mg²⁺, Mn²⁺, Fe²⁺, Fe³⁺, Fe⁴⁺, Cu²⁺, K⁺, and Na⁺, or a combination thereof. Alternatively, the one or more metal ions are selected from transition metal ions or a combination thereof. In some embodiments wherein 2 or more metal ions are bound to a hydrocarbon biosynthetic polypeptide, the metal ions that are bound are the same. For example, a hydrocarbon biosynthetic polypeptide of the invention is bound to two Fe²⁺ ions or bound to two Fe³⁺ ions. In other embodiments, wherein 2 or more metal ions are bound to a polypeptide, the metal ions that are bound are not the same. For example, a hydrocarbon biosynthetic polypeptide of the invention is bound to a Fe²⁺ ion and a Mn²⁺ ion; to a Fe²⁺ ion and a Zn²⁺ ion; to a Fe²⁺ ion and a Fe³⁺ ion; to a Fe²⁺ ion and a Fe⁴⁺ ion; to a Fe³⁺ and a Fe⁴⁺ ion; or to a Fe²⁺ ion and a Cu²⁺ ion.

Each of the bound metal ions can be independently quadrivalent, trivalent, divalent or univalent. For example, the first of the metal ions at the metal center can be a trivalent metal ion whereas the second of the metal ions can be a divalent metal ion. Alternatively, both the first and the second metal ions bound to a hydrocarbon biosynethetic polypeptide can be divalent metal ions. For example, both metal ions that are bound to a hydrocarbon biosynthetic polypeptide are Fe²⁺. In other non-limiting examples, the metal ions that are bound to a hydrocarbon biosynthetic polypeptide of the invention can be a Fe²⁺ and a Fe³⁺, a Fe²⁺ and a Fe⁴⁺, a Fe³⁺ and a Fe⁴⁺, or a Fe²⁺ and a Mn²⁺.

In certain aspects, the invention pertains to polypeptides having a three-dimensional structure of a decarbonylase enzyme. In exemplary embodiments, the decarbonylase enzyme comprises a metal center. In particular embodiments, the metal center of the decarbonylase enzyme comprises 2 or more metal ions, which can be different metal ions or can be the same metal ions. In certain circumstances, the decarbonylase enzyme is, for example, a Prochlorococcus marinus MIT9313 enzyme, NP_{—}895059 (SEQ ID NO: 18). In some embodiments, a hydrocarbon biosynthetic polypeptide of the invention has or comprises atomic coordinates of Protein Data Bank Accession No. 2oc5A. In certain specific embodiments, a hydrocarbon biosynthetic polypeptide of the invention comprises the metal-center three-dimensional structure of an enzyme that has the atomic coordinates of PDB Accession No. 2oc5A. In certain related embodiments, the putative metal-center amino acid residues of a hydrocarbon biosynthetic polypeptide of the invention can be fitted to the known metal-center amino acid residues of a hydrocarbon biosynthetic polypeptide with defined tertiary structure, using a suitable homology modeling technique that is known in the art. For example, the known hydrocarbon biosynthetic polypeptide is a Prochlorococcus marinus MIT9313 enzyme, NP_{—}895059 (SEQ ID NO: 18), which has the atomic coordinates of PDB Accession No. 2oc5A. An exemplary homology modeling technique familiar to those of ordinary skill in the art is the Swiss-Model Workplace, available on the World Wide Web at http://swissmodel.expasy.org.

In other embodiments, the hydrocarbon biosynthetic polypeptide comprises the amino acid sequence of: (i) SEQ ID NO: 38 or SEQ ID NO: 39 or SEQ ID NO: 40; or (ii) SEQ ID NO: 41 and any one of (a) SEQ ID NO: 38, (b) SEQ ID NO: 39, and (c) SEQ ID NO: 40; or (iii) SEQ ID NO: 42 or SEQ ID NO: 43 or SEQ ID NO: 44 or SEQ ID NO: 45. In certain embodiments, the polypeptide has decarbonylase activity.

In another aspect, the invention features a method of producing a hydrocarbon, the method comprising expressing in a host cell a polynucleotide comprising a nucleotide sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1, 3, 5, 7, 9, 11, 13, 15, 17, 19, 21, 23, 25, 27, 29, 31, 33, or 35. In some embodiments, the nucleotide sequence is SEQ ID NO: 1, 3, 5, 7, 9, 11, 13, 15, 17, 19, 21, 23, 25, 27, 29, 31, 33, or 35. In some embodiments, the method further comprises providing the polypeptide, wherein the polypeptide interacts with the host cell to produce a hydrocarbon. In some embodiments, the method further comprises isolating the hydrocarbon from the host cell.

In other embodiments, the nucleotide sequence hybridizes to a complement of SEQ ID NO: 1, 3, 5, 7, 9, 11, 13, 15, 17, 19, 21, 23, 25, 27, 29, 31, 33, or 35, or to a fragment thereof, for example, under low stringency, medium stringency, high stringency, or very high stringency conditions.

In other embodiments, the nucleotide sequence encodes a polypeptide comprising: (i) the amino acid sequence of SEQ ID NO: 2, 4, 6, 8, 10, 12, 14, 16, 18, 20, 22, 24, 26, 28, 30, 32, 34, or 36; or (ii) the amino acid sequence of SEQ ID NO: 2, 4, 6, 8, 10, 12, 14, 16, 18, 20, 22, 24, 26, 28, 30, 32, 34, or 36 with one or more amino acid substitutions, additions, insertions, or deletions. In some embodiments, the polypeptide comprises the amino acid sequence of SEQ ID NO: 2, 4, 6, 8, 10, 12, 14, 16, 18, 20, 22, 24, 26, 28, 30, 32, 34, or 36 with one or more conservative amino acid substitutions. In some embodiments, the polypeptide has decarbonylase activity.

In other embodiments, the nucleotide sequence encodes a polypeptide having the same biological activity as a polypeptide comprising the amino acid sequence of SEQ ID NO: 2, 4, 6, 8, 10, 12, 14, 16, 18, 20, 22, 24, 26, 28, 30, 32, 34, or 36. In some embodiments, the nucleotide sequence is SEQ ID NO: 1, 3, 5, 7, 9, 11, 13, 15, 17, 19, 21, 23, 25, 27, 29, 31, 33, or 35 or a fragment thereof. In other embodiments, the nucleotide sequence hybridizes to a complement of SEQ ID NO: 1, 3, 5, 7, 9, 11, 13, 15, 17, 19, 21, 23, 25, 27, 29, 31, 33, or 35 or to a fragment thereof, for example, under low stringency, medium stringency, high stringency, or very high stringency conditions. In some embodiments, the biological activity is decarbonylase activity.

In other embodiments, the nucleotide sequence encodes a polypeptide comprising SEQ ID NO: 37 or 115, or comprising SEQ ID NO: 37 or 115 with one or more amino acid substitutions, additions, insertions, or deletions. In some embodiments, the polypeptide comprises the amino acid sequence of SEQ ID NO: 37 or 115 with one or more conservative amino acid substitutions. In certain embodiments, the nucleotide sequence encodes a polypeptide having the three-dimensional atomic coordinates of Protein Data Bank Accession No. 2oc5A. Alternatively, the nucleotide sequence encodes a polypeptide having the three-dimensional metal center structure of an enzyme with the atomic coordinates of Protein Data Bank Accession No. 2oc5A. In certain other embodiments, the nucleotide sequence encodes a polypeptide, the putative metal-center amino acid residues of which can be fitted to the metal-center amino acid residues of a known hydrocarbon biosynthetic polypeptide, using known homology modeling techniques. In some embodiments, the polypeptide has decarbonylase activity.

In another aspect, the nucleotide sequence encodes a polypeptide having the same biological activity as a polypeptide comprising SEQ ID NO: 37 or 115. In some embodiments, the nucleotide sequence encodes a polypeptide having the same biological activity as a polypeptide having the three-dimensional atomic coordinates of Protein Data Bank Accession No. 2oc5A. In certain other embodiments, the nucleotide sequence encodes a polypeptide having the same biological activity as a polypeptide, the putative metal-center amino acid residues of which can be fitted to the metal-center amino acid residues of a known hydrocarbon biosynthetic polypeptide, using known homology modeling techniques. In some embodiment, the biological activity is decarbonylase activity.

In some embodiments, the method comprises transforming a host cell with a recombinant vector comprising a nucleotide sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1, 3, 5, 7, 9, 11, 13, 15, 17, 19, 21, 23, 25, 27, 29, 31, 33, or 35. In some embodiments, the method comprises transforming a host cell with a recombinant vector comprising a nucleotide sequence encoding a polypeptide comprising SEQ ID NO: 37 or 115. In certain other embodiments, the method comprises transforming a host cell with a recombinant vector comprising a nucleotide sequence encoding a polypeptide having the three-dimensional metal center structure of an enzyme with the atomic coordinates of Protein Data Bank Accession No. 2oc5A. Alternatively, the method comprises transforming a host cell with a recombinant vector comprising a nucleotide sequence encoding a polypeptide, the putative metal-center amino acid residues of which can be fitted to the metal-center amino acid residues of a hydrocarbon biosynthetic polypeptide of known tertiary structure, using suitable homology modeling techniques.

In some embodiments, the recombinant vector further comprises a promoter operably linked to the nucleotide sequence. In some embodiments, the promoter is a developmentally-regulated, an organelle-specific, a tissue-specific, an inducible, a constitutive, or a cell-specific promoter. In particular embodiments, the recombinant vector comprises at least one sequence selected from the group consisting of (a) a regulatory sequence operatively coupled to the nucleotide sequence; (b) a selection marker operatively coupled to the nucleotide sequence; (c) a marker sequence operatively coupled to the nucleotide sequence; (d) a purification moiety operatively coupled to the nucleotide sequence; (e) a secretion sequence operatively coupled to the nucleotide sequence; and (f) a targeting sequence operatively coupled to the nucleotide sequence. In certain embodiments, the nucleotide sequence is stably incorporated into the genomic DNA of the host cell, and the expression of the nucleotide sequence is under the control of a regulated promoter region.

In any of the aspects described herein, the host cell can be selected from the group consisting of a mammalian cell, plant cell, insect cell, yeast cell, fungus cell, filamentous fungi cell, and bacterial cell.

In some embodiments, the host cell is a Gram-positive bacterial cell. In other embodiments, the host cell is a Gram-negative bacterial cell.

In some embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In particular embodiments, the host cell is a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell. In other embodiments, the host cell is an Actinomycetes cell.

In some embodiments, the host cell is a CHO cell, a COS cell, a VERO cell, a BHK cell, a HeLa cell, a Cv1 cell, an MDCK cell, a 293 cell, a 3T3 cell, or a PC12 cell.

In particular embodiments, the host cell is an E. coli cell, such as a strain B, a strain C, a strain K, or a strain W E. coli cell.

In other embodiments, the host cell is a cyanobacterial host cell. In particular embodiments, the cyanobacterial host cell is a cell listed in Table 1. An exemplary cyanobacterial host cell is a Synechococcus sp. PCC7002 cell. Another exemplary cyanobacterial host cell is a Synechocytis sp. PCC6803 cell. Yet another exemplary cyanobacterial host cell is a Synechococcus elongatus PCC7942 cell.

In some embodiments, the hydrocarbon is secreted from by the host cell.

In certain embodiments, the host cell over expresses a substrate described herein. In some embodiments, the method further includes transforming the host cell with a nucleic acid that encodes an enzyme described herein, and the host cell over expresses a substrate described herein. In other embodiments, the method further includes culturing the host cell in the presence of at least one substrate described herein. In some embodiments, the substrate is a fatty acid derivative, an acyl-ACP, a fatty acid, an acyl-CoA, a fatty aldehyde, a fatty alcohol, or a fatty ester. In certain embodiments, the alkanes and alkenes can be made in a malonyl-CoA-independent fashion.

In some embodiments, the fatty acid derivative substrate is an unsaturated fatty acid derivative substrate, a monounsaturated fatty acid derivative substrate, or a saturated fatty acid derivative substrate. In other embodiments, the fatty acid derivative substrate is a straight chain fatty acid derivative substrate, a branched chain fatty acid derivative substrate, or a fatty acid derivative substrate that includes a cyclic moiety.

In certain embodiments of the aspects described herein, the hydrocarbon produced is an alkane. In some embodiments, the alkane is a C3-C25 alkane. For example, the alkane is a C3, C4, C5, C6, C7, C8, C9, C10, C11, C12, C13, C14, C15, C16, C17, C18, C19, C20, C21, C22, C23, C24, or C25 alkane. In some embodiments, the alkane is tridecane, methyltridecane, nonadecane, methylnonadecane, heptadecane, methylheptadecane, pentadecane, or methylpentadecane.

In some embodiments, the alkane is a straight chain alkane, a branched chain alkane, or a cyclic alkane.

In certain embodiments, the method further comprises culturing the host cell in the presence of a saturated fatty acid derivative, and the hydrocarbon produced is an alkane. In certain embodiments, the saturated fatty acid derivative is a C6-C26 fatty acid derivative substrate. For example, the fatty acid derivative substrate is a C6, C7, C8, C9, C10, C11, C12, C13, C14, C15, C16, C17, C18, C19, C20, C21, C22, C23, C24, C25, or a C26 fatty acid derivative substrate. In particular embodiments, the fatty acid derivative substrate is 2-methylicosanal, icosanal, octadecanal, tetradecanal, 2-methyloctadecanal, stearaldehyde, or palmitaldehyde.

In some embodiments, the method further includes isolating the alkane from the host cell or from the culture medium. In other embodiments, the method further includes cracking or refining the alkane.

In certain embodiments of the aspects described herein, the hydrocarbon produced is an alkene. In some embodiments, the alkene is a C3-C25 alkene. For example, the alkene is a C3, C4, C5, C6, C7, C8, C9, C10, C11, C12, C13, C14, C15, C16, C17, C18, C19, C20, C21, C22, C23, C24, or C25 alkene. In some embodiments, the alkene is pentadecene, heptadecene, methylpentadecene, or methylheptadecene.

In some embodiments, the alkene is a straight chain alkene, a branched chain alkene, or a cyclic alkene.

In certain embodiments, the method further comprises culturing the host cell in the presence of an unsaturated fatty acid derivative, and the hydrocarbon produced is an alkene. In certain embodiments, the unsaturated fatty acid derivative is a C6-C26 fatty acid derivative substrate. For example, the fatty acid derivative substrate is a C6, C7, C8, C9, C10, C11, C12, C13, C14, C15, C16, C17, C18, C19, C20, C21, C22, C23, C24, C25, or a C26 unsaturated fatty acid derivative substrate. In particular embodiments, the fatty acid derivative substrate is octadecenal, hexadecenal, methylhexadecenal, or methyloctadecenal.

In another aspect, the invention features a genetically engineered microorganism comprising an exogenous control sequence stably incorporated into the genomic DNA of the microorganism. In one embodiment, the control sequence is integrated upstream of a polynucleotide comprising a nucleotide sequence having at least about 70% sequence identity to SEQ ID NO: 1, 3, 5, 7, 9, 11, 13, 15, 17, 19, 21, 23, 25, 27, 29, 31, 33, or 35. In some embodiments, the nucleotide sequence has at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1, 3, 5, 7, 9, 11, 13, 15, 17, 19, 21, 23, 25, 27, 29, 31, 33, or 35. In some embodiments, the nucleotide sequence is SEQ ID NO: 1, 3, 5, 7, 9, 11, 13, 15, 17, 19, 21, 23, 25, 27, 29, 31, 33, or 35. In other embodiments, the nucleotide sequence encodes a polypeptide comprising SEQ ID NO: 37 or 115.

In some embodiments, the polynucleotide is endogenous to the microorganism. In some embodiments, the microorganism expresses an increased level of a hydrocarbon relative to a wild-type microorganism. In some embodiments, the microorganism is a cyanobacterium.

In another aspect, the invention features a method of making a hydrocarbon, the method comprising culturing a genetically engineered microorganism described herein under conditions suitable for gene expression, and isolating the hydrocarbon.

In another aspect, the invention features a method of making a hydrocarbon, comprising contacting a substrate with (i) a polypeptide having at least 70% identity to the amino acid sequence of SEQ ID NO: 2, 4, 6, 8, 10, 12, 14, 16, 18, 20, 22, 24, 26, 28, 30, 32, 34, or 36, or a variant thereof; (ii) a polypeptide encoded by a nucleotide sequence having at least 70% identity to SEQ ID NO: 1, 3, 5, 7, 9, 11, 13, 15, 17, 19, 21, 23, 25, 27, 29, 31, 33, or 35, or a variant thereof; (iii) a polypeptide comprising the amino acid sequence of SEQ ID NO: 38, 39, or 40; (iv) a polypeptide comprising the amino acid sequence of SEQ ID NO: 41 and any one of (a) SEQ ID NO: 38, (b) SEQ ID NO: 39, and (c) SEQ ID NO: 40; or (v) SEQ ID NO: 42, 43, 44, or 45. In some embodiments, the polypeptide has decarbonylase activity.

In yet another aspect, the invention features a method of making a hydrocarbon, comprising contacting a substrate with a polypeptide comprising SEQ ID NO: 37 or 115, or comprising a variant of SEQ ID NO: 37 or 115. In some embodiments, the polypeptide comprises a conserved variant of SEQ ID NO: 37 or 115. In certain embodiments, the invention features a method of making a hydrocarbon comprising contacting a substrate with a polypeptide comprising a three-dimensional metal center structure of an enzyme with the atomic coordinates of Protein Data Bank Accession No. 2oc5A. The invention also features a method of making a hydrocarbon comprising contacting a substrate with a polypeptide, the putative metal-center amino acid residues of which can be fitted to the metal-center amino acid residues of a known hydrocarbon biosynthetic polypeptide, using homology modeling techniques. In some embodiments, the polypeptide has decarbonylase activity.

In some embodiments, the polypeptide has at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 4, 6, 8, 10, 12, 14, 16, 18, 20, 22, 24, 26, 28, 30, 32, 34, or 36. In some embodiments, the polypeptide has the amino acid sequence of SEQ ID NO: 2, 4, 6, 8, 10, 12, 14, 16, 18, 20, 22, 24, 26, 28, 30, 32, 34, or 36. In other embodiments, the polypeptide comprises SEQ ID NO: 37 or 115. In other embodiments, the polypeptide has a three-dimensional metal center structure of an enzyme with the atomic coordinates of Protein Data Bank Accession No. 2oc5A.

In some embodiments, the polypeptide is encoded by a nucleotide sequence having at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1, 3, 5, 7, 9, 11, 13, 15, 17, 19, 21, 23, 25, 27, 29, 31, 33, or 35. In some embodiments, the polypeptide is encoded by a nucleotide sequence having SEQ ID NO: 1, 3, 5, 7, 9, 11, 13, 15, 17, 19, 21, 23, 25, 27, 29, 31, 33, or 35.

In some embodiments, the biological substrate is a fatty acid derivative, an acyl-ACP, a fatty acid, an acyl-CoA, a fatty aldehyde, a fatty alcohol, or a fatty ester.

In some embodiments, the substrate is a saturated fatty acid derivative, and the hydrocarbon is an alkane, for example, a C3-C25 alkane. For example, the alkane is a C3, C4, C5, C6, C7, C8, C9, C10, C11, C12, C13, C14, C15, C16, C17, C18, C19, C20, C21, C22, C23, C24, or C25 alkane. In some embodiments, the alkane is tridecane, methyltridecane, nonadecane, methylnonadecane, heptadecane, methylheptadecane, pentadecane, or methylpentadecane.

In some embodiments, the alkane is a straight chain alkane, a branched chain alkane, or a cyclic alkane.

In some embodiments, the saturated fatty acid derivative is 2-methylicosanal, icosanal, octadecanal, tetradecanal, 2-methyloctadecanal, stearaldehyde, or palmitaldehyde.

In some embodiments, the polypeptide has at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 4, 6, 8, 10, 12, 14, 16, 18, 20, 22, 24, 26, 28, 30, 32, 34, or 36. In some embodiments, the polypeptide has the amino acid sequence of SEQ ID NO: 2, 4, 6, 8, 10, 12, 14, 16, 18, 20, 22, 24, 26, 28, 30, 32, 34, or 36.

In other embodiments, the biological substrate is an unsaturated fatty acid derivative, and the hydrocarbon is an alkene, for example, a C3-C25 alkene. For example, the alkene is a C3, C4, C5, C6, C7, C8, C9, C10, C11, C12, C13, C14, C15, C16, C17, C18, C19, C20, C21, C22, C23, C24, or C25 alkene. In some embodiments, the alkene is pentadecene, heptadecene, methylpentadecene, or methylheptadecene.

In some embodiments, the alkene is a straight chain alkene, a branched chain alkene, or a cyclic alkene.

In some embodiments, the unsaturated fatty acid derivative is octadecenal, hexadecenal, methylhexadecenal, or methyloctadecenal.

In some embodiments, the polypeptide is encoded by a nucleotide sequence having at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1, 3, 5, 7, 9, 11, 13, 15, 17, 19, 21, 23, 25, 27, 29, 31, 33, or 35. In some embodiments, the polypeptide is encoded by a nucleotide sequence having SEQ ID NO: 1, 3, 5, 7, 9, 11, 13, 15, 17, 19, 21, 23, 25, 27, 29, 31, 33, or 35.

In another aspect, the invention features a hydrocarbon produced by any of the methods or microorganisms described herein. In particular embodiments, the hydrocarbon is an alkane or an alkene having a δ13C of about −15.4 or greater. For example, the alkane or alkene has a δ13C of about −15.4 to about 10.9, for example, about −13.92 to about 13.84. In other embodiments, the alkane or alkene has an fM14C of at least about 1.003. For example, the alkane or alkene has an fM14C of at least about 1.01 or at least about 1.5. In some embodiments, the alkane or alkene has an fM14C of about 1.111 to about 1.124.

In another aspect, the invention features a biofuel that includes a hydrocarbon produced by any of the methods or microorganisms described herein. In particular embodiments, the hydrocarbon is an alkane or alkene having a δ13C of about 15.4 or greater. For example, the alkane or alkene has a δ13C of about −15.4 to about 10.9, for example, about −13.92 to about 13.84. In other embodiments, the alkane or alkene has an fM14C of at least about 1.003. For example, the alkane or alkene has an fM14C of at least about 1.01 or at least about 1.5. In some embodiments, the alkane or alkene has an fM14C of about 1.111 to about 1.124. In some embodiments, the biofuel is diesel, gasoline, or jet fuel.

In another aspect, the invention features an isolated nucleic acid consisting of no more than about 500 nucleotides of SEQ ID NO: 1, 3, 5, 7, 9, 11, 13, 15, 17, 19, 21, 23, 25, 27, 29, 31, 33, or 35. In some embodiments, the nucleic acid consists of no more than about 300 nucleotides, no more than about 350 nucleotides, no more than about 400 nucleotides, no more than about 450 nucleotides, no more than about 550 nucleotides, no more than about 600 nucleotides, or no more than about 650 nucleotides, of SEQ ID NO: 1, 3, 5, 7, 9, 11, 13, 15, 17, 19, 21, 23, 25, 27, 29, 31, 33, or 35. In some embodiments, the nucleic acid encodes a polypeptide having decarbonylase activity.

In another aspect, the invention features an isolated nucleic acid encoding a polypeptide comprising SEQ ID NO: 37 or 115, wherein the nucleic acid consists of no more than about 700 nucleotides, no more than about 600 nucleotides, no more than about 550 nucleotides, no more than about 500 nucleotides, no more than about 450 nucleotides. In a related aspect, the invention features an isolated nucleic acid encoding a polypeptide having a metal center tertiary structure of an enzyme with the atomic coordinates of Protein Data Bank Accession No. 2oc5A, wherein the nucleic acid consists of no more than about 700 nucleotides, no more than about 600 nucleotides, no more than about 550 nucleotides, no more than about 500 nucleotides, or no more than about 450 nucleotides. In yet another aspect, the invention features an isolated nucleic acid encoding a polypeptide, the putative metal-center amino acid residues of which can be fitted to the metal-center amino acid residues of a known hydrocarbon biosynthetic polypeptide, using suitable homology modeling techniques, wherein the nucleic acid consists of no more than about 700 nucleotides, no more than about 600 nucleotides, no more than about 550 nucleotides, no more than about 500 nucleotides, or no more than about 450 nucleotides. An example of a known hydrocarbon biosynthetic polypeptide is a Prochlorococcus marinus MIT9313 enzyme, NP_{—}595059. In some embodiments, the nucleic acid encodes a polypeptide having decarbonylase activity.

In another aspect, the invention features an isolated nucleic acid consisting of no more than about 99%, no more than about 98%, no more than about 97%, no more than about 96%, no more than about 95%, no more than about 94%, no more than about 93%, no more than about 92%, no more than about 91%, no more than about 90%, no more than about 85%, or no more than about 80% of the nucleotides of SEQ ID NO: 1, 3, 5, 7, 9, 11, 13, 15, 17, 19, 21, 23, 25, 27, 29, 31, 33, or 35. In some embodiments, the nucleic acid encodes a polypeptide having decarbonylase activity.

In another aspect, the invention features an isolated polypeptide consisting of no more than about 200, no more than about 175, no more than about 150, or no more than about 100 of the amino acids of SEQ ID NO: 2, 4, 6, 8, 10, 12, 14, 16, 18, 20, 22, 24, 26, 28, 30, 32, 34, or 36. In some embodiments, the polypeptide has decarbonylase activity.

In another aspect, the invention features an isolated polypeptide consisting of no more than about 99%, no more than about 98%, no more than about 97%, no more than about 96%, no more than about 95%, no more than about 94%, no more than about 93%, no more than about 92%, no more than about 91%, no more than about 90%, no more than about 85%, or no more than about 80% of the amino acids of SEQ ID NO: 2, 4, 6, 8, 10, 12, 14, 16, 18, 20, 22, 24, 26, 28, 30, 32, 34, or 36. In some embodiments, the polypeptide has decarbonylase activity.

### BRIEF DESCRIPTION OF THE DRAWINGS

FIG. 1A is a GC/MS trace of hydrocarbons produced by Prochlorococcus marinus CCMP1986 cells. FIG. 1B is a mass fragmentation pattern of the peak at 7.55 min of FIG. 1A.

FIG. 2A is a GC/MS trace of hydrocarbons produced by Nostoc punctiforme PCC73102 cells. FIG. 2B is a mass fragmentation pattern of the peak at 8.73 min of FIG. 2A.

FIG. 3A is a GC/MS trace of hydrocarbons produced by Gloeobaceter violaceus ATCC29082 cells. FIG. 3B is a mass fragmentation pattern of the peak at 8.72 min of FIG. 3A.

FIG. 4A is a GC/MS trace of hydrocarbons produced by Synechocystic sp. PCC6803 cells. FIG. 4B is a mass fragmentation pattern of the peak at 7.36 min of FIG. 4A.

FIG. 5A is a GC/MS trace of hydrocarbons produced by Synechocystis sp. PCC6803 wild type cells. FIG. 5B is a GC/MS trace of hydrocarbons produced by Synechocystis sp. PCC6803 cells with a deletion of the sll0208 and sll0209 genes.

FIG. 5A is a GC/MS trace of hydrocarbons produced by Synechocystis sp. PCC6803 wild type cells. FIG. FIG. 5A is a GC/MS trace of hydrocarbons produced by Synechocystis sp. PCC6803 wild type cells. FIG. 5B is a GC/MS trace of hydrocarbons produced by Synechocystis sp. PCC6803 cells with a deletion of the sll0208 and sll0209 genes.

FIG. 6A is a GC/MS trace of hydrocarbons produced by E. coli MG1655 wild type cells. FIG. 6B is a GC/MS trace of hydrocarbons produced by E. coli MG1655 cells expressing Synechococcus elongatus PCC7942 YP_{—}400611 (Synpcc7942_{—}1594) (SEQ ID NO: 66).

FIG. 7 is a GC/MS trace of hydrocarbons produced by E. coli cells expressing Cyanothece sp. ATCC51142 cce_{—}1430 (YP_{—}001802846) (SEQ ID NO: 70).

FIG. 8A is a GC/MS trace of hydrocarbons produced by E. coli cells expressing Synechococcus elongatus PCC7942 YP_{—}400611 (Synpcc7942_{—}1594) (SEQ ID NO: 66) and Synechococcus elongatus PCC7942 YP_{—}400610 (Synpcc7942_{—}1593) (SEQ ID NO: 1). FIG. 8B depicts mass fragmentation patterns of the peak at 6.98 min of FIG. 8A and of pentadecane. FIG. 8C depicts mass fragmentation patterns of the peak at 8.12 min of FIG. 8A and of 8-heptadecene.

FIG. 9 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Nostoc punctiforme PCC73102 Npun02004178 (ZP_{—}00108838) (SEQ ID NO: 5).

FIG. 10 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Synechocystis sp. PCC6803 sll0208 (NP_{—}442147) (SEQ ID NO: 3).

FIG. 11 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Nostoc sp. PCC7210 alr5283 (NP_{—}489323) (SEQ ID NO: 7).

FIG. 12 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Acaryochloris marina MBIC11017 AM1_{—}4041 (YP_{—}001518340) (SEQ ID NO: 47).

FIG. 13 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Thermosynechococcus elongatus BP-1 tll1313 (NP_{—}682103) (SEQ ID NO: 48).

FIG. 14 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Synechococcus sp. JA-3-3Ab CYA_{—}0415 (YP_{—}473897) (SEQ ID NO: 49).

FIG. 15 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Gloeobacter violaceus PCC7421 gll3146 (NP_{—}926092) (SEQ ID NO: 15).

FIG. 16 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Prochlorococcus marinus MIT9313 PM1231 (NP_{—}895059) (SEQ ID NO: 50).

FIG. 17 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Prochlorococcus marinus CCMP1986 PMM0532 (NP_{—}892650) (SEQ ID NO: 19).

FIG. 18 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Prochlorococcus marinus NATL2A PMN2A_{—}1863 (YP_{—}293054) (SEQ ID NO: 52).

FIG. 19 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Synechococcus sp. RS9917 RS9917_{—}09941 (ZP_{—}01079772) (SEQ ID NO: 53).

FIG. 20 is a GC/MS trace of hydrocarbons produced by E. coli MG1655 cells expressing Synechococcus elongatus PCC7942 YP_{—}400611 (Synpcc7942_{—}1594) (SEQ ID NO: 66) and codon-optimized Synechococcus sp. RS9917 RS9917_{—}12945 (ZP_{—}01080370) (SEQ ID NO: 54).

FIG. 21 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Cyanothece sp. ATCC51142 cce_{—}0778 (YP_{—}001802195) (SEQ ID NO: 27).

FIG. 22 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Cyanothece sp. PCC7425 Cyan7425_{—}0398 (YP_{—}002481151) (SEQ ID NO: 29).

FIG. 23 is a GC/MS trace of hydrocarbons produced by E. coli MG1655 cells expressing Synechococcus elongatus PCC7942 YP_{—}400611 (Synpcc7942_{—}1594) (SEQ ID NO: 66) and Cyanothece sp. PCC7425 Cyan7425_{—}2986 (YP_{—}002483683) (SEQ ID NO: 31).

FIG. 24A is a GC/MS trace of hydrocarbons produced by E. coli MG1655 cells expressing Prochlorococcus marinus CCMP1986 PMM0533 (NP_{—}892651) (SEQ ID NO: 72). FIG. 24B is a GC/MS trace of hydrocarbons produced by E. coli MG1655 cells expressing Prochlorococcus marinus CCMP1986 PMM0533 (NP_{—}892651) (SEQ ID NO: 72) and Prochlorococcus mariunus CCMP 1986 PMM0532 (NP_{—}892650) (SEQ ID NO: 19).

FIG. 25A is a GC/MS trace of hydrocarbons produced by E. coli MG1655 ΔfadE lacZ::P_{trc} ′tesA-fadD cells. FIG. 25B is a GC/MS trace of hydrocarbons produced by E. coli MG1655 ΔfadE lacZ::P_{trc} ′tesA-fadD cells expressing Synechococcus elongatus PCC7942 YP_{—}400611 (Synpcc7942_{—}1594) (SEQ ID NO: 66) and Acaryochloris marina MBIC11017 AM1_{—}4041 (YP_{—}001518340) (SEQ ID NO: 9).

FIG. 26A is a GC/MS trace of hydrocarbons produced by E. coli MG1655 ΔfadE lacZ::P_{trc} ′tesA-fadD cells expressing Synechocystis sp. PCC6803 sll0209 (NP_{—}442146) (SEQ ID NO: 68). FIG. 26B is a GC/MS trace of hydrocarbons produced by E. coli MG1655 ΔfadE lacZ::P_{trc} ′tesA-fadD cells expressing Synechocystis sp. PCC6803 sll0209 (NP_{—}442146) (SEQ ID NO: 68) and Synechocystis sp. PCC6803 sll0208 (NP_{—}442147) (SEQ ID NO: 3).

FIG. 27A is a GC/MS trace of hydrocarbons produced by E. coli MG1655 ΔfadD lacZ::P_{trc}-′tesA cells expressing M. smegmatis strain MC2 155 MSMEG_{—}5739 (YP_{—}889972) (SEQ ID NO: 86). FIG. 27B is a GC/MS trace of hydrocarbons produced by E. coli MG1655 ΔfadD lacZ::P_{trc}-′tesA cells expressing M. smegmatis strain MC2 155 MSMEG_{—}5739 (YP_{—}889972) (SEQ ID NO: 86) and Nostoc punctiforme PCC73102 Npun02004178 (ZP_{—}00108838) (SEQ ID NO: 5).

FIG. 28 is a graphic representation of hydrocarbons produced by E. coli MG1655 ΔfadD lacZ::P_{trc}-′tesA cells expressing M. smegmatis strain MC2 155 MSMEG_{—}5739 (YP_{—}889972) (SEQ ID NO: 86) either alone or in combination with Nostoc sp. PCC7120 alr5283 (SEQ ID NO: 7), Nostoc punctiforme PCC73102 Npun02004178 (SEQ ID NO: 5), P. mariunus CCMP1986 PMM0532 (SEQ ID NO: 19), G. violaceus PCC7421 gll3146 (SEQ ID NO: 15), Synechococcus sp. RS991709941 (SEQ ID NO: 23), Synechococcus sp. RS9917_{—}12945 (SEQ ID NO: 25), or A. marina MBIC11017 AM1_{—}4041 (SEQ ID NO: 9).

FIG. 29A is a representation of the three-dimensional structure of a class I ribonuclease reductase subunit β protein, RNRβ. FIG. 29B is a representation of the three-dimensional structure of Prochlorococcus marinus MIT9313 PM1231 (NP_{—}895059) (SEQ ID NO: 18). FIG. 29C is a representation of the three-dimensional structure of the active site of Prochlorococcus marinus MIT9313 PM1231 (NP_{—}895059) (SEQ ID NO: 18).

FIG. 30A is a GC/MS trace of hydrocarbons produced by E. coli MG1655 cells expressing Nostoc punctiforme PCC73102 Npun02004178 (ZP_{—}00108838) (SEQ ID NO: 5). FIG. 30B is a GC/MS trace of hydrocarbons produced by E. coli MG1655 cells expressing Nostoc punctiforme PCC73102 Npun02004178 (ZP_{—}00108838) Y123F variant. FIG. 30C is a GC/MS trace of hydrocarbons produced by E. coli MG1655 cells expressing Nostoc punctiforme PCC73102 Npun02004178 (ZP_{—}00108838) Y126F variant.

FIG. 31 depicts GC/MS traces of hydrocarbons produced in vitro using Nostoc punctiforme PCC73102 Npun02004178 (ZP_{—}00108838) (SEQ ID NO: 6) and octadecanal (A); Npun02004178 (ZP_{—}00108838) (SEQ ID NO: 6), octadecanal, spinach ferredoxin reductase, and NADPH (B); octadecanal, spinach ferredoxin, spinach ferredoxin reductase, and NADPH(C); or Npun02004178 (ZP_{—}00108838) (SEQ ID NO: 6), spinach ferredoxin, and spinach ferredoxin (D).

FIG. 32 depicts GC/MS traces of hydrocarbons produced in vitro using Nostoc punctiforme PCC73102 Npun02004178 (ZP_{—}00108838) (SEQ ID NO: 6), NADPH, octadecanal, and either (A) spinach ferredoxin and spinach ferredoxin reductase; (B) N. punctiforme PCC73102 Npun02003626 (ZP_{—}00109192) (SEQ ID NO: 89) and N. punctiforme PCC73102 Npun02001001 (ZP_{—}00111633) (SEQ ID NO: 91); (C) Npun02003626 (ZP_{—}00109192) (SEQ ID NO: 89) and N. punctiforme PCC73102 Npun02003530 (ZP_{—}00109422) (SEQ ID NO: 93); or (D) Npun02003626 (ZP_{—}00109192) (SEQ ID NO: 89) and N. punctiforme PCC73102 Npun02003123 (ZP_{—}00109501) (SEQ ID NO: 95).

FIG. 33A is a GC/MS trace of hydrocarbons produced in vitro using octadecanoyl-CoA, Synechococcus elongatus PCC7942 YP_{—}400611 (Synpcc7942_{—}1594) (SEQ ID NO: 67), NADH, and Mg²⁺. FIG. 33B is a GC/MS trace of hydrocarbons produced in vitro using octadecanoyl-CoA, Synechococcus elongatus PCC7942 YP_{—}400611 (Synpcc7942_{—}1594) (SEQ ID NO: 67), NADPH, and Mg²⁺. FIG. 33C is a GC/MS trace of hydrocarbons produced in vitro using octadecanoyl-CoA, Synechococcus elongatus PCC7942 YP_{—}400611 (Synpcc7942_{—}1594) (SEQ ID NO: 67) and NADPH.

FIG. 34A is a GC/MS trace of hydrocarbons produced in vitro using octadecanoyl-CoA, labeled NADPH, Synechococcus elongatus PCC7942 YP_{—}400611 (Synpcc7942_{—}1594) (SEQ ID NO: 67), and unlabeled NADPH. FIG. 34B is a GC/MS trace of hydrocarbons produced in vitro using octadecanoyl-CoA, labeled NADPH, Synechococcus elongatus PCC7942 YP_{—}400611 (Synpcc7942_{—}1594) (SEQ ID NO: 67), and S-(4-²H)NADPH. FIG. 34C is a GC/MS trace of hydrocarbons produced in vitro using octadecanoyl-CoA, labeled NADPH, Synechococcus elongatus PCC7942 YP_{—}400611 (Synpcc7942_{—}1594) (SEQ ID NO: 67), and R-(4-²H)NADPH.

FIG. 35 is a GC/MS trace of hydrocarbons in the cell-free supernatant produced by E. coli MG1655 ΔfadE cells in Che-9 media expressing Synechococcus elongatus PCC7942 YP_{—}400611 (Synpcc7942_{—}1594) (SEQ ID NO: 66).

FIG. 36 is a GC/MS trace of hydrocarbons in the cell-free supernatant produced by E. coli MG1655 ΔfadE cells in Che-9 media expressing Synechococcus elongatus PCC7942 YP_{—}400611 (Synpcc7942_{—}1594) (SEQ ID NO: 66) and Nostoc punctiforme PCC73102 Npun02004178 (ZP_{—}00108838) (SEQ ID NO: 5).

FIG. 37 is a GC/MS trace of hydrocarbons produced by E. coli MG1655 cells expressing Nostoc sp. PCC7120 alr5283 (NP_{—}489323) (SEQ ID NO: 7) and Nostoc sp. PCC7120 alr5284 (NP_{—}489324) (SEQ ID NO: 82).

FIG. 38 is a list of examples of homologs of Synechococcus elongatus PCC7942 YP_{—}400610 (Synpcc7942_{—}1593) (SEQ ID NO: 1) from a metagenomic database.

FIG. 39 is a list of examples of homologs of Synechococcus elongatus PCC7942 YP_{—}400611 (Synpcc7942_{—}1594) (SEQ ID NO: 66) from a metagenomic database.

FIG. 40 is a table identifying various genes that can be expressed, over expressed, or attenuated to increase production of particular substrates.

FIG. 41 depicts the amino acid residues present within 6 Angstrom (Å) of the metal center of Prochlorococcus marinus MIT9313 PMT1231, NP_{—}895059 (SEQ ID NO: 18). The black ions are the irons of Prochlorococcus marinus MIT9313 PMT1231, whereas the grey residues are amino acid residues within about 6 Å of the di-iron center. Representative types of amino acid residues are also identified.

FIG. 42 depicts a Swiss-Model three-dimensional homology overlay of putative metal-center amino acid residues of Synechococcus sp. RS9917 enzyme ZP_{—}01080370 (SEQ ID NO: 26) with the known di-iron center amino acid residues (i.e., those within 6 Å of the irons) of Prochlorococcus marinus MIT9313 PMT1231, NP_{—}895059 (SEQ ID NO: 18). The black ions are the irons of Prochlorococcus marinus MIT9313 PMT1231, the light grey residues are the amino acid residues of Prochlorococcus marinus MIT9313 PMT1231 that are within 6 Å of its di-iron center, whereas the dark grey residues are the putative metal-center amino acid residues of ZP_{—}01080370 that are fitted to the amino acid residues within 6 Å of the di-iron center of Prochlorococcus marinus MIT9313 PMT1231.

FIG. 43 depicts a Swiss-Model three-dimensional homology overlay of putative metal-center amino acid residues of Synechocytis sp. PCC 6803 enzyme ZP_{—}442147 (SEQ ID NO: 4) with the known di-iron center amino acid residues of Prochlorococcus marinus MIT9313 PMT1231, NP_{—}895059 (SEQ ID NO: 18).

FIG. 44 depicts a Swiss-Model three-dimensional homology overlay of putative metal-center amino acid residues of Nostoc sp. PCC 7120 NP_{—}489323 (SEQ ID NO: 8) with the known di-iron center amino acid residues of Prochlorococcus marinus MIT9313 PMT1231, NP_{—}895059 (SEQ ID NO: 18).

FIG. 45 depicts a Swiss-Model three-dimensional homology overlay of putative metal-center amino acid residues of Thermosynechococcus elongatus BP-1 enzyme NP_{—}682103 (SEQ ID NO: 12) with the known di-iron center amino acid residues of Prochlorococcus marinus MIT9313 PMT1231, NP_{—}895059 (SEQ ID NO: 18).

FIG. 46 depicts a Swiss-Model three-dimensional homology overlay of putative metal-center amino acid residues of Prochlorococcus marinus subsp. pastoris str. CCMP 1986 enzyme NP_{—}892650 (SEQ ID NO: 20) with the known di-iron center amino acid residues of Prochlorococcus marinus MIT9313 PMT1231, NP_{—}895059 (SEQ ID NO: 18).

FIG. 47 depicts a Swiss-Model three-dimensional homology overlay of putative metal-center amino acid residues of Gloeobacter violaceus PCC7421 enzyme NP_{—}926092 (SEQ ID NO: 16) with the known di-iron center amino acid residues of Prochlorococcus marinus MIT9313 PMT1231, NP_{—}895059 (SEQ ID NO: 18).

FIG. 48 depicts a Swiss-Model three-dimensional homology overlay of putative metal-center amino acid residues of Synechococcus elongatus PCC 6301 enzyme YP_{—}170760 (SEQ ID NO: 36) with the known di-iron center amino acid residues of Prochlorococcus marinus MIT9313 PMT1231, NP_{—}895059 (SEQ ID NO: 18).

FIG. 49 depicts a Swiss-Model three-dimensional homology overlay of putative metal-center amino acid residues of Prochlorococcus marinus str. NATL2A enzyme YP_{—}293054 (SEQ ID NO: 22) with the known di-iron center amino acid residues of Prochlorococcus marinus MIT9313 PMT1231, NP_{—}895059 (SEQ ID NO: 18).

FIG. 50 depicts a Swiss-Model three-dimensional homology overlay of putative metal-center amino acid residues of the Synechococcus elongatus PCC 7942 enzyme YP_{—}400610 (SEQ ID NO: 2) with the known di-iron center amino acid residues of Prochlorococcus marinus MIT9313 PMT1231, NP_{—}895059 (SEQ ID NO: 18).

FIG. 51 depicts a Swiss-Model three-dimensional homology overlay of putative metal-center amino acid residues of Synechococcus sp. JA-3-3Ab enzyme YP_{—}473897 (SEQ ID NO: 14) with the known di-iron center amino acid residues of Prochlorococcus marinus MIT9313 PMT1231, NP_{—}895059 (SEQ ID NO: 18).

FIG. 52 depicts a Swiss-Model three-dimensional homology overlay of putative metal-center amino acid residues of Acaryochloris marina MBIC11017 enzyme YP_{—}001518340 (SEQ ID NO: 10) with the known di-iron center amino acid residues of Prochlorococcus marinus MIT9313 PMT1231 (NP_{—}895059) (SEQ ID NO: 18).

FIG. 53 depicts a Swiss-Model three-dimensional homology overlay of putative metal-center amino acid residues of Cyanothece sp. ATCC 51142 enzyme ZP_{—}001802195 (SEQ ID NO: 28) with the known di-iron center amino acid residues of Prochlorococcus marinus MIT9313 PMT1231, NP_{—}895059 (SEQ ID NO: 18).

FIG. 54 depicts a Swiss-Model three-dimensional homology overlay of putative metal-center amino acid residues of Synechococcus sp. RS9917 enzyme YP_{—}01079772 (SEQ ID NO: 24) with the known di-iron center amino acid residues of Prochlorococcus marinus MIT9313 PMT1231, NP_{—}895059 (SEQ ID NO: 18).

FIG. 55 depicts a Swiss-Model three-dimensional homology overlay of putative metal-center amino acid residues of Cyanothece sp. PCC 7425 enzyme YP_{—}00241151 (SEQ ID NO: 30) with the known di-iron center amino acid residues of Prochlorococcus marinus MIT9313 PMT1231 (NP_{—}895059) (SEQ ID NO: 18).

FIG. 56 depicts a Swiss-Model three-dimensional homology overlay of putative metal-center amino acid residues of Cyanothece sp. PCC 7425 enzyme YP_{—}002483683 (SEQ ID NO: 32) with the known di-iron center amino acid residues of Prochlorococcus marinus MIT9313 PMT1231, NP_{—}895059 (SEQ ID NO: 18).

FIG. 57 depicts an amino-acid sequence alignment of certain cyanobacterial alkane biosynthetic gene homologs listed in Table I. The putative metal ion-bound amino acid residues are marked by a “*” above the aligned sequences. The amino acid residues of Prochlorococcus marinus MIT9313 PMT1231, NP_{—}895059 (SEQ ID NO: 18) that are within about 6 Å of the di-ion center and the putative metal-center amino acid residues (e.g., those that were fitted to the known metal-center amino acid residues of PMT1231 using Swiss-Model homology modeling) of the other enzymes are marked with boxes.

FIG. 58 depicts time courses of hydrocarbon production using the methods and microorganisms described herein.

FIG. 59 is a GC/MS trace of hydrocarbons produced by aldehyde decarbonylase from Nostoc punctiforme PCC73102.

FIG. 60 is a GC/MS trace of hydrocarbons produced by aldehyde decarbonylase from Nostoc punctiforme PCC73102.

### DETAILED DESCRIPTION

Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides that have the same activity (e.g., that catalyze the same fundamental chemical reaction).

The accession numbers referenced herein are derived from the NCBI database (National Center for Biotechnology Information) maintained by the National Institute of Health, U.S.A. Unless otherwise indicated, the accession numbers are as provided in the database as of November 2009.

EC numbers are established by the Nomenclature Committee of the International Union of Biochemistry and Molecular Biology (NC-IUBMB) (available at http://www.chem.qmul.ac.ukhubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of March 2008.

The articles “a” and “an” are used herein to refer to one or to more than one (i.e., to at least one) of the grammatical object of the article. By way of example, “an element” means one element or more than one element.

The term “about” is used herein to mean a value ±20% of a given numerical value. Thus, “about 60%” means a value of between 60±(20% of 60) (i.e., between 48 and 70).

As used herein, the term “aldehyde” means a hydrocarbon having the formula RCHO characterized by an unsaturated carbonyl group (C═O). In a preferred embodiment, the aldehyde is any aldehyde made from a fatty acid or fatty acid derivative. In one embodiment, the R group is at least about 1, 2, 3, 4, 5, 6, 7, 8, 9, 10, 11, 12, 13, 14, 15, 16, 17, 18, 19, or 20 carbons in length.

As used herein, an “aldehyde biosynthetic gene” or an “aldehyde biosynthetic polynucleotide” is a nucleic acid that encodes an aldehyde biosynthetic polypeptide.

As used herein, an “aldehyde biosynthetic polypeptide” is a polypeptide that is a part of the biosynthetic pathway of an aldehyde. Such polypeptides can act on a biological substrate to yield an aldehyde. In some instances, the aldehyde biosynthetic polypeptide has reductase activity.

As used herein, the term “alkane” means a hydrocarbon containing only single carbon-carbon bonds.

As used herein, an “alkane biosynthetic gene” or an “alkane biosynthetic polynucleotide” is a nucleic acid that encodes an alkane biosynthetic polypeptide.

As used herein, an “alkane biosynthetic polypeptide” is a polypeptide that is a part of the biosynthetic pathway of an alkane. Such polypeptides can act on a biological substrate to yield an alkane. In some instances, the alkane biosynthetic polypeptide has decarbonylase activity.

As used herein, an “alkene biosynthetic gene” or an “alkene biosynthetic polynucleotide” is a nucleic acid that encodes an alkene biosynthetic polypeptide.

As used herein, an “alkene biosynthetic polypeptide” is a polypeptide that is a part of the biosynthetic pathway of an alkene. Such polypeptides can act on a biological substrate to yield an alkene. In some instances, the alkene biosynthetic polypeptide has decarbonylase activity.

As used herein, the term “attenuate” means to weaken, reduce or diminish. For example, a polypeptide can be attenuated by modifying the polypeptide to reduce its activity (e.g., by modifying a nucleotide sequence that encodes the polypeptide).

As used herein, the term “biodiesel” means a biofuel that can be a substitute of diesel, which is derived from petroleum. Biodiesel can be used in internal combustion diesel engines in either a pure form, which is referred to as “neat” biodiesel, or as a mixture in any concentration with petroleum-based diesel. Biodiesel can include esters or hydrocarbons, such as aldehydes and alkanes.

As used therein, the term “biofuel” refers to any fuel derived from biomass. Biofuels can be substituted for petroleum based fuels. For example, biofuels are inclusive of transportation fuels (e.g., gasoline, diesel, jet fuel, etc.), heating fuels, and electricity-generating fuels. Biofuels are a renewable energy source.

As used herein, the term “biomass” refers to a carbon source derived from biological material. Biomass can be converted into a biofuel. One exemplary source of biomass is plant matter. For example, corn, sugar cane, or switchgrass can be used as biomass. Another non-limiting example of biomass is animal matter, for example cow manure. Biomass also includes waste products from industry, agriculture, forestry, and households. Examples of such waste products that can be used as biomass are fermentation waste, straw, lumber, sewage, garbage, and food leftovers. Biomass also includes sources of carbon, such as carbohydrates (e.g., monosaccharides, disaccharides, or polysaccharides).

As used herein, the phrase “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO2). These include, for example, various monosaccharides, such as glucose, fructose, mannose, and galactose; oligosaccharides, such as fructo-oligosaccharide and galacto-oligosaccharide; polysaccharides such as xylose and arabinose; disaccharides, such as sucrose, maltose, and turanose; cellulosic material, such as methyl cellulose and sodium carboxymethyl cellulose; saturated or unsaturated fatty acid esters, such as succinate, lactate, and acetate; alcohols, such as ethanol or mixtures thereof. The carbon source can also be a product of photosynthesis, including, but not limited to, glucose. A preferred carbon source is biomass. Another preferred carbon source is glucose.

As used herein, a “cloud point lowering additive” is an additive added to a composition to decrease or lower the cloud point of a solution.

As used herein, the phrase “cloud point of a fluid” means the temperature at which dissolved solids are no longer completely soluble. Below this temperature, solids begin precipitating as a second phase giving the fluid a cloudy appearance. In the petroleum industry, cloud point refers to the temperature below which a solidified material or other heavy hydrocarbon crystallizes in a crude oil, refined oil, or fuel to form a cloudy appearance. The presence of solidified materials influences the flowing behavior of the fluid, the tendency of the fluid to clog fuel filters, injectors, etc., the accumulation of solidified materials on cold surfaces (e.g., a pipeline or heat exchanger fouling), and the emulsion characteristics of the fluid with water.

A nucleotide sequence is “complementary” to another nucleotide sequence if each of the bases of the two sequences matches (i.e., is capable of forming Watson Crick base pairs). The term “complementary strand” is used herein interchangeably with the term “complement”. The complement of a nucleic acid strand can be the complement of a coding strand or the complement of a non-coding strand.

As used herein, the term “conditions sufficient to allow expression” means any conditions that allow a host cell to produce a desired product, such as a polypeptide, aldehyde, or alkane described herein. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source, such as glucose, fructose, cellulose, or the like, that can be metabolized by a host cell directly. In addition, enzymes can be used in the medium to facilitate the mobilization (e.g., the depolymerization of starch or cellulose to fermentable sugars) and subsequent metabolism of the carbon source.

To determine if conditions are sufficient to allow expression, a host cell can be cultured, for example, for about 4, 8, 12, 24, 36, or 48 hours. During and/or after culturing, samples can be obtained and analyzed to determine if the conditions allow expression. For example, the host cells in the sample or the medium in which the host cells were grown can be tested for the presence of a desired product. When testing for the presence of a product, assays, such as, but not limited to, TLC, HPLC, GC/FID, GC/MS, LC/MS, MS, can be used.

It is understood that the polypeptides described herein may have additional conservative or non-essential amino acid substitutions, which do not have a substantial effect on the polypeptide functions. Whether or not a particular substitution will be tolerated (i.e., will not adversely affect desired biological properties, such as decarboxylase activity) can be determined as described in Bowie et al., Science (1990) 247:1306 1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

As used herein, “control element” means a transcriptional control element. Control elements include promoters and enhancers. The term “promoter element,” “promoter,” or “promoter sequence” refers to a DNA sequence that functions as a switch that activates the expression of a gene. If the gene is activated, it is said to be transcribed or participating in transcription. Transcription involves the synthesis of mRNA from the gene. A promoter, therefore, serves as a transcriptional regulatory element and also provides a site for initiation of transcription of the gene into mRNA. Control elements interact specifically with cellular proteins involved in transcription (Maniatis et al., Science 236:1237, 1987).

As used herein, the term “ester synthase” means a peptide capable of producing fatty esters. More specifically, an ester synthase is a peptide which converts a thioester to a fatty ester. In a preferred embodiment, the ester synthase converts a thioester (e.g., acyl-CoA) to a fatty ester.

In an alternate embodiment, an ester synthase uses a thioester and an alcohol as substrates to produce a fatty ester. Ester synthases are capable of using short and long chain thioesters as substrates. In addition, ester synthases are capable of using short and long chain alcohols as substrates.

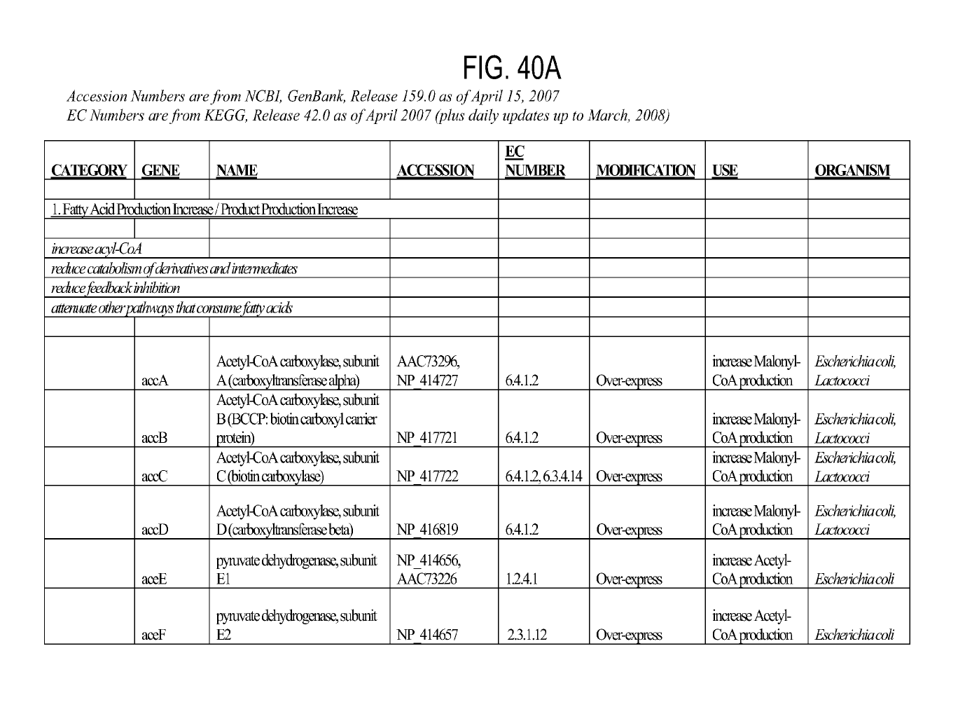

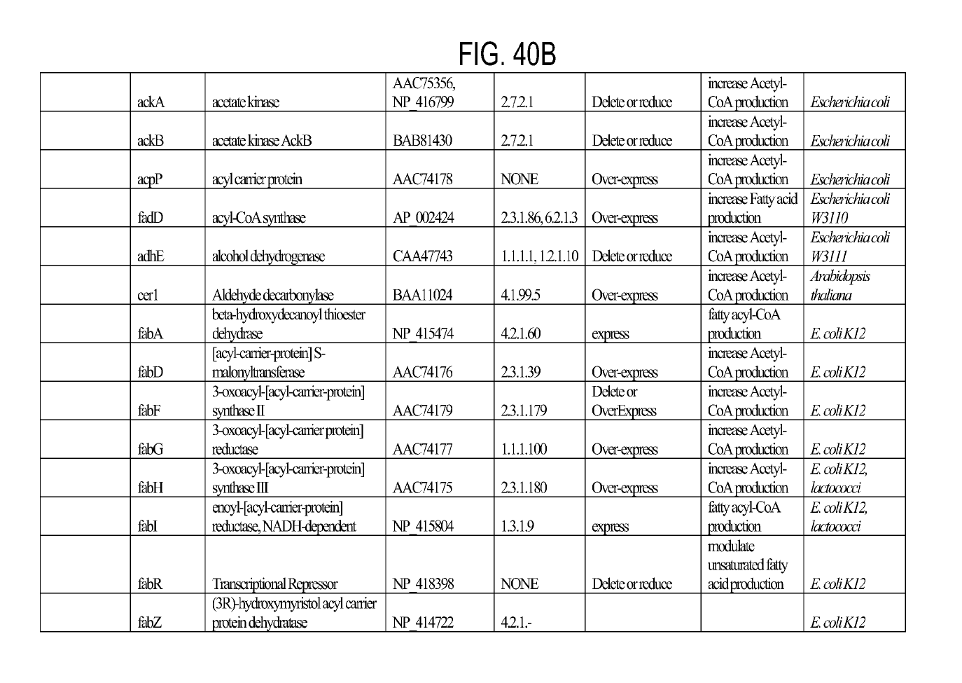

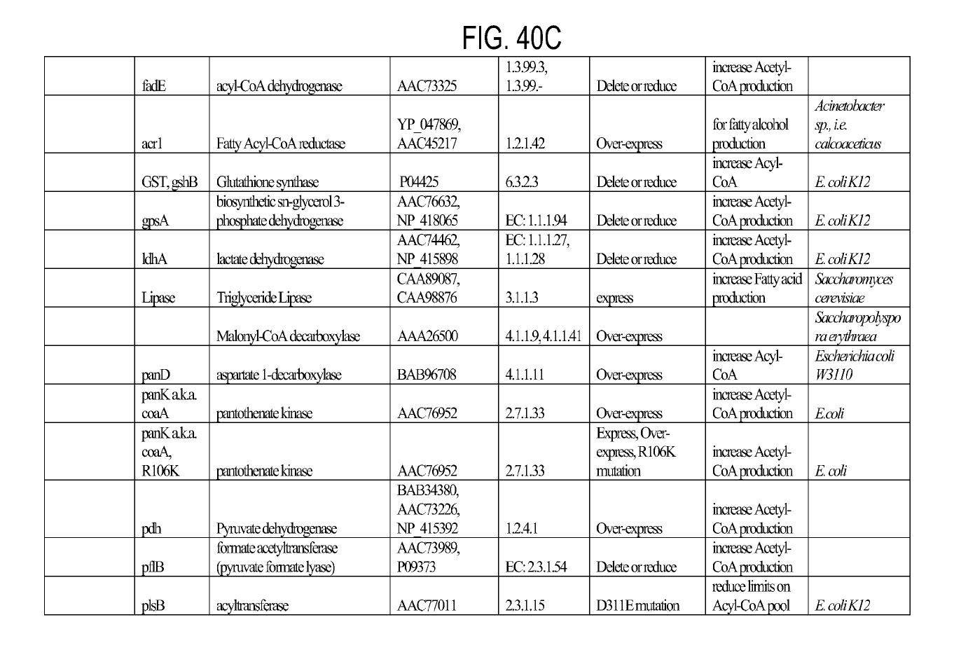

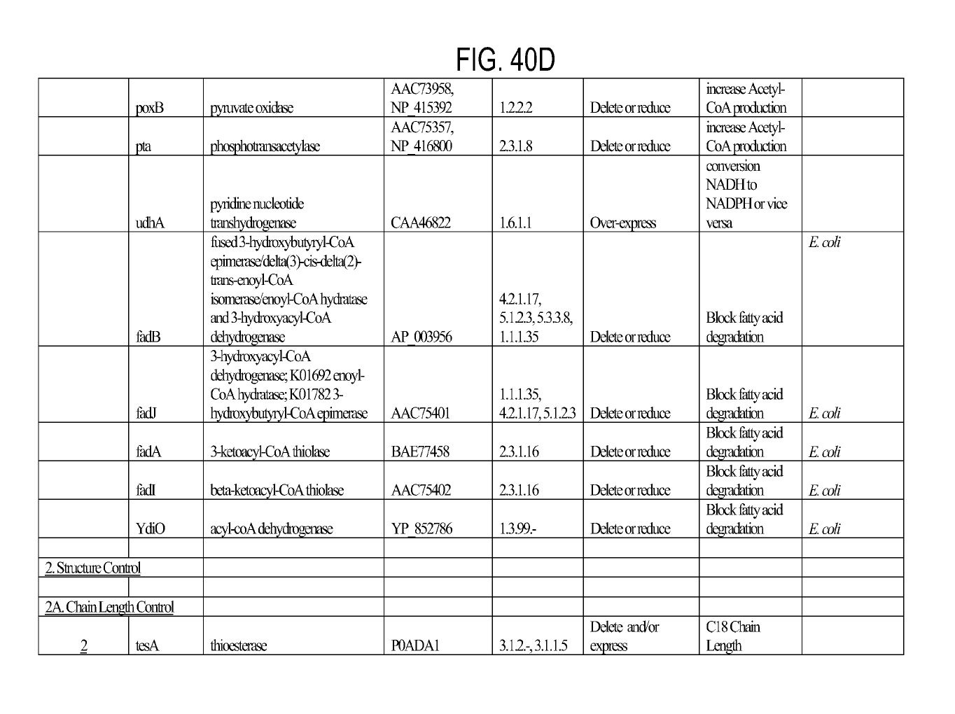

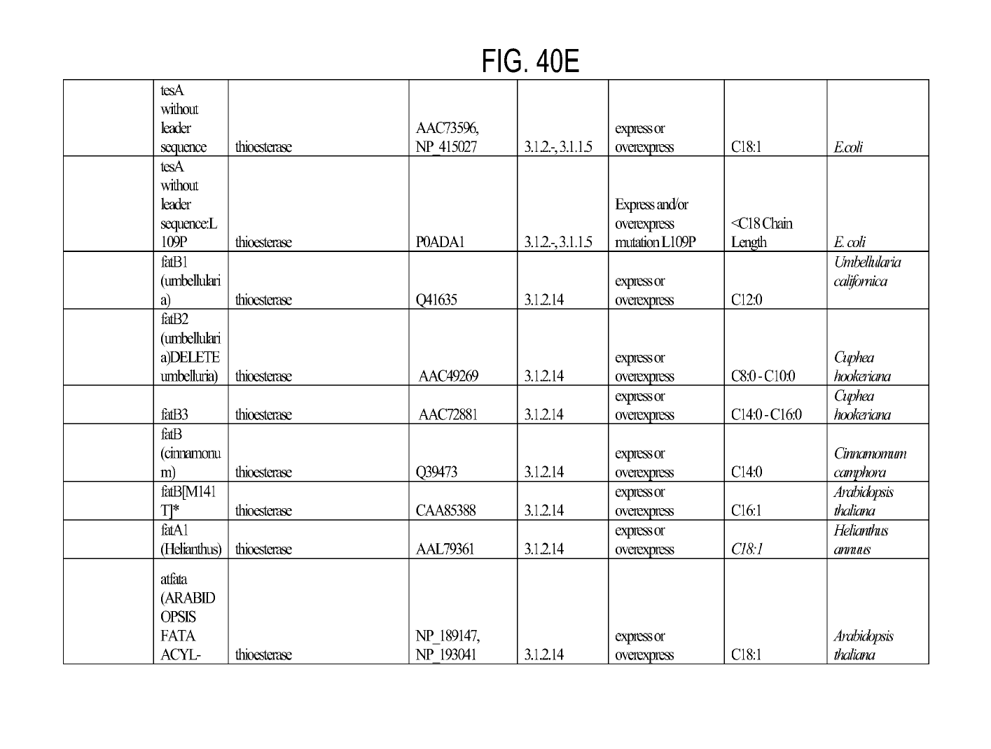

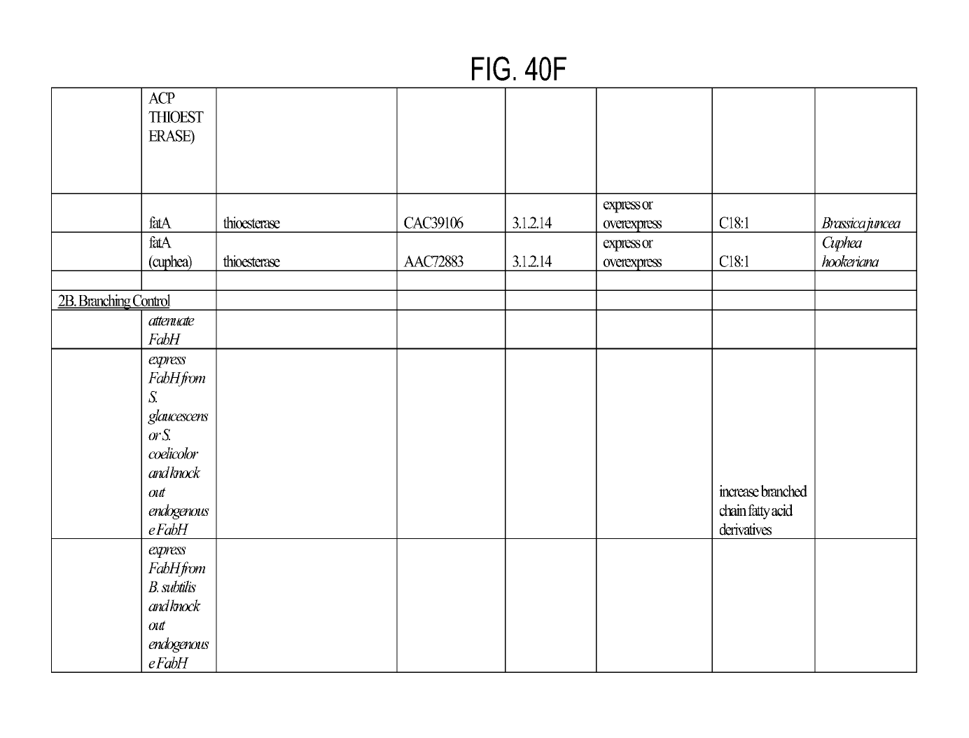

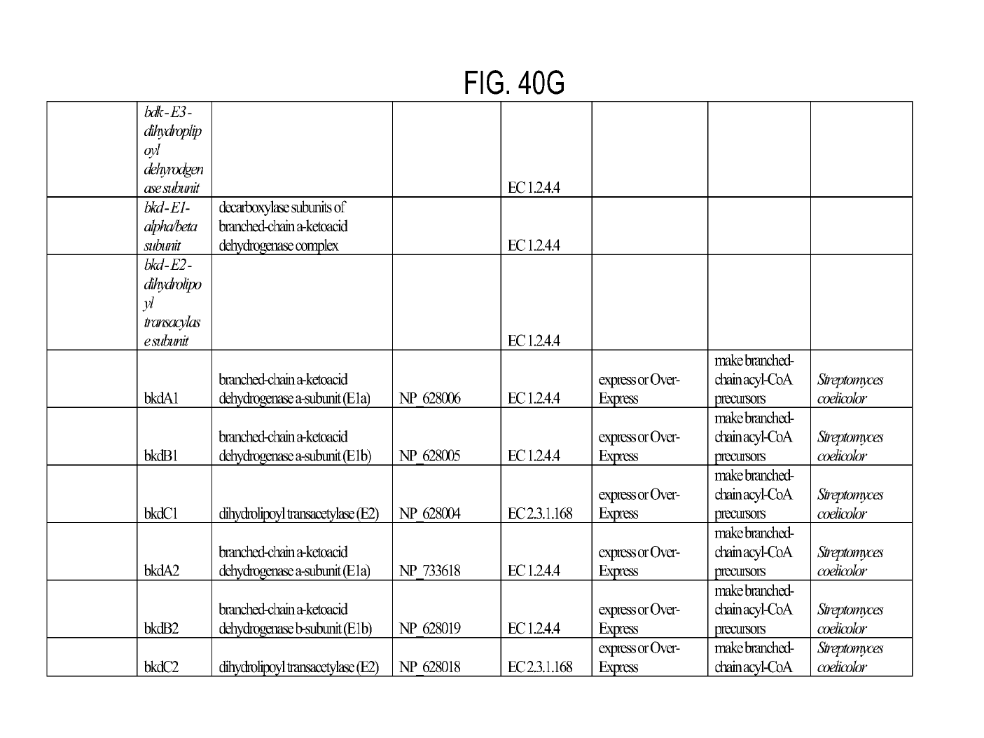

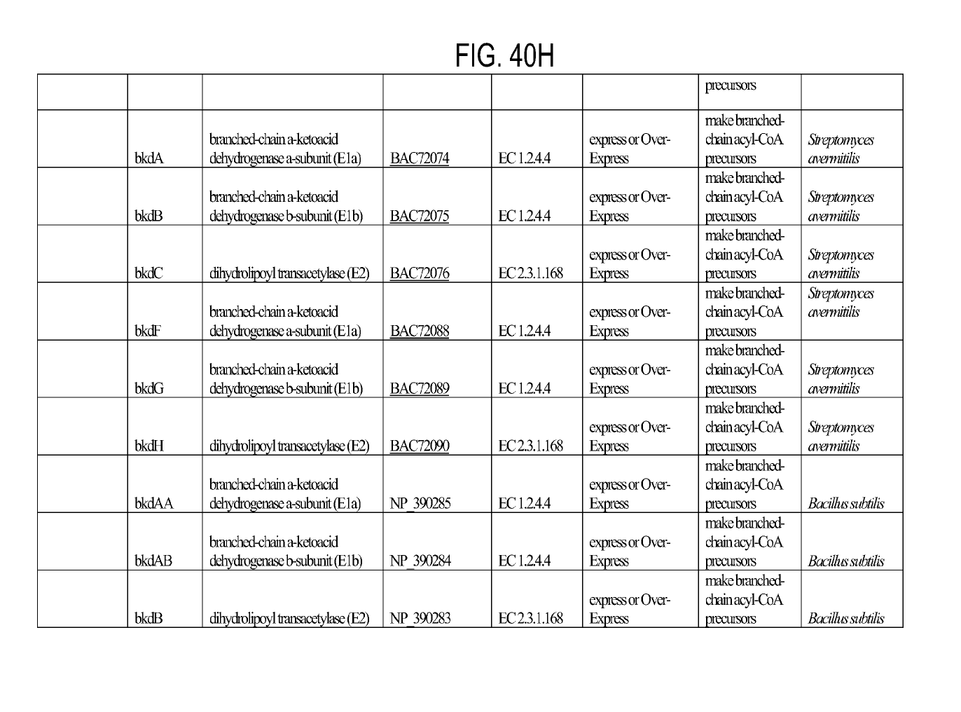

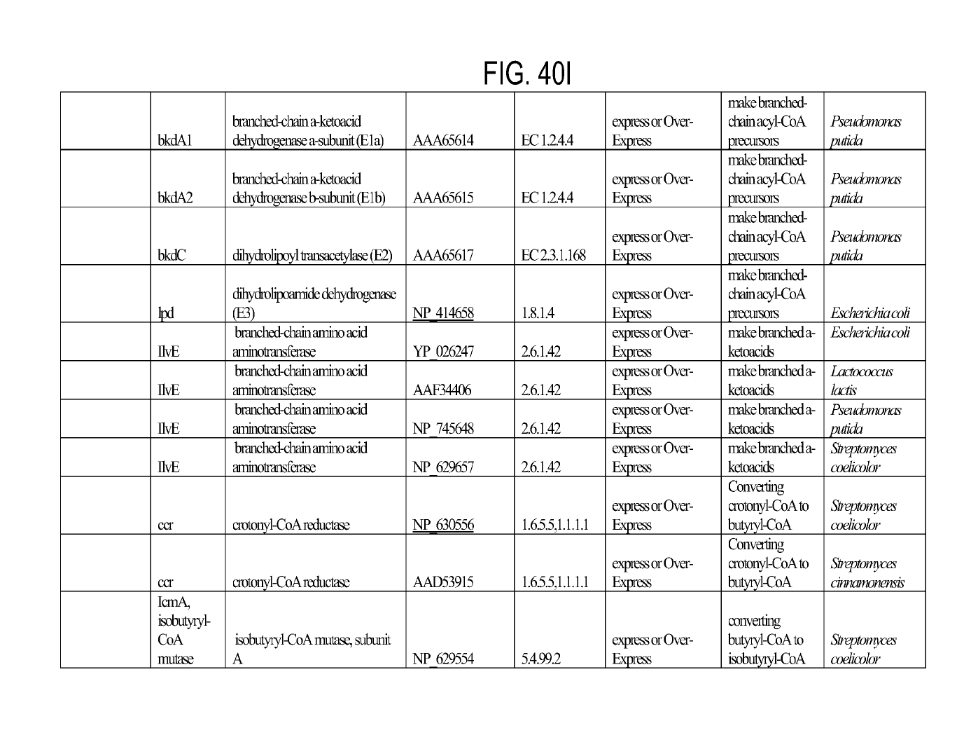

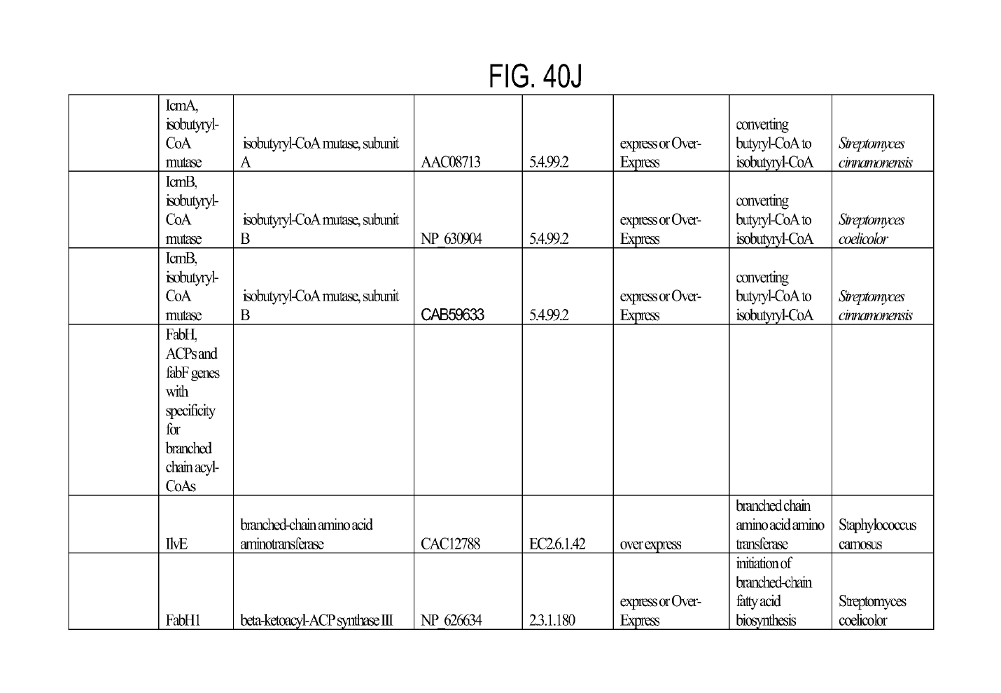

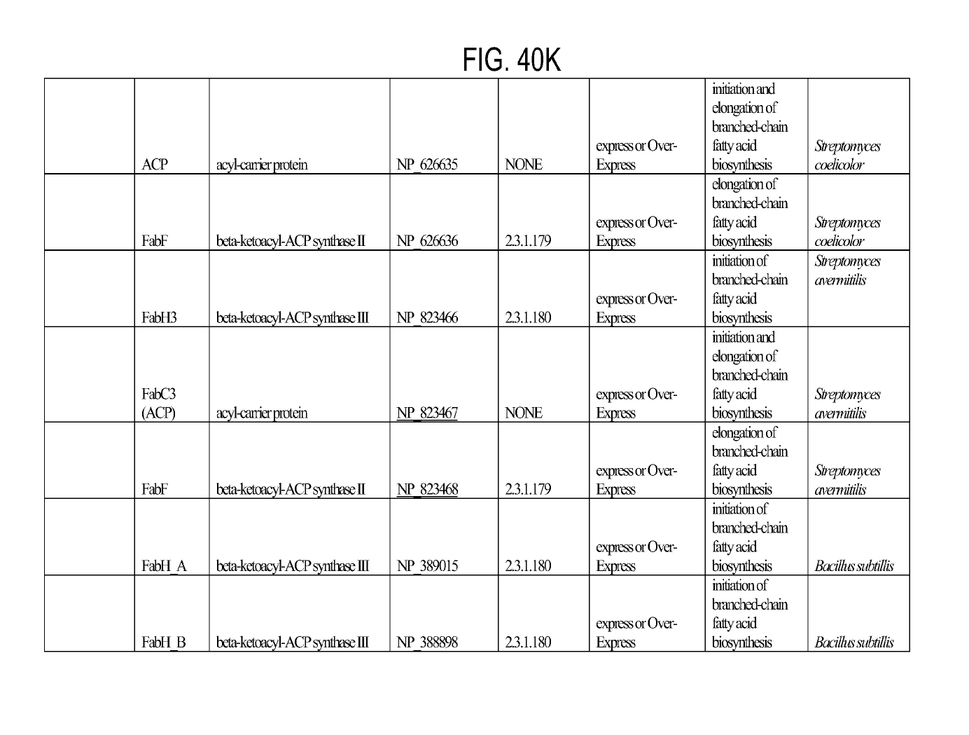

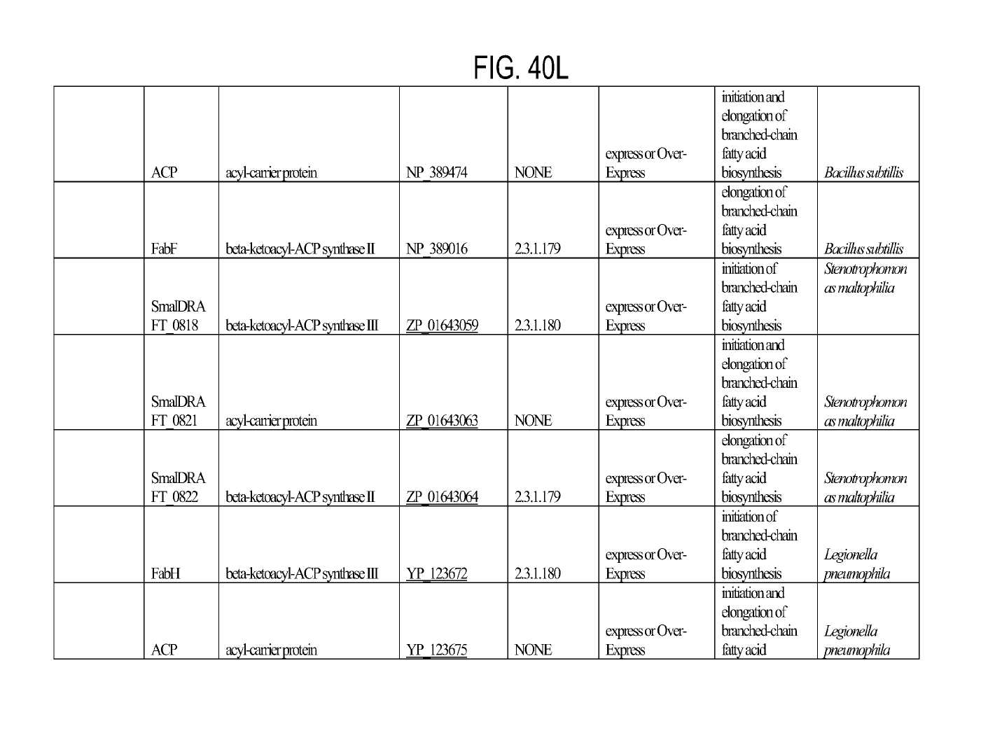

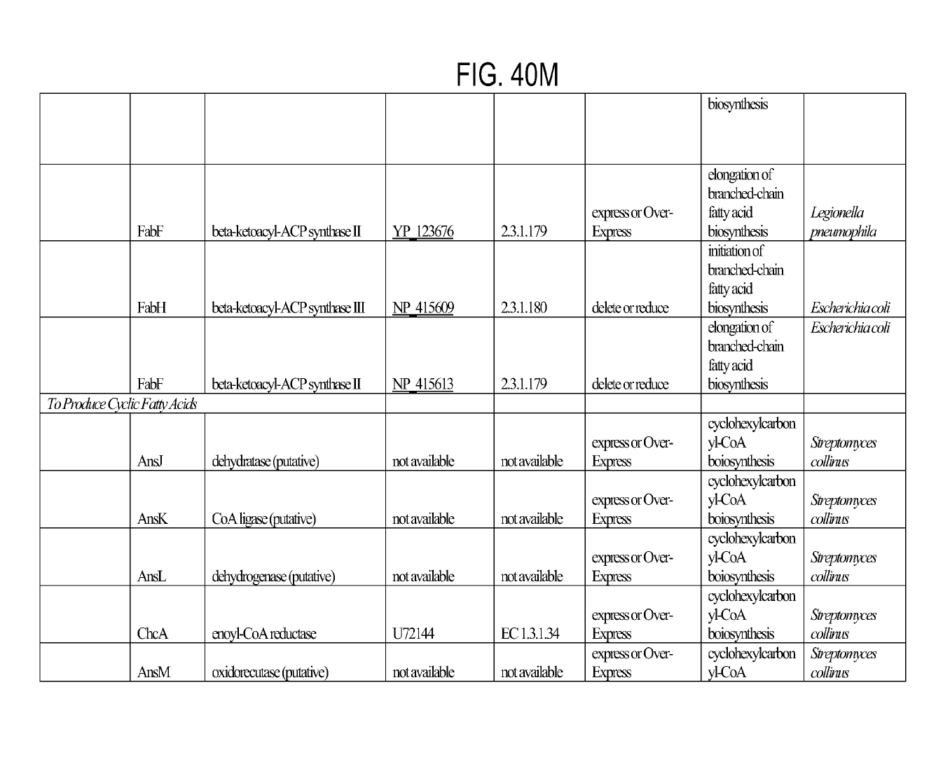

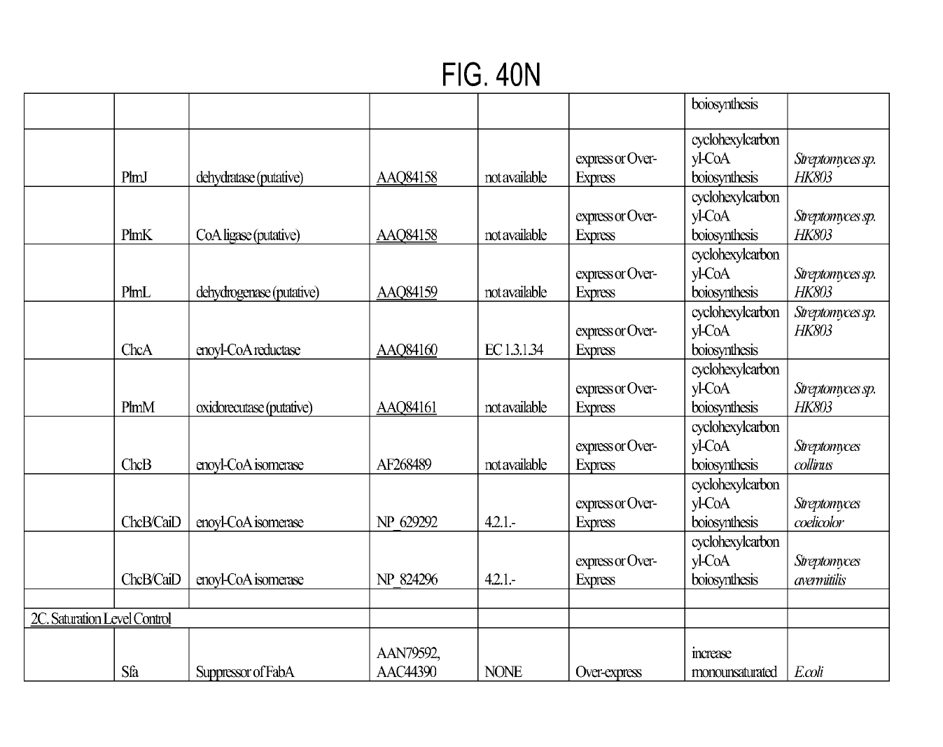

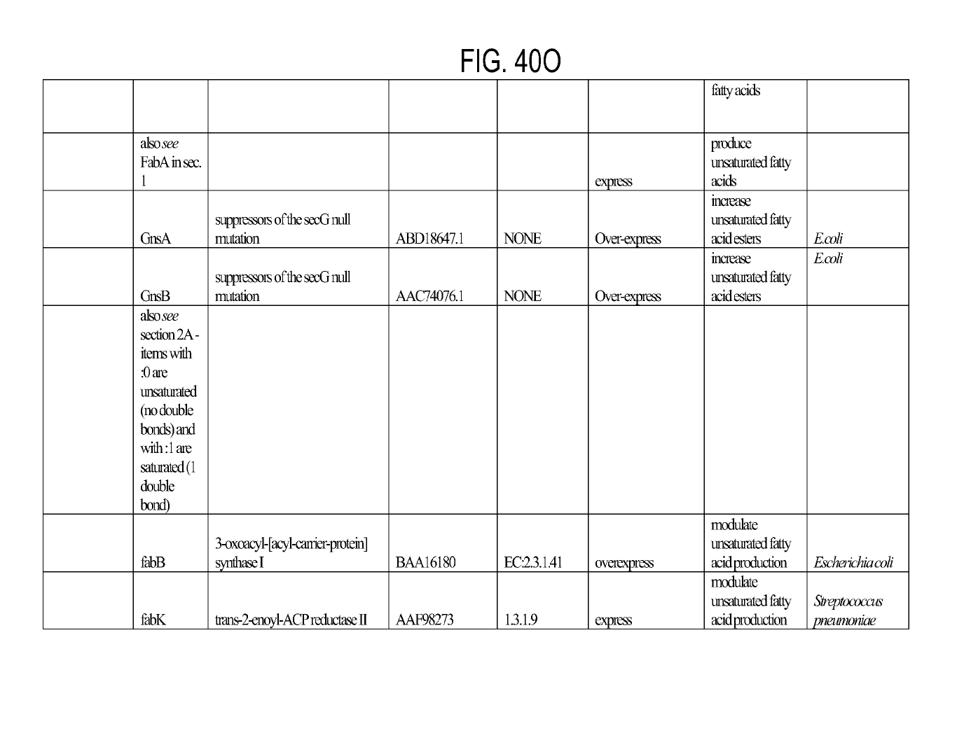

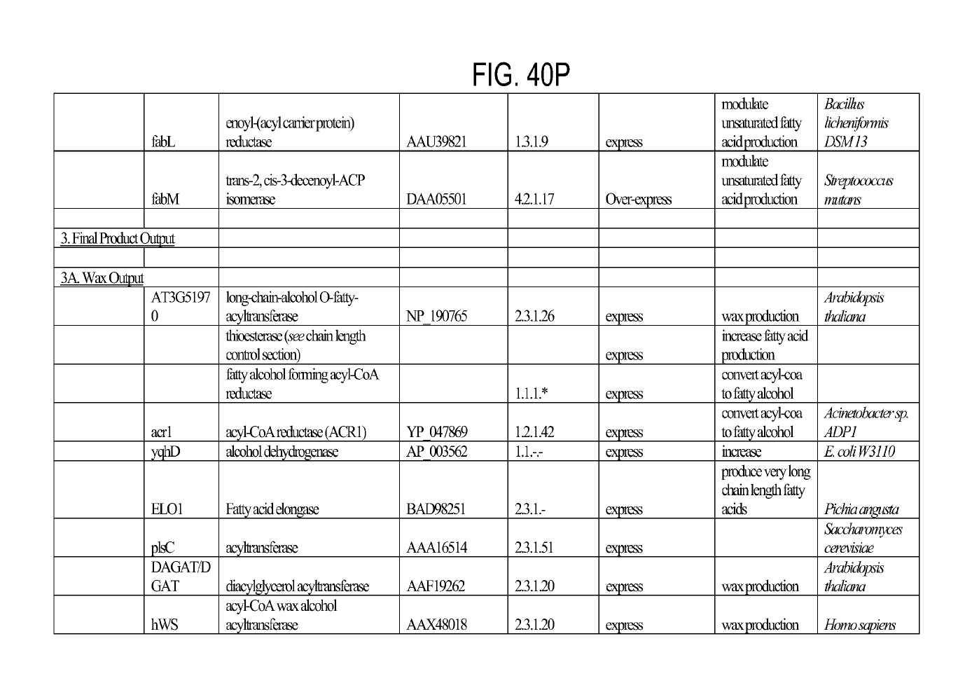

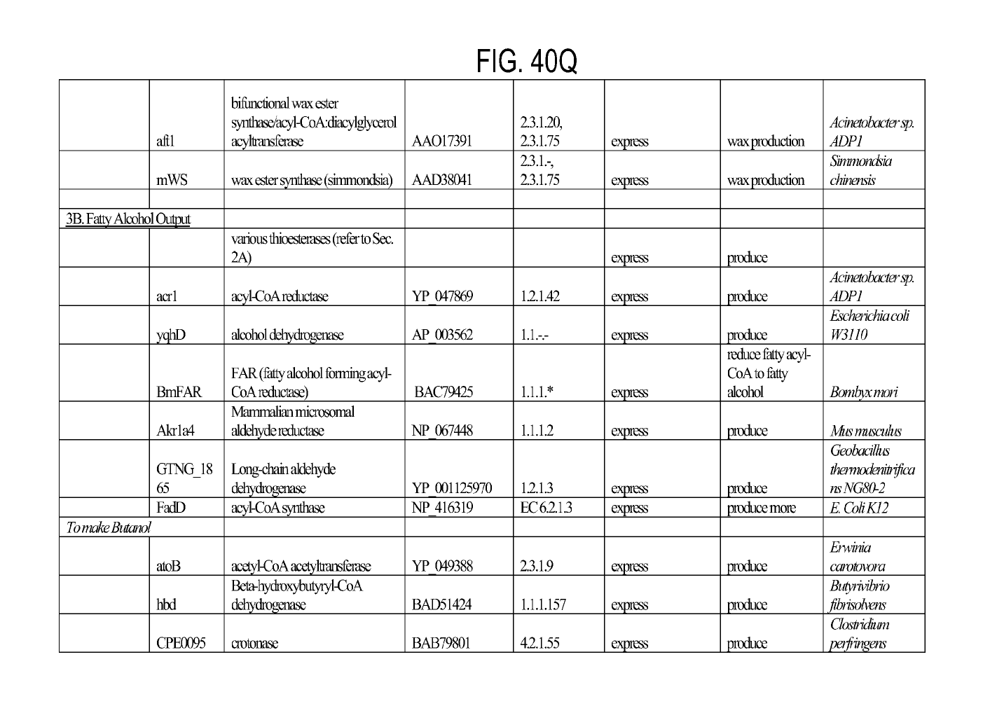

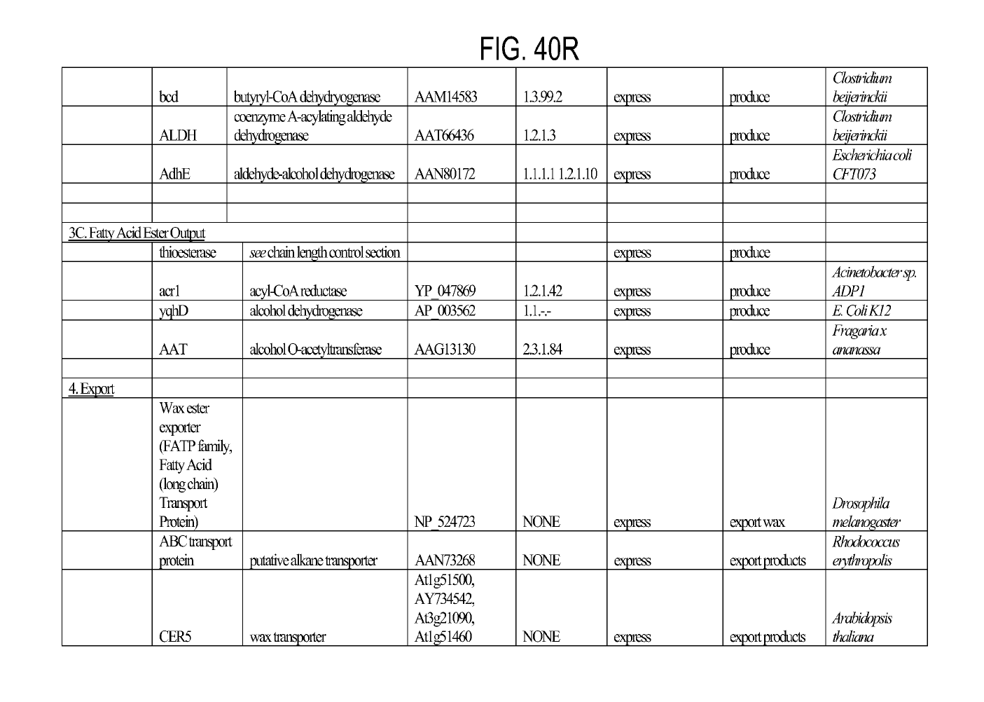

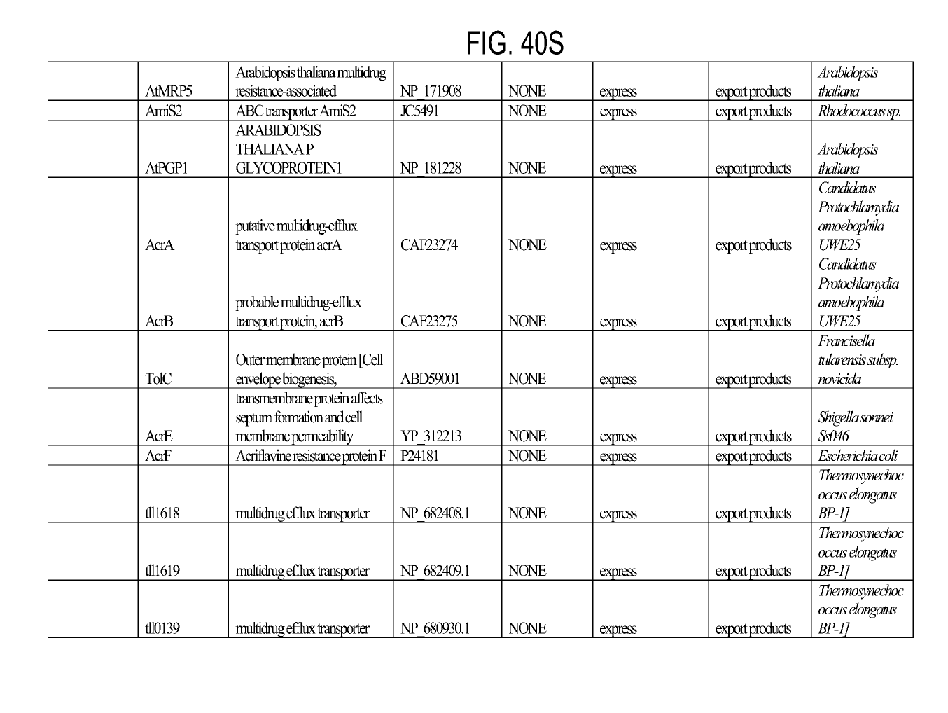

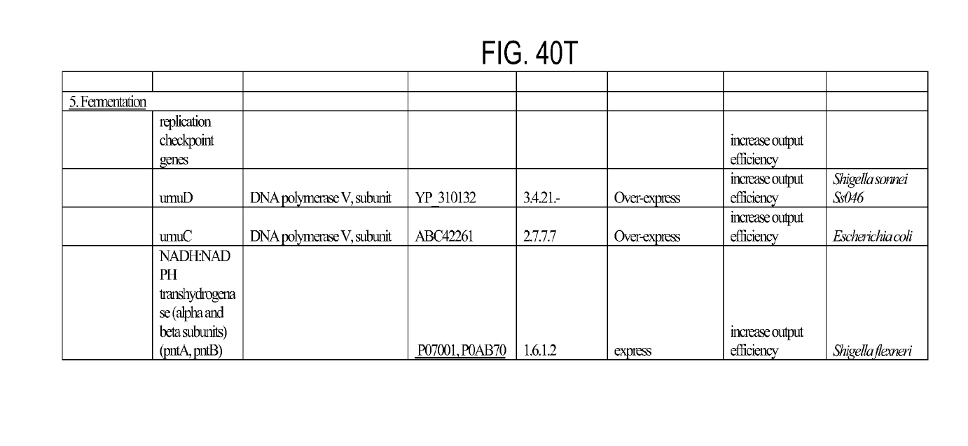

Non-limiting examples of ester synthases are wax synthases, wax-ester synthases, acyl CoA:alcohol transacylases, acyltransferases, and fatty acyl-coenzyme A:fatty alcohol acyltransferases. Exemplary ester synthases are classified in enzyme classification number EC 2.3.1.75. Exemplary GenBank Accession Numbers are provided in FIG. 40.

As used herein, the term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. In a preferred embodiment, the fatty acid is made from a fatty acid biosynthetic pathway.

As used herein, the term “fatty acid biosynthetic pathway” means a biosynthetic pathway that produces fatty acids. The fatty acid biosynthetic pathway includes fatty acid enzymes that can be engineered, as described herein, to produce fatty acids, and in some embodiments can be expressed with additional enzymes to produce fatty acids having desired carbon chain characteristics.

As used herein, the term “fatty acid derivative” means products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered as described herein to produce fatty acid derivatives, and in some examples can be expressed with additional enzymes to produce fatty acid derivatives having desired carbon chain characteristics. Exemplary fatty acid derivatives include for example, fatty acids, acyl-CoA, fatty aldehyde, short and long chain alcohols, hydrocarbons, fatty alcohols, and esters (e.g., waxes, fatty acid esters, or fatty esters).

As used herein, the term “fatty acid derivative enzymes” means all enzymes that may be expressed or over expressed in the production of fatty acid derivatives. These enzymes are collectively referred to herein as fatty acid derivative enzymes. These enzymes may be part of the fatty acid biosynthetic pathway. Non-limiting examples of fatty acid derivative enzymes include fatty acid synthases, thioesterases, acyl-CoA synthases, acyl-CoA reductases, alcohol dehydrogenases, alcohol acyltransferases, fatty alcohol-forming acyl-CoA reductase, ester synthases, aldehyde biosynthetic polypeptides, and alkane biosynthetic polypeptides. Fatty acid derivative enzymes convert a substrate into a fatty acid derivative. In some examples, the substrate may be a fatty acid derivative which the fatty acid derivative enzyme converts into a different fatty acid derivative.

As used herein, the term “fatty alcohol forming peptides” means a peptide capable of catalyzing the conversion of acyl-CoA to fatty alcohol, including fatty alcohol forming acyl-CoA reductase (FAR, EC 1.1.1.*), acyl-CoA reductase (EC 1.2.1.50), or alcohol dehydrogenase (EC 1.1.1.1). Additionally, one of ordinary skill in the art will appreciate that some fatty alcohol forming peptides will catalyze other reactions as well. For example, some acyl-CoA reductase peptides will accept other substrates in addition to fatty acids. Such non-specific peptides are, therefore, also included. Nucleic acid sequences encoding fatty alcohol forming peptides are known in the art, and such peptides are publicly available. Exemplary GenBank Accession Numbers are provided in FIG. 40.

As used herein, “fatty acid enzyme” means any enzyme involved in fatty acid biosynthesis. Fatty acid enzymes can be expressed or over expressed in host cells to produce fatty acids. Non-limiting examples of fatty acid enzymes include fatty acid synthases and thioesterases.

As used herein, the term “fatty ester” means an ester. In a preferred embodiment, a fatty ester is any ester made from a fatty acid, for example a fatty acid ester. In one embodiment, a fatty ester contains an A side (i.e., the carbon chain attached to the carboxylate oxygen) and a B side (i.e., the carbon chain comprising the parent carboxylate).

In a preferred embodiment, when the fatty ester is derived from the fatty acid biosynthetic pathway, the A side is contributed by an alcohol, and the B side is contributed by a fatty acid. Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. The B side of the ester is at least about 4, 6, 8, 10, 12, 14, 16, 18, 20, 22, 24, or 26 carbons in length. The A side and/or the B side can be straight or branched chain. The branched chains may have one or more points of branching. In addition, the branched chains may include cyclic branches. Furthermore, the A side and/or B side can be saturated or unsaturated. If unsaturated, the A side and/or B side can have one or more points of unsaturation.

In one embodiment, the fatty ester is produced biosynthetically. In this embodiment, first the fatty acid is “activated.” Non-limiting examples of “activated” fatty acids are acyl-CoA, acyl-ACP, and acyl phosphate. Acyl-CoA can be a direct product of fatty acid biosynthesis or degradation. In addition, acyl-CoA can be synthesized from a free fatty acid, a CoA, or an adenosine nucleotide triphosphate (ATP). An example of an enzyme which produces acyl-CoA is acyl-CoA synthase.

After the fatty acid is activated, it can be readily transferred to a recipient nucleophile. Exemplary nucleophiles are alcohols, thiols, or phosphates.

In one embodiment, the fatty ester is a wax. The wax can be derived from a long chain alcohol and a long chain fatty acid. In another embodiment, the fatty ester can be derived from a fatty acyl-thioester and an alcohol. In another embodiment, the fatty ester is a fatty acid thioester, for example fatty acyl Coenzyme A (CoA). In other embodiments, the fatty ester is a fatty acyl panthothenate, an acyl carrier protein (ACP), or a fatty phosphate ester. Fatty esters have many uses. For example, fatty esters can be used as a biofuel.

As used herein, “fraction of modern carbon” or “fM” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the 14C/12C isotope ratio HOxI (referenced to AD 1950). This is roughly equivalent to decay-corrected pre-Industrial Revolution wood. For the current living biosphere (plant material), fM is approximately 1.1.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444 453, algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5.

As used herein, a “host cell” is a cell used to produce a product described herein (e.g., an aldehyde or alkane described herein). A host cell can be modified to express or overexpress selected genes or to have attenuated expression of selected genes. Non-limiting examples of host cells include plant, animal, human, bacteria, cyanobacteria, yeast, or filamentous fungi cells.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2.×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified.

The term “isolated” as used herein with respect to nucleic acids, such as DNA or RNA, refers to molecules separated from other DNAs or RNAs, respectively, that are present in the natural source of the nucleic acid. Moreover, an “isolated nucleic acid” includes nucleic acid fragments, such as fragments that are not naturally occurring. The term “isolated” is also used herein to refer to polypeptides, which are isolated from other cellular proteins, and encompasses both purified endogenous polypeptides and recombinant polypeptides. The term “isolated” as used herein also refers to a nucleic acid or polypeptide that is substantially free of cellular material, viral material, or culture medium when produced by recombinant DNA techniques. The term “isolated” as used herein also refers to a nucleic acid or polypeptide that is substantially free of chemical precursors or other chemicals when chemically synthesized.

As used herein, the “level of expression of a gene in a cell” refers to the level of mRNA, pre-mRNA nascent transcript(s), transcript processing intermediates, mature mRNA(s), and/or degradation products encoded by the gene in the cell.

As used herein, the term “microorganism” means prokaryotic and eukaryotic microbial species from the domains Archaea, Bacteria and Eucarya, the latter including yeast and filamentous fungi, protozoa, algae, or higher Protista. The term “microbial cell”, as used herein, means a cell from a microorganism.

As used herein, the term “nucleic acid” refers to polynucleotides, such as deoxyribonucleic acid (DNA), and, where appropriate, ribonucleic acid (RNA). The term also includes analogs of either RNA or DNA made from nucleotide analogs, and, as applicable to the embodiment being described, single (sense or antisense) and double-stranded polynucleotides, ESTs, chromosomes, cDNAs, mRNAs, and rRNAs.

As used herein, the term “operably linked” means that a selected nucleotide sequence (e.g., encoding a polypeptide described herein) is in proximity with a promoter to allow the promoter to regulate expression of the selected nucleotide sequence. In addition, the promoter is located upstream of the selected nucleotide sequence in terms of the direction of transcription and translation. By “operably linked” is meant that a nucleotide sequence and a regulatory sequence(s) are connected in such a way as to permit gene expression when the appropriate molecules (e.g., transcriptional activator proteins) are bound to the regulatory sequence(s).

The term “or” is used herein to mean, and is used interchangeably with, the term “and/or,” unless context clearly indicates otherwise.

As used herein, “overexpress” means to express or cause to be expressed a nucleic acid, polypeptide, or hydrocarbon in a cell at a greater concentration than is normally expressed in a corresponding wild-type cell. For example, a polypeptide can be “overexpressed” in a recombinant host cell when the polypeptide is present in a greater concentration in the recombinant host cell compared to its concentration in a non-recombinant host cell of the same species.

As used herein, “partition coefficient” or “P,” is defined as the equilibrium concentration of a compound in an organic phase divided by the concentration at equilibrium in an aqueous phase (e.g., fermentation broth). In one embodiment of a bi-phasic system described herein, the organic phase is formed by the aldehyde or alkane during the production process. However, in some examples, an organic phase can be provided, such as by providing a layer of octane, to facilitate product separation. When describing a two phase system, the partition characteristics of a compound can be described as log P. For example, a compound with a log P of 1 would partition 10:1 to the organic phase. A compound with a log P of −1 would partition 1:10 to the organic phase. By choosing an appropriate fermentation broth and organic phase, an aldehyde or alkane with a high log P value can separate into the organic phase even at very low concentrations in the fermentation vessel.

As used herein, the term “purify,” “purified,” or “purification” means the removal or isolation of a molecule from its environment by, for example, isolation or separation. “Substantially purified” molecules are at least about 60% free, preferably at least about 75% free, and more preferably at least about 90% free from other components with which they are associated. As used herein, these terms also refer to the removal of contaminants from a sample. For example, the removal of contaminants can result in an increase in the percentage of aldehydes or alkanes in a sample. For example, when aldehydes or alkanes are produced in a host cell, the aldehydes or alkanes can be purified by the removal of host cell proteins. After purification, the percentage of aldehydes or alkanes in the sample is increased.

The terms “purify,” “purified,” and “purification” do not require absolute purity. They are relative terms. Thus, for example, when aldehydes or alkanes are produced in host cells, a purified aldehyde or purified alkane is one that is substantially separated from other cellular components (e.g., nucleic acids, polypeptides, lipids, carbohydrates, or other hydrocarbons). In another example, a purified aldehyde or purified alkane preparation is one in which the aldehyde or alkane is substantially free from contaminants, such as those that might be present following fermentation. In some embodiments, an aldehyde or an alkane is purified when at least about 50% by weight of a sample is composed of the aldehyde or alkane. In other embodiments, an aldehyde or an alkane is purified when at least about 60%, 70%, 80%, 85%, 90%, 92%, 95%, 98%, or 99% or more by weight of a sample is composed of the aldehyde or alkane.

As used herein, the term “recombinant polypeptide” refers to a polypeptide that is produced by recombinant DNA techniques, wherein generally DNA encoding the expressed polypeptide or RNA is inserted into a suitable expression vector and that is in turn used to transform a host cell to produce the polypeptide or RNA.

As used herein, the term “substantially identical” (or “substantially homologous”) is used to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities.

As used herein, the term “synthase” means an enzyme which catalyzes a synthesis process. As used herein, the term synthase includes synthases, synthetases, and ligases.

As used herein, the term “transfection” means the introduction of a nucleic acid (e.g., via an expression vector) into a recipient cell by nucleic acid-mediated gene transfer.

As used herein, “transformation” refers to a process in which a cell's genotype is changed as a result of the cellular uptake of exogenous nucleic acid. This may result in the transformed cell expressing a recombinant form of an RNA or polypeptide. In the case of antisense expression from the transferred gene, the expression of a naturally-occurring form of the polypeptide is disrupted.

As used herein, a “transport protein” is a polypeptide that facilitates the movement of one or more compounds in and/or out of a cellular organelle and/or a cell.

As used herein, a “variant” of polypeptide X refers to a polypeptide having the amino acid sequence of polypeptide X in which one or more amino acid residues is altered. The variant may have conservative changes or nonconservative changes. Guidance in determining which amino acid residues may be substituted, inserted, or deleted without affecting biological activity may be found using computer programs well known in the art, for example, LASERGENE software (DNASTAR).

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. A splice variant may have significant identity to a reference polynucleotide, but will generally have a greater or fewer number of polynucleotides due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

As used herein, the term “vector” refers to a nucleic acid molecule capable of transporting another nucleic acid to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids,” which refer generally to circular double stranded DNA loops that, in their vector form, are not bound to the chromosome. In the present specification, “plasmid” and “vector” are used interchangeably, as the plasmid is the most commonly used form of vector. However, also included are such other forms of expression vectors that serve equivalent functions and that become known in the art subsequently hereto.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

The invention provides compositions and methods of producing aldehydes, fatty alcohols, and hydrocarbons (such as alkanes, alkenes, and alkynes) from substrates, for example, an acyl-ACP, a fatty acid, an acyl-CoA, a fatty aldehyde, or a fatty alcohol substrate (e.g., as described in PCT/US08/058,788, specifically incorporated by reference herein). Such aldehydes, alkanes, and alkenes are useful as biofuels (e.g., substitutes for gasoline, diesel, jet fuel, etc.), specialty chemicals (e.g., lubricants, fuel additive, etc.), or feedstock for further chemical conversion (e.g., fuels, polymers, plastics, textiles, solvents, adhesives, etc.). The invention is based, in part, on the identification of genes that are involved in aldehyde, alkane, and alkene biosynthesis.

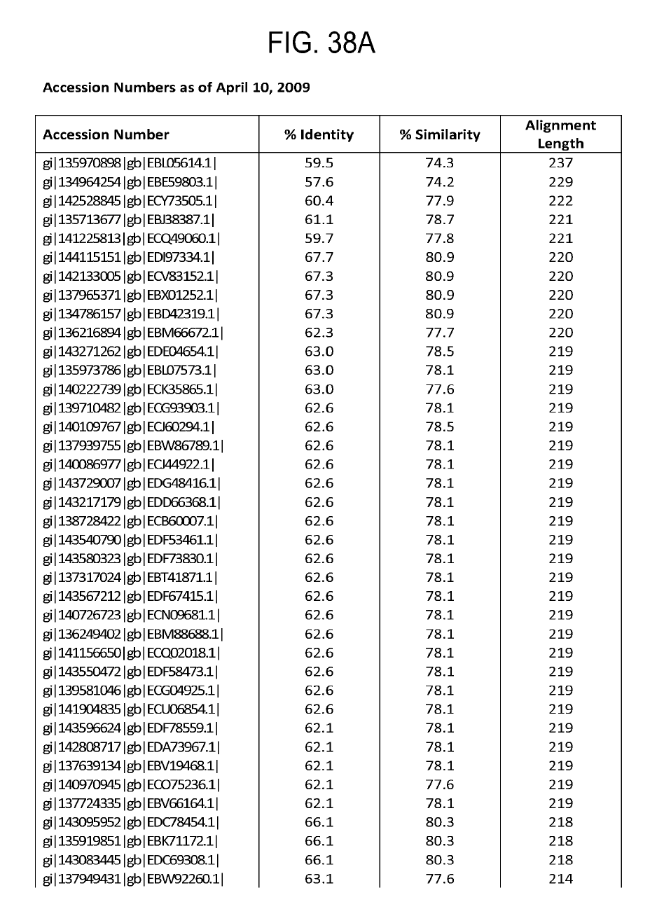

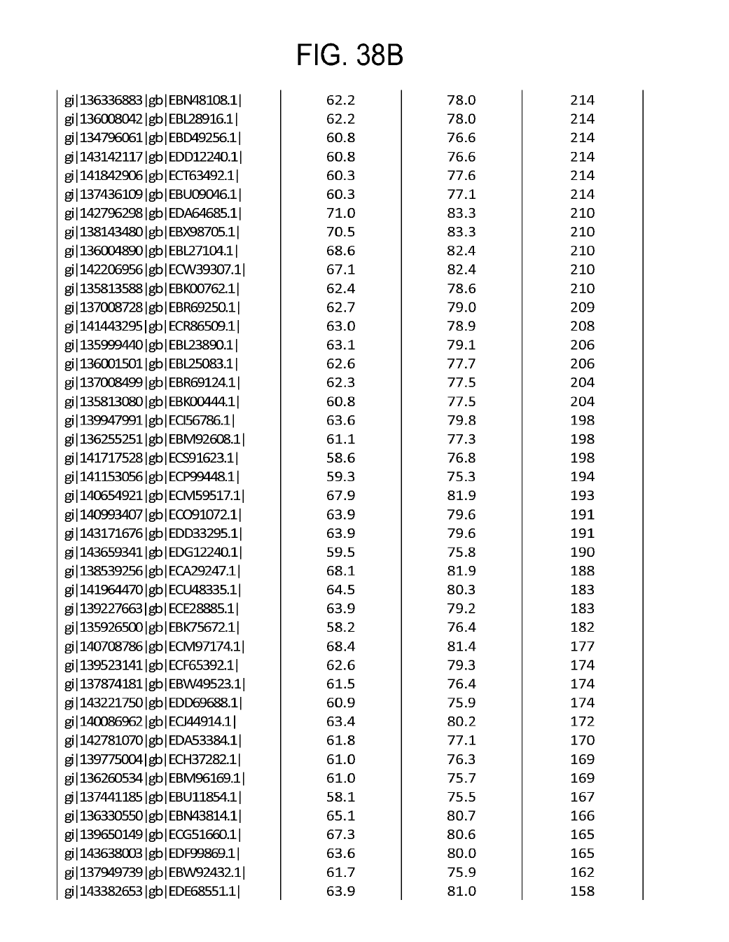

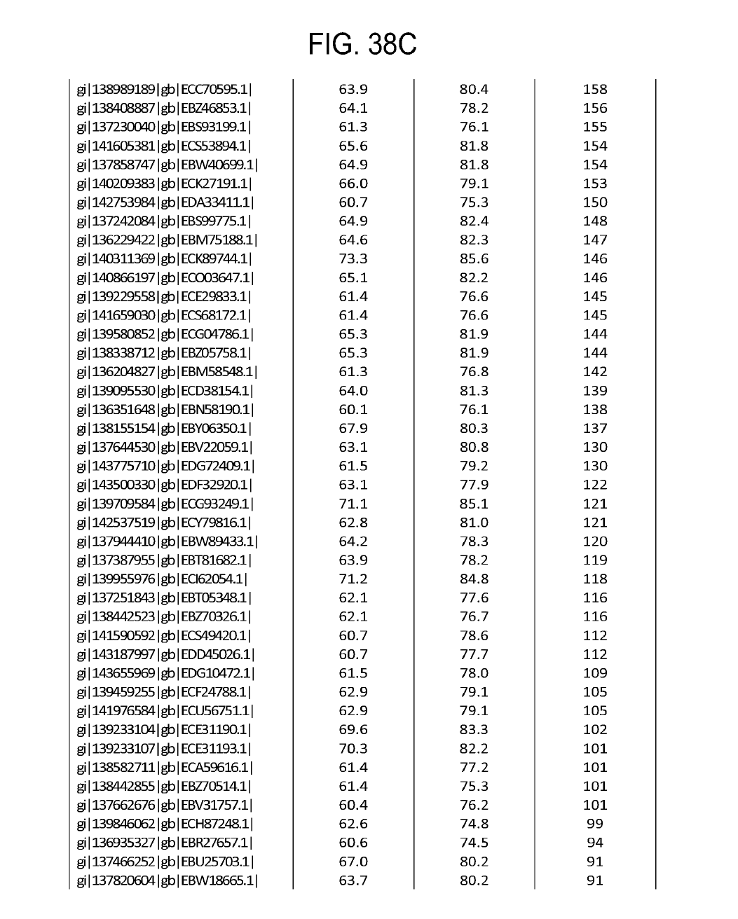

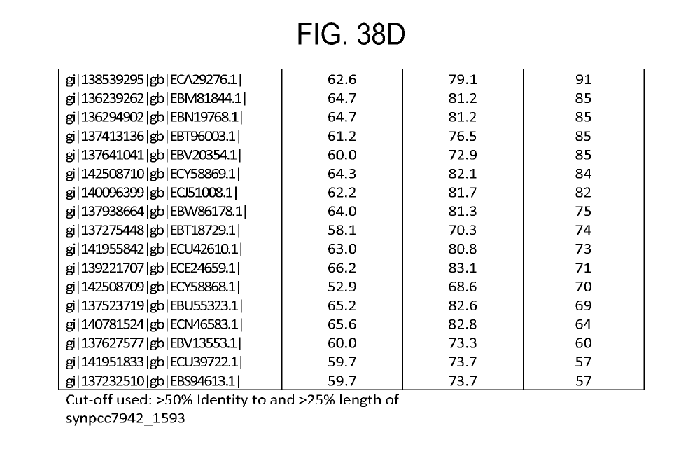

Such alkane and alkene biosynthetic genes include, for example, Synechococcus elongatus PCC7942 Synpcc7942_{—}1593 (SEQ ID NO: 1), Synechocystis sp. PCC6803 sll0208 (SEQ ID NO: 3), Nostoc punctiforme PCC 73102 Npun02004178 (SEQ ID NO: 5), Nostoc sp. PCC 7120 alr5283 (SEQ ID NO: 7), Acaryochloris marina MBIC11017 AM1_{—}4041 (SEQ ID NO: 9), Thermosynechococcus elongatus BP-1 tll1313 (SEQ ID NO: 11), Synechococcus sp. JA-3-3A CYA_{—}0415 (SEQ ID NO: 13), Gloeobacter violaceus PCC 7421 gll3146 (SEQ ID NO: 15), Prochlorococcus marinus MIT9313 PM123 (SEQ ID NO: 17), Prochlorococcus marinus subsp. pastoris str. CCMP1986 PMM0532 (SEQ ID NO: 19), Prochlorococcus marinus str. NATL2A PMN2A_{—}1863 (SEQ ID NO: 21), Synechococcus sp. RS9917 RS9917_{—}09941 (SEQ ID NO: 23), Synechococcus sp. RS9917 RS9917_{—}12945 (SEQ ID NO: 25), Cyanothece sp. ATCC51142 cce_{—}0778 (SEQ ID NO: 27), Cyanothece sp. PCC7245 Cyan7425DRAFT_{—}1220 (SEQ ID NO: 29), Cyanothece sp. PCC7245 cce_{—}0778 (SEQ ID NO: 31), Anabaena variabilis ATCC29413 YP_{—}323043 (Ava_{—}2533) (SEQ ID NO: 33), and Synechococcus elongatus PCC6301 YP_{—}170760 (syc0050_d) (SEQ ID NO: 35). Other alkane and alkene biosynthetic genes are listed in Table 1 and FIG. 38.

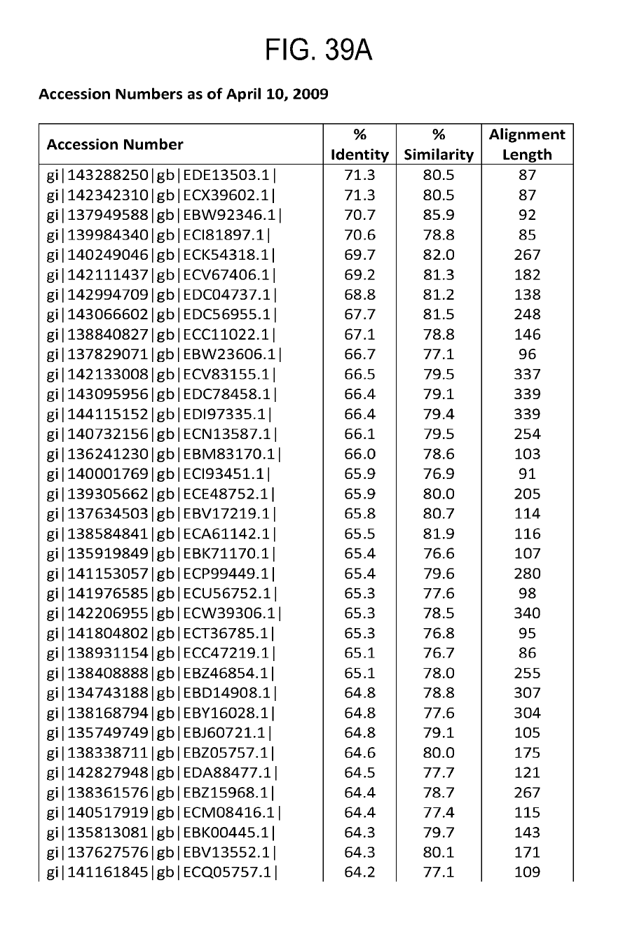

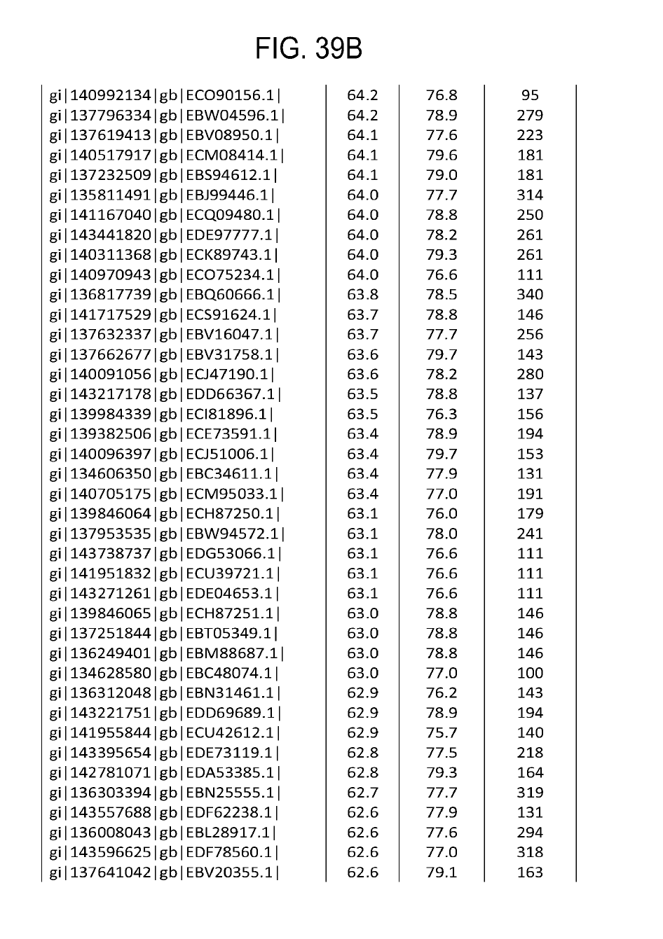

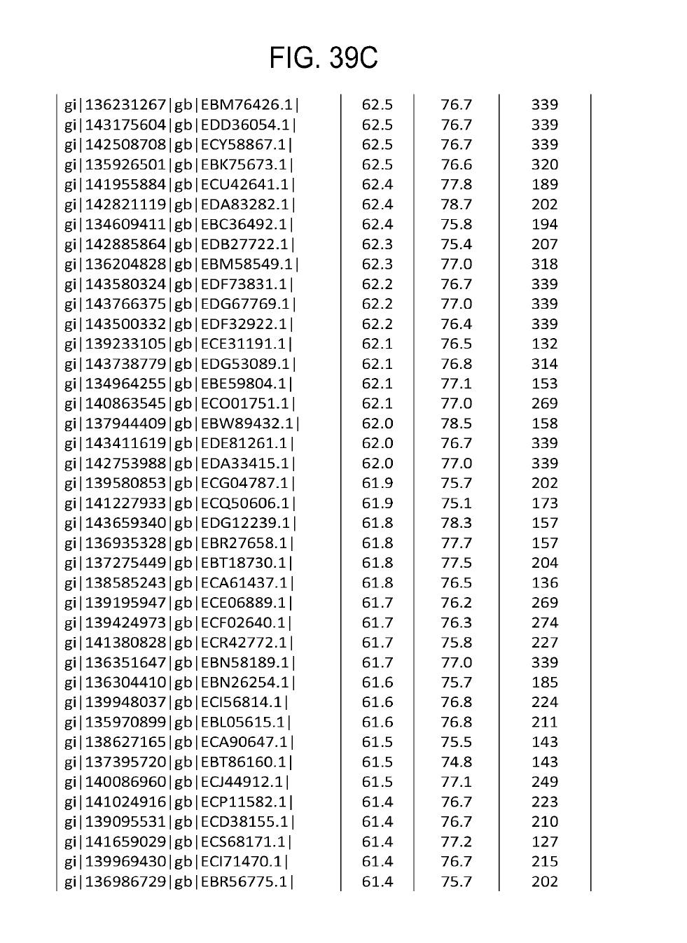

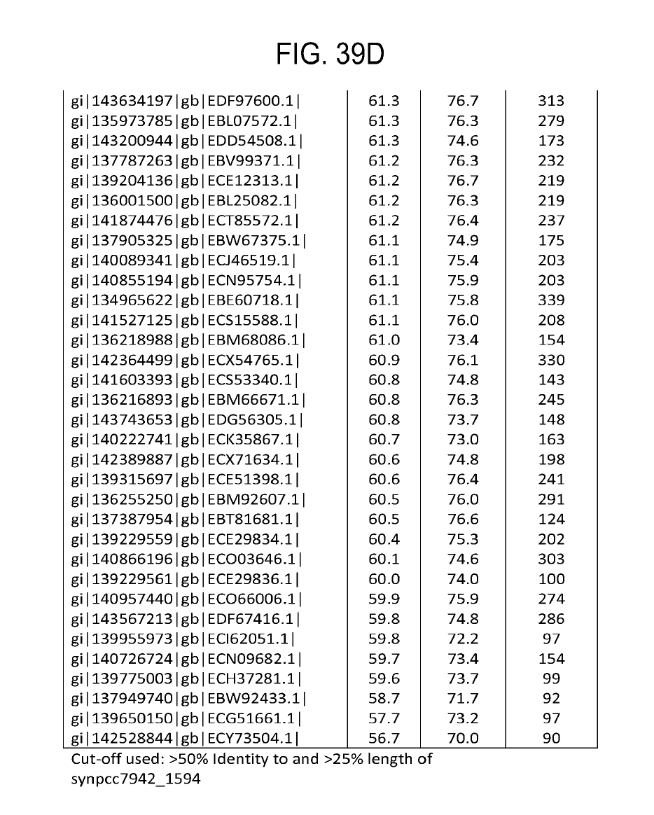

Aldehyde biosynthetic genes include, for example, Synechococcus elongatus PCC7942 Synpcc7942_{—}1594 (SEQ ID NO: 66), Synechocystis sp. PCC6803 sll0209 (SEQ ID NO: 68), Cyanothece sp. ATCC51142 cce_{—}1430 (SEQ ID NO: 70), Prochlorococcus marinus subsp. pastoris str. CCMP1986 PMM0533 (SEQ ID NO: 72), Gloeobacter violaceus PCC7421 NP_{—}926091 (gll3145) (SEQ ID NO: 74), Nostoc punctiforme PCC73102 ZP_{—}00108837 (Npun02004176) (SEQ ID NO: 76), Anabaena variabilis ATCC29413 YP_{—}323044 (Ava_{—}2534) (SEQ ID NO: 78), Synechococcus elongatus PCC6301 YP_{—}170761 (syc0051_d) (SEQ ID NO: 80), and Nostoc sp. PCC 7120 alr5284 (SEQ ID NO: 82). Other aldehyde biosynthetic genes are listed in Table 1 and FIG. 39.

Using the methods described herein, aldehydes, fatty alcohols, alkanes, and alkenes can be prepared using one or more aldehyde, alkane, and/or alkene biosynthetic genes or polypeptides described herein, or variants thereof, utilizing host cells or cell-free methods.

TABLE 1
Aldehyde and alkane biosynthetic gene homologs in cyanobacterial genomes

| | Alkane Biosynth. | | Aldehyde Biosynth. | |
|---|---|---|---|---|
| | Gene | | Gene | |
| Cyanobacterium | Accession No. | % ID | Accession No. | % ID |
| Synechococcus elongatus PCC 7942 | YP_400610 | 100 | YP_400611 | 100 |
| Synechococcus elongatus | YP_170760 | 100 | YP_170761 | 100 |
| PCC 6301 | | | | |
| Microcoleus chthonoplastes | EDX75019 | 77 | EDX74978 | 70 |
| PCC 7420 | | | | |
| Arthrospira maxima CS-328 | EDZ94963 | 78 | EDZ94968 | 68 |
| Lyngbya sp. PCC 8106 | ZP_01619575 | 77 | ZP_01619574 | 69 |
| Nodularia spumigena | ZP_01628096 | 77 | ZP_01628095 | 70 |
| CCY9414 | | | | |
| Trichodesmium erythraeum | YP_721979 | 76 | YP_721978 | 69 |
| IMS101 | | | | |
| Microcystis aeruginosa | YP_001660323 | 75 | YP_001660322 | 68 |
| NIES-843 | | | | |
| Microcystis aeruginosa PCC 7806 | CAO90780 | 74 | CAO90781 | 67 |
| Nostoc sp. PCC 7120 | NP_489323 | 74 | NP_489324 | 72 |
| Nostoc azollae 0708 | EEG05692 | 73 | EEG05693 | 70 |
| Anabaena variabilis ATCC | YP_323043 | 74 | YP_323044 | 73 |
| 29413 | | | | |
| Crocosphaera watsonii WH 8501 | ZP_00514700 | 74 | ZP_00516920 | 67 |
| Synechocystis sp. PCC 6803 | NP_442147 | 72 | NP_442146 | 68 |
| Synechococcus sp. PCC 7335 | EDX86803 | 73 | EDX87870 | 67 |
| Cyanothece sp. ATCC 51142 | YP_001802195 | 73 | YP_001802846 | 67 |
| Cyanothece sp. CCY0110 | ZP_01728578 | 72 | ZP_01728620 | 68 |
| Nostoc punctiforme PCC 73102 | ZP_00108838 | 72 | ZP_00108837 | 71 |
| Acaryochloris marina | YP_001518340 | 71 | YP_001518341 | 66 |
| MBIC11017 | | | | |
| Cyanothece sp. PCC 7425 | YP_002481151 | 71 | YP_002481152 | 70 |
| Cyanothece sp. PCC 8801 | ZP_02941459 | 70 | ZP_02942716 | 69 |
| Thermosynechococcus | NP_682103 | 70 | NP_682102 | 70 |
| elongatus BP-1 | | | | |
| Synechococcus sp. JA-2- | YP_478639 | 68 | YP_478638 | 63 |
| 3B′a (2-13) | | | | |
| Synechococcus sp. RCC307 | YP_001227842 | 67 | YP_001227841 | 64 |
| Synechococcus sp. WH 7803 | YP_001224377 | 68 | YP_001224378 | 65 |
| Synechococcus sp. WH 8102 | NP_897829 | 70 | NP_897828 | 65 |
| Synechococcus sp. WH 7805 | ZP_01123214 | 68 | ZP_01123215 | 65 |
| uncultured marine type-A | ABD96376 | 70 | ABD96375 | 65 |
| Synechococcus GOM 3O12 | | | | |
| Synechococcus sp. JA-3-3Ab | YP_473897 | 68 | YP_473896 | 62 |
| uncultured marine type-A | ABD96328 | 70 | ABD96327 | 65 |
| Synechococcus GOM 306 | | | | |
| uncultured marine type-A | ABD 96275 | 68 | ABD96274 | 65 |
| Synechococcus GOM 3M9 | | | | |
| Synechococcus sp. CC9311 | YP_731193 | 63 | YP_731192 | 63 |
| uncultured marine type-A | ABB92250 | 69 | ABB92249 | 64 |
| Synechococcus 5B2 | | | | |
| Synechococcus sp. WH 5701 | ZP_01085338 | 66 | ZP_01085337 | 67 |
| Gloeobacter violaceus PCC | NP_926092 | 63 | NP_926091 | 67 |
| 7421 | | | | |
| Synechococcus sp. RS9916 | ZP_01472594 | 69 | ZP_01472595 | 66 |
| Synechococcus sp. RS9917 | ZP_01079772 | 68 | ZP_01079773 | 65 |
| Synechococcus sp. CC9605 | YP_381055 | 66 | YP_381056 | 66 |
| Cyanobium sp. PCC 7001 | EDY39806 | 64 | EDY38361 | 64 |
| Prochlorococcus marinus str. | YP_001016795 | 63 | YP_001016797 | 66 |
| MIT 9303 | | | | |
| Prochlorococcus marinus str. | NP_895059 | 63 | NP_895058 | 65 |
| MIT 9313 | | | | |
| Synechococcus sp. CC9902 | YP_377637 | 66 | YP_377636 | 65 |
| Prochlorococcus marinus str. | YP_001090782 | 62 | YP_001090783 | 62 |
| MIT 9301 | | | | |
| Synechococcus sp. BL107 | ZP_01469468 | 65 | ZP_01469469 | 65 |
| Prochlorococcus marinus str. | YP_001008981 | 62 | YP_001008982 | 61 |
| AS9601 | | | | |
| Prochlorococcus marinus str. | YP_397029 | 62 | YP_397030 | 61 |
| MIT9312 | | | | |
| Prochlorococcus marinus | NP_892650 | 60 | NP_892651 | 63 |
| subsp. pastoris str. | | | | |
| CCMP1986 | | | | |
| Prochlorococcus marinus str. | YP_001550420 | 61 | YP_001550421 | 63 |
| MIT 9211 | | | | |
| Cyanothece sp. PCC 7425 | YP_002483683 | 59 | — | |
| Prochlorococcus marinus str. | YP_293054 | 59 | YP_293055 | 62 |
| NATL2A | | | | |
| Prochlorococcus marinus str. | YP_001014415 | 59 | YP_001014416 | 62 |
| NATL1A | | | | |
| Prochlorococcus marinus | NP_874925 | 59 | NP_874926 | 64 |
| subsp. marinus str. | | | | |
| CCMP1375 | | | | |
| Prochlorococcus marinus str. | YP_001010912 | 57 | YP_001010913 | 63 |
| MIT 9515_05961 | | | | |
| Prochlorococcus marinus str. | YP_001483814 | 59 | YP_001483815 | 62 |
| MIT 9215_06131 | | | | |
| Synechococcus sp. RS9917 | ZP_01080370 | 43 | — | |
| uncultured marine type-A | | | ABD96480 | 65 |
| Synechococcus GOM 5D20 | | | | |

### Aldehyde, Alkane, and Alkene Biosynthetic Genes and Variants

The methods and compositions described herein include, for example, alkane or alkene biosynthetic genes having the nucleotide sequence of SEQ ID NO: 1, 3, 5, 7, 9, 11, 13, 15, 17, 19, 21, 23, 25, 27, 29, 31, 33, or 35, as well as polynucleotide variants thereof. In some instances, the alkane or alkene biosynthetic gene encodes one or more of the amino acid motifs described herein. For example, the alkane or alkene biosynthetic gene can encode a polypeptide comprising SEQ ID NO: 38, 39, 40, 42, 43, 44, or 45. The alkane or alkene biosynthetic gene can also include a polypeptide comprising SEQ ID NO: 41 and also any one of SEQ ID NO: 38, 39, or 40. The methods and compositions described herein also include, for example, alkane or alkene biosynthetic genes encoding a polypeptide comprising SEQ ID NO: 37 or 115, or encoding variants, fragments, analogs or derivatives of SEQ ID NO: 37 or 115.

The methods and compositions described herein also include, for example, aldehyde biosynthetic genes having the nucleotide sequence of SEQ ID NO: 66, 68, 70, 72, 74, 76, 78, 80, or 82, as well as polynucleotide variants thereof. In some instances, the aldehyde biosynthetic gene encodes one or more of the amino acid motifs described herein. For example, the aldehyde biosynthetic gene can encode a polypeptide comprising SEQ ID NO: 55, 56, 57, 58, 59, 60, 61, 62, 63, 64, or 65.

The variants can be naturally occurring or created in vitro. In particular, such variants can be created using genetic engineering techniques, such as site directed mutagenesis, random chemical mutagenesis, Exonuclease III deletion procedures, and standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be created using error prone PCR (see, e.g., Leung et al., Technique 1:11-15, 1989; and Caldwell et al., PCR Methods Applic. 2:28-33, 1992). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized (e.g., an aldehyde or alkane biosynthetic polynucleotide sequence), are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized (e.g., an aldehyde or alkane biosynthetic polynucleotide sequence), 30 pmole of each PCR primer, a reaction buffer comprising 50 mM KCl, 10 mM Tris HCl (pH 8.3), and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 mM, 45° C. for 1 min, and 72° C. for 1 min. However, it will be appreciated that these parameters can be varied as appropriate. The mutagenized nucleic acids are then cloned into an appropriate vector and the activities of the polypeptides encoded by the mutagenized nucleic acids are evaluated.

Variants can also be created using oligonucleotide directed mutagenesis to generate site-specific mutations in any cloned DNA of interest. Oligonucleotide mutagenesis is described in, for example, Reidhaar-Olson et al., Science 241:53-57, 1988. Briefly, in such procedures a plurality of double stranded oligonucleotides bearing one or more mutations to be introduced into the cloned DNA are synthesized and inserted into the cloned DNA to be mutagenized (e.g., an aldehyde or alkane biosynthetic polynucleotide sequence). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another method of generating variants is sexual PCR mutagenesis. In sexual PCR mutagenesis, forced homologous recombination occurs between DNA molecules of different, but highly related, DNA sequence in vitro as a result of random fragmentation of the DNA molecule based on sequence homology. This is followed by fixation of the crossover by primer extension in a PCR reaction. Sexual PCR mutagenesis is described in, for example, Stemmer, PNAS, USA 91:10747-10751, 1994.

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., an aldehyde or alkane biosynthetic polynucleotide sequence) in one of these strains will eventually generate random mutations within the DNA. Mutator strains suitable for use for in vivo mutagenesis are described in, for example, PCT Publication No. WO 91/16427.

Variants can also be generated using cassette mutagenesis. In cassette mutagenesis, a small region of a double stranded DNA molecule is replaced with a synthetic oligonucleotide “cassette” that differs from the native sequence. The oligonucleotide often contains a completely and/or partially randomized native sequence.

Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., PNAS, USA 89:7811-7815, 1992.

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res. 11:1548-1552, 1993. Random and site-directed mutagenesis are described in, for example, Arnold, Curr. Opin. Biotech. 4:450-455, 1993.

In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

Polynucleotide variants also include nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine and 5-methyl-2′-deoxycytidine or 5-bromo-2′-doxycytidine for deoxycytidine. Modifications of the sugar moiety include modification of the 2′ hydroxyl of the ribose sugar to form 2′-deoxy, 2′-fluoro, 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. (See, e.g., Summerton et al., Antisense Nucleic Acid Drug Dev. (1997) 7:187-195; and Hyrup et al., Bioorgan. Med. Chem. (1996) 4:5-23.) In addition, the deoxyphosphate backbone can be replaced with, for example, a phosphorothioate or phosphorodithioate backbone, a phosphoroamidite, or an alkyl phosphotriester backbone.

The aldehyde and alkane biosynthetic polypeptides Synpcc7942_{—}1594 (SEQ ID NO: 67) and Synpcc7942_{—}1593 (SEQ ID NO: 2) have homologs in other cyanobacteria (nonlimiting examples are listed in Table 1). Thus, any polynucleotide sequence encoding a homolog listed in Table 1, or a variant thereof, can be used as an aldehyde or alkane/alkene biosynthetic polynucleotide in the methods described herein. Each cyanobacterium listed in Table 1 has copies of both genes. The level of sequence identity of the gene products ranges from 61% to 73% for Synpcc7942_{—}1594 (SEQ ID NO: 67) and from 43% to 78% for Synpcc7942_{—}1593 (SEQ ID NO: 2).

It has been discovered that hydrocarbon biosynthetic polypeptides share a certain spatially conserved amino acid sequence motif comprising SEQ ID NO: 37. Exemplary alkane/alkene biosynthetic polypeptide of the invention include SEQ ID NO: 105, SEQ ID NO: 106, SEQ ID NO: 107, and SEQ ID NO: 108.

An additional amino acid sequence has been discovered that may play a role in the activity levels of hydrocarbon biosynthetic polypeptides. The motif comprises SEQ ID NO: 115. Without wishing to be bound by any particular theory, the location of certain residues and their interaction within the primary, secondary, tertiary, and/or quaternary structure of the enzyme have a direct effect on the activity of the enzyme. For example, the proximity of a residue to a metal center(s) of a hydrocarbon biosynthetic polypeptide likely influences its involvement in in vivo activity of the enzyme. Exemplary alkane/alkene biosynthetic polypeptides of the invention include SEQ ID NO: 151, SEQ ID NO: 152, SEQ ID NO: 153, and SEQ ID NO: 154. Without wishing to be bound by any particular theory, it is believed that polypeptides of SEQ ID NO: 151-154 have decarbolylase activity that is at least the same as that of wild type decarbonylase enzymes. Preferably, polypeptides of SEQ ID NO: 151-154 have improved decarbonylase activity when compared with wild type decarbonylase enzymes.

In one aspect the invention features a polypeptide consisting of SEQ ID NO: 37 or 115. In certain embodiments, 1 or more, for example, 2 or more, 3 or more, 4 or more, or 5 or more of the amino acid residues of SEQ ID NO: 37 or 115 interact with, bond to, or chelate 1 or more metal ions, for example, 2 or more, or 3 or more metal ions. The metal ions can be important for the enzymatic activity of the polypeptide to which they are bound. In some embodiments, that enzymatic activity is a decarbonylase activity of a polypeptide comprising SEQ ID NO: 37 or 115. In a particular embodiment, 6 amino acid residues of SEQ ID NO: 37 or 115 chelate 2 metal ions.

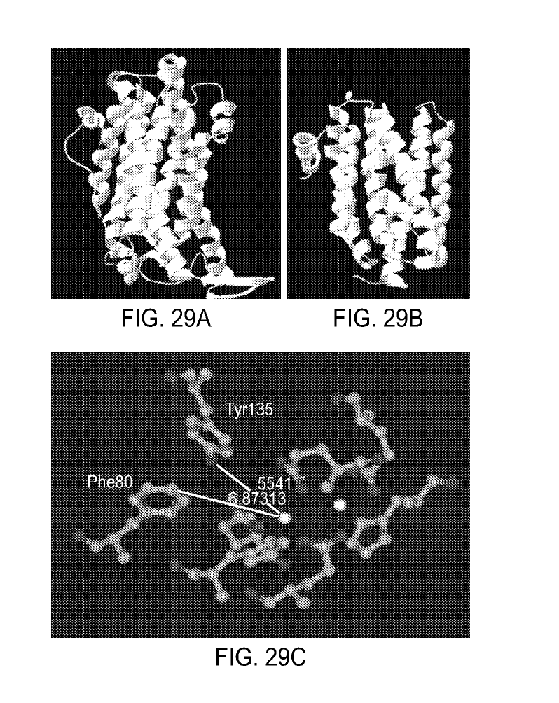

An alkane/alkene biosynthetic polypeptide of invention can have a metal center, which comprises one or more proximately-situated metal ions. For example, two metal ions can be seen in a three-dimensional crystallographic structure of Prochlorococcus marinus MIT9313 PMT1231, NP_{—}895059 (SEQ ID NO: 18) (Protein Data Bank atomic coordinates Accession No. 2oc5A) in FIG. 29B. Alternatively, the putative metal center of an alkane/alkene biosynthetic polypeptide of the invention can be fitted to the metal center of an alkane/alkene biosynthetic polypeptide of known tertiary structure, such as, for example, the Prochlorococcus marinus MIT9313 PMT1231, NP_{—}895059.

In certain aspects, the metal center may comprise 1, 2, 3, or more metal ions. In an exemplary embodiment, an alkane/alkene biosynthetic polypeptide comprises a metal center with 2 metal atoms. The metal ions may suitably be selected from the group consisting of: Zn²⁺, Mn²⁺, Fe²⁺, Mg²⁺, Na⁺, K⁺, and Cu²⁺, or a combination thereof. Alternatively, the metal ions can be selected from transition metal ions or a combination thereof. Each of the metal ions can independently be quadrivalent, trivalent, divalent or univalent. For example, the first of the metal ions can be a trivalent metal ion whereas the second can be a divalent metal ion. Alternatively, both the first and the second metal ions can be bivalent metal ions. Moreover, both the first and the second metal ions can be trivalent metal ions. An exemplary alkane/alkene biosynthetic polypeptide of the invention, which comprises SEQ ID NO: 37 or 115, has two Fe²⁺ ions at the metal center. An alternative exemplary alkane/alkene biosynthetic polypeptide of the invention comprises a Fe²⁺ and a Mn²⁺ at the metal center. Other alternative exemplary alkane/alkene biosynthetic polypeptides of the invention comprise a Fe²⁺ and a Fe³⁺, or a Fe²⁺ and a Fe⁴⁺, or a Fe³⁺ and a Fe⁴⁺ at the metal center.

It has also been discovered that certain other amino acid residues, while not bound to or directly interact with the metal ions, are located within close proximity to the metal center, and thus potentially contribute to the formation and maintenance of the three-dimensional metal center structure. The amino acid residues that are within about 10 Å, for example, about 8 Å, about 6 Å, or about 5 Å, of the metal ions, as well as those that are bound to the metal ions, are collectively termed “metal-center amino acid residues” herein. For example, it has been identified that two alanine residues, two tyrosine residues, a valine residue, an aspartic acid residue, a phenylalanine residue, and a glutamine residue, which are not bound to the two irons, as well as four glutamic acid residues and two histidine residues, which are bound to the two irons, are metal-center amino acid residues of Prochlorococcus marinus MIT9313 PMT1231, NP_{—}895059.

In related aspects, one or more amino acid residues bound to the metal ions are within the linear amino acid sequence of SEQ ID NO: 37 or 115. In some embodiments, the amino acid residues bound to the metal ions may be one or more of glutamic acid residues and histidine residues.

In a particular embodiment, the metal-bound amino acid residues are present in a motif comprising SEQ ID NO: 96. In some embodiments, the motif sequence SEQ ID NO: 37 comprises SEQ ID NO: 96. In certain other embodiments, one or more amino acid residues that are bound to the metal ions are not amino acid residues of SEQ ID NO: 37. In other embodiments, the amino acid sequence SEQ ID NO: 37 does not comprise amino acid residues that chelate the metal ions. Alternatively, the amino acid sequence SEQ ID NO: 37 does not comprise SEQ ID NO: 96, such that the metal-bound amino acid residues of SEQ ID NO: 84 are independent of SEQ ID NO: 37.

Further homologs of the aldehyde biosynthetic polypeptide Synpcc7942_{—}1594 (SEQ ID NO: 67) are listed in FIG. 39, and any polynucleotide sequence encoding a homolog listed in FIG. 39, or a variant thereof, can be used as an aldehyde biosynthetic polynucleotide in the methods described herein. Further homologs of the alkane biosynthetic polypeptide Synpcc7942_{—}1593 (SEQ ID NO: 2) are listed in FIG. 38, and any polynucleotide sequence encoding a homolog listed in FIG. 38, or a variant thereof, can be used as an alkane biosynthetic polynucleotide in the methods described herein.

In certain instances, an aldehyde, alkane, and/or alkene biosynthetic gene is codon optimized for expression in a particular host cell. For example, for expression in E. coli, one or more codons can be optimized as described in, e.g., Grosjean et al., Gene 18:199-209 (1982). In an exemplary embodiment, an alkane/alkene biosynthetic gene encoding an alkane/alkene biosynthetic polypeptide is codon optimized for expression in E. coli., wherein the alkane/alkene biosynthetic polypeptide comprises SEQ ID NO: 37 or 115. In another embodiment, the alkane/alkene biosynthetic polypeptide has a metal center tertiary structure of an enzyme with the atomic coordinates of Protein Data Bank Accession No. 2oc5A. In yet another embodiment, the alkane/alkene biosynthetic polypeptide comprises putative metal-center amino acid residues, which can be fitted to the metal-center amino acid residues of an alkane/alkene biosynthetic polypeptide of known structure. An exemplary alkane/alkene biosynthetic polypeptide of known structure is Prochlorococcus marinus MIT9313 PMT1231.

### Aldehyde, Alkane, and Alkene Biosynthetic Polypeptides and Variants

The methods and compositions described herein also include alkane or alkene biosynthetic polypeptides having the amino acid sequence of SEQ ID NO: 2, 4, 6, 8, 10, 12, 14, 16, 18, 20, 22, 24, 26, 28, 30, 32, 34, or 36, as well as polypeptide variants thereof. In some instances, an alkane or alkene biosynthetic polypeptide is one that includes one or more of the amino acid motifs described herein. For example, the alkane or alkene biosynthetic polypeptide can include the amino acid sequence of SEQ ID NO: 38, 39, 40, 42, 43, 44, or 45. The alkane or alkene biosynthetic polypeptide can also include the amino acid sequence of SEQ ID NO: 41 and also any one of SEQ ID NO: 38, 39, or 40.

The method and compositions described herein include alkane/alkene biosynthetic polypeptides comprising an amino acid sequence of SEQ ID NO: 37 or 115. Moreover, the method and compositions described herein also include alkane/alkene biosynthetic polypeptides comprising a variant, preferably a conservative variant, of SEQ ID NO: 37 or 115. In certain aspects, the alkane/alkene biosynthetic polypeptide comprises a metal center tertiary structure of an enzyme with the atomic coordinates of Protein Data Bank Accession No. 2oc5A. In other aspects, the alkane/alkene biosynthetic polypeptides comprise putative metal-center amino acid residues, which can be fitted to the metal-center amino acid residues of an alkane/alkene biosynthetic polypeptide of known structure. An exemplary alkane/alkene biosynthetic polypeptide of known structure is Prochlorococcus marinus MIT9313 PMT1231, NP_{—}895059.

The methods and compositions described herein also include aldehyde biosynthetic polypeptides having the amino acid sequence of SEQ ID NO: 67, 69, 71, 73, 75, 77, 79, 81, or 83, as well as polypeptide variants thereof. In some instances, an aldehyde biosynthetic polypeptide is one that includes one or more of the amino acid motifs described herein. For example, the aldehyde biosynthetic polypeptide can include the amino acid sequence of SEQ ID NO: 55, 56, 57, 58, 59, 60, 61, 62, 63, 64, or 65.

Aldehyde, alkane, and alkene biosynthetic polypeptide variants can be variants in which one or more amino acid residues are substituted with a conserved or non-conserved amino acid residue, preferably a conserved amino acid residue. Such substituted amino acid residue may or may not be one encoded by the genetic code.

Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Other polypeptide variants are those in which one or more amino acid residues include a substituent group. Still other polypeptide variants are those in which the polypeptide is associated with another compound, such as a compound to increase the half-life of the polypeptide (e.g., polyethylene glycol).

Additional polypeptide variants are those in which additional amino acids are fused to the polypeptide, such as a leader sequence, a secretory sequence, a proprotein sequence, or a sequence which facilitates purification, enrichment, or stabilization of the polypeptide.

In some instances, an alkane or alkene biosynthetic polypeptide variant retains the same biological function as a polypeptide having the amino acid sequence of SEQ ID NO: 2, 4, 6, 8, 10, 12, 14, 16, 18, 20, 22, 24, 26, 28, 30, 32, 34, or 36 (e.g., retains alkane or alkene biosynthetic activity) and has an amino acid sequence substantially identical thereto.

In related instances, an alkane or alkene biosynthetic polypeptide variant retains the same biological function of a polypeptide having the amino acid sequence of SEQ ID NO: 37 or 115 and comprises an amino acid sequence substantially identical to SEQ ID NO: 37 or 115. In yet another aspect, an alkane/alkene biosynthetic polypeptide variant has the same biological function of a polypeptide with the three-dimensional atomic coordinates of Protein Data Bank Accession No. 2oc5A.

In other instances, the alkane or alkene biosynthetic polypeptide variants have at least about 50%, at least about 55%, at least about 60%, at least about 65%, at least about 70%, at least about 75%, at least about 80%, at least about 85%, at least about 90%, at least about 95%, or more than about 95% homology to the amino acid sequence of SEQ ID NO: 2, 4, 6, 8, 10, 12, 14, 16, 18, 20, 22, 24, 26, 28, 30, 32, 34, or 36. In another embodiment, the polypeptide variants include a fragment comprising at least about 5, 10, 15, 20, 25, 30, 35, 40, 50, 75, 100, or 150 consecutive amino acids thereof.

In certain embodiments, the alkane or alkene biosynthetic polypeptide variant comprises SEQ ID NO: 37 or 115. In one aspect, the amino acid sequence SEQ ID NO: 37 is about 60 to about 185 amino acid residues in length, for example, about 70 to about 170 amino acid residues in length, about 60 to about 160 amino acid residues in length, or about 50 to about 150 amino acid residues in length. An exemplary alkane/alkene biosynthetic polypeptide of the invention comprises a 108-mer SEQ ID NO: 37.

In some instances, an aldehyde biosynthetic polypeptide variant retains the same biological function as a polypeptide having the amino acid sequence of SEQ ID NO: 67, 69, 71, 73, 75, 77, 79, 81, or 83 (e.g., retains aldehyde biosynthetic activity) and has an amino acid sequence substantially identical thereto.

In yet other instances, the aldehyde biosynthetic polypeptide variants have at least about 50%, at least about 55%, at least about 60%, at least about 65%, at least about 70%, at least about 75%, at least about 80%, at least about 85%, at least about 90%, at least about 95%, or more than about 95% homology to the amino acid sequence of SEQ ID NO: 67, 69, 71, 73, 75, 77, 79, 81, or 83. In another embodiment, the polypeptide variants include a fragment comprising at least about 5, 10, 15, 20, 25, 30, 35, 40, 50, 75, 100, or 150 consecutive amino acids thereof.

The polypeptide variants or fragments thereof can be obtained by isolating nucleic acids encoding them using techniques described herein or by expressing synthetic nucleic acids encoding them. Alternatively, polypeptide variants or fragments thereof can be obtained through biochemical enrichment or purification procedures. The sequence of polypeptide variants or fragments can be determined by proteolytic digestion, gel electrophoresis, and/or microsequencing. The sequence of the alkane or alkene biosynthetic polypeptide variants or fragments can then be compared to the amino acid sequence of SEQ ID NO: 2, 4, 6, 8, 10, 12, 14, 16, 18, 20, 22, 24, 26, 28, 30, 32, 34, or 36 using any of the programs described herein. The sequence of the aldehyde biosynthetic polypeptide variants or fragments can be compared to the amino acid sequence of SEQ ID NO: 67, 69, 71, 73, 75, 77, 79, 81, or 83 using any of the programs described herein.

The polypeptide variants and fragments thereof can be assayed for aldehyde-, fatty alcohol-, alkane-, and/or alkene-producing activity using routine methods. For example, the polypeptide variants or fragment can be contacted with a substrate (e.g., a fatty acid derivative substrate or other substrate described herein) under conditions that allow the polypeptide variant to function. A decrease in the level of the substrate or an increase in the level of an aldehyde, alkane, or alkene can be measured to determine aldehyde-, fatty alcohol-, alkane-, or alkene-producing activity, respectively.

### Anti-Aldehyde, Anti-Fatty Alcohol, Anti-Alkane, and Anti-Alkene Biosynthetic Polypeptide Antibodies

The aldehyde, fatty alcohol, alkane, and alkene biosynthetic polypeptides described herein can also be used to produce antibodies directed against aldehyde, fatty alcohol, alkane, and alkene biosynthetic polypeptides. Such antibodies can be used, for example, to detect the expression of an aldehyde, fatty alcohol, alkane, or alkene biosynthetic polypeptide using methods known in the art. The antibody can be, e.g., a polyclonal antibody; a monoclonal antibody or antigen binding fragment thereof; a modified antibody such as a chimeric antibody, reshaped antibody, humanized antibody, or fragment thereof (e.g., Fab′, Fab, F(ab′)₂); or a biosynthetic antibody, e.g., a single chain antibody, single domain antibody (DAB), Fv, single chain Fv (scFv), or the like.

Methods of making and using polyclonal and monoclonal antibodies are described, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition).

### Substrates

The compositions and methods described herein can be used to produce aldehydes, fatty alcohols, alkanes, and/or alkenes from an appropriate substrate. While not wishing to be bound by a particular theory, it is believed that the alkane or alkene biosynthetic polypeptides described herein produce alkanes or alkenes from substrates via a decarbonylation mechanism. In some instances, the substrate is a fatty acid derivative, e.g., a fatty aldehyde, and an alkane having particular branching patterns and carbon chain length can be produced from a fatty acid derivative, e.g., a fatty aldehyde, having those particular characteristics. In other instances, the substrate is an unsaturated fatty acid derivative, e.g., an unsaturated fatty aldehyde, and an alkene having particular branching patterns and carbon chain length can be produced from an unsaturated fatty acid derivative, e.g., an unsaturated fatty aldehyde, having those particular characteristics.

While not wishing to be bound by a particular theory, it is believed that the aldehyde biosynthetic polypeptides described herein produce aldehydes from substrates via a reduction mechanism. In certain instances, the substrate is an acyl-ACP.

While not wishing to be bound by a particular theory, it is believed that the fatty alcohols described herein are produced from substrates via a reduction mechanism. In certain instances, the substrate is a fatty aldehyde.

Accordingly, each step within a biosynthetic pathway that leads to the production of these substrates can be modified to produce or overproduce the substrate of interest. For example, known genes involved in the fatty acid biosynthetic pathway, the fatty aldehyde pathway, and the fatty alcohol pathway can be expressed, overexpressed, or attenuated in host cells to produce a desired substrate (see, e.g., PCT/US08/058,788, specifically incorporated by reference herein). Exemplary genes are provided in FIG. 40.

### Synthesis of Substrates

Fatty acid synthase (FAS) is a group of polypeptides that catalyze the initiation and elongation of acyl chains (Marrakchi et al., Biochemical Society, 30:1050-1055, 2002). The acyl carrier protein (ACP) along with the enzymes in the FAS pathway control the length, degree of saturation, and branching of the fatty acid derivatives produced. The fatty acid biosynthetic pathway involves the precursors acetyl-CoA and malonyl-CoA. The steps in this pathway are catalyzed by enzymes of the fatty acid biosynthesis (fab) and acetyl-CoA carboxylase (acc) gene families (see, e.g., Heath et al., Prog. Lipid Res. 40(6):467-97 (2001)). In certain embodiments, the host cells can be engineered to produce alkanes/alkenes in a malonyl-CoA-independent fashion.

Host cells can be engineered to express fatty acid derivative substrates by recombinantly expressing or overexpressing acetyl-CoA and/or malonyl-CoA synthase genes. For example, to increase acetyl-CoA production, one or more of the following genes can be expressed in a host cell: pdh, panK, aceEF (encoding the E1p dehydrogenase component and the E2p dihydrolipoamide acyltransferase component of the pyruvate and 2-oxoglutarate dehydrogenase complexes), fabH, fabD, fabG, acpP, and fabF. Exemplary GenBank accession numbers for these genes are: pdh (BAB34380, AAC73227, AAC73226), panK (also known as coaA, AAC76952), aceEF (AAC73227, AAC73226), fabH (AAC74175), fabD (AAC74176), fabG (AAC74177), acpP (AAC74178), fabF (AAC74179). Additionally, the expression levels of fadE, gpsA, ldhA, pflb, adhE, pta, poxB, ackA, and/or ackB can be attenuated or knocked-out in an engineered host cell by transformation with conditionally replicative or non-replicative plasmids containing null or deletion mutations of the corresponding genes or by substituting promoter or enhancer sequences. Exemplary GenBank accession numbers for these genes are: fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430). The resulting host cells will have increased acetyl-CoA production levels when grown in an appropriate environment.

Malonyl-CoA overexpression can be effected by introducing accABCD (e.g., accession number AAC73296, EC 6.4.1.2) into a host cell. Fatty acids can be further overexpressed in host cells by introducing into the host cell a DNA sequence encoding a lipase (e.g., accession numbers CAA89087, CAA98876).

In addition, inhibiting PlsB can lead to an increase in the levels of long chain acyl-ACP, which will inhibit early steps in the pathway (e.g., accABCD, fabH, and fabl). The plsB (e.g., accession number AAC77011) D311E mutation can be used to increase the amount of available acyl-CoA.

In addition, a host cell can be engineered to overexpress a sfa gene (suppressor of fabA, e.g., accession number AAN79592) to increase production of monounsaturated fatty acids (Rock et al., J. Bacteriology 178:5382-5387, 1996).

In some instances, host cells can be engineered to express, overexpress, or attenuate expression of a thioesterase to increase fatty acid substrate production. The chain length of a fatty acid substrate is controlled by thioesterase. In some instances, a tes or fat gene can be overexpressed. In other instances, C₁₀ fatty acids can be produced by attenuating thioesterase C₁₈ (e.g., accession numbers AAC73596 and P0ADA1), which uses C_{18:1}-ACP, and expressing thioesterase C₁₀ (e.g., accession number Q39513), which uses C₁₀-ACP. This results in a relatively homogeneous population of fatty acids that have a carbon chain length of 10. In yet other instances, C₁₄ fatty acids can be produced by attenuating endogenous thioesterases that produce non-C₁₄ fatty acids and expressing the thioesterases that use C₁₄-ACP (for example, accession number Q39473). In some situations, C₁₂ fatty acids can be produced by expressing thioesterases that use C₁₂-ACP (for example, accession number Q41635) and attenuating thioesterases that produce non-C₁₂ fatty acids. Acetyl-CoA, malonyl-CoA, and fatty acid overproduction can be verified using methods known in the art, for example, by using radioactive precursors, HPLC, and GC-MS subsequent to cell lysis. Non-limiting examples of thioesterases that can be used in the methods described herein are listed in Table 2.

TABLE 2
Thioesterases

| | | | Preferential |
|---|---|---|---|
| Accession | | | Product |
| No. | Source Organism | Gene | Produced |
| AAC73596 | E. coli | tesA without | C_{18:1} |
| | | leader | |
| | | sequence | |
| AAC73555 | E. coli | tesB | |
| Q41635; | Umbellularia California | fatB | C_{12:0} |
| AAA34215 | | | |
| Q39513; | Cuphea hookeriana | fatB2 | C_{8:0}-C_{10:0} |
| AAC49269 | | | |
| AAC49269; | Cuphea hookeriana | fatB3 | C_{14:0}-C_{16:0} |
| AAC72881 | | | |
| Q39473; | Cinnamonum camphorum | fatB | C_{14:0} |
| AAC49151 | | | |
| CAA85388 | Arabidopsis thaliana | fatB [M141T]* | C_{16:1} |
| NP 189147; | Arabidopsis thaliana | fatA | C_{18:1} |
| NP 193041 | | | |
| CAC39106 | Bradyrhiizobium japonicum | fatA | C_{18:1} |
| AAC72883 | Cuphea hookeriana | fatA | C_{18:1} |
| AAL79361 | Helianthus annus | fatA1 | |

*Mayer et al., BMC Plant Biology 7: 1-11, 2007

### Formation of Branched Aldehydes, Fatty Alcohols, Alkanes, and Alkenes

Aldehydes, fatty alcohols, alkanes, an alkenes can be produced that contain branch points by using branched fatty acid derivatives as substrates. For example, although E. coli naturally produces straight chain fatty acid derivatives (sFAs), E. coli can be engineered to produce branched chain fatty acid derivatives (brFAs) by introducing and expressing or overexpressing genes that provide branched precursors in the E. coli (e.g., bkd, ilv, icm, and fab gene families). Additionally, a host cell can be engineered to express or overexpress genes encoding proteins for the elongation of brFAs (e.g., ACP, FabF, etc.) and/or to delete or attenuate the corresponding host cell genes that normally lead to sFAs.

The first step in forming brFAs is the production of the corresponding α-keto acids by a branched-chain amino acid aminotransferase. Host cells may endogenously include genes encoding such enzymes or such genes can be recombinantly introduced. E. coli, for example, endogenously expresses such an enzyme, IlvE (EC 2.6.1.42; GenBank accession YP_{—}026247). In some host cells, a heterologous branched-chain amino acid aminotransferase may not be expressed. However, E. coli IlvE or any other branched-chain amino acid aminotransferase (e.g., IlvE from Lactococcus lactis (GenBank accession AAF34406), IlvE from Pseudomonas putida (GenBank accession NP_{—}745648), or IlvE from Streptomyces coelicolor (GenBank accession NP_{—}629657)), if not endogenous, can be introduced and recombinantly expressed.

The second step is the oxidative decarboxylation of the α-ketoacids to the corresponding branched-chain acyl-CoA. This reaction can be catalyzed by a branched-chain α-keto acid dehydrogenase complex (bkd; EC 1.2.4.4.) (Denoya et al., J. Bacteriol. 177:3504, 1995), which consists of E1α/β (decarboxylase), E2 (dihydrolipoyl transacylase), and E3 (dihydrolipoyl dehydrogenase) subunits. These branched-chain α-keto acid dehydrogenase complexes are similar to pyruvate and α-ketoglutarate dehydrogenase complexes. Any microorganism that possesses brFAs and/or grows on branched-chain amino acids can be used as a source to isolate bkd genes for expression in host cells, for example, E. coli. Furthermore, E. coli has the E3 component as part of its pyruvate dehydrogenase complex (lpd, EC 1.8.1.4, GenBank accession NP_{—}414658). Thus, it can be sufficient to express only the E1 α/β and E2 bkd genes. Table 3 lists non-limiting examples of bkd genes from several microorganisms that can be recombinantly introduced and expressed in a host cell to provide branched-chain acyl-CoA precursors.

TABLE 3
Bkd genes from selected microorganisms

| Organism | Gene | GenBank Accession No. |
|---|---|---|
| Streptomyces coelicolor | bkdA1 (E1α) | NP_628006 |
| | bkdB1 (E1β) | NP_628005 |
| | bkdC1 (E2) | NP_638004 |
| Streptomyces coelicolor | bkdA2 (E1α) | NP_733618 |
| | bkdB2 (E1β) | NP_628019 |
| | bkdC2 (E2) | NP_628018 |
| Streptomyces avermitilis | bkdA (E1a) | BAC72074 |
| | bkdB (E1b) | BAC72075 |
| | bkdC (E2) | BAC72076 |
| Streptomyces avermitilis | bkdF (E1α) | BAC72088 |
| | bkdG (E1β) | BAC72089 |
| | bkdH (E2) | BAC72090 |
| Bacillus subtilis | bkdAA (E1α) | NP_390288 |
| | bkdAB (E1β) | NP_390288 |
| | bkdB (E2) | NP_390288 |
| Pseudomonas putida | bkdA1 (E1α) | AAA65614 |
| | bkdA2 (E1β) | AAA65615 |
| | bkdC (E2) | AAA65617 |

In another example, isobutyryl-CoA can be made in a host cell, for example in E. coli, through the coexpression of a crotonyl-CoA reductase (Ccr, EC 1.6.5.5, 1.1.1.1) and isobutyryl-CoA mutase (large subunit IcmA, EC 5.4.99.2; small subunit IcmB, EC 5.4.99.2) (Han and Reynolds, J. Bacteriol. 179:5157, 1997). Crotonyl-CoA is an intermediate in fatty acid biosynthesis in E. coli and other microorganisms. Non-limiting examples of ccr and icm genes from selected microorganisms are listed in Table 4.

TABLE 4
Ccr and icm genes from selected microorganisms

| Organism | Gene | GenBank Accession No. |
|---|---|---|
| Streptomyces coelicolor | Ccr | NP_630556 |
| | icmA | NP_629554 |
| | icmB | NP_630904 |
| Streptomyces | ccr | AAD53915 |
| cinnamonensis | icmA | AAC08713 |
| | icmB | AJ246005 |

In addition to expression of the bkd genes, the initiation of brFA biosynthesis utilizes β-ketoacyl-acyl-carrier-protein synthase III (FabH, EC 2.3.1.41) with specificity for branched chain acyl-CoAs (Li et al., J. Bacteriol. 187:3795-3799, 2005). Non-limiting examples of such FabH enzymes are listed in Table 5. fabH genes that are involved in fatty acid biosynthesis of any brFA-containing microorganism can be expressed in a host cell. The Bkd and FabH enzymes from host cells that do not naturally make brFA may not support brFA production. Therefore, bkd and fabH can be expressed recombinantly. Vectors containing the bkd and fabH genes can be inserted into such a host cell. Similarly, the endogenous level of Bkd and FabH production may not be sufficient to produce brFA. In this case, they can be overexpressed. Additionally, other components of the fatty acid biosynthesis pathway can be expressed or overexpressed, such as acyl carrier proteins (ACPs) and β-ketoacyl-acyl-carrier-protein synthase II (fabF, EC 2.3.1.41) (non-limiting examples of candidates are listed in Table 5). In addition to expressing these genes, some genes in the endogenous fatty acid biosynthesis pathway can be attenuated in the host cell (e.g., the E. coli genes fabH (GenBank accession # NP_{—}415609) and/or fabF (GenBank accession # NP_{—}415613)).

TABLE 5
FabH, ACP and fabF genes from selected microorganisms with brFAs

| | | GenBank |
|---|---|---|
| Organism | Gene | Accession No. |
| Streptomyces coelicolor | fabH1 | NP_626634 |
| | ACP | NP_626635 |
| | fabF | NP_626636 |
| Streptomyces avermitilis | fabH3 | NP_823466 |
| | fabC3 (ACP) | NP_823467 |
| | fabF | NP_823468 |
| Bacillus subtilis | fabH_A | NP_389015 |
| | fabH_B | NP_388898 |
| | ACP | NP_389474 |
| | fabF | NP_389016 |
| Stenotrophomonas | SmalDRAFT_0818 (FabH) | ZP_01643059 |
| maltophilia | SmalDRAFT_0821 (ACP) | ZP_01643063 |
| | SmalDRAFT_0822 (FabF) | ZP_01643064 |
| Legionella pneumophila | FabH | YP_123672 |
| | ACP | YP_123675 |
| | fabF | YP_123676 |

### Formation of Cyclic Aldehydes, Fatty Alcohols, Alkanes, and Alkenes

Cyclic aldehydes, fatty alcohols, alkanes, and alkenes can be produced by using cyclic fatty acid derivatives as substrates. To produce cyclic fatty acid derivative substrates, genes that provide cyclic precursors (e.g., the ans, chc, and plm gene families) can be introduced into the host cell and expressed to allow initiation of fatty acid biosynthesis from cyclic precursors. For example, to convert a host cell, such as E. coli, into one capable of synthesizing co-cyclic fatty acid derivatives (cyFA), a gene that provides the cyclic precursor cyclohexylcarbonyl-CoA (CHC-CoA) (Cropp et al., Nature Biotech. 18:980-983, 2000) can be introduced and expressed in the host cell. Non-limiting examples of genes that provide CHC-CoA in E. coli include: ansJ, ansK, ansL, chcA, and ansM from the ansatrienin gene cluster of Streptomyces collinus (Chen et al., Eur. J. Biochem. 261: 98-107, 1999) or plmJ, plmK, plmL, chcA, and plmM from the phoslactomycin B gene cluster of Streptomyces sp. HK803 (Palaniappan et al., J. Biol. Chem. 278:35552-35557, 2003) together with the chcB gene (Patton et al., Biochem. 39:7595-7604, 2000) from S. collinus, S. avermitilis, or S. coelicolor (see Table 6). The genes listed in Table 5 can then be expressed to allow initiation and elongation of ω-cyclic fatty acids. Alternatively, the homologous genes can be isolated from microorganisms that make cyFA and expressed in a host cell (e.g., E. coli).

TABLE 6
Genes for the synthesis of CHC-CoA

| | | GenBank |
|---|---|---|
| Organism | Gene | Accession No. |
| Streptomyces collinus | ansJK | U72144* |
| | ansL | |
| | chcA | |
| | ansM | |
| | chcB | AF268489 |
| Streptomyces sp. HK803 | pmlJK | AAQ84158 |
| | pmlL | AAQ84159 |
| | chcA | AAQ84160 |
| | pmlM | AAQ84161 |
| Streptomyces coelicolor | chcB/caiD | NP_629292 |
| Streptomyces avermitilis | chcB/caiD | NP_629292 |

*Only chcA is annotated in GenBank entry U72144, ansJKLM are according to Chen et al. (Eur. J. Biochem. 261: 98-107, 1999).

The genes listed in Table 5 (fabH, ACP, and fabF) allow initiation and elongation of co-cyclic fatty acid derivatives because they have broad substrate specificity. If the coexpression of any of these genes with the genes listed in Table 6 does not yield cyFA, then fabH, ACP, and/or fabF homologs from microorganisms that make cyFAs (e.g., those listed in Table 7) can be isolated (e.g., by using degenerate PCR primers or heterologous DNA sequence probes) and coexpressed.

TABLE 7
Non-limiting examples of microorganisms that contain ω-cyclic
fatty acids

| Organism | Reference |
|---|---|
| Curtobacterium pusillum | ATCC19096 |
| Alicyclobacillus acidoterrestris | ATCC49025 |
| Alicyclobacillus acidocaldarius | ATCC27009 |
| Alicyclobacillus cycloheptanicus* | Moore, J. Org. Chem. 62: |
| | pp. 2173, 1997. |

*Uses cycloheptylcarbonyl-CoA and not cyclohexylcarbonyl-CoA as precursor for cyFA biosynthesis.

### Aldehyde, Fatty Alcohol, and Alkene Saturation Levels

The degree of saturation in fatty acid derivatives can be controlled by regulating the degree of saturation of fatty acid derivative intermediates. The sfa, gns, and fab families of genes can be expressed or overexpressed to control the saturation of fatty acids. FIG. 40 lists non-limiting examples of genes in these gene families that may be used in the methods and host cells described herein.

Host cells can be engineered to produce unsaturated fatty acids by engineering the host cell to overexpress fabB or by growing the host cell at low temperatures (e.g., less than 37° C.). FabB has preference to cis-δ3decenoyl-ACP and results in unsaturated fatty acid production in E. coli. Overexpression of fabB results in the production of a significant percentage of unsaturated fatty acids (de Mendoza et al., J. Biol. Chem. 258:2098-2101, 1983). The gene fabB may be inserted into and expressed in host cells not naturally having the gene. These unsaturated fatty acid derivatives can then be used as intermediates in host cells that are engineered to produce fatty acid derivatives, such as fatty aldehydes, fatty alcohols, or alkenes.

In other instances, a repressor of fatty acid biosynthesis, for example, fabR (GenBank accession NP_{—}418398), can be deleted, which will also result in increased unsaturated fatty acid production in E. coli (Zhang et al., J. Biol. Chem. 277:15558, 2002). Similar deletions may be made in other host cells. A further increase in unsaturated fatty acid derivatives may be achieved, for example, by overexpressing fabM (trans-2, cis-3-decenoyl-ACP isomerase, GenBank accession DAA05501) and controlled expression of fabK (trans-2-enoyl-ACP reductase II, GenBank accession NP_{—}357969) from Streptococcus pneumoniae (Marrakchi et al., J. Biol. Chem. 277: 44809, 2002), while deleting E. coli fabI (trans-2-enoyl-ACP reductase, GenBank accession NP_{—}415804). In some examples, the endogenous fabF gene can be attenuated, thus increasing the percentage of palmitoleate (C16:1) produced.

### Other Substrates

Other substrates that can be used to produce aldehydes, fatty alcohols, alkanes, and alkenes in the methods described herein are acyl-ACP, acyl-CoA, a fatty aldehyde, or a fatty alcohol, which are described in, for example, PCT/US08/058,788. Exemplary genes that can be altered to express or overexpress these substrates in host cells are listed in FIG. 40. Other exemplary genes are described in PCT/US08/058,788.

### Genetic Engineering of Host Cells to Produce Aldehydes, Fatty Alcohols, Alkanes, and Alkenes

Various host cells can be used to produce aldehydes, fatty alcohols, alkanes, and/or alkenes, as described herein. A host cell can be any prokaryotic or eukaryotic cell. For example, a polypeptide described herein can be expressed in bacterial cells (such as E. coli), insect cells, yeast or mammalian cells (such as Chinese hamster ovary cells (CHO) cells, COS cells, VERO cells, BHK cells, HeLa cells, Cv1 cells, MDCK cells, 293 cells, 3T3 cells, or PC12 cells). Other exemplary host cells include cells from the members of the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Schizosaccharomyces, Yarrowia, or Streptomyces. Yet other exemplary host cells can be a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhizomucor miehei cell, a Mucor michei cell, a Streptomyces lividans cell, a Streptomyces murinus cell, or an Actinomycetes cell.

Other nonlimiting examples of host cells are those listed in Table 1.

In a preferred embodiment, the host cell is an E. coli cell. In a more preferred embodiment, the host cell is from E. coli strains B, C, K, or W. Other suitable host cells are known to those skilled in the art.

Various methods well known in the art can be used to genetically engineer host cells to produce aldehydes, fatty alcohols, alkanes and/or alkenes. The methods include the use of vectors, preferably expression vectors, containing a nucleic acid encoding an aldehyde, fatty alcohol, alkane, and/or alkene biosynthetic polypeptide described herein, or a polypeptide variant or fragment thereof. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are thereby replicated along with the host genome. Moreover, certain vectors, such as expression vectors, are capable of directing the expression of genes to which they are operatively linked. In general, expression vectors used in recombinant DNA techniques are often in the form of plasmids. However, other forms of expression vectors, such as viral vectors (e.g., replication defective retroviruses, adenoviruses, and adeno-associated viruses), can also be used.

The recombinant expression vectors described herein include a nucleic acid described herein in a form suitable for expression of the nucleic acid in a host cell. The recombinant expression vectors can include one or more control sequences, selected on the basis of the host cell to be used for expression. The control sequence is operably linked to the nucleic acid sequence to be expressed. Such control sequences are described, for example, in Goeddel, Gene Expression Technology: Methods in Enzymology 185, Academic Press, San Diego, Calif. (1990). Control sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors described herein can be introduced into host cells to produce polypeptides, including fusion polypeptides, encoded by the nucleic acids as described herein.

Recombinant expression vectors can be designed for expression of an aldehyde, fatty alcohol, alkane, and/or alkene biosynthetic polypeptide or variant in prokaryotic or eukaryotic cells (e.g., bacterial cells, such as E. coli, insect cells (using baculovirus expression vectors), yeast cells, or mammalian cells). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example, by using T7 promoter regulatory sequences and T7 polymerase.

Expression of polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech Inc; Smith et al., Gene (1988) 67:31-40), pMAL (New England Biolabs, Beverly, Mass.), and pRITS (Pharmacia, Piscataway, N.J.), which fuse glutathione S-transferase (GST), maltose E binding protein, or protein A, respectively, to the target recombinant polypeptide.

Examples of inducible, non-fusion E. coli expression vectors include pTrc (Amann et al., Gene (1988) 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant polypeptide expression is to express the polypeptide in a host cell with an impaired capacity to proteolytically cleave the recombinant polypeptide (see Gottesman, Gene Expression Technology: Methods in Enzymology 185, Academic Press, San Diego, Calif. (1990) 119-128). Another strategy is to alter the nucleic acid sequence to be inserted into an expression vector so that the individual codons for each amino acid are those preferentially utilized in the host cell (Wada et al., Nucleic Acids Res. (1992) 20:2111-2118). Such alteration of nucleic acid sequences can be carried out by standard DNA synthesis techniques.

In another embodiment, the host cell is a yeast cell. In this embodiment, the expression vector is a yeast expression vector. Examples of vectors for expression in yeast S. cerevisiae include pYepSec1 (Baldari et al., EMBO J. (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.), and picZ (Invitrogen Corp, San Diego, Calif.).

Alternatively, a polypeptide described herein can be expressed in insect cells using baculovirus expression vectors. Baculovirus vectors available for expression of proteins in cultured insect cells (e.g., Sf 9 cells) include, for example, the pAc series (Smith et al., Mol. Cell Biol. (1983) 3:2156-2165) and the pVL series (Lucklow et al., Virology (1989) 170:31-39).

In yet another embodiment, the nucleic acids described herein can be expressed in mammalian cells using a mammalian expression vector. Examples of mammalian expression vectors include pCDM8 (Seed, Nature (1987) 329:840) and pMT2PC (Kaufman et al., EMBO J. (1987) 6:187-195). When used in mammalian cells, the expression vector's control functions can be provided by viral regulatory elements. For example, commonly used promoters are derived from polyoma, Adenovirus 2, cytomegalovirus and Simian Virus 40. Other suitable expression systems for both prokaryotic and eukaryotic cells are described in chapters 16 and 17 of Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989.

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to antibiotics) can be introduced into the host cells along with the gene of interest. Selectable markers include those that confer resistance to drugs, such as ampacillin, kanamycin, chloramphenicol, or tetracycline. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

In certain methods, an aldehyde biosynthetic polypeptide and an alkane or alkene biosynthetic polypeptide are co-expressed in a single host cell. In alternate methods, an aldehyde biosynthetic polypeptide and an alcohol dehydrogenase polypeptide are co-expressed in a single host cell.

### Transport Proteins

Transport proteins can export polypeptides and hydrocarbons (e.g., aldehydes, alkanes, and/or alkenes) out of a host cell. Many transport and efflux proteins serve to excrete a wide variety of compounds and can be naturally modified to be selective for particular types of hydrocarbons.

Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabditis elegans, Arabidopsis thalania, Alkaligenes eutrophus, and Rhodococcus erythropolis. Exemplary ABC transport proteins that can be used are listed in FIG. 40 (e.g., CER5, AtMRP5, AmiS2, and AtPGP1). Host cells can also be chosen for their endogenous ability to secrete hydrocarbons. The efficiency of hydrocarbon production and secretion into the host cell environment (e.g., culture medium, fermentation broth) can be expressed as a ratio of intracellular product to extracellular product. In some examples, the ratio can be about 5:1, 4:1, 3:1, 2:1, 1:1, 1:2, 1:3, 1:4, or 1:5.

### Fermentation

The production and isolation of aldehydes, fatty alcohols, alkanes and/or alkenes can be enhanced by employing suitable fermentation techniques. One method for maximizing production while reducing costs is increasing the percentage of the carbon source that is converted to hydrocarbon products.

During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311:1113, 2006; Venturi FEMS Microbio. Rev. 30:274-291, 2006; and Reading et al., FEMS Microbiol. Lett. 254:1-11, 2006) can be used to activate checkpoint genes, such as p53, p21, or other checkpoint genes.

Genes that can be activated to stop cell replication and growth in E. coli include umuDC genes. The overexpression of umuDC genes stops the progression from stationary phase to exponential growth (Murli et al., J. of Bact. 182:1127, 2000). UmuC is a DNA polymerase that can carry out translesion synthesis over non-coding lesions—the mechanistic basis of most UV and chemical mutagenesis. The umuDC gene products are involved in the process of translesion synthesis and also serve as a DNA sequence damage checkpoint. The umuDC gene products include UmuC, UmuD, umuD′, UmuD′₂C, UmuD′₂, and UmuD₂. Simultaneously, product-producing genes can be activated, thus minimizing the need for replication and maintenance pathways to be used while an aldehyde, alkane and/or alkene is being made. Host cells can also be engineered to express umuC and umuD from E. coli in pBAD24 under the prpBCDE promoter system through de novo synthesis of this gene with the appropriate end-product production genes.

The percentage of input carbons converted to aldehydes, fatty alcohols, alkanes and/or alkenes can be a cost driver. The more efficient the process is (i.e., the higher the percentage of input carbons converted to aldehydes, fatty alcohols, alkanes and/or alkenes), the less expensive the process will be. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other hydrocarbon products and carbon sources. Typical efficiencies in the literature are approximately less than 5%. Host cells engineered to produce aldehydes, alkanes and/or alkenes can have greater than about 1, 3, 5, 10, 15, 20, 25, and 30% efficiency. In one example, host cells can exhibit an efficiency of about 10% to about 25%. In other examples, such host cells can exhibit an efficiency of about 25% to about 30%. In other examples, host cells can exhibit greater than 30% efficiency.

The host cell can be additionally engineered to express recombinant cellulosomes, such as those described in PCT application number PCT/US2007/003736. These cellulosomes can allow the host cell to use cellulosic material as a carbon source. For example, the host cell can be additionally engineered to express invertases (EC 3.2.1.26) so that sucrose can be used as a carbon source. Similarly, the host cell can be engineered using the teachings described in U.S. Pat. Nos. 5,000,000; 5,028,539; 5,424,202; 5,482,846; and 5,602,030; so that the host cell can assimilate carbon efficiently and use cellulosic materials as carbon sources.

In one example, the fermentation chamber can enclose a fermentation that is undergoing a continuous reduction. In this instance, a stable reductive environment can be created. The electron balance can be maintained by the release of carbon dioxide (in gaseous form). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of aldehydes, alkanes and/or alkenes.

For small scale production, the engineered host cells can be grown in batches of, for example, around 100 ml, 500 ml, 1 L, 2 L, 5 L, or 10 L; fermented; and induced to express desired aldehydes, fatty alcohols, alkanes and/or alkenes based on the specific genes encoded in the appropriate plasmids. For example, E. coli BL21(DE3) cells harboring pBAD24 (with ampicillin resistance and the aldehyde, fatty alcohol, alkane, or alkene synthesis pathway) as well as pUMVC1 (with kanamycin resistance and the acetyl CoA/malonyl CoA overexpression system) can be incubated overnight in 2 L flasks at 37° C. shaken at >200 rpm in 500 ml LB medium supplemented with 75 μg/ml ampicillin and 50 μg/ml kanamycin until cultures reach an OD₆₀₀ of >0.8. Upon achieving an OD₆₀₀ of >0.8, the cells can be supplemented with 25 mM sodium proprionate (pH 8.0) to activate the engineered gene systems for production and to stop cellular proliferation by activating UmuC and UmuD proteins. Induction can be performed for 6 hrs at 30° C. After incubation, the media can be examined for aldehydes, fatty alcohols, alkanes and/or alkenes using GC-MS.

For large scale production, the engineered host cells can be grown in batches of 10 L, 100 L, 1000 L, or larger; fermented; and induced to express desired aldehydes, fatty alcohols, alkanes and/or alkenes based on the specific genes encoded in the appropriate plasmids. For example, E. coli BL21(DE3) cells harboring pBAD24 (with ampicillin resistance and the aldehyde and/or alkane synthesis pathway) as well as pUMVC1 (with kanamycin resistance and the acetyl-CoA/malonyl-CoA overexpression system) can be incubated from a 500 ml seed culture for 10 L fermentations (5 L for 100 L fermentations, etc.) in LB media (glycerol free) with 50 μg/ml kanamycin and 75 μg/ml ampicillin at 37° C., and shaken at >200 rpm until cultures reach an OD₆₀₀ of >0.8 (typically 16 hrs). Media can be continuously supplemented to maintain 25 mM sodium proprionate (pH 8.0) to activate the engineered gene systems for production and to stop cellular proliferation by activating umuC and umuD proteins. Media can be continuously supplemented with glucose to maintain a concentration 25 g/100 ml.

After the first hour of induction, aliquots of no more than 10% of the total cell volume can be removed each hour and allowed to sit without agitation to allow the aldehydes, alkanes and/or alkenes to rise to the surface and undergo a spontaneous phase separation. The aldehyde, fatty alcohols, alkane and/or alkene component can then be collected, and the aqueous phase returned to the reaction chamber. The reaction chamber can be operated continuously. When the OD₆₀₀ drops below 0.6, the cells can be replaced with a new batch grown from a seed culture.

### Producing Aldehydes, Fatty Alcohols, Alkanes and Alkenes Using Cell-Free Methods

In some methods described herein, an aldehyde, fatty alcohols, alkane and/or alkene can be produced using a purified polypeptide described herein and a substrate described herein. For example, a host cell can be engineered to express aldehyde, fatty alcohols, alkane and/or alkene biosynthetic polypeptide or variant as described herein. The host cell can be cultured under conditions suitable to allow expression of the polypeptide. Cell free extracts can then be generated using known methods. For example, the host cells can be lysed using detergents or by sonication. The expressed polypeptides can be purified using known methods. After obtaining the cell free extracts, substrates described herein can be added to the cell free extracts and maintained under conditions to allow conversion of the substrates to aldehydes, fatty alcohols, alkanes and/or alkenes. The aldehydes, fatty alcohols, alkanes and/or alkenes can then be separated and purified using known techniques.

### Post-Production Processing

The aldehydes, fatty alcohols, alkanes and/or alkenes produced during fermentation can be separated from the fermentation media. Any known technique for separating aldehydes, fatty alcohols, alkanes and/or alkenes from aqueous media can be used. One exemplary separation process is a two phase (bi-phasic) separation process. This process involves fermenting the genetically engineered host cells under conditions sufficient to produce an aldehyde, fatty alcohols, alkane and/or alkene, allowing the aldehyde, fatty alcohols, alkane and/or alkene to collect in an organic phase, and separating the organic phase from the aqueous fermentation broth. This method can be practiced in both a batch and continuous fermentation setting.

Bi-phasic separation uses the relative immiscibility of aldehydes, fatty alcohols, alkanes and/or alkenes to facilitate separation. Immiscible refers to the relative inability of a compound to dissolve in water and is defined by the compound's partition coefficient. One of ordinary skill in the art will appreciate that by choosing a fermentation broth and organic phase, such that the aldehyde, alkane and/or alkene being produced has a high logP value, the aldehyde, alkane and/or alkene can separate into the organic phase, even at very low concentrations, in the fermentation vessel.

The aldehydes, fatty alcohols, alkanes and/or alkenes produced by the methods described herein can be relatively immiscible in the fermentation broth, as well as in the cytoplasm. Therefore, the aldehyde, fatty alcohols, alkane and/or alkene can collect in an organic phase either intracellularly or extracellularly. The collection of the products in the organic phase can lessen the impact of the aldehyde, fatty alcohols, alkane and/or alkene on cellular function and can allow the host cell to produce more product.

The methods described herein can result in the production of homogeneous compounds wherein at least about 60%, 70%, 80%, 90%, or 95% of the aldehydes, fatty alcohols, alkanes and/or alkenes produced will have carbon chain lengths that vary by less than about 6 carbons, less than about 4 carbons, or less than about 2 carbons. These compounds can also be produced with a relatively uniform degree of saturation. These compounds can be used directly as fuels, fuel additives, specialty chemicals, starting materials for production of other chemical compounds (e.g., polymers, surfactants, plastics, textiles, solvents, adhesives, etc.), or personal care product additives. These compounds can also be used as feedstock for subsequent reactions, for example, hydrogenation, catalytic cracking (via hydrogenation, pyrolisis, or both), to make other products.

In some embodiments, the aldehydes, fatty alcohols, alkanes and/or alkenes produced using methods described herein can contain between about 50% and about 90% carbon; or between about 5% and about 25% hydrogen. In other embodiments, the aldehydes, fatty alcohols, alkanes and/or alkenes produced using methods described herein can contain between about 65% and about 85% carbon; or between about 10% and about 15% hydrogen.

### Fuel Compositions and Specialty Chemical Compositions

The aldehydes, fatty alcohols, alkanes and/or alkenes described herein can be used as or converted into a fuel or as a specialty chemical. One of ordinary skill in the art will appreciate that, depending upon the intended purpose of the fuel or specialty chemical, different aldehydes, fatty alcohols, alkanes and/or alkenes can be produced and used. For example, a branched aldehyde, fatty alcohol, alkane and/or alkene may be desirable for automobile fuel that is intended to be used in cold climates. In addition, when the aldehydes, fatty alcohols, alkanes and/or alkenes described herein are used as a feedstock for fuel or specialty chemical production, one of ordinary skill in the art will appreciate that the characteristics of the aldehyde, fatty alcohol, alkane and/or alkene feedstock will affect the characteristics of the fuel or specialty chemical produced. Hence, the characteristics of the fuel or specialty chemical product can be selected for by producing particular aldehydes, fatty alcohols, alkanes and/or alkenes for use as a feedstock.

Using the methods described herein, biofuels having desired fuel qualities can be produced from aldehydes, fatty alcohols, alkanes and/or alkenes. Biologically produced aldehydes, fatty alcohols, alkanes and/or alkenes represent a new source of biofuels, which can be used as jet fuel, diesel, or gasoline. Some biofuels made using aldehydes, fatty alcohols, alkanes and/or alkenes have not been produced from renewable sources and are new compositions of matter. These new fuels or specialty chemicals can be distinguished from fuels or specialty chemicals derived from petrochemical carbon on the basis of dual carbon-isotopic fingerprinting. Additionally, the specific source of biosourced carbon (e.g., glucose vs. glycerol) can be determined by dual carbon-isotopic fingerprinting (see, e.g., U.S. Pat. No. 7,169,588, in particular col. 4, line 31, to col. 6, line 8).

The aldehydes, fatty alcohols, alkanes and/or alkenes and the associated biofuels, specialty chemicals, and mixtures can be distinguished from their petrochemical derived counterparts on the basis of ¹⁴C (f_{M}) and dual carbon-isotopic fingerprinting. In some examples, the aldehyde, fatty alcohol, alkane and/or alkene in the biofuel composition can have a fraction of modern carbon (f_{M} ¹⁴C) of, for example, at least about 1.003, 1.010, or 1.5.

In some examples, a biofuel composition can be made that includes aldehydes, fatty alcohols, alkanes and/or alkenes having δ¹³C of from about −15.4 to about −10.9, where the aldehydes, fatty alcohols, alkanes and/or alkenes account for at least about 85% of biosourced material (i.e., derived from a renewable resource, such as biomass, cellulosic materials, and sugars) in the composition.

The ability to distinguish these biologically derived products is beneficial in tracking these materials in commerce. For example, fuels or specialty chemicals comprising both biologically derived and petroleum-based carbon isotope profiles can be distinguished from fuels and specialty chemicals made only of petroleum-based materials. Thus, the aldehydes, fatty alcohols, alkanes and/or alkenes described herein can be followed in commerce or identified in commerce as a biofuel on the basis of their unique profile. In addition, other competing materials can be identified as being biologically derived or derived from a petrochemical source.

Fuel additives are used to enhance the performance of a fuel or engine. For example, fuel additives can be used to alter the freezing/gelling point, cloud point, lubricity, viscosity, oxidative stability, ignition quality, octane level, and/or flash point. In the United States, all fuel additives must be registered with Environmental Protection Agency. The names of fuel additives and the companies that sell the fuel additives are publicly available by contacting the EPA or by viewing the agency's website. One of ordinary skill in the art will appreciate that the aldehyde- and/or alkane-based biofuels described herein can be mixed with one or more fuel additives to impart a desired quality.

The aldehyde, fatty alcohols, alkane and/or alkene-based biofuels described herein can be mixed with other fuels, such as various alcohols, such as ethanol and butanol, and petroleum-derived products, such as gasoline, diesel, or jet fuel.

In some examples, the mixture can include at least about 10%, 15%, 20%, 30%, 40%, 50%, or 60% by weight of the aldehyde, fatty alcohols, alkane, or alkene. In other examples, a biofuel composition can be made that includes at least about 5%, 10%, 20%, 30%, 40%, 50%, 60%, 70%, 80%, 85%, 90% or 95% of an aldehyde, fatty alcohols, alkane, or alkene that includes a carbon chain that is 8, 10, 12, 13, 14, 15, 16, 17, 18, 19, 20, 21 or 22 carbons in length. Such biofuel compositions can additionally include at least one additive selected from a cloud point lowering additive that can lower the cloud point to less than about 5° C., or 0° C.; a surfactant; a microemulsion; at least about 5%, 10%, 15%, 20%, 30%, 40%, 50%, 60%, 70%, 80%, 85%, 90%, or 95% diesel fuel from triglycerides; petroleum-derived gasoline; or diesel fuel from petroleum. The invention is further described in the following examples, which do not limit the scope of the invention described in the claims.

### Example 1

This example describes the detection and verification of alkane biosynthesis in selected cyanobacteria.

Seven cyanobacteria, whose complete genome sequences are publicly available, were selected for verification and/or detection of alkane biosynthesis: Synechococcus elongatus PCC7942, Synechococcus elongatus PCC6301, Anabaena variabilis ATCC29413, Synechocystis sp. PCC6803, Nostoc punctiforme PCC73102, Gloeobacter violaceus ATCC 29082, and Prochlorococcus marinus CCMP 1986. Only the first three cyanobacterial strains from this list had previously been reported to contain alkanes (Han et al., J. Am. Chem. Soc. 91:5156-5159 (1969); Fehler et al., Biochem. 9:418-422 (1970)). The strains were grown photoautotrophically in shake flasks in 100 ml of the appropriate media (listed in Table 8) for 3-7 days at 30° C. at a light intensity of approximately 3,500 lux. Cells were extracted for alkane detection as follows: cells from 1 ml culture volume were centrifuged for 1 min at 13,000 rpm, the cell pellets were resuspended in methanol, vortexed for 1 min and then sonicated for 30 min. After centrifugation for 3 min at 13,000 rpm, the supernatants were transferred to fresh vials and analyzed by GC-MS. The samples were analyzed on either 30 m DP-5 capillary column (0.25 mm internal diameter) or a 30 m high temperature DP-5 capillary column (0.250 mm internal diameter) using the following method.

After a 1 μl splitless injection (inlet temperature held at 300° C.) onto the GC/MS column, the oven was held at 100° C. for 3 mins. The temperature was ramped up to 320° C. at a rate of 20° C./min. The oven was held at 320° C. for an additional 5 min. The flow rate of the carrier gas helium was 1.3 ml/min. The MS quadrapole scanned from 50 to 550 m/z. Retention times and fragmentation patterns of product peaks were compared with authentic references to confirm peak identity.

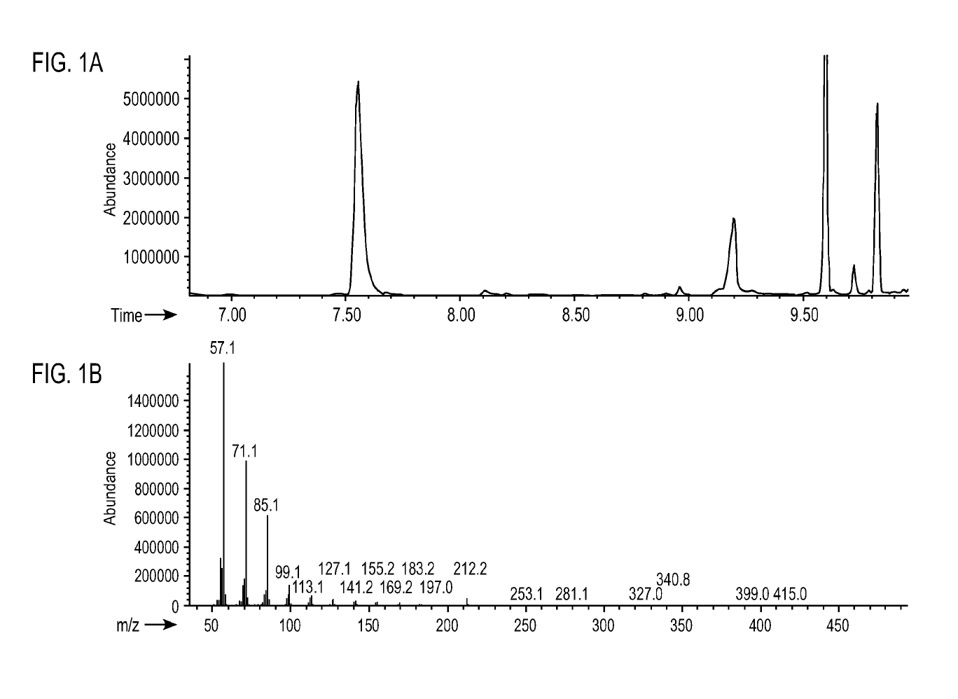

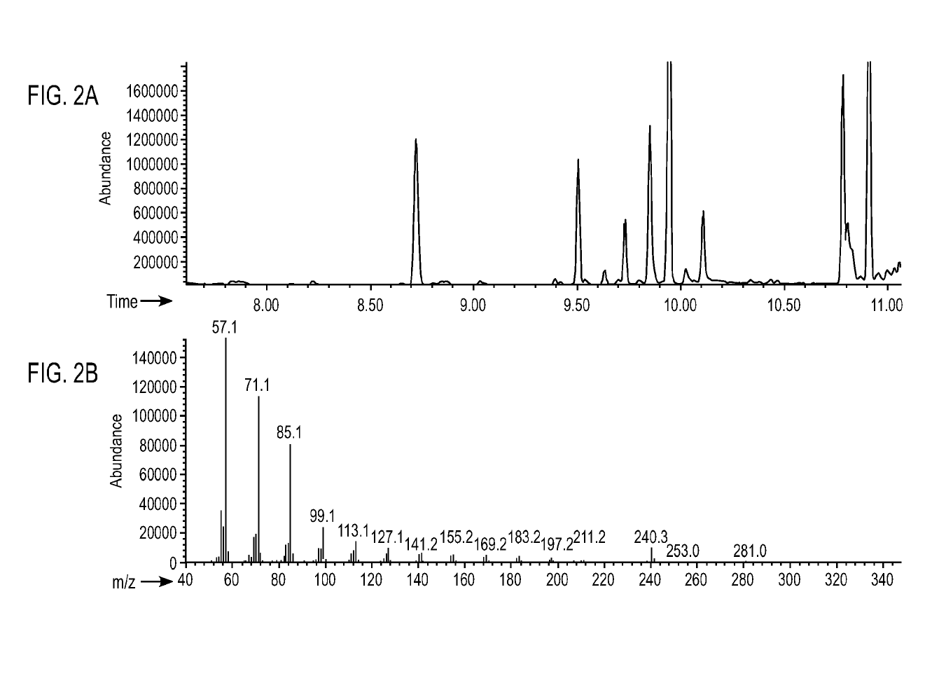

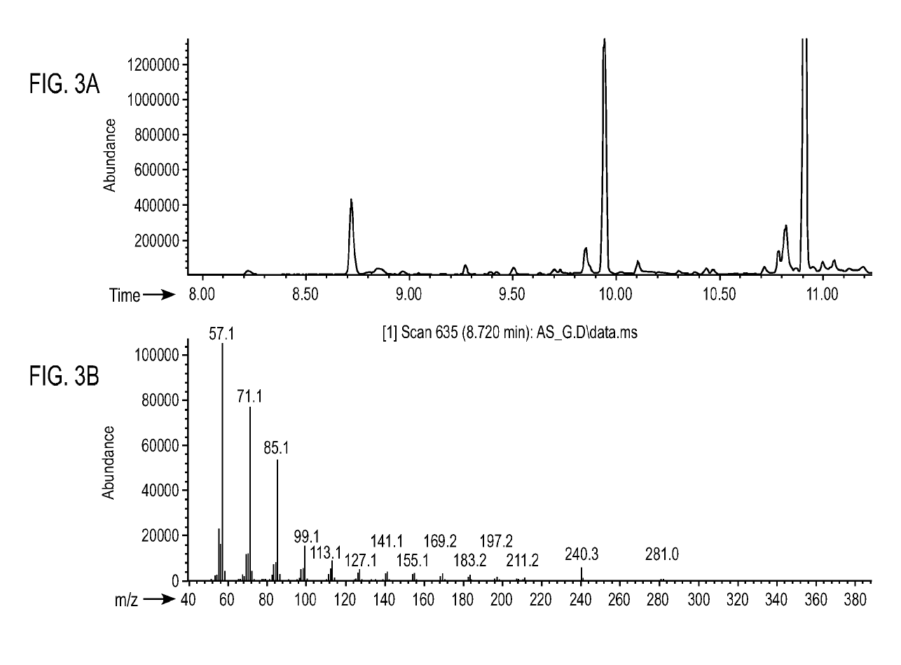

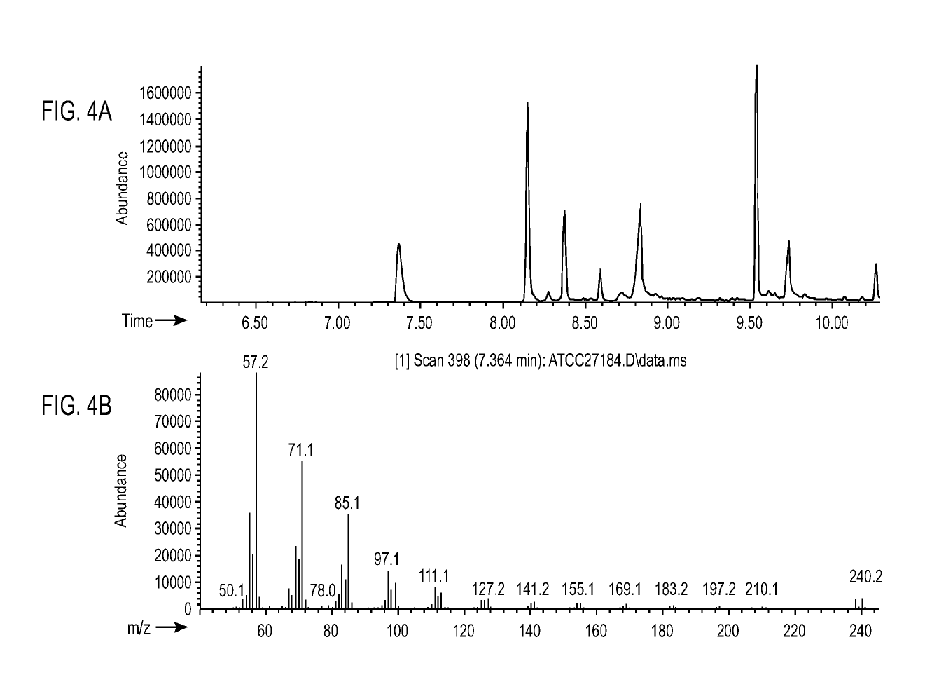

Out of the seven strains, six produced mainly heptadecane and one produced pentadecane (P. marinus CCMP1986); one of these strains produced methyl-heptadecane in addition to heptadecane (A. variabilis ATCC29413) (see Table 8). Therefore, alkane biosynthesis in three previously reported cyanobacteria was verified, and alkane biosynthesis was detected in four cyanobacteria that were not previously known to produce alkanes: P. marinus CCMP1986 (see FIG. 1), N. punctiforme PCC73102 (see FIG. 2), G. violaceus ATCC 29082 (see FIG. 3) and Synechocystis sp. PCC6803 (see FIG. 4).

FIG. 1A depicts the GC/MS trace of Prochlorococcus marinus CCMP1986 cells extracted with methanol. The peak at 7.55 min had the same retention time as pentadecane (Sigma). In FIG. 1B, the mass fragmentation pattern of the pentadecane peak is shown. The 212 peak corresponds to the molecular weight of pentadecane.

FIG. 2A depicts the GC/MS trace of Nostoc punctiforme PCC73102 cells extracted with methanol. The peak at 8.73 min has the same retention time as heptadecane (Sigma). In FIG. 2B, the mass fragmentation pattern of the heptadecane peak is shown. The 240 peak corresponds to the molecular weight of heptadecane.

FIG. 3A depicts the GC/MS trace of Gloeobaceter violaceus ATCC29082 cells extracted with methanol. The peak at 8.72 min has the same retention time as heptadecane (Sigma). In FIG. 3B, the mass fragmentation pattern of the heptadecane peak is shown. The 240 peak corresponds to the molecular weight of heptadecane.

FIG. 4A depicts the GC/MS trace of Synechocystic sp. PCC6803 cells extracted with methanol. The peak at 7.36 min has the same retention time as heptadecane (Sigma). In FIG. 4B, the mass fragmentation pattern of the heptadecane peak is shown. The 240 peak corresponds to the molecular weight of heptadecane.

TABLE 8
Hydrocarbons detected in selected cyanobacteria

| | | | | Alkanes | |
|---|---|---|---|---|---|
| Cyanobacterium | ATCC# | Genome | Medium | Reported | Verified² |
| Synechococcus | 27144 | 2.7 Mb | BG-11 | C17:0 | C17:0, C15:0 |
| elongatus | | | | | |
| PCC7942 | | | | | |
| Synechococcus | 33912 | 2.7 Mb | BG-11 | C17:0 | C17:0, C15:0 |
| elongatus | | | | | |
| PCC6301 | | | | | |
| Anabaena | 29413 | 6.4 Mb | BG-11 | C17:0, 7- or 8- | C17:0, Me-C17:0 |
| variabilis | | | | Me-C17:0 | |
| Synechocystis sp. | 27184 | 3.5 Mb | BG-11 | None | C17:0, C15:0 |
| PCC6803 | | | | | |
| Prochlorococcus | — | 1.7 Mb | — | None | C15:0 |
| marinus | | | | | |
| CCMP1986¹ | | | | | |
| Nostoc | 29133 | 9.0 Mb | ATCC819 | None | C17:0 |
| punctiforme | | | | | |
| PCC73102 | | | | | |
| Gloeobacter violaceus | 29082 | 4.6 Mb | BG11 | None | C17:0 |

¹cells for extraction were a gift from Jacob Waldbauer (MIT)
²major hydrocarbon is in bold

Genomic analysis yielded two genes that were present in the alkane-producing strains. The Synechococcus elongatus PCC7942 homologs of these genes are depicted in Table 9 and are Synpcc7942_{—}1593 (SEQ ID NO: 1) and Synpcc7942_{—}1594 (SEQ ID NO: 66).

TABLE 9
Alkane-producing cyanobacterial genes

| | | Genbank | Inter | |
|---|---|---|---|---|
| Gene Object ID | Locus Tag | accession | Pro | Note |
| 637800026 | Synpcc7942_1593 | YP_400610 | IPR009078 | ferritin/ |
| | | | IPR003251 | ribonucleotide |
| | | | | reductase-like |
| | | | | rubreryhtrin |
| 637800027 | Synpcc7942_1594 | YP_400611 | IPR000408 | predicted |
| | | | IPR016040 | dehydrogenase |
| | | | IPR002198 | NAP(P)-binding |
| | | | | short chain dehydrogenase |

### Example 2

This example demonstrates that the deletion of the sll0208 and sll0209 genes in Synechocystis sp. PCC6803 results in the loss of alkane biosynthesis.

The genes encoding the putative decarbonylase (sll0208; NP_{—}442147) (SEQ ID NO: 3) and aldehyde-generating enzyme (sll0209; NP_{—}442146) (SEQ ID NO: 68) of Synechocystis sp. PCC6803 were deleted as follows. Approximately 1 kb of upstream and downstream flanking DNA were amplified using primer sll0208/9-KO1 (CGCGGATCCCTTGATTCTACTGCGGCGAGT (SEQ ID NO: 97)) with primer sll0208/9-KO2 (CACGCACCTAGGTTCACACTCCCATGGTATAACAGGGGCGTTGGACTCCTGTG (SEQ ID NO: 98)) and primer sll0208/9-KO3 (GTTATACCATGGGAGTGTGAACCTAGGTGCGTGGCCGACAGGATAGGG-CGTGT (SEQ ID NO: 99)) with primer sll0208/9-KO4 (CGCGGATCCAACGCATCCTCACTAGTCGGG (SEQ ID NO: 100)), respectively. The PCR products were used in a cross-over PCR with primers sll0208/9-KO1 and sll0208/9-KO4 to amplify the approximately 2 kb sll0208/sll0209 deletion cassette, which was cloned into the BamHI site of the cloning vector pUC19. A kanamycin resistance cassette (aph, KanR) was then amplified from plasmid pRL27 (Larsen et al., Arch. Microbiol. 178:193 (2002)) using primers Kan-aph-F (CATGCCATGGAAAGCCACGTTGTGTCTCAAAATCTCTG (SEQ ID NO: 101)) and Kan-aph-R(CTAGTCTAGAGCGCTGAGGTCTGCCTCGTGAA (SEQ ID NO: 102)), which was then digested with NcoI and XbaI and cloned into the NcoI and AvrII sites of the sll0208/sll0209 deletion cassette, creating a sll0208/sll0209-deletion KanR-insertion cassette in pUC19. The cassette-containing vector, which does not replicate in cyanobacteria, was transformed into Synechocystis sp. PCC6803 (Zang et al., 2007, J. Microbiol., vol. 45, pp. 241) and transformants (e.g., chromosomal integrants by double-homologous recombination) were selected on BG-11 agar plates containing 100 μg/ml Kanamycin in a light-equipped incubator at 30° C. Kanamycin resistant colonies were restreaked once and then subjected to genotypic analysis using PCR with diagnostic primers.

Confirmed deletion-insertion mutants were cultivated in 12 ml of BG11 medium with 50 μg/ml Kanamycin for 4 days at 30° C. in a light-equipped shaker-incubator. 1 ml of broth was then centrifuged (1 min at 13,000 g) and the cell pellets were extracted with 0.1 ml methanol. After extraction, the samples were again centrifuged and the supernatants were subjected to GC-MS analysis as described in Example 1.

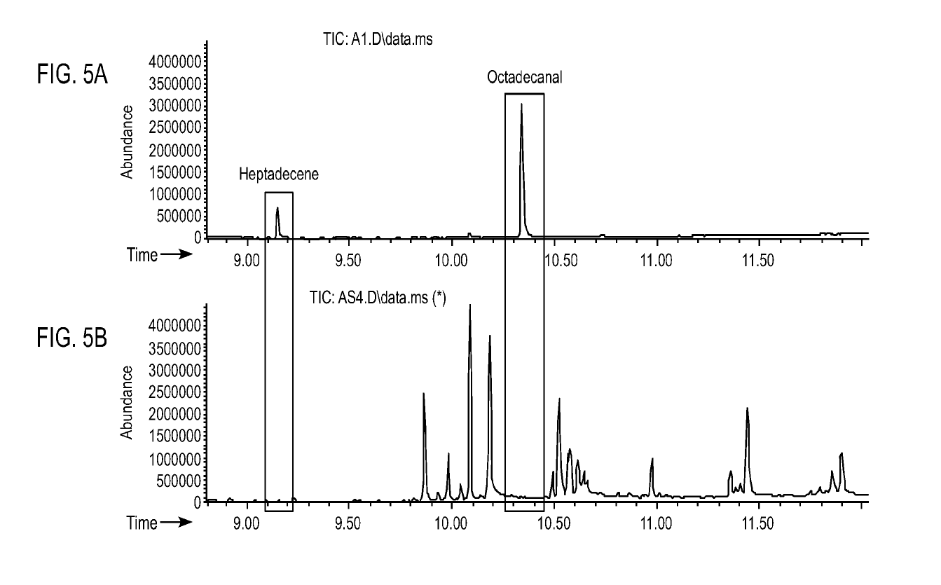

As shown in FIG. 5, the Synechocystis sp. PCC6803 strains in which the sll0208 and sll0209 genes were deleted lost their ability to produce heptadecene and octadecenal. This result demonstrates that the sll0208 and sll0209 genes in Synechocystis sp. PCC6803 and the orthologous genes in other cyanobacteria (see Table 1) are responsible for alkane and fatty aldehyde biosynthesis in these organisms.

### Example 3

This example demonstrates the production of fatty aldehydes and fatty alcohols in E. coli through heterologous expression of Synechococcus elongatus PCC7942 orf1594.

The genomic DNA encoding Synechococcus elongatus PCC7942 orf1594 (YP_{—}400611; putative aldehyde-generating enzyme) (SEQ ID NO: 65) was amplified and cloned into the NcoI and EcoRI sites of vector OP-80 (pCL1920 derivative) under the control of the P_{trc} promoter. The resulting construct (“OP80-PCC7942_{—}1594”) was transformed into E. coli MG1655 and the cells were grown at 37° C. in M9 minimal media with 1% (w/v) glucose as carbon source and supplemented with 100 μg/ml spectinomycin. When the culture reached OD₆₀₀ of 0.8-1.0, it was induced with 1 mM IPTG and cells were grown for an additional 18-20 h at 37° C. Cells from 0.5 ml of culture were extracted with 0.5 ml of ethyl acetate. After sonication for 60 min, the sample was centrifuged at 15,000 rpm for 5 min. The solvent layer was analyzed by GC-MS as described in Example 1.

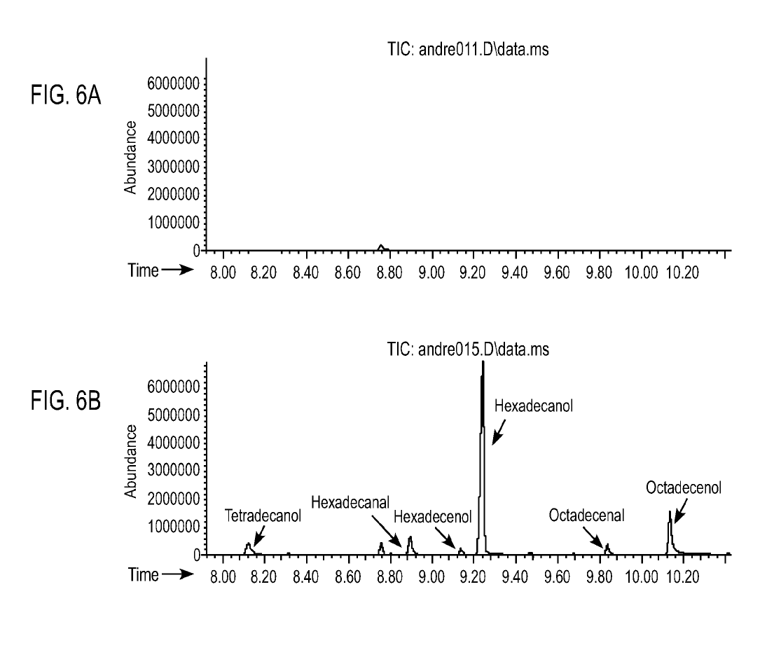

As shown in FIG. 6, E. coli cells transformed with the Synechococcus elongatus PCC7942 orf1594-bearing vector produced the following fatty aldehydes and fatty alcohols: hexadecanal, octadecenal, tetradecenol, hexadecenol, hexadecanol and octadecenol. This result indicates that PCC7942 orf1594 generates aldehydes in vivo as possible substrates for decarbonylation, and may reduce acyl-ACPs as substrates, which are the most abundant form of activated fatty acids in wild type E. coli cells. Therefore, the enzyme was named Acyl-ACP reductase. In vivo, the fatty aldehydes apparently are further reduced to the corresponding fatty alcohols by an endogenous E. coli aldehyde reductase activity.

### Example 4

This example demonstrates the production of fatty aldehydes and fatty alcohols in E. coli through heterologous expression of Cyanothece sp. ATCC51142 cce_{—}1430.

The genomic DNA encoding Cyanothece sp. ATCC51142 cce_{—}1430 (YP_{—}001802846; putative aldehyde-generating enzyme) (SEQ ID NO: 70) was amplified and cloned into the NcoI and EcoRI sites of vector OP-80 (pCL1920 derivative) under the control of the P_{trc} promoter. The resulting construct was transformed into E. coli MG1655 and the cells were grown at 37° C. in M9 minimal media with 1% (w/v) glucose as carbon source and supplemented with 100 μg/ml spectinomycin. The cells were cultured and extracted as in Example 3 and analyzed by GC-MS as described in Example 26.

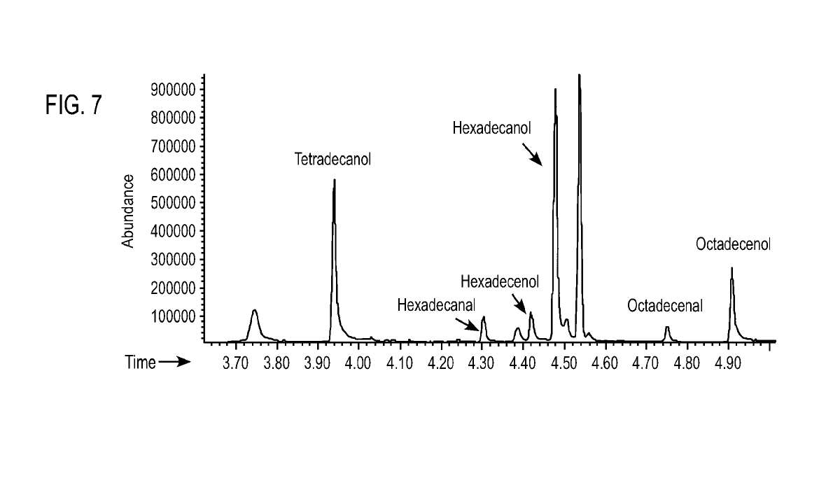

As shown in FIG. 7, E. coli cells transformed with the Cyanothece sp. ATCC51142 cce_{—}1430-bearing vector produced the following fatty aldehydes and fatty alcohols: hexadecanal, octadecenal, tetradecenol, hexadecenol, hexadecanol and octadecenol. This result indicates that ATCC51142 cce_{—}1430 generates aldehydes in vivo as possible substrates for decarbonylation, and may reduce acyl-ACPs as substrates, which are the most abundant form of activated fatty acids in wild type E. coli cells. Therefore, this enzyme is also an Acyl-ACP reductase.

### Example 5

This example demonstrates the production of alkanes and alkenes in E. coli through heterologous expression of Synechococcus elongatus PCC7942 orf1594 and Synechococcus elongatus PCC7942 orf1593.

The genomic DNA encoding Synechococcus elongatus PCC7942 orf1593 (YP_{—}400610; putative decarbonylase) (SEQ ID NO: 1) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

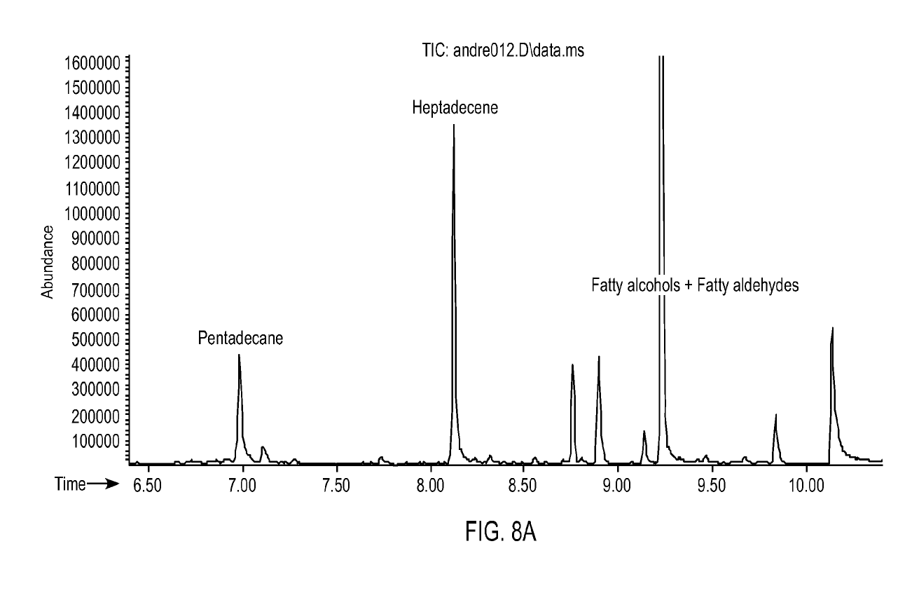

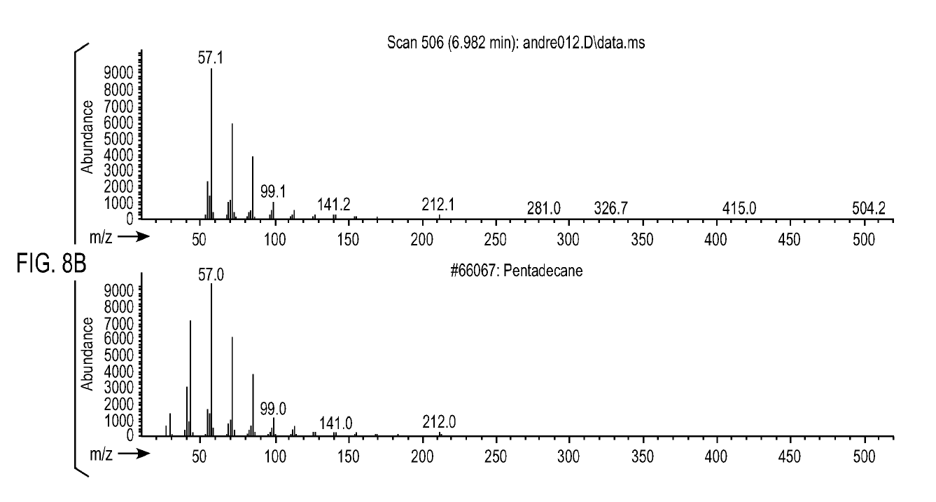

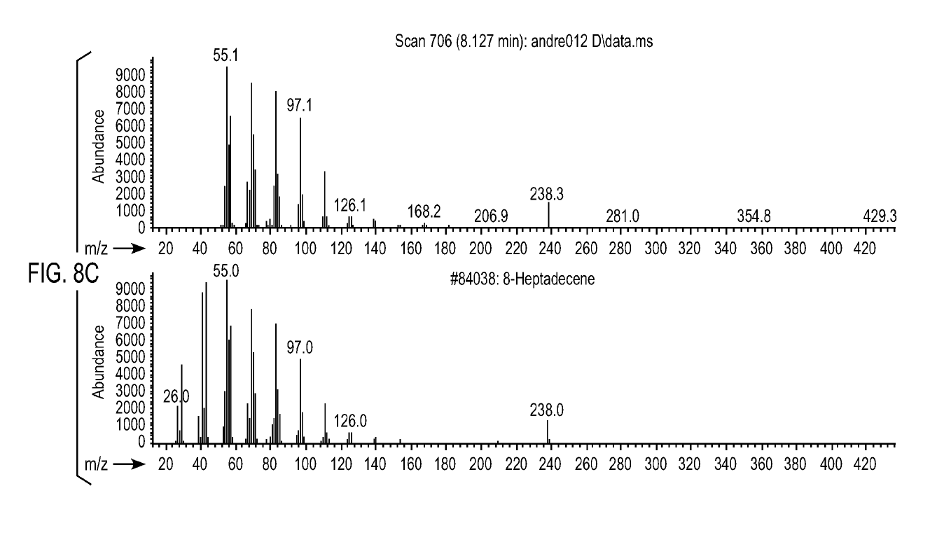

As shown in FIG. 8, E. coli cells cotransformed with the S. elongatus PCC7942_{—}1594 and S. elongatus PCC7942_{—}1593-bearing vectors produced the same fatty aldehydes and fatty alcohols as in Example 3, but also pentadecane and heptadecene. This result indicates that PCC7942_{—}1593 in E. coli converts hexadecanal and octadecenal to pentadecane and heptadecene, respectively, and therefore is an active fatty aldehyde decarbonylase and a suitable alkane/alkene biosynthetic polypeptide.

### Example 6

This example demonstrates the production of alkanes and alkenes in E. coli through heterologous expression of Synechococcus elongatus PCC7942 orf1594 and Nostoc punctiforme PCC73102 Npun02004178.

The genomic DNA encoding Nostoc punctiforme PCC73102 Npun02004178 (ZP_{—}00108838; putative decarbonylase) (SEQ ID NO: 5) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

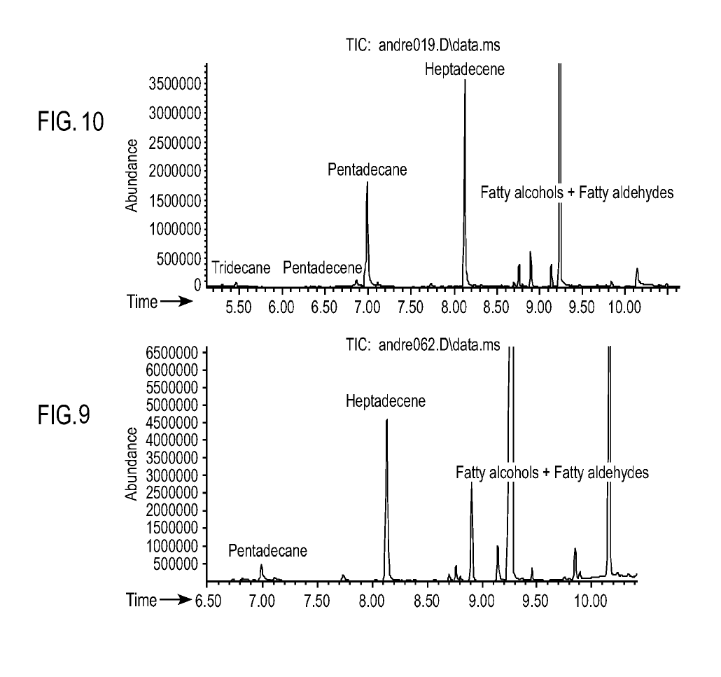

As shown in FIG. 9, E. coli cells cotransformed with the S. elongatus PCC7942_{—}1594 and N. punctiforme PCC73102 Npun02004178-bearing vectors produced the same fatty aldehydes and fatty alcohols as in Example 3, but also tridecane, pentadecene, pentadecane and heptadecene. This result indicates that Npun02004178 in E. coli converts tetradecanal, hexadecenal, hexadecanal and octadecenal to tridecane, pentadecene, pentadecane and heptadecene, respectively, and therefore is an active fatty aldehyde decarbonylase and a suitable alkane/alkene biosynthetic polypeptide.

### Example 7

This example demonstrates the production of alkanes and alkenes in E. coli through heterologous expression of Synechococcus elongatus PCC7942 orf1594 and Synechocystis sp. PCC6803 sll0208.

The genomic DNA encoding Synechocystis sp. PCC6803 sll0208 (NP_{—}442147; putative decarbonylase) (SEQ ID NO: 3) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

As shown in FIG. 10, E. coli cells cotransformed with the S. elongatus PCC7942_{—}1594 and Synechocystis sp. PCC6803 sll0208-bearing vectors produced the same fatty aldehydes and fatty alcohols as in Example 3, but also pentadecane and heptadecene. This result indicates that Npun02004178 in E. coli converts hexadecanal and octadecenal to pentadecane and heptadecene, respectively, and therefore is an active fatty aldehyde decarbonylase and a suitable alkane/alkene biosynthetic polypeptide.

### Example 8

This example demonstrates the production of alkanes and alkenes in E. coli through heterologous expression of Synechococcus elongatus PCC7942 orf1594 and Nostoc sp. PCC7210 alr5283.

The genomic DNA encoding Nostoc sp. PCC7210 alr5283 (NP_{—}489323; putative decarbonylase) (SEQ ID NO: 7) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

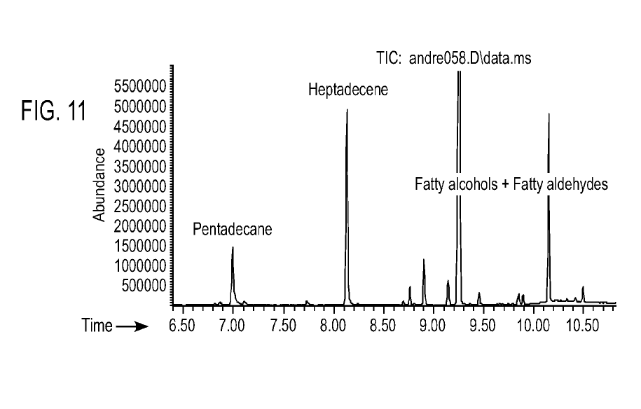

As shown in FIG. 11, E. coli cells cotransformed with the S. elongatus PCC7942_{—}1594 and Nostoc sp. PCC7210 alr5283-bearing vectors produced the same fatty aldehydes and fatty alcohols as in Example 3, but also pentadecane and heptadecene. This result indicates that alr5283 in E. coli converts hexadecanal and octadecenal to pentadecane and heptadecene, respectively, and therefore is an active fatty aldehyde decarbonylase and a suitable alkane/alkene biosynthetic polypeptide.

### Example 9

This example demonstrates the production of alkanes and alkenes in E. coli through heterologous expression of Synechococcus elongatus PCC7942 orf1594 and Acaryochloris marina MBIC11017 AM1_{—}4041.

The genomic DNA encoding Acaryochloris marina MBIC11017 AM1_{—}4041 (YP_{—}001518340; putative decarbonylase) (SEQ ID NO: 9) was codon optimized for expression in E. coli (SEQ ID NO: 47), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

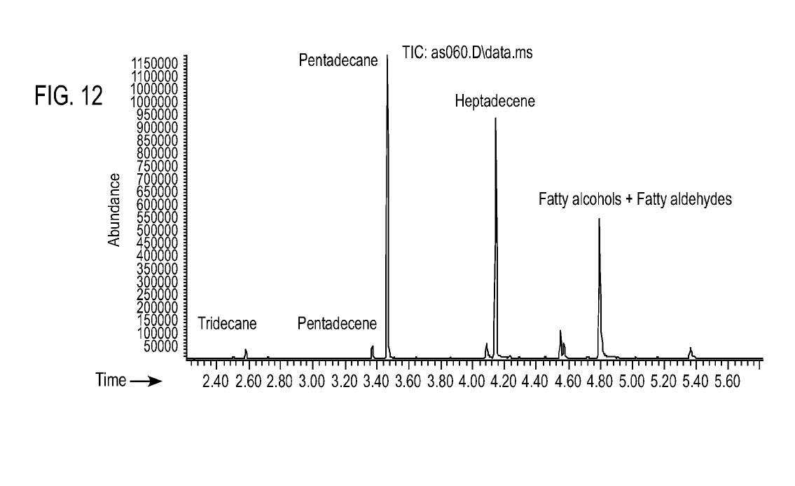

As shown in FIG. 12, E. coli cells cotransformed with the S. elongatus PCC7942_{—}1594 and A. marina MBIC11017 AM1_{—}4041-bearing vectors produced the same fatty aldehydes and fatty alcohols as in Example 3, but also tridecane, pentadecene, pentadecane and heptadecene. This result indicates that AM1_{—}4041 in E. coli converts tetradecanal, hexadecenal, hexadecanal and octadecenal to tridecane, pentadecene, pentadecane and heptadecene, respectively, and therefore is an active fatty aldehyde decarbonylase and a suitable alkane/alkene biosynthetic polypeptide.

### Example 10

This example demonstrates the production of alkanes and alkenes in E. coli through heterologous expression of Synechococcus elongatus PCC7942 orf1594 and Thermosynechococcus elongatus BP-1 tll1313.

The genomic DNA encoding Thermosynechococcus elongatus BP-1 tll1313 (NP_{—}682103; putative decarbonylase) (SEQ ID NO: 11) was codon optimized for expression in E. coli (SEQ ID NO: 48), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

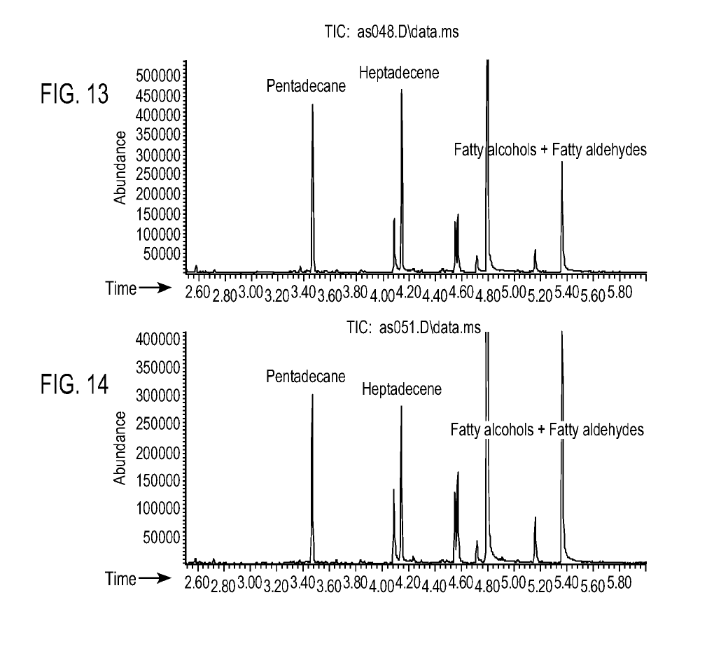

As shown in FIG. 13, E. coli cells cotransformed with the S. elongatus PCC7942_{—}1594 and T. elongatus BP-1 tll1313-bearing vectors produced the same fatty aldehydes and fatty alcohols as in Example 3, but also pentadecane and heptadecene. This result indicates that tll1313 in E. coli converts hexadecanal and octadecenal to pentadecane and heptadecene, respectively, and therefore is an active fatty aldehyde decarbonylase and a suitable alkane/alkene biosynthetic polypeptide.

### Example 11

This example demonstrates the production of alkanes and alkenes in E. coli through heterologous expression of Synechococcus elongatus PCC7942 orf1594 and Synechococcus sp. JA-3-3Ab CYA_{—}0415.

The genomic DNA encoding Synechococcus sp. JA-3-3Ab CYA_{—}0415 (YP_{—}473897; putative decarbonylase) (SEQ ID NO: 13) was codon optimized for expression in E. coli (SEQ ID NO: 49), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 n/ml carbenicillin. The cells were cultured and extracted as in Example 3 and analyzed by GC-MS as described in Example 26.

As shown in FIG. 14, E. coli cells cotransformed with the S. elongatus PCC7942_{—}1594 and Synechococcus sp. JA-3-3Ab CYA_{—}0415-bearing vectors produced the same fatty aldehydes and fatty alcohols as in Example 3, but also pentadecane and heptadecene. This result indicates that Npun02004178 in E. coli converts hexadecanal and octadecenal to pentadecane and heptadecene, respectively, and therefore is an active fatty aldehyde decarbonylase and a suitable alkane/alkene biosynthetic polypeptide.

### Example 12

This example demonstrates the production of alkanes and alkenes in E. coli through heterologous expression of Synechococcus elongatus PCC7942 orf1594 and Gloeobacter violaceus PCC7421 gll3146.

The genomic DNA encoding Gloeobacter violaceus PCC7421 gll3146 (NP_{—}926092; putative decarbonylase) (SEQ ID NO: 15) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

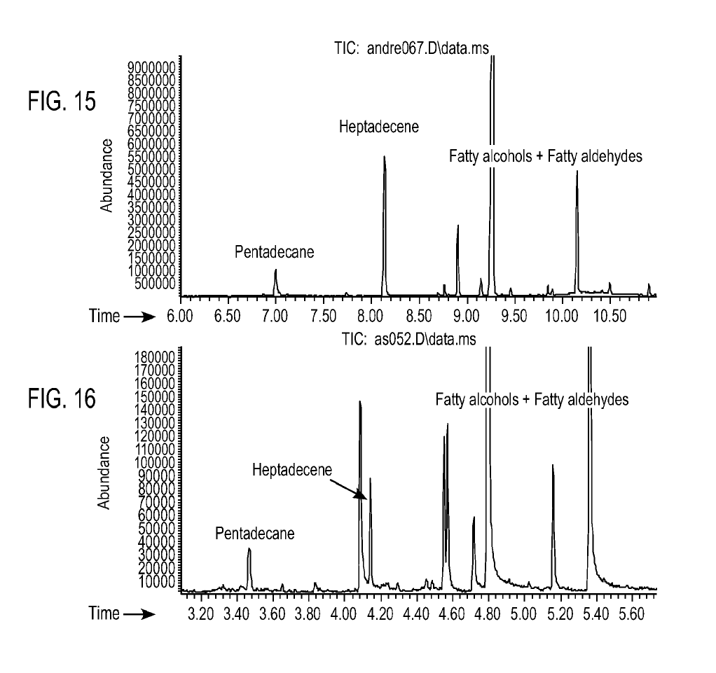

As shown in FIG. 15, E. coli cells cotransformed with the S. elongatus PCC7942_{—}1594 and G. violaceus PCC7421 gll3146-bearing vectors produced the same fatty aldehydes and fatty alcohols as in Example 3, but also pentadecane and heptadecene. This result indicates that gll3146 in E. coli converts hexadecanal and octadecenal to pentadecane and heptadecene, respectively, and therefore is an active fatty aldehyde decarbonylase and a suitable alkane/alkene biosynthetic polypeptide.

### Example 13

This example demonstrates the production of alkanes and alkenes in E. coli through heterologous expression of Synechococcus elongatus PCC7942 orf1594 and Prochlorococcus marinus MIT9313 PM1231.

The genomic DNA encoding Prochlorococcus marinus MIT9313 PM1231 (NP_{—}895059; putative decarbonylase) (SEQ ID NO: 17) was codon optimized for expression in E. coli (SEQ ID NO: 50), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

As shown in FIG. 16, E. coli cells cotransformed with the S. elongatus PCC7942_{—}1594 and P. marinus MIT9313 PM1231-bearing vectors produced the same fatty aldehydes and fatty alcohols as in Example 3, but also pentadecane and heptadecene. This result indicates that PM1231 in E. coli converts hexadecanal and octadecenal to pentadecane and heptadecene, respectively, and therefore is an active fatty aldehyde decarbonylase and a suitable alkane/alkene biosynthetic polypeptide.

### Example 14

This example demonstrates the production of alkanes and alkenes in E. coli through heterologous expression of Synechococcus elongatus PCC7942 orf1594 and Prochlorococcus marinus CCMP1986 PMM0532.

The genomic DNA encoding Prochlorococcus marinus CCMP1986 PMM0532 (NP_{—}892650; putative decarbonylase) (SEQ ID NO: 19) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

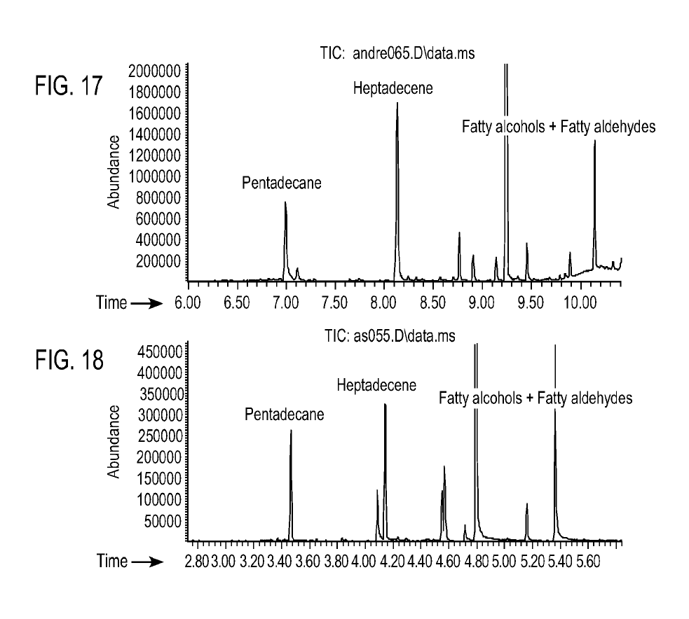

As shown in FIG. 17, E. coli cells cotransformed with the S. elongatus PCC7942_{—}1594 and P. marinus CCMP1986 PMM0532-bearing vectors produced the same fatty aldehydes and fatty alcohols as in Example 3, but also pentadecane and heptadecene. This result indicates that PMM0532 in E. coli converts hexadecanal and octadecenal to pentadecane and heptadecene, respectively, and therefore is an active fatty aldehyde decarbonylase and a suitable alkane/alkene biosynthetic polypeptide.

### Example 15

This example demonstrates the production of alkanes and alkenes in E. coli through heterologous expression of Synechococcus elongatus PCC7942 orf1594 and Prochlorococcus mariunus NATL2A PMN2A_{—}1863.

The genomic DNA encoding Prochlorococcus mariunus NATL2A PMN2A_{—}1863 (YP_{—}293054; putative decarbonylase) (SEQ ID NO: 21) was codon optimized for expression in E. coli (SEQ ID NO: 52), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 mg/ml carbenicillin. The cells were cultured and extracted as in Example 3 and analyzed by GC-MS as described in Example 26.

As shown in FIG. 18, E. coli cells cotransformed with the S. elongatus PCC7942_{—}1594 and P. mariunus NATL2A PMN2A_{—}1863-bearing vectors produced the same fatty aldehydes and fatty alcohols as in Example 3, but also pentadecane and heptadecene. This result indicates that PMN2A_{—}1863 in E. coli converts hexadecanal and octadecenal to pentadecane and heptadecene, respectively, and therefore is an active fatty aldehyde decarbonylase and a suitable alkane/alkene biosynthetic polypeptide.

### Example 16

This example demonstrates the production of alkanes and alkenes in E. coli through heterologous expression of Synechococcus elongatus PCC7942 orf1594 and Synechococcus sp. RS9917 RS9917_{—}09941.

The genomic DNA encoding Synechococcus sp. RS9917 RS9917_{—}09941 (ZP_{—}01079772; putative decarbonylase) (SEQ ID NO: 23) was codon optimized for expression in E. coli (SEQ ID NO: 53), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

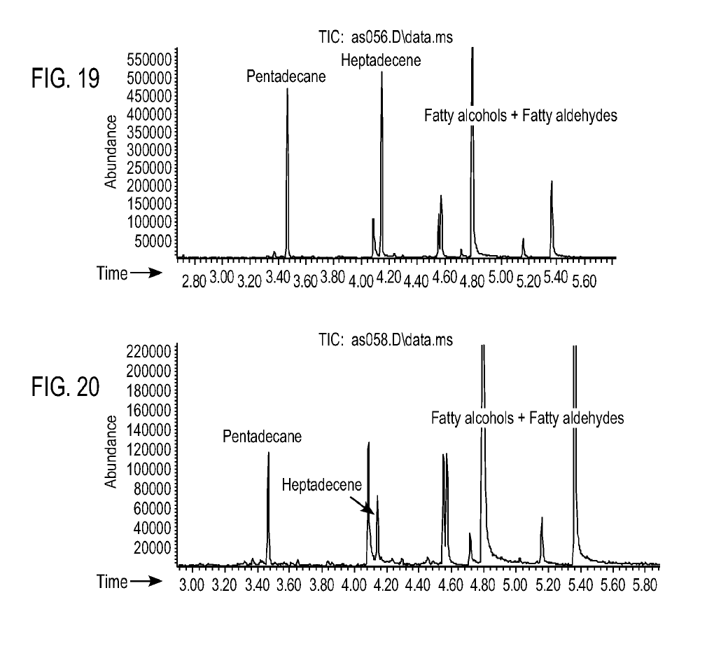

As shown in FIG. 19, E. coli cells cotransformed with the S. elongatus PCC7942_{—}1594 and Synechococcus sp. RS9917 RS9917_{—}09941-bearing vectors produced the same fatty aldehydes and fatty alcohols as in Example 3, but also pentadecane and heptadecene. This result indicates that RS991709941 in E. coli converts hexadecanal and octadecenal to pentadecane and heptadecene, respectively, and therefore is an active fatty aldehyde decarbonylase and a suitable alkane/alkene biosynthetic polypeptide.

### Example 17

This example demonstrates the production of alkanes and alkenes in E. coli through heterologous expression of Synechococcus elongatus PCC7942 orf1594 and Synechococcus sp. RS9917 RS9917_{—}12945.

The genomic DNA encoding Synechococcus sp. RS9917 RS9917_{—}12945 (ZP_{—}01080370; putative decarbonylase) (SEQ ID NO: 25) was codon optimized for expression in E. coli (SEQ ID NO: 54), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

As shown in FIG. 20, E. coli cells cotransformed with the S. elongatus PCC7942_{—}1594 and Synechococcus sp. RS9917 RS9917_{—}12945-bearing vectors produced the same fatty aldehydes and fatty alcohols as in Example 3, but also pentadecane and heptadecene. This result indicates that RS9917_{—}12945 in E. coli converts hexadecanal and octadecenal to pentadecane and heptadecene, respectively, and therefore is an active fatty aldehyde decarbonylase and a suitable alkane/alkene biosynthetic polypeptide.

### Example 18

This example demonstrates the production of alkanes and alkenes in E. coli through heterologous expression of Synechococcus elongatus PCC7942 orf1594 and Cyanothece sp. ATCC51142 cce_{—}0778.

The genomic DNA encoding Cyanothece sp. ATCC51142 cce_{—}0778 (YP_{—}001802195; putative decarbonylase) (SEQ ID NO: 27) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

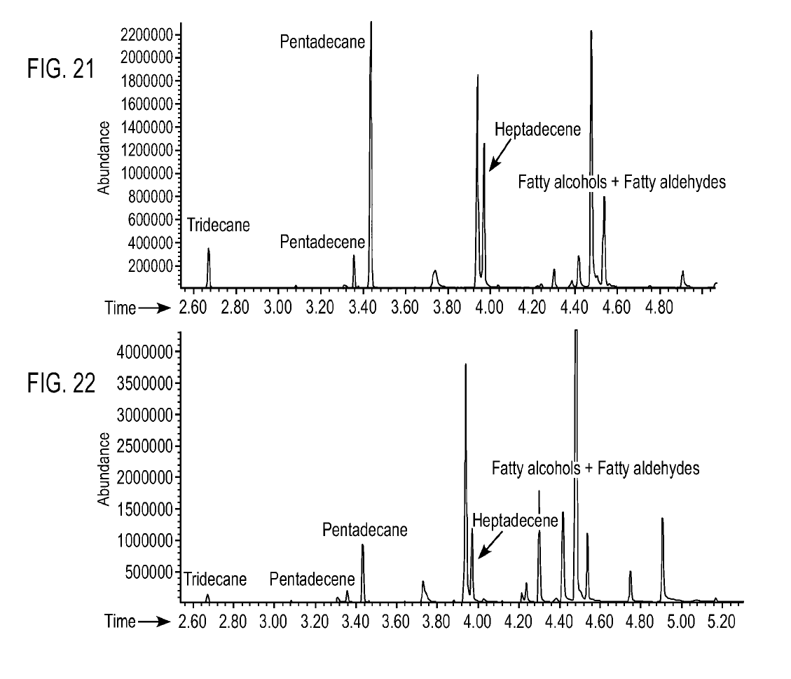

As shown in FIG. 21, E. coli cells cotransformed with the S. elongatus PCC7942_{—}1594 and Cyanothece sp. ATCC51142 cce_{—}0778-bearing vectors produced the same fatty aldehydes and fatty alcohols as in Example 3, but also tridecane, pentadecene, pentadecane and heptadecene. This result indicates that ATCC51142 cce_{—}0778 in E. coli converts tetradecanal, hexadecenal, hexadecanal and octadecenal to tridecane, pentadecene, pentadecane and heptadecene, respectively, and therefore is an active fatty aldehyde decarbonylase and a suitable alkane/alkene biosynthetic polypeptide.

### Example 19

This example demonstrates the production of alkanes and alkenes in E. coli through heterologous expression of Synechococcus elongatus PCC7942 orf1594 and Cyanothece sp. PCC7425 Cyan7425_{—}0398.

The genomic DNA encoding Cyanothece sp. PCC7425 Cyan7425_{—}0398 (YP_{—}002481151; putative decarbonylase) (SEQ ID NO: 29) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

As shown in FIG. 22, E. coli cells cotransformed with the S. elongatus PCC7942_{—}1594 and Cyanothece sp. PCC7425 Cyan7425_{—}0398-bearing vectors produced the same fatty aldehydes and fatty alcohols as in Example 3, but also tridecane, pentadecene, pentadecane and heptadecene. This result indicates that Cyan7425_{—}0398 in E. coli converts tetradecanal, hexadecenal, hexadecanal and octadecenal to tridecane, pentadecene, pentadecane and heptadecene, respectively, and therefore is an active fatty aldehyde decarbonylase and a suitable alkane/alkene biosynthetic polypeptide.

### Example 20

This example demonstrates the production of alkanes and alkenes in E. coli through heterologous expression of Synechococcus elongatus PCC7942 orf1594 and Cyanothece sp. PCC7425 Cyan7425_{—}2986.

The genomic DNA encoding Cyanothece sp. PCC7425 Cyan7425_{—}2986 (YP_{—}002483683; putative decarbonylase) (SEQ ID NO: 31) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

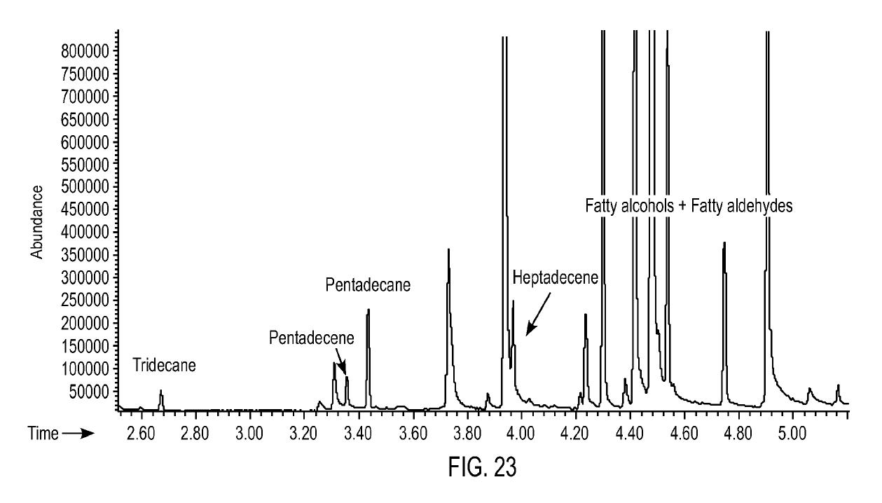

As shown in FIG. 23, E. coli cells cotransformed with the S. elongatus PCC7942_{—}1594 and Cyanothece sp. PCC7425 Cyan7425_{—}2986-bearing vectors produced the same fatty aldehydes and fatty alcohols as in Example 3, but also tridecane, pentadecene, pentadecane and heptadecene. This result indicates that Cyan7425_{—}2986 in E. coli converts tetradecanal, hexadecenal, hexadecanal and octadecenal to tridecane, pentadecene, pentadecane and heptadecene, respectively, and therefore is an active fatty aldehyde decarbonylase and a suitable alkane/alkene biosynthetic polypeptide.

### Example 21

This example demonstrates the production of alkanes and alkenes in E. coli through heterologous expression of Prochlorococcus marinus CCMP1986 PMM0533 and Prochlorococcus mariunus CCMP1986 PMM0532.

The genomic DNA encoding P. mariunus CCMP1986 PMM0533 (NP_{—}892651; putative aldehyde-generating enzyme) (SEQ ID NO: 72) and Prochlorococcus mariunus CCMP1986 PMM0532 (NP_{—}892650; putative decarbonylase) (SEQ ID NO: 19) were amplified and cloned into the NcoI and EcoRI sites of vector OP-80 and the NdeI and XhoI sites of vector OP-183, respectively. The resulting constructs were separately transformed and cotransformed into E. coli MG1655 and the cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

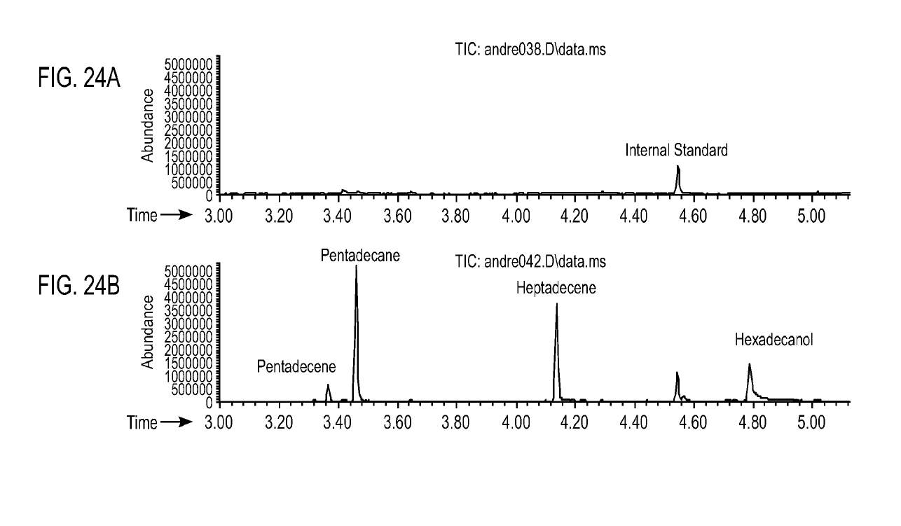

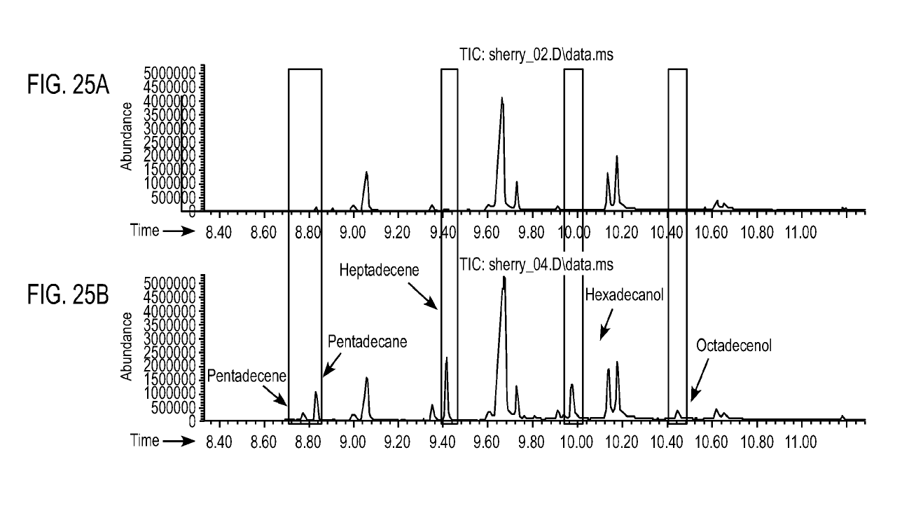

As shown in FIG. 24A, E. coli cells transformed with only the P. mariunus CCMP1986 PMM0533-bearing vector did not produce any fatty aldehydes or fatty alcohols. However, E. coli cells cotransformed with PMM0533 and PMM0532-bearing vectors produced hexadecanol, pentadecane and heptadecene (FIG. 24B). This result indicates that PMM0533 only provides fatty aldehyde substrates for the decarbonylation reaction when it interacts with a decarbonylase, such as PMM0532.

### Example 22

This example demonstrates the production of alkanes and alkenes in a fatty acyl-CoA-producing E. coli strain through heterologous expression of Synechococcus elongatus PCC7942 orf1594 and Acaryochloris marina MBIC11017 AM1_{—}4041.

The genomic DNA encoding Acaryochloris marina MBIC11017 AM1_{—}4041 (YP_{—}001518340; putative fatty aldehyde decarbonylase) (SEQ ID NO: 9) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 ΔfadE lacZ::P_{trc} ′tesA-fadD. This strain expresses a cytoplasmic version of the E. coli thioesterase, ′TesA, and the E. coli acyl-CoA synthetase, FadD, under the control of the P_{trc} promoter, and therefore produces fatty acyl-CoAs. The cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

As shown in FIG. 25, these E. coli cells cotransformed with S. elongatus PCC7942_{—}1594 and A. marina MBIC11017 AM1_{—}4041 also produced alkanes and fatty alcohols. This result indicates that S. elongatus PCC7942_{—}1594 is able to use acyl-CoA as a substrate to produce hexadecenal, hexadecanal and octadecenal, which is then converted into pentadecene, pentadecane and heptadecene, respectively, by A. marina MBIC 11017 AM1_{—}4041.

### Example 23

This example demonstrates the production of alkanes and alkenes in a fatty acyl-CoA-producing E. coli strain through heterologous expression of Synechocystis sp. PCC6803 sll0209 and Synechocystis sp. PCC6803 sll0208.

The genomic DNA encoding Synechocystis sp. PCC6803 sll0208 (NP_{—}442147; putative fatty aldehyde decarbonylase) (SEQ ID NO: 3) was synthesized and cloned into the NdeI and XhoI sites of vector OP-183 (pACYC derivative) under the control of the P_{trc} promoter. The genomic DNA encoding Synechocystis sp. PCC6803 sll0209 (NP_{—}442146; acyl-ACP reductase) (SEQ ID NO: 68) was synthesized and cloned into the NcoI and EcoRI sites of vector OP-183 (pACYC derivative) under the control of the P_{trc} promoter. The resulting constructs were cotransformed with into E. coli MG1655 ΔfadE lacZ::P_{trc} ′tesA-fadD. This strain expresses a cytoplasmic version of the E. coli thioesterase, ′TesA, and the E. coli acyl-CoA synthetase, FadD, under the control of the P_{trc} promoter, and therefore produces fatty acyl-CoAs. The cells were grown at 37° C. in M9 minimal media supplemented with 100 μg/ml spectinomycin and 100 μg/ml carbenicillin. The cells were cultured and extracted as in Example 3 and analyzed by GC-MS as described in Example 26.

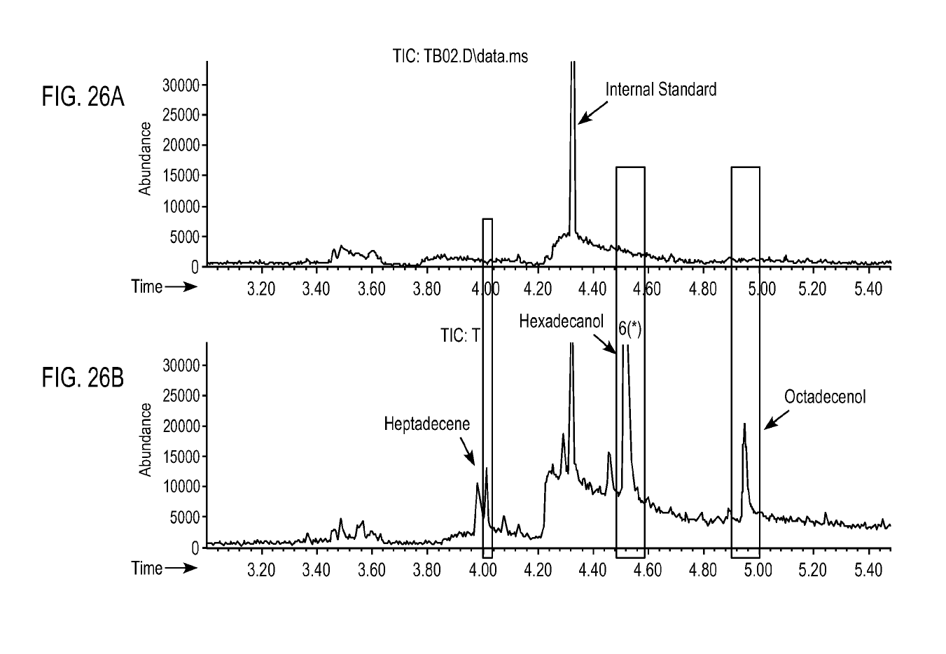

As shown in FIG. 26, these E. coli cells transformed with Synechocystis sp. PCC6803 sll0209 did not produce any fatty aldehydes or fatty alcohols. However, when cotransfouned with Synechocystis sp. PCC6803 sll0208 and sll0209, they produced alkanes, fatty aldehydes and fatty alcohols. This result indicates that Synechocystis sp. PCC6803 sll0209 is able to use acyl-CoA as a substrate to produce fatty aldehydes such as tetradecanal, hexadecanal and octadecenal, but only when coexpressed with a fatty aldehyde decarbonylase. The fatty aldehydes apparently are further reduced to the corresponding fatty alcohols, tetradecanol, hexadecanol and octadecenol, by an endogenous E. coli aldehyde reductase activity. In this experiment, octadecenal was converted into heptadecene by Synechocystis sp. PCC6803 sll0208.

### Example 24

This example demonstrates the production of alkanes and alkenes in a fatty aldehyde-producing E. coli strain through heterologous expression of Nostoc punctiforme PCC73102 Npun02004178 and several of its homologs.

The genomic DNA encoding Nostoc punctiforme PCC73102 Npun02004178 (ZP_{—}00108838; putative fatty aldehyde decarbonylase) (SEQ ID NO: 5) was amplified and cloned into the NdeI and XhoI sites of vector OP-183 (pACYC derivative) under the control of the P_{trc} promoter. The genomic DNA encoding Mycobacterium smegmatis strain MC2 155 orf MSMEG_{—}5739 (YP_{—}889972, putative carboxylic acid reductase) (SEQ ID NO: 86) was amplified and cloned into the NcoI and EcoRI sites of vector OP-180 (pCL1920 derivative) under the control of the P_{trc} promoter. The two resulting constructs were cotransformed into E. coli MG1655 ΔfadD lacZ::P_{trc}-′tesA. In this strain, fatty aldehydes were provided by MSMEG_{—}5739, which reduces free fatty acids (formed by the action of ′TesA) to fatty aldehydes. The cells were grown at 37° C. in M9 minimal media supplemented with 100 μg/ml spectinomycin and 100 μg/ml carbenicillin. The cells were cultured and extracted as in Example 3 and analyzed by GC-MS as described in Example 1.

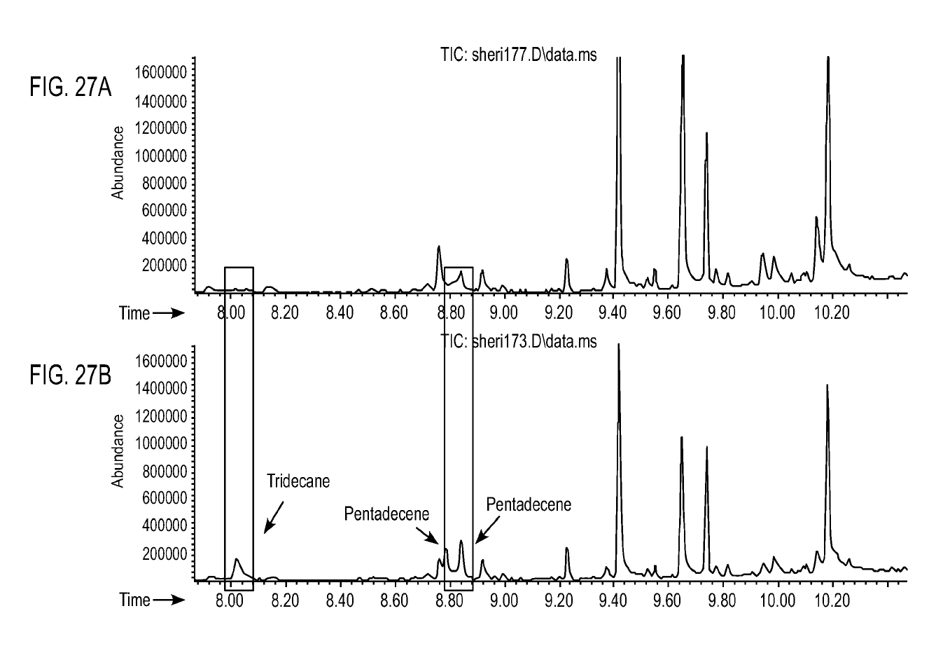

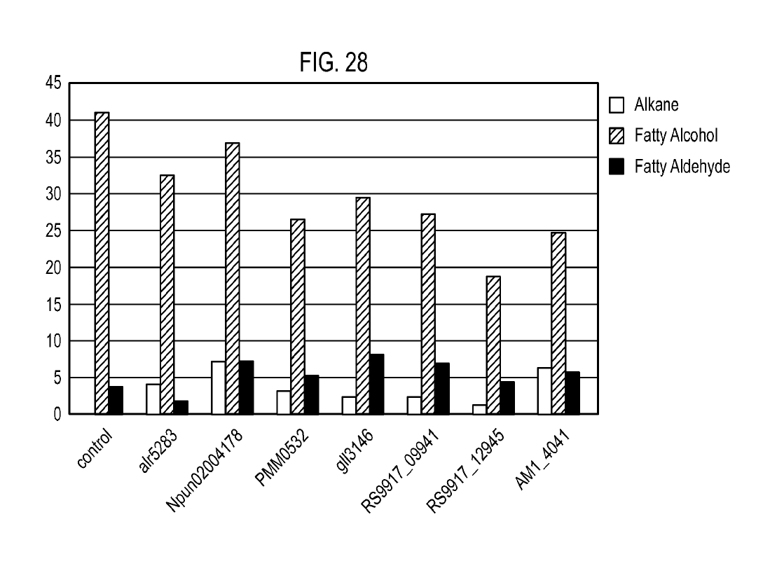

As shown in FIG. 27, these E. coli cells cotransformed with the N. punctiforme PCC73102 Npun02004178 and M. smegmatis strain MC2 155 MSMEG_{—}5739-bearing vectors produced tridecane, pentadecene and pentadecane. This result indicates that Npun02004178 in E. coli converts tetradecanal, hexadecenal and hexadecanol provided by the carboxylic acid reductase MSMEG_{—}5739 to tridecane, pentadecene and pentadecane. As shown in FIG. 28, in the same experimental set-up, the following fatty aldehyde decarbonylases also converted fatty aldehydes provided by MSMEG_{—}5739 to the corresponding alkanes when expressed in E. coli MG1655 ΔfadD lacZ::P_{trc}-′tesA: Nostoc sp. PCC7210 alr5283 (SEQ ID NO: 7), P. mariunus CCMP1986 PMM0532 (SEQ ID NO: 19), G. violaceus PCC7421 gll3146 (SEQ ID NO: 15), Synechococcus sp. RS9917_{—}09941 (SEQ ID NO: 23), Synechococcus sp. RS9917_{—}12945 (SEQ ID NO: 25), and A. marina MBIC11017 AM1_{—}4041 (SEQ ID NO: 9).

### Example 25

This example demonstrates that site-directed mutagenesis of conserved histidines to phenylalanines in Nostoc punctiforme PCC73102 Npun02004178 does not abolish catalytic function of cyanobacterial fatty aldehyde decarbonylases, which belong to the class of non-heme diiron proteins.

As discussed in Example 13, the hypothetical protein PM1231 from Prochlorococcus marinus MIT9313 (SEQ ID NO: 18) is an active fatty aldehyde decarbonylase. Based on the three-dimensional structure of PM1231, which is available at 1.8 Å resolution (PDB 2oc5A) (see FIG. 29B), cyanobacterial fatty aldehyde decarbonylases have structural similarity with non-heme diiron proteins, in particular with class I ribonuclease reductase subunit β proteins, RNRβ (Stubbe and Riggs-Gelasco, TIBS 1998, vol. 23., pp. 438) (see FIG. 29A). Class Ia and Ib RNRβ contains a diferric tyrosyl radical that mediates the catalytic activity of RNRα (reduction of ribonucleotides to deoxyribonucleotides). In E. coli RNRβ, this tyrosine is in position 122 and is in close proximity to one of the active site's iron molecules. Structural alignment showed that PM1231 contained a phenylalanine in the same position as RNRb tyr122, suggesting a different catalytic mechanism for cyanobacterial fatty aldehyde decarbonylases. However, an aligment of all decarbonylases showed that two tyrosine residues were completely conserved in all sequences, tyr135 and tyr138 with respect to PM1231, with tyr135 being in close proximity (5.5 Å) to one of the active site iron molecules (see FIG. 29C). To examine whether either of the two conserved tyrosine residues is involved in the catalytic mechanism of cyanobacterial fatty aldehyde decarbonylases, these residues were replaced with phenylalanine in Npun02004178 (tyr 123 and tyr126) as follows.

The genomic DNA encoding S. elongatus PCC7942 ORF1594 (SEQ ID NO: 66) was cloned into the NcoI and EcoRI sites of vector OP-80 (pCL1920 derivative) under the control of the P_{trc} promoter. The genomic DNA encoding N. punctiforme PCC73102 Npun02004178 (SEQ ID NO: 5) was also cloned into the NdeI and XhoI sites of vector OP-183 (pACYC 177 derivative) under the control of the P_{trc} promoter. The latter construct was used as a template to introduce a mutation at positions 123 and 126 of the decarbonylase protein, changing the tyrosines to phenylalanines using the primers GTTTTGCGATCGCAGCATTTAACATTTACATCCCCGTTGCCGACG (SEQ ID NO: 103) and GTTTTGCGATCGCAGCATATAACATTTTCATCCCCGTTGCCGACG (SEQ ID NO: 104), respectively. The resulting constructs were then transformed into E. coli MG1655. The cells were grown at 37° C. in M9 minimal media supplemented with 1% glucose (w/v), and 100 μg/ml carbenicillin and spectinomycin. The cells were cultured and extracted as in Example 3.

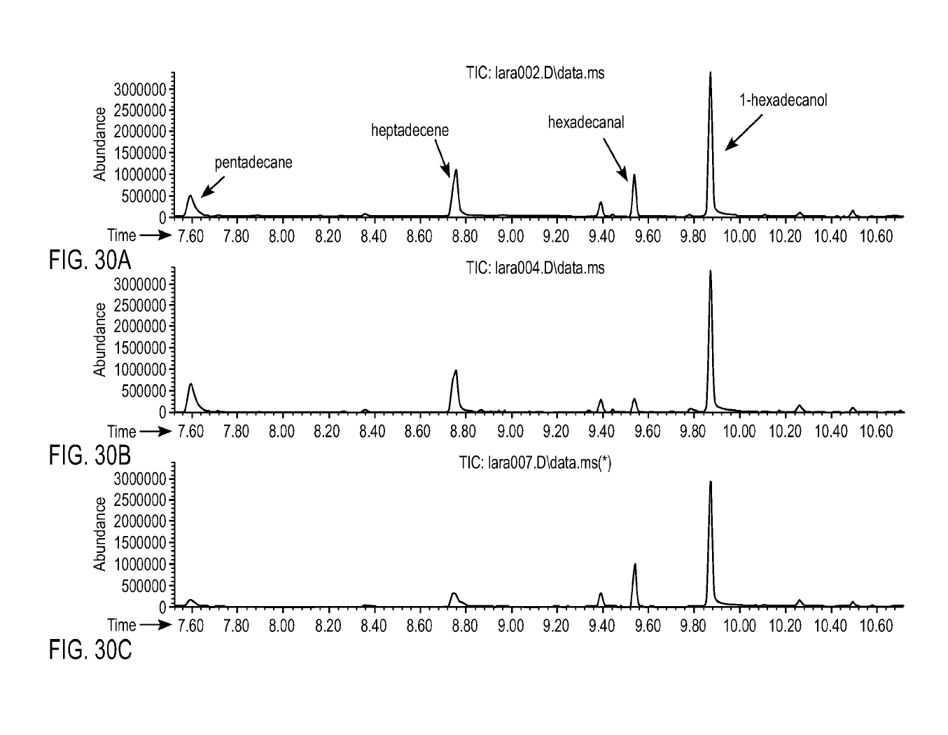

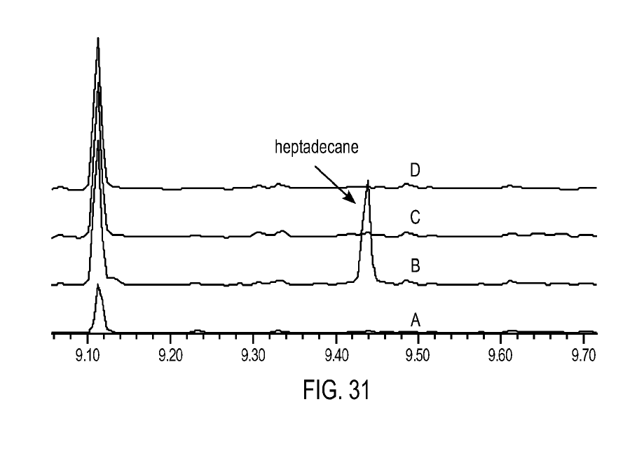

As shown in FIG. 30, the two Npun02004178 Tyr to Phe protein variants were active and produced alkanes when coexpressed with S. elongatus PCC7942 ORF1594. This result indicates that in contrast to class Ia and Ib RNRβ proteins, the catalytic mechanism of fatty aldehyde decarbonylases does not involve a tyrosyl radical.

### Example 26

This example demonstrates the biochemical characterization of Nostoc punctiforme PCC73102 Npun02004178.

The genomic DNA encoding N. punctiforme PCC73102 Npun02004178 (SEQ ID NO: 5) was cloned into the NdeI and XhoI sites of vector pET-15b under the control of the T7 promoter. The resulting Npun02004178 protein contained an N-terminal His-tag. An E. coli BL21 strain (DE3) (Invitrogen) was transformed with the plasmid by routine chemical transformation techniques. Protein expression was carried out by first inoculating a colony of the E. coli strain in 5 ml of LB media supplemented with 100 mg/L of carbenicillin and shaken overnight at 37° C. to produce a starter culture. This starter cultures was used to inoculate 0.5 L of LB media supplemented with 100 mg/L of carbenecillin. The culture was shaken at 37° C. until an OD₆₀₀ value of 0.8 was reached, and then IPTG was added to a final concentration of 1 mM. The culture was then shaken at 37° C. for approximately 3 additional h. The culture was then centrifuged at 3,700 rpm for 20 min at 4° C. The pellet was then resuspended in 10 ml of buffer containing 100 mM sodium phosphate buffer at pH 7.2 supplemented with Bacterial ProteaseArrest (GBiosciences). The cells were then sonicated at 12 W on ice for 9 s with 1.5 s of sonication followed by 1.5 s of rest. This procedure was repeated 5 times with one min intervals between each sonication cycle. The cell free extract was centrifuged at 10,000 rpm for 30 min at 4° C. 5 ml of Ni-NTA (Qiagen) was added to the supernatant and the mixture was gently stirred at 4° C. The slurry was passed over a column removing the resin from the lysate. The resin was then washed with 30 ml of buffer containing 100 mM sodium phosphate buffer at pH 7.2 plus 30 mM imidazole. Finally, the protein was eluted with 10 ml of 100 mM sodium phosphate buffer at pH 7.2 plus 250 mM imidazole. The protein solution was dialyzed with 200 volumes of 100 mM sodium phosphate buffer at pH 7.2 with 20% glycerol. Protein concentration was determined using the Bradford assay (Biorad). 5.6 mg/ml of Npun02004178 protein was obtained.

To synthesize octadecanal for the decarbonylase reaction, 500 mg of octadecanol (Sigma) was dissolved in 25 ml of dichloromethane. Next, 200 mg of pyridinium chlorochromate (TCI America) was added to the solution and stirred overnight. The reaction mixture was dried under vacuum to remove the dichloromethane. The remaining products were resuspended in hexane and filtered through Whatman filter paper. The filtrate was then dried under vacuum and resuspended in 5 ml of hexane and purified by silica flash chromatography. The mixture was loaded onto the gravity fed column in hexane and then washed with two column volumes of hexane. The octadecanal was then eluted with an 8:1 mixture of hexane and ethyl acetate. Fractions containing octadecanal were pooled and analyzed using the GC/MS methods described below. The final product was 95% pure as determined by this method.

To test Npun02004178 protein for decarbonylation activity, the following enzyme assays were set-up. 200 μl reactions were set up in 100 mM sodium phosphate buffer at pH 7.2 with the following components at their respective final concentrations: 30 μM of purified Npun02004178 protein, 200 μM octadecanal, 0.11 μg/ml spinach ferredoxin (Sigma), 0.05 units/mL spinach ferredoxin reductase (Sigma), and 1 mM NADPH (Sigma). Negative controls included the above reaction without Npun02004178, the above reaction without octadecanal, and the above reaction without spinach ferredoxin, ferredoxin reductase and NADPH. Each reaction was incubated at 37° C. for 2 h before being extracted with 100 W ethyl acetate. Samples were analyzed by GC/MS using the following parameters: run time: 13.13 min; column: HP-5-MS Part No. 19091S-433E (length of 30 meters; I.D.: 0.25 mm narrowbore; film: 0.25ìM); inject: 1ìl Agilent 6850 inlet; inlet: 300 C splitless; carrier gas: helium; flow: 1.3 ml/min; oven temp: 75° C. hold 5 min, 320 at 40° C./min, 320 hold 2 min; det: Agilent 5975B VL MSD; det. temp: 330° C.; scan: 50-550 M/Z. Heptadecane from Sigma was used as an authentic reference for determining compound retention time and fragmentation pattern.

As shown in FIG. 31, in-vitro conversion of octadecanal to heptadecane was observed in the presence of Npun02004178. The enzymatic decarbonylation of octadecanal by Npun02004178 was dependent on the addition of spinach ferredoxin reducatase, ferredoxin and NADPH.

Next, it was determined whether cyanobacterial ferredoxins and ferredoxin reductases can replace the spinach proteins in the in-vitro fatty aldehyde decarbonylase assay. The following four genes were cloned separately into the NdeI and XhoI sites of pET-15b: N. punctiforme PCC73102 Npun02003626 (ZP_{—}00109192, ferredoxin oxidoreductase petH without the n-terminal allophycocyanin linker domain) (SEQ ID NO: 88), N. punctiforme PCC73102 Npun02001001 (ZP_{—}00111633, ferredoxin 1) (SEQ ID NO: 90), N. punctiforme PCC73102 Npun02003530 (ZP_{—}00109422, ferredoxin 2) (SEQ ID NO: 92) and N. punctiforme PCC73102 Npun02003123 (ZP_{—}00109501, ferredoxin 3) (SEQ ID NO: 94). The four proteins were expressed and purified as described above. 1 mg/ml of each ferredoxin and 4 mg/ml of the ferredoxin oxidoreductase was obtained. The three cyanobacterial ferredoxins were tested with the cyanobacterial ferredoxin oxidoreductase using the enzymatic set-up described earlier with the following changes. The final concentration of the ferredoxin reductase was 60 μg/ml and the ferredoxins were at 50 μg/ml. The extracted enzymatic reactions were by GC/MS using the following parameters: run time: 6.33 min; column: J&W 122-5711 DB-5ht (length of 15 meters; I.D.: 0.25 mm narrowbore; film: 0.10 μM); inject: 1 μl Agilent 6850 inlet; inlet: 300° C. splitless; carrier gas: helium; flow: 1.3 ml/min; oven temp: 100° C. hold 0.5 min, 260 at 30° C./min, 260 hold 0.5 min; det: Agilent 5975B VL MSD; det. temp: 230° C.; scan: 50-550 M/Z.

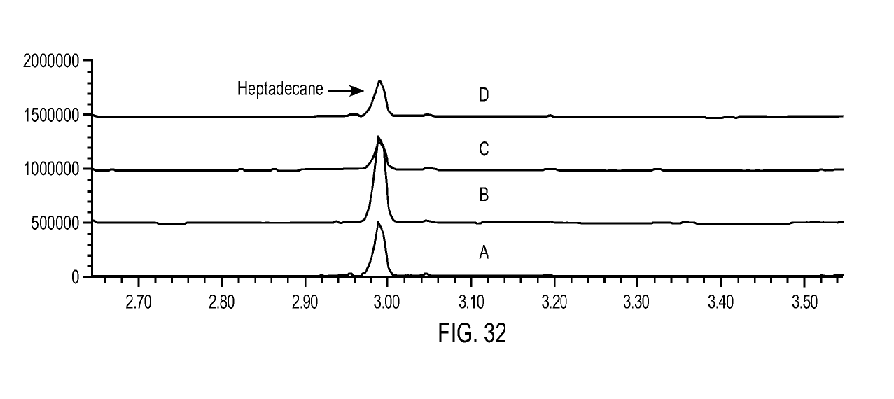

As shown in FIG. 32, Npun02004178-dependent in-vitro conversion of octadecanal to heptadecane was observed in the presence of NADPH and the cyanobacterial ferredoxin oxidoreductase and any of the three cyanobacterial ferredoxins.

### Example 27

This example demonstrates the biochemical characterization of Synechococcus elongatus PCC7942 orf1594.

The genomic DNA encoding S. elongatus PCC7492 orf1594 (SEQ ID NO: 66) was cloned into the NcoI and XhoI sites of vector pET-28b under the control of the T7 promoter. The resulting PCC7942_orf1594 protein contained a C-terminal His-tag. An E. coli BL21 strain (DE3) (Invitrogen) was transformed with the plasmid and PCC7942_orf1594 protein was expressed and purified as described in Example 22. The protein solution was stored in the following buffer: 50 mM sodium phosphate, pH 7.5, 100 mM NaCl, 1 mM THP, 10% glycerol. Protein concentration was determined using the Bradford assay (Bio-Rad). 2 mg/ml of PCC7942 orf1594 protein was obtained.

To test PCC7942_orf1594 protein for acyl-ACP or acyl-CoA reductase activity, the following enzyme assays were set-up. 100 μl reactions were set-up in 50 mM Tris-HCl buffer at pH 7.5 with the following components at their respective final concentrations: 10 μM of purified PCC7942_orf1594 protein, 0.01-1 mM acyl-CoA or acyl-ACP, 2 mM MgCl₂, 0.2-2 mM NADPH. The reactions were incubated for 1 h at 37° C. and where stopped by adding 100 μl ethyl acetate (containing 5 mg/l 1-octadecene as internal standard). Samples were vortexed for 15 min and centrifuged at max speed for 3 min for phase separation. 80 μl of the top layer were transferred into GC glass vials and analyzed by GC/MS as described in Example 26. The amount of aldehyde formed was calculated based on the internal standard.

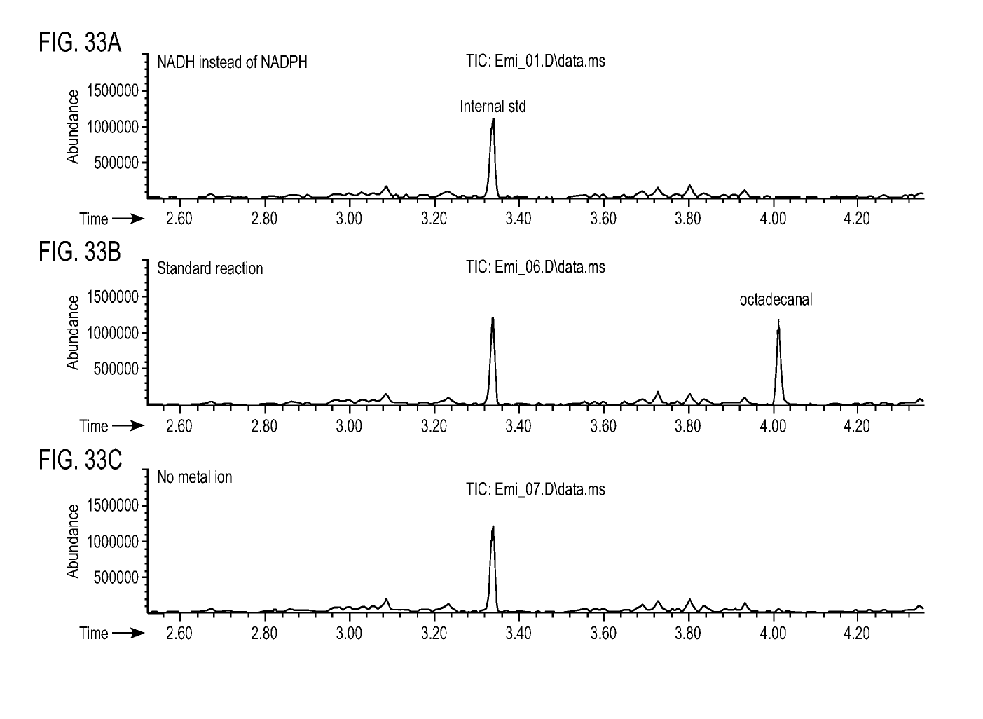

As shown in FIG. 33, PCC7942_orf1594 was able to reduce octadecanoyl-CoA to octadecanal. Reductase activity required divalent cations such as Mg²⁺, Mn²⁺ or Fe²⁺ and NADPH as electron donor. NADH did not support reductase activity. PCC7942_orf1594 was also able to reduce octadecenoyl-CoA and octadecenoyl-ACP to octadecenal. The Kₘ values for the reduction of octadecanoyl-CoA, octadecenoyl-CoA and octadecenoyl-ACP in the presence of 2 mM NADPH were determined as 45±20 μM, 82±22 μM and 7.8±2 μM, respectively. These results demonstrate that PCC7942_orf1594, in vitro, reduces both acyl-CoAs and acyl-ACPs and that the enzyme apparently has a higher affinity for acyl-ACPs as compared to acyl-CoAs. The Kₘ value for NADPH in the presence of 0.5 mM octadecanoyl-CoA for PCC7942_orf1594 was determined as 400±80 μM.

Next, the stereospecific hydride transfer from NADPH to a fatty aldehyde catalyzed by PCC7942_orf1594 was examined. Deutero-NADPH was prepared according to the following protocol. 5 mg of NADP⁺ and 3.6 mg of D-glucose-1-d was added to 2.5 ml of 50 mM sodium phosphate buffer (pH 7.0). Enzymatic production of labeled NADPH was initiated by the addition of 5 units of glucose dehydrogenase from either Bacillus megaterium (USB Corporation) for the production of R-(4-²H)NADPH or Thermoplasma acidophilum (Sigma) for the production of S-(4-²H)NADPH. The reaction was incubated for 15 min at 37° C., centrifuge-filtered using a 10 KDa MWCO Amicon Ultra centrifuge filter (Millipore), flash frozen on dry ice, and stored at −80° C.

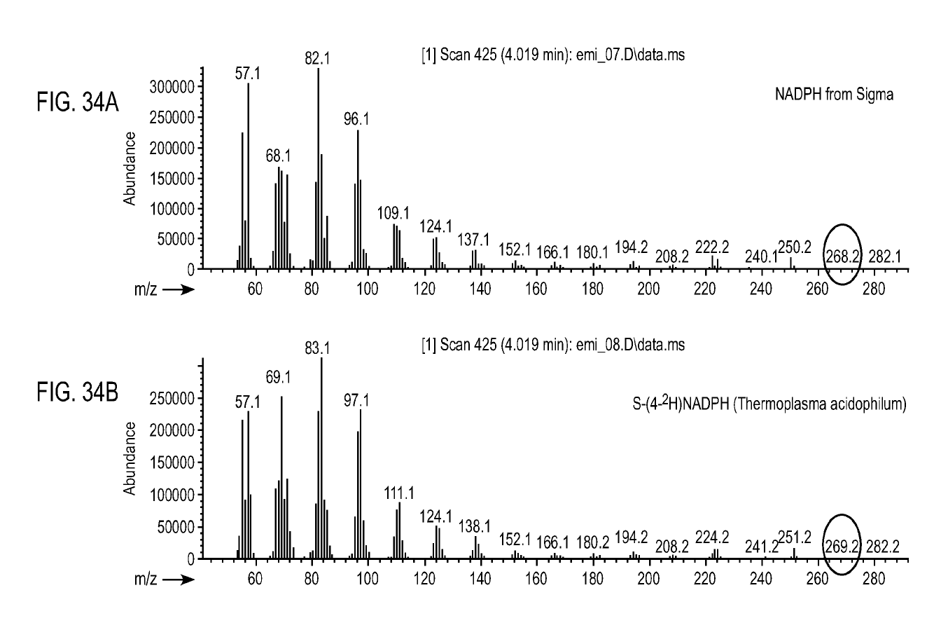

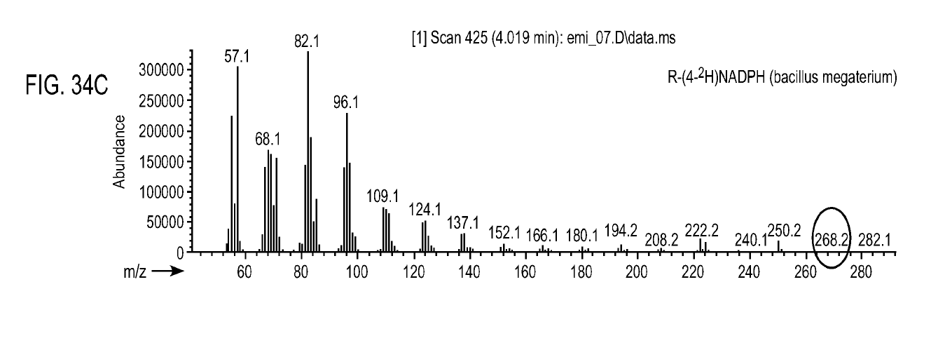

The in vitro assay reaction contained 50 mM Tris-HCl (pH 7.5), 10 μM of purified PCC7942_orf1594 protein, 1 mM octadecanoyl-CoA, 2 mM MgCl₂, and 50 μl deutero-NADPH (prepared as described above) in a total volume of 100 μl. After a 1 h incubation, the product of the enzymatic reaction was extracted and analyzed as described above. The resulting fatty aldehyde detected by GC/MS was octadecanal (see FIG. 34). Because hydride transfer from NADPH is stereospecific, both R-(4-²H)NADPH and S-(4-²H)NADPH were synthesized. Octadecanal with a plus one unit mass was observed using only the S-(4-²H)NADPH. The fact that the fatty aldehyde was labeled indicates that the deuterated hydrogen has been transferred from the labeled NADPH to the labeled fatty aldehyde. This demonstrates that NADPH is used in this enzymatic reaction and that the hydride transfer catalyzed by PCC7942_orf1594 is stereospecific.

### Example 28

This example demonstrates the intracellular and extracellular production of fatty aldehydes and fatty alcohols in E. coli through heterologous expression of Synechococcus elongatus PCC7942 orf1594.

The genomic DNA encoding Synechococcus elongatus PCC7942 orf1594 (YP_{—}400611; acyl-ACP reductase) (SEQ ID NO: 66) was amplified and cloned into the NcoI and EcoRI sites of vector OP-80 (pCL1920 derivative) under the control of the P_{trc} promoter. The resulting construct was cotransformed into E. coli MG1655 ΔfadE and the cells were grown at 37° C. in 15 ml Che-9 minimal media with 3% (w/v) glucose as carbon source and supplemented with 100 μg/ml spectinomycin and carbenicillin, respectively. When the culture reached OD₆₀₀ of 0.8-1.0, it was induced with 1 mM IPTG and cells were grown for an additional 24-48 h at 37° C. Che-9 minimal medium is defined as: 6 g/L Na₂HPO₄, 3 g/L KH₂PO₄, 0.5 g/L NaCl, 2 g/L NH₄Cl, 0.25 g/L MgSO₄×7H₂O, 11 mg/L CaCl₂, 27 mg/L Fe₃Cl×6H₂O, 2 mg/L ZnCl×4H₂O, 2 mg/L Na₂MoO₄×2H₂O, 1.9 mg/L CuSO₄×5H₂O, 0.5 mg/L H₃BO₃, 1 mg/L thiamine, 200 mM Bis-Tris (pH 7.25) and 0.1% (v/v) Triton-X100. When the culture reached OD₆₀₀ of 1.0-1.2, it was induced with 1 mM IPTG and cells were allowed to grow for an additional 40 hrs at 37° C. Cells from 0.5 ml of culture were extracted with 0.5 mL of ethyl acetate for total hydrocarbon production as described in Example 26. Additionally, cells and supernatant were separated by centrifugation (4,000 g at RT for 10 min) and extracted separately.

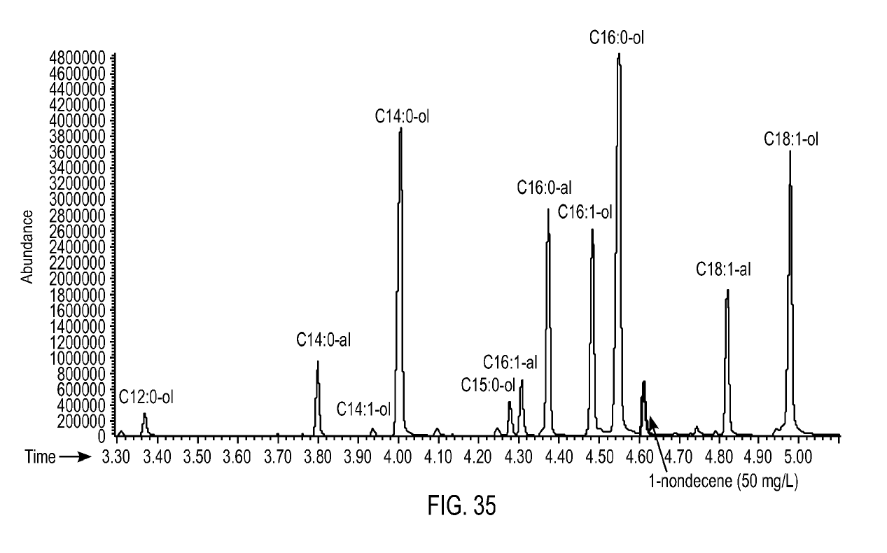

The culture produced 620 mg/L fatty aldehydes (tetradecanal, heptadecenal, heptadecanal and octadecenal) and 1670 mg/L fatty alcohols (dodecanol, tetradecenol, tetradecanol, heptadecenol, heptadecanol and octadecenol). FIG. 35 shows the chromatogram of the extracted supernatant. It was determined that 73% of the fatty aldehydes and fatty alcohols were in the cell-free supernatant.

### Example 29

This example demonstrates the intracellular and extracellular production of alkanes and alkenes in E. coli through heterologous expression of Synechococcus elongatus PCC7942 orf1594 and Nostoc punctiforme PCC73102 Npun02004178.

The genomic DNA encoding Synechococcus elongatus PCC7942 orf1594 (YP_{—}400611; acyl-ACP reductase) (SEQ ID NO: 66) was amplified and cloned into the NcoI and EcoRI sites of vector OP-80 (pCL1920 derivative) under the control of the P_{trc} promoter. The genomic DNA encoding Nostoc punctiforme PCC73102 Npun02004178 (ZP_{—}00108838; fatty aldehyde decarbonylase) (SEQ ID NO: 5) was amplified and cloned into the NdeI and XhoI sites of vector OP-183 (pACYC derivative) under the control of the P_{trc} promoter. The resulting constructs were cotransformed into E. coli MG1655 ΔfadE and the cells were grown at 37° C. in 15 ml Che9 minimal media with 3% (w/v) glucose as carbon source and supplemented with 100 μg/ml spectinomycin and carbenicillin, respectively. The cells were grown, separated from the broth, extracted, and analyzed as described in Example 28.

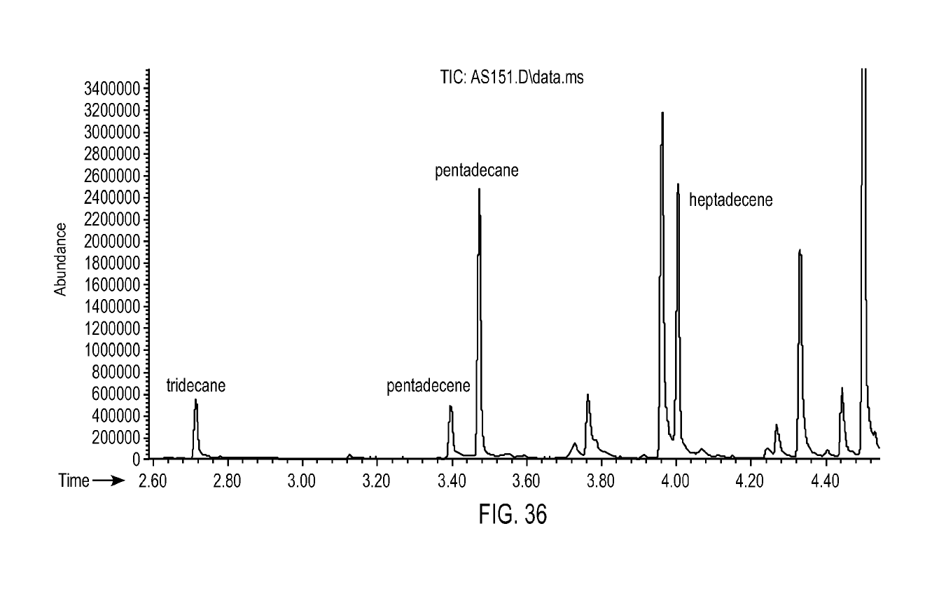

The culture produced 323 mg/L alkanes and alkenes (tridecane, pentadecene, pentadecane and heptadecene), 367 mg/L fatty aldehydes (tetradecanal, heptadecenal, heptadecanal and octadecenal) and 819 mg/L fatty alcohols (tetradecanol, heptadecenol, heptadecanol and octadecenol). FIG. 36 shows the chromatogram of the extracted supernatant. It was determined that 86% of the alkanes, alkenes, fatty aldehydes and fatty alcohols were in the cell-free supernatant.

### Example 30

This example demonstrates the production of alkanes and alkenes in E. coli through heterologous expression of Nostoc sp. PCC7210 alr5284 and Nostoc sp. PCC7210 alr5283.

The genomic DNA encoding Nostoc sp. PCC7210 alr5284 (NP_{—}489324; putative aldehyde-generating enzyme) (SEQ ID NO: 82) was amplified and cloned into the NcoI and EcoRI sites of vector OP-80 (pCL1920 derivative) under the control of the P_{trc} promoter. The genomic DNA encoding Nostoc sp. PCC7210 alr5283 (NP_{—}489323; putative decarbonylase) (SEQ ID NO: 7) was amplified and cloned into the NdeI and XhoI sites of vector OP-183 (pACYC derivative) under the control of the P_{trc} promoter. The resulting constructs were cotransformed into E. coli MG1655 and the cells were grown at 37° C. in 15 ml Che9 minimal media with 3% (w/v) glucose as carbon source and supplemented with 100 μg/ml spectinomycin and carbenicillin, respectively (as described in Example 28). Cells from 0.5 ml of culture were extracted and analyzed as described in Example 3 and analyzed by GC-MS as described in Example 26.

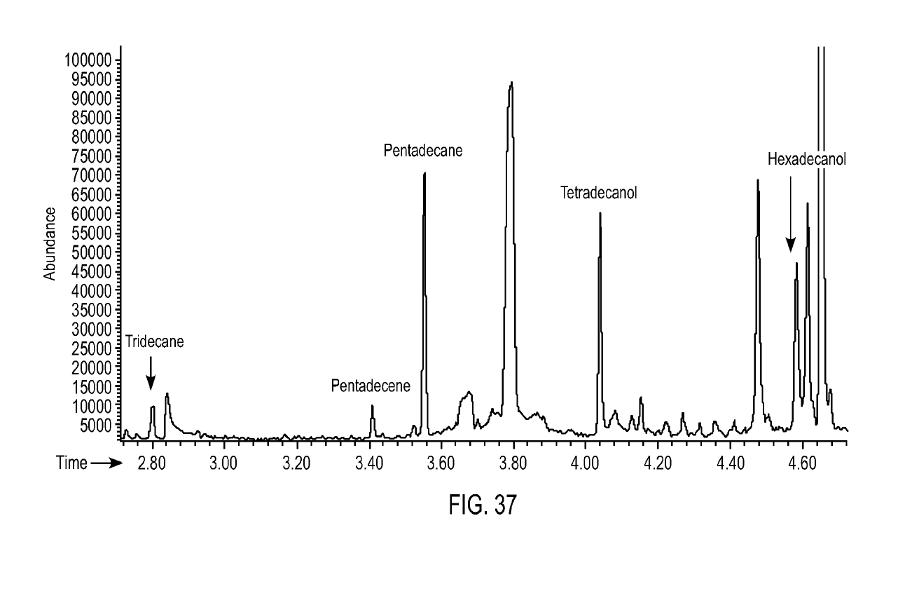

As shown in FIG. 37, E. coli cells cotransformed with the Nostoc sp. PCC7210 alr5284 and Nostoc sp. PCC7210 alr5283-bearing vectors produced tridecane, pentadecene, pentadecane, tetradecanol and hexadecanol. This result indicates that coexpression of Nostoc sp. PCC7210 alr5284 and alr5283 is sufficient for E. coli to produce fatty alcohols, alkanes and alkenes.

### Example 31

This example demonstrates the discovery of a structurally conserved motif.

Based on the known X-ray crystallographic structure of Prochlorococcus marinus MIT9313 PMT1231, NP_{—}895059 (SEQ ID NO: 18), a three-dimensional homology model of putative hydrocarbon biosynthetic polypeptides can be created using the methods described in Arnold K., Bordoli L., Kopp J., and Schwede T., The SWISS-MODEL Workspace: A web-based environment form protein structure homology modeling. Bioinformatics, 22, 195-201.

At the website http://swissmodel.expasy.org, automated mode was selected. Amino acid sequences of putative hydrocarbon biosynthetic polypeptides were entered into the appropriate query. “2oc5A,” which represents the Protein Data Bank atomic coordinates accession number for Prochlorococcus marinus MIT9313 PMT1231, was entered as a “specific template” under the query “Use a specific template.” The command “Summit Modeling Request” was implemented and the predicted structure of the putative hydrocarbon biosynthetic polypeptides were obtained in reference to the 2oc5A structural coordinates.

Using the “SwissPDB-Viewer” function, putative alkane/alkene biosynthetic polypeptides were viewed as they were individually superimposed or fitted to the PDB Accession No. 2oc5A coordinates, with the di-iron center of Prochlorococcus marinus MIT9313 PMT1231 visible. All amino acid residues of the putative hydrocarbon biosynthetic polypeptides within 6 Å of the di-iron center of MIT9313 PMT1231 enzyme were identified.

To predict whether a putative hydrocarbon biosynthetic polypeptide has decarbonylase activity, the amino acid sequence of the polypeptide was examined to ascertain the presence of a motif selected from the following four polypeptides: SEQ ID NO: 105, SEQ ID NO: 106, SEQ ID NO: 107, and SEQ ID NO: 108.

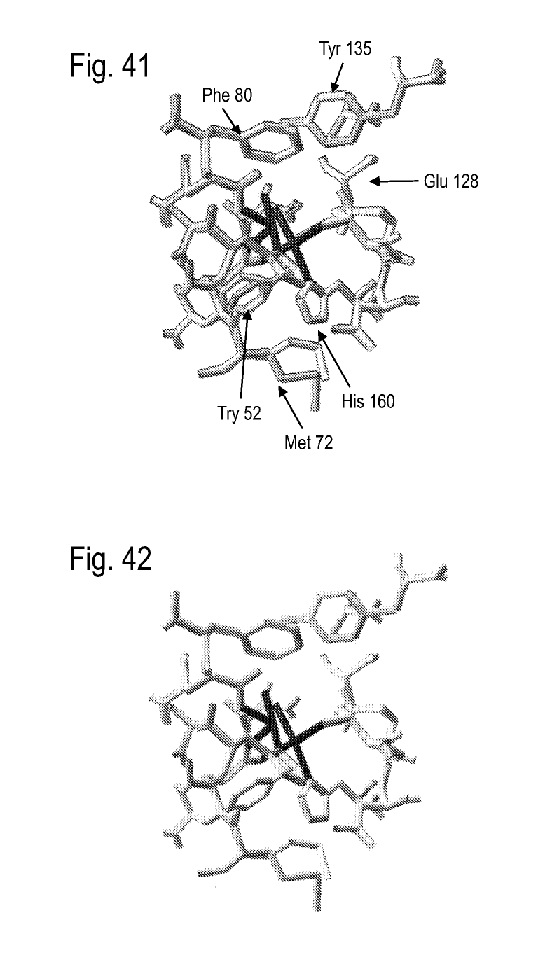

Using this method, Synechococcus sp. RS9917 enzyme ZP_{—}01080370, the amino acid sequence of which is about 43% identical to that of Prochlorococcus marinus MIT9313 PMT1231, was identified as an active hydrocarbon biosynthetic polypeptide. FIG. 41 depicts an X-ray crystallographic metal-center structure of Prochlorococcus marinus MIT9313 PMT1231 enzyme NP_{—}895059 overlaid on the putative metal-center amino acid residues of RS9917 enzyme ZP_{—}01080370. Accordingly, despite the low sequence homology, Synechococcus sp. RS9917 enzyme ZP_{—}01080370 contained one of the motifs described above and was predicted to have decarbonylase activity. In confirmation, Synechococcus sp. RS9917 enzyme ZP_{—}01080370 was found to be an active fatty aldehyde decarbonylase in Example 17.

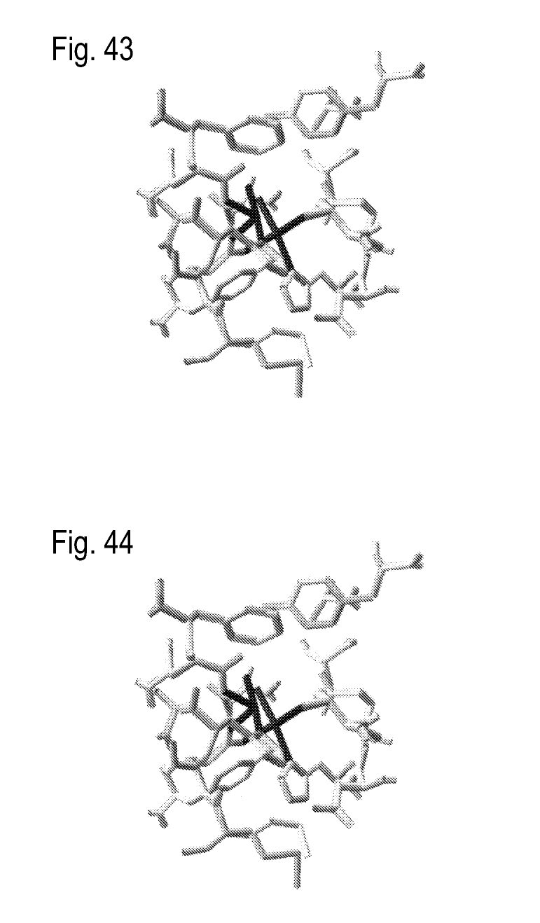

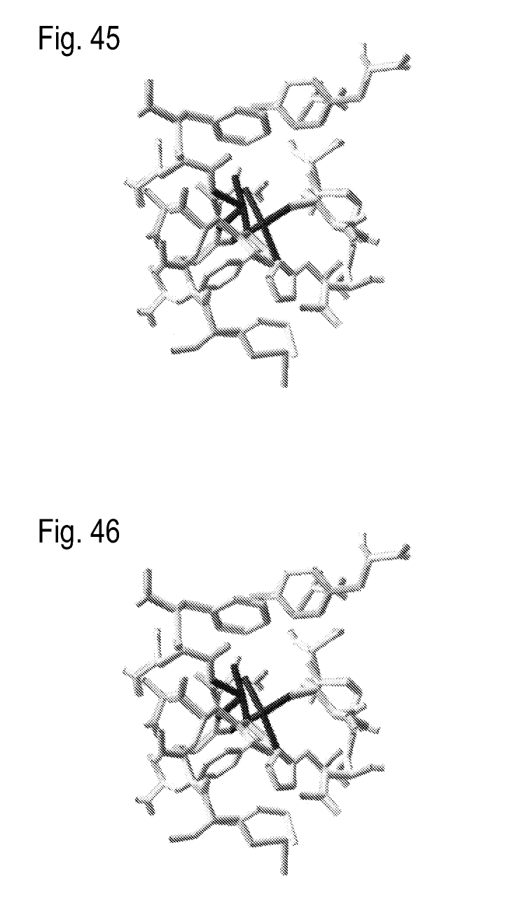

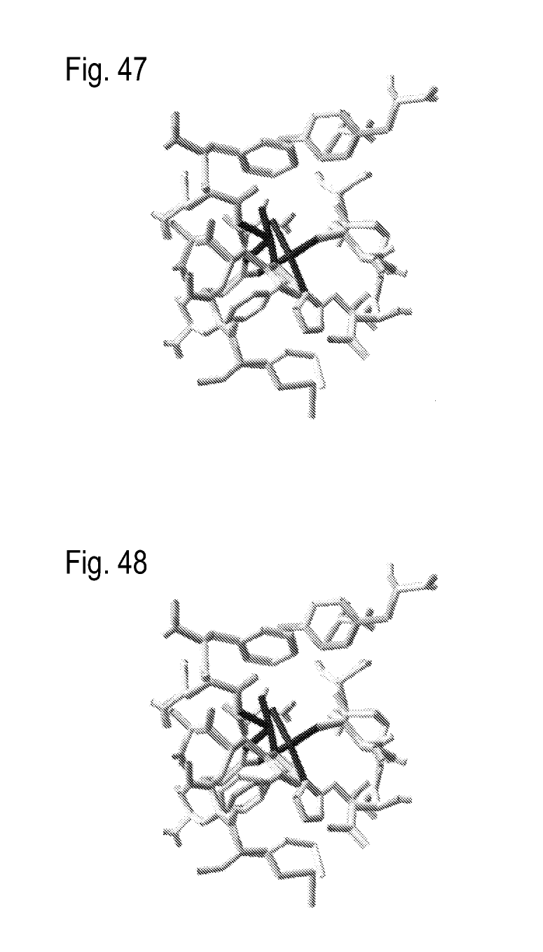

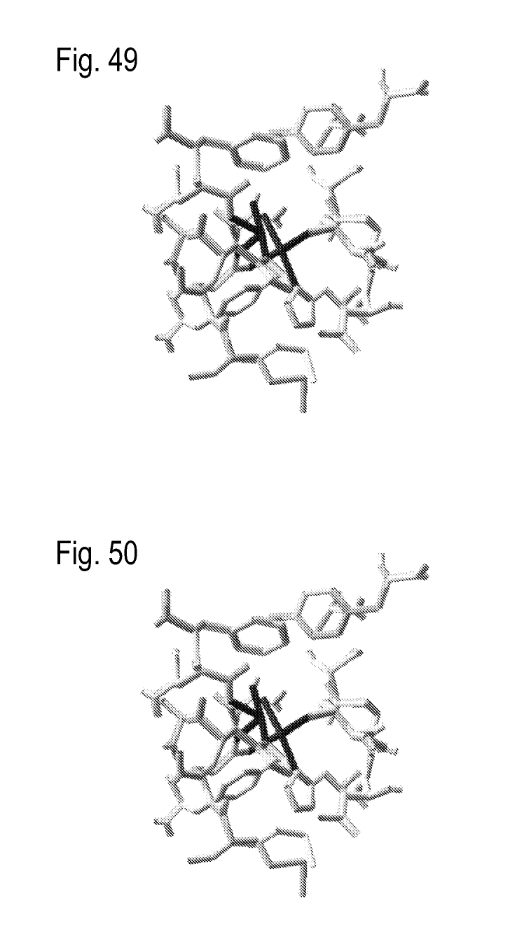

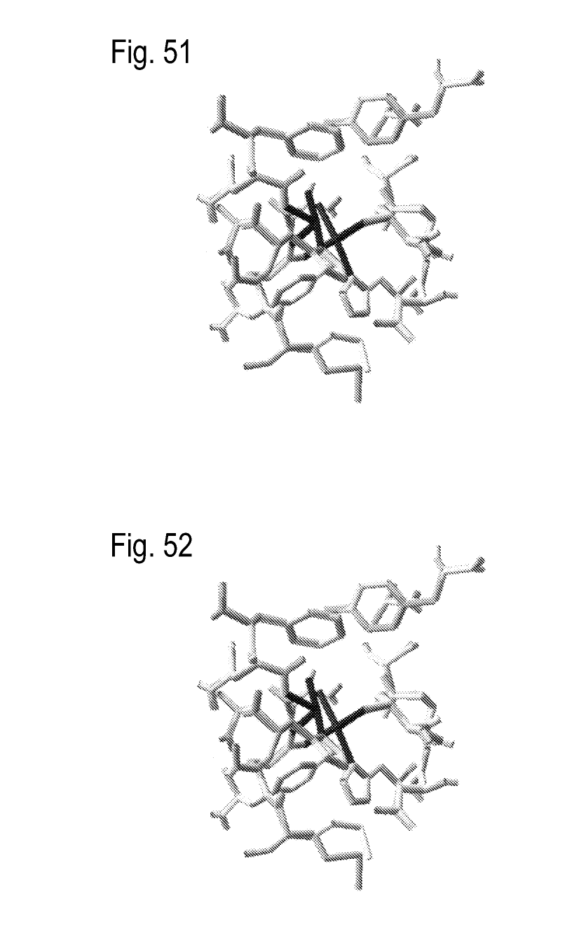

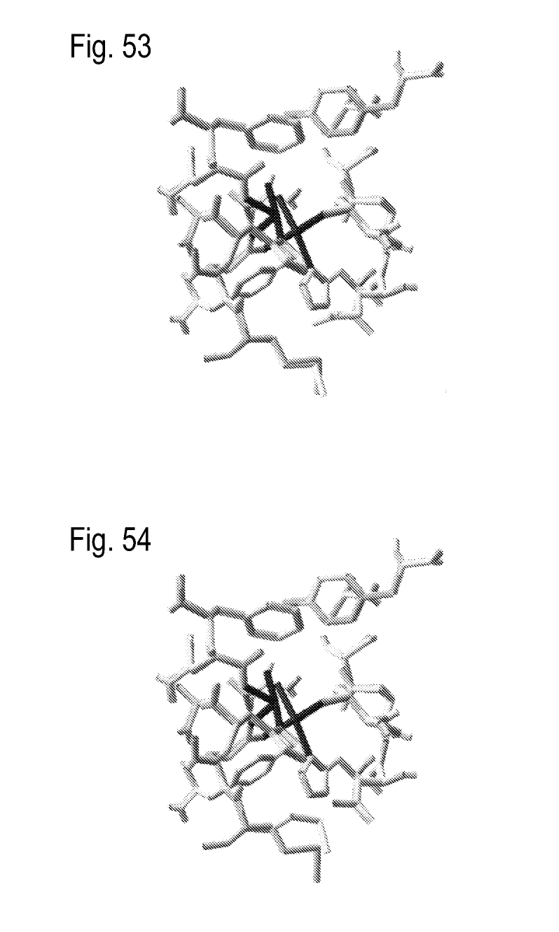

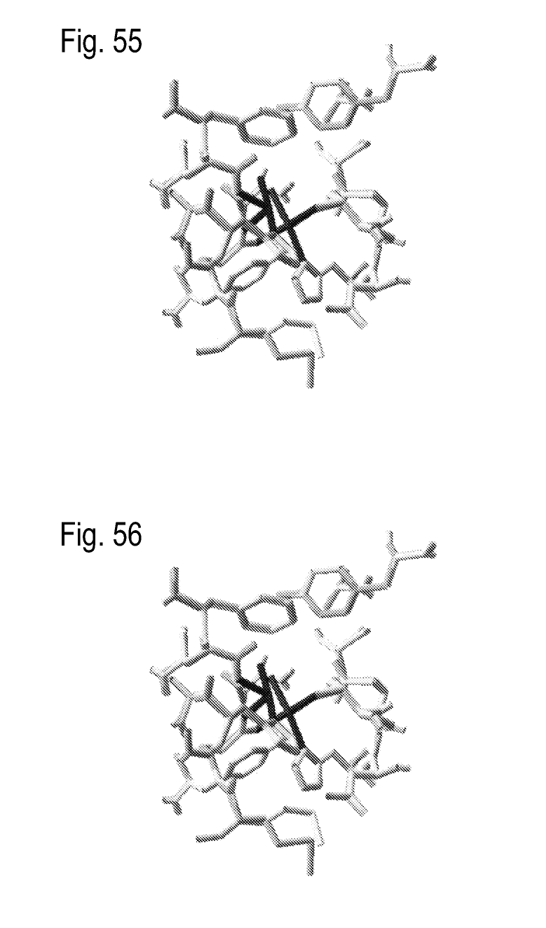

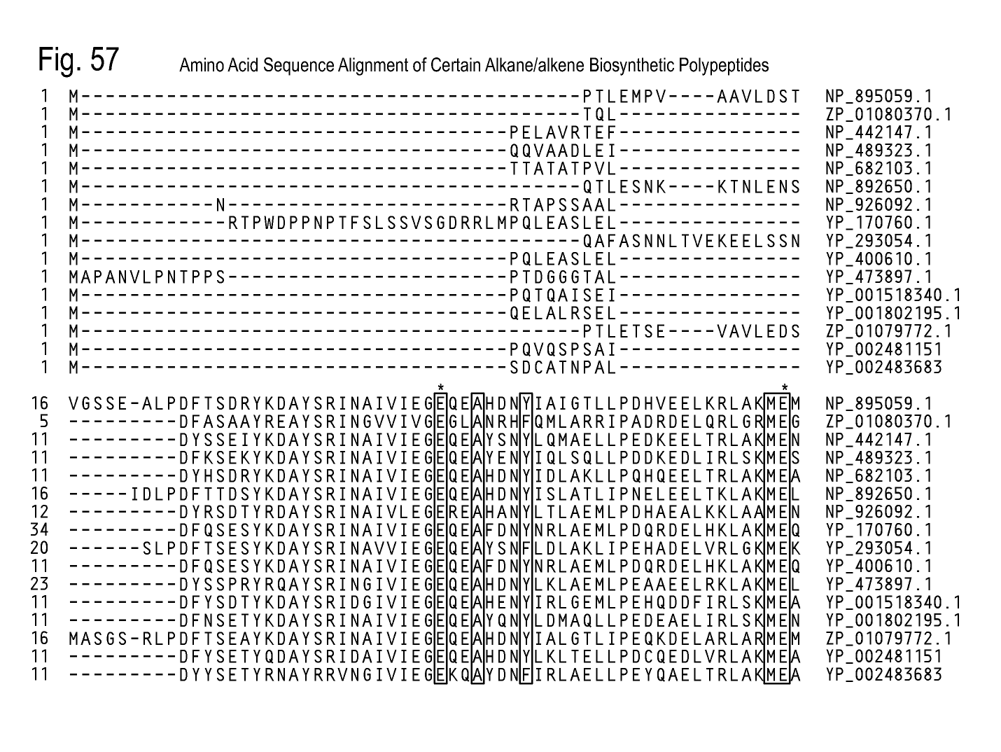

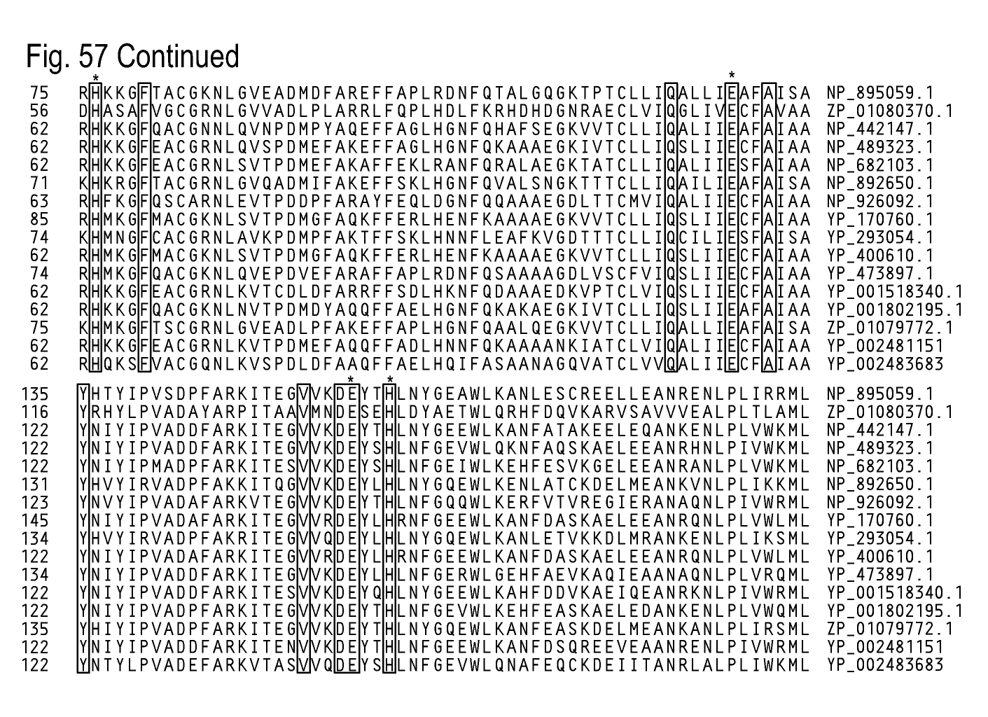

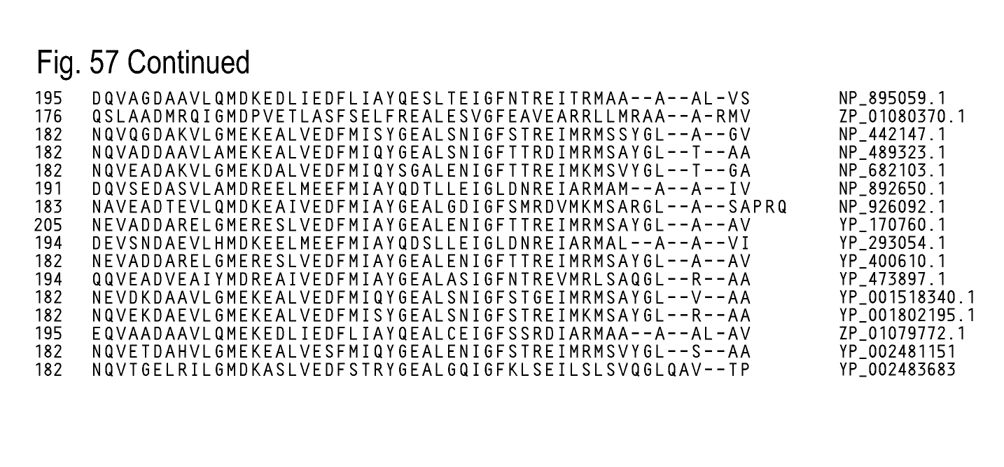

Moreover, using this method, the putative metal-center amino acid residues of Synechococcus sp. PCC 7335 enzyme NP_{—}442147, Nostoc sp. PCC 7120 enzyme NP_{—}489323, Thermosynechococcus elongatus BP-1 enzyme NP_{—}682103, Prochlorococcus marinus subsq. pastoris str. CCMP1986 enzyme NP_{—}892650, Gloeobacter violaceus PCC 7421 enzyme NP_{—}926092, Synechococcus elongatus PCC 6301 enzyme YP_{—}170760, Prochlorococcus marinus str. NAL2A enzyme YP_{—}293054, Synechococcus elongatus PCC 7942 enzyme YP_{—}400610, Synechococcus sp. JA-3-3Ab enzyme YP_{—}473897, Acaryochloris marina MBIC11017 enzyme YP_{—}001518340, Cyanothece sp. ATCC 51142 enzyme ZP_{—}001802195, Nostoc punctiforme PCC 73102 enzyme ZP_{—}00108838, Synechococcus sp. RS9917 enzyme ZP_{—}01079772, Cyanothece sp. PCC 7425 enzyme ZP_{—}3137219, and Cyanothece sp. PCC7425 enzyme ZP_{—}03139316 were likewise individually superimposed on the known metal-center amino acid residues of MIT9313 PMT1231, NP_{—}895059 (see, FIGS. 42-56, respectively). This confirmed that the listed enzymes, while having between about 43% to 100% sequence homology (see, amino acid sequence alignment in FIG. 57) to the reference sequence NP_{—}895059, are nonetheless active and suitable alkane/alkene biosynthetic polypeptides, which have decarbonylase activity, just like the reference enzyme Prochlorococcus marinus MIT9313 PMT1231, NP_{—}895059. In confirmation, Nostoc punctiforme PCC 73102 enzyme ZP_{—}00108838, Synechococcus sp. PCC 7335 enzyme NP_{—}442147, Nostoc sp. PCC 7120 enzyme NP_{—}489323, Acaryochloris marina MBIC11017 enzyme YP_{—}001518340, Thermosynechococcus elongatus BP-1 enzyme NP_{—}682103, Synechococcus sp. JA-3-3Ab enzyme YP_{—}473897, Gloeobacter violaceus PCC 7421 enzyme NP_{—}926092, Prochlorococcus marinus subsq. pastoris str. CCMP1986 enzyme NP_{—}892650, Prochlorococcus marinus str. NAL2A enzyme YP_{—}293054, Synechococcus sp. RS9917 enzyme ZP_{—}01079772, Cyanothece sp. ATCC 51142 enzyme ZP_{—}001802195, Cyanothece sp. PCC 7425 enzyme ZP_{—}3137219, and Cyanothece sp. PCC7425 enzyme ZP_{—}03139316 were demonstrated to be active fatty aldehyde decarbonylases in Examples 6, 8, 9, 10, 11, 12, 14, 15, 16, 18, 19 and 20, respectively.

### Example 32

This example demonstrates the production of alkanes in the cyanobacterium Synechoccus sp. PCC7002.

A vector is constructed for homologous recombination into the Synechococcus sp. PCC7002 plasmid pAQ1 (genbank accession NC_{—}0050525) using 500 bp homologous regions corresponding to positions 3301-3800 and 3801-4300 of pAQ1. As a selectable marker, a spectinomycin resistance cassette such as one derived from plasmid pCL1920 or a vector OP-80, containing an aminoglycoside 3′ adenylyltransferase, aad, a suitable promoter, the gene to be inserted and a suitable terminator, is introduced to a position between the homologous regions.

For gene expression, the promoter and a ribosome binding site of an aminoglycoside phosphotransferase, aph, for example, ones derived from the plasmid pACYC 177 (New England Biolabs, Inc., Ipswich, Mass.), as well as suitable unique cloning sites, for example, the NdeI and/or EcoRI sites, are introduced into the plasmid so that a heterologous gene or operons can be inserted. The heterologous gene or operon can be prepared by known gene synthesis methods. A complete integration cassette is constructed and cloned into a pUC19 vector (New England Biolabs, Inc., Ipswich, Mass.). The resulting plasmid is termed pLS9-7002, which can be used for cloning and expressing foreign genes, and for delivery and stable in vivo integration into Synechococcus sp. PCC7002 plasmid pAQ1.

Next, a pathway for expressing alkanes in Synechococcus sp. PCC7002 is constructed. A synthetic operon can be created and cloned into the NdeI and EcoRI sites of pLS9-7002 downstream of the aph promoter and the ribosome binding site. The resulting plasmid is transformed into Synechococcus sp. PCC7002 in accordance with a method described by Stevens and Porter (PNAS 1980, vol. 77, pp. 6052-56). Stable integrants are selected using ATCC 1047 medium supplemented with 15 μg/ml spectinomycin. Each liter of ATCC 1047 medium contains 40 mg of MgSO₄×7 H₂O, 20 mg of CaCl₂×2 H₂O, 750 mg of NaNO₃, 2 mg of K₂HPO₄, 3.0 mg of citric acid, 3.0 mg of ferric ammonium citrate, 0.5 mg of EDTA, 20 mg of Na₂CO₃, 2.86 mg of H₃BO₃, 1.81 mg of MnCl₂, 0.22 mg of ZnSO₄, 0.04 mg of Na₂MoO₄, 0.08 mg of CuSO₄, 0.05 mg of Co(NO₃)₂, 0.02 mg of vitamin B12, 10 g of agar and 750 ml of sea water. Spectinomycin-resistant colonies are streaked and restreaked several times on ATCC medium 1047 containing spectinomycin, and isogenic integration of the operon is verified by PCR using the following primers: pAQ1-U (ATGTCTGACAAGGGGTTTGACCCCT (SEQ ID NO: 109)); and pAQ1-D (GCACATCCTTATCCAATTGCTCTAG (SEQ ID NO: 110)).

Complete isogenic ADC-AAR integrants are then selected and cultured in 50 ml liquid ATCC 1047 medium containing spectinomycin in 500 ml shaker flasks with appropriate aeration and illumination at 30° C. for up to seven days. Culture aliquots are extracted at various time points using methanol and the extracts are analyzed for alkanes production as described in Example 1.

Using this method, alkanes are produced by Synechococcus sp. PCC7002.

### Example 33

This example demonstrates the overproduction of alkanes in Synechococcus elongatus PCC7942.

A vector is constructed to accomplish homologous recombination into the Synechococcus elongatus PCC7942 genome (genbank accession CP_{—}000100), using 800 bp homologous regions corresponding to positions 2577844-2578659 and 2578660-2579467 of CP_{—}000100. This chromosomal location is known as neutral site one (NS1) (see Mackey et al., Meth. Mol. Biol. 2007, vol. 362, pp. 115-129). A selectable marker, a spectinomycin-resistance cassette containing aminoglycoside 3′ adenylyltransferase, aad, a promoter, the gene of interest, and a terminator, for example one derived from plasmid pCL1920, are introduced to a position between the homologous regions. Suitable unique cloning sites, for example NdeI and EcoRI restriction sites, are introduced to allow for insertion of a heterologous gene or operon. This integration cassette is constructed by suitable gene synthesis methods and cloned into pUC19 vector (New England Biolabs, Inc., Ipswich, Mass.) for maintenance and delivery. The resulting plasmid is called pLS9-7942-NS1, which allows cloning and expression of a foreign gene as well as delivery and stable integration of the foreign gene into the Synechococcus elongatus PCC7942 genome.

Next, a pathway expressing alkane is constructed in Synechococcus elongatus PCC7942. An operon including a ptrc promoter and a ribosome binding site is created and cloned into the NdeI or EcoRI site of pLS9-7942-NS1. The resulting plasmid is transformed into Synechococcus elongatus PCC7942 in accordance to a method described by Mackey et al (Meth. Mol. Biol. 2007, vol. 362, pp. 115-129).

Stable integrants are identified and selected using a BG-11 medium supplemented with 4 μg/ml spectinomycin. Each liter of the BG-11 medium contains 75 mg of MgSO₄×7H₂O, 36 mg of CaCl₂×2 H₂O, 1.5 g of NaNO₃, 40 mg of K₂HPO₄, 6.0 mg of citric acid, 6.0 mg of ferric ammonium citrate, 1.0 mg of EDTA, 20 mg of Na₂CO₃, 2.86 mg of H₃BO₃, 1.81 mg of MnCl₂, 0.22 mg of ZnSO₄, 0.04 mg of Na₂MoO₄, 0.08 mg of CuSO₄, 0.05 mg of Co(NO₃)₂ and 10 g of agar. Spectinomycin-resistant colonies are streaked and restreaked several times on BG-11 medium plates containing spectinomycin. Isogenic integration of the operon is confirmed by PCR using primers: NS1-U: GATCAAACAGGTGCAGCAGCAACTT (SEQ ID NO: 111), and NS1-D: ATTCTTGACAAGCGATCGCGGTCAC (SEQ ID NO: 112).

The verified complete isogenic integrants are next cultured in 50 ml liquid BG-11 medium containing spectinomycin in 500 ml shaker flasks with appropriate aeration and illumination at 30° C. for 5 to 7 days. Culture aliquots are extracted at various time points with methanol and the extracts are analyzed for alkane production as described in Example 1.

Alkanes are being overproduced by Synechococcus elongatus PCC7942.

### Example 34

This example demonstrates the overproduction of alkanes in the cyanobacterium Synechocystis sp. PCC6803.

A vector is constructed for homologous recombination into the Synechocystis sp. PCC6803 genome (genbank accession BA_{—}000022) using 1300 to 1700 bp homologous regions corresponding to positions 2299015-2300690 and 2300691-2302056 of BA_{—}000022, respectively. This chromosomal location is known in the art as neutral site RS1/2 (see, Shao et al., Appl. Environ. Microbiol. 2002, vol. 68, pp. 5026-33). A suitable selectable marker, such as a kanamycin-resistance cassette containing an aminoglycoside phosphotransferase, aph, for example, one derived from plasmid pACYC177 (New England Biolabs, Inc., Ipswich, Mass.), a promoter, the gene of interest, and a terminator is inserted between the homologous regions. Additionally, suitable unique cloning sites, such as NdeI and XbaI sites are introduced such that a heterologous gene or operon can be inserted. The integration cassette can be constructed by gene synthesis and cloned into a pUC19 vector for maintenance and delivery. The resulting plasmid is called pLS9-6803-RS, which allows cloning and expression of a foreign gene as well as delivery and stable integration of the desired gene into the Synechocystis sp. PCC6803 genome.

Next, a pathway for alkane production is constructed in Synechocystis sp. PCC6803. The operon including a ptrc promoter and a ribosome binding site is created and cloned into the NdeI or XbaI site of pLS9-6803-RS. The resulting plasmid is transformed into Synechococcus elongatus PCC7942 in accordance with a method described by Zang et al. (J. Microbiol., 2007, vol. 45, pp. 241-45).

Stable integrants are identified and verified using BG-11 medium supplemented with 10 □g/ml kanamycin. Each liter of BG-11 medium contains 75 mg of MgSO4×7H2O, 36 mg of CaCl2×2H2O, 1.5 g of NaNO3, 40 mg of K2HPO4, 6.0 mg of citric acid, 6.0 mg of ferric ammonium citrate, 1.0 mg of EDTA, 20 mg of Na2CO3, 2.86 mg of H3BO3, 1.81 mg of MnCl2, 0.22 mg of ZnSO4, 0.04 mg of Na2MoO4, 0.08 mg of CuSO4, 0.05 mg of Co(NO3)2 and 10 g of agar. Kanamycin resistant colonies are streaked and restreaked several times on plates of BG-11 medium containing kanamycin. Isogenic integration of the ADC-AAR operon is verified by PCR using primers: RS1: ATTCAATACACCCCCCTAGCCGATC (SEQ ID NO: 113), and RS2: TAAGGGTGGTGGGAAAAATGGGCCA (SEQ ID NO: 114)

Complete isogenic ADC-AAR integrants are next cultured in 50 ml liquid BG-11 medium containing kanamycin in 500 ml shaker flasks with appropriate aeration and illumination at 30° C. for 5 to 7 days. Culture aliquots are extracted at various time points using methanol and the extracts are analyzed for alkane production as described in Example 1.

Alkanes are being overproduced by Synechocystis sp. PCC6803.

### Example 35

This example demonstrates malonyl-CoA independent production of alkanes in E. coli.

Certain protists such as Euglena gracilis have been found to be capable of malonyl-CoA independent fatty acid biosynthesis. The biosynthetic machinery for that pathway has been located to the mitochondria and is thought to reverse the direction of β-oxidation process. It uses acetyl-CoA as priming substrate as well as elongating substrate to produce C₈ to C₁₈ fatty acids (see, Inui et al., 1894, Eur. J. Biochem, vol. 142, pp. 121-126). The key enzymes in this machinery have been found. Trans-2-enoyl-CoA reductases (TER) catalyzes the irreversible reduction of trans-2-enoyl-CoA to acyl-CoA, and thereby driving the otherwise reversible pathway in the reductive direction. In contrast, the irreversible acyl-CoA dehydrogenase FadE serves the exact opposite role, driving the reaction in the oxidation direction. One TER gene from Euglena gracilis and various other eukaryotic and prokaryotic homologs have been identified (see Hoffmeister et al., 2005, J. Biol. Chem, vol. 280, pp. 4329-4338; Tucci and Martin, 2007, FEBS Lett., vol. 581, pp. 1561-1566). In vitro, the known TER enzyme from Euglena gracilis has been shown to reduce trans-2-butenoyl-CoA (i.e., C₄) and trans-2-hexenoyl-CoA (i.e., C₆) to the respective acyl-CoAs. Currently, little is known about the other pathway enzymes in Euglena gracilis.

A pathway that creates a flux exclusively from acetyl-CoA precursors to acyl-CoA, mimicking the pathway in the Euglena gracilis mitochondria, can be engineered in E. coli using different sets of enzymes, so long as four enzymatic activities are provided. The four activities can be accomplished by the following enzymes: (1) a non-decarboxylating, condensing thiolase, (2) a 3-ketoacyl-CoA reductase, or a 3-hydroxyacyl-CoA dehydrogenase, (3) a 3-hydroxyacyl-CoA hydratase or a enoyl-CoA dehydratase, and (4) a trans-2-enoyl-CoA reductase. Enzymes having sufficiently relaxed chain length specificity are selected to synthesize acyl-CoAs of longer chain length, e.g. C₁₂ or C₁₄ acyl-CoAs.

A plasmid encoding all four enzymatic activities can be constructed. A synthetic operon of E. coli fadA (YP_{—}026272) (non-decarboxylating thiolase) and fadB (NP_{—}418288) (3-hydroxyacyl-CoA dehydrogenase and enoyl-CoA dehydratase) and E. gracilis ter (Q5EU90) (trans-2-enoyl-CoA reductase, codon optimized to be without its 5′-sequence encoding a transit peptide) is constructed and cloned into, for example, a pACYC-based plasmid, downstream of a ptrc promoter.

In an alternative construct, to accomplish the four enzymatic activities, instead of the E. coli fadA and fadB genes, the E. coli fadI (NP_{—}416844) and fadj (NP_{—}416843) genes or the corresponding orthologs from other organisms can be used. Moreover, instead of the Euglena gracilis ter gene, the corresponding orthologs from other organisms or the E. coli fabl (NP_{—}415804) gene can be used. This is because, although FabI normally reduces trans-2-enoyl-ACPs, it has been reported to be active with trans-2-enoyl-CoAs (Bergler et al., 1993, J. Biol. Chem., vol. 269, pp. 5493-5496).

A compatible plasmid, for example, one based on pCL1920, is constructed to harbor a synthetic operon under the control of a suitable promoter, such as, for example, a ptrc promoter. This plasmid is co-transformed with the pACYC-ptrc_fadAB-ter plasmid or the pACYC-ptrc fadAB-fabI plasmid into an E. coli ΔfadE strain. The resulting two strains are cultured, extracted and analyzed for alkane production as described in Example 1.

Although the exact substrate specificity of Euglena gracilis TER is not known, the two strains described above both successfully produce alkanes.

Strains expressing aldehyde biosynthetic genes from Synechococcus elongatus PCC7942 (YP_{—}400611) can be expected to derive a portion of the alkane produced from malonyl-CoA-dependent acyl-ACP precursors. The aldehyde biosynthetic enzyme from Synechoccus elongatus PCC7942, YP_{—}400611, efficiently converts acyl-ACPs to fatty aldehydes when overexpressed in E. coli. This enzyme has higher specific activity for acyl-ACPs as compared to its specificity for acyl-CoAs.

In order to increase the proportion or exclusively produce alkanes derived from a malonyl-CoA-independent pathway, an alternative aldehyde biosynthetic enzyme that has lower activity towards acyl-ACPs than the one from Synechococcus elongatus PCC 7942 enzyme YP_{—}400611 can be employed instead. One example is the aldehyde biosynthetic enzyme from Synechocystis sp. PCC6803, NP_{—}442146 (SEQ ID NO: 69), which, when overexpressed in E. coli, converts acyl-CoAs but not acyl-ACPs to fatty aldehydes.

For example, a plasmid containing a synthetic operon from Synechocystis sp. PCC6803 under the control of a suitable promoter, for example, a ptrc promoter, is constructed. The plasmid is co-transformed with the pACYC-ptrc_fadAB-ter plasmid or the pACYC-ptrc_fadAB-fabI plasmid into an E. coli ΔfadE strain. These strains are cultured, extracted and analyzed for alkane production as described in Example 1.

The strain constructed in accordance to this example produces alkanes mainly from a malonyl-CoA independent pathway.

Other aldehyde biosynthetic enzyme orthologs from other cyanobacteria (e.g., those listed in Table 1, herein) that reduce acyl-CoAs with high efficiency while reducing acyl-ACPs with low efficiency can also be used to produce alkanes or alkenes, mainly from a malonyl-CoA independent pathway.

### Example 36

This example demonstrates the production of Alkanes/Alkenes from E. coli MG1566 2 with OP80-PCC7942_{—}1594 and OP183-MBIC11017 AM1_{—}4041.

A fermentation step was performed in a 5-liter fermentors. The engineered cells were taken from a frozen stock and were cultured in a defined medium containing 3 g/L of KH₂PO₄, 6 g/L of Na₂HPO₄.2H₂O, 2 g/L of (NH₄)₂SO₄, 0.246 g/L of MgSO₄ heptahydrate, 20 g/L of glucose, 200 mM of Bis-Tris buffer (pH 7.2), 1.0 ml/L of trace minerals and 1 mg/L thiamine. The first trace minerals solution was composed of 27 g/L of FeCl₃.6H₂O, 2 g/L of ZnCl₂.4H₂O, 2 g/L of CaCl₂.6H₂O, 2 g/L of Na₂MoO₄.2H₂O, 1.9 g/L of CuSO₄.5H₂O, 0.5 g/L of H₃BO₃, and 40 ml/L of concentrated HCl. After the cells from the frozen stock were cultured overnight, 50 ml of the culture was used to inoculate 1 liter of production medium in a fermentor with suitable temperature, pH, agitation, aeration and dissolved oxygen control. This medium contains 0.5 g/L of (NH₄)₂SO₄, 3 g/L of KH₂PO₄, 0.5 g/L of MgSO₄.7H₂O, 0.15 g/L of ferric citrate, 10 ml/L of a trace minerals solution, 1.25 ml/L of vitamin solution, 5 g/L of Bacto casaminoacids and 10 g/L of glucose. The second trace minerals solution was composed of 2 g/L of ZnCl₂.4H₂O, 2 g/L of CaCl₂.6H₂O, 2 g/L of Na₂MoO₄.2H₂O, 1.9 g/L of CuSO₄.5H₂O, 0.5 g/L of H₃BO₃, and 100 ml/L of concentrated HCl. The vitamin solution was composed of 0.42 g/L of riboflavin, 5.4 g/L of pantothenic acid, 6 g/L of niacin, 1.4 g/L of pyridoxine, 0.06 g/L of biotin, and 0.04 g/L of folic maintained by adding NH₄OH, which also acted as a nitrogen source for cell growth.

The culture medium was continuously monitored. When it reached an OD₆₀₀ of about 5, the pathway was induced by the addition of 1 mM isopropyl 13-D-1-thiogalactopyranoside (IPTG). At the same time a feed containing 60% glucose, 3.9 g/L MgSO₄.7H₂O, 1.6 g/L KH₂PO₄ and 0.135 g/L ferric citrate was supplied to the fermentor. The feed was supplied exponentially until it reached a maximum of 7.5 g glucose/L/h and was kept constant for the remaining of the run. The fermentation step was maintained for about 72 hours. The final volume of the fermentation was measured to be 3.7 liter.

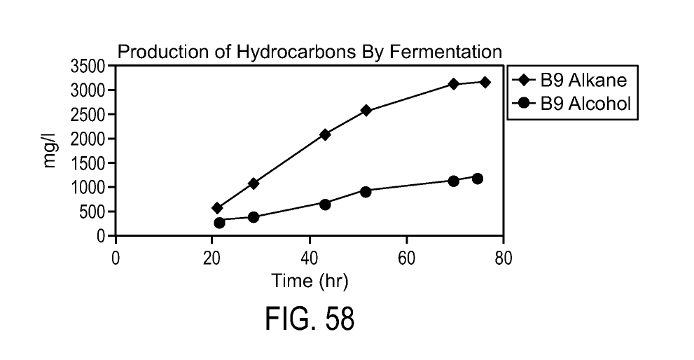

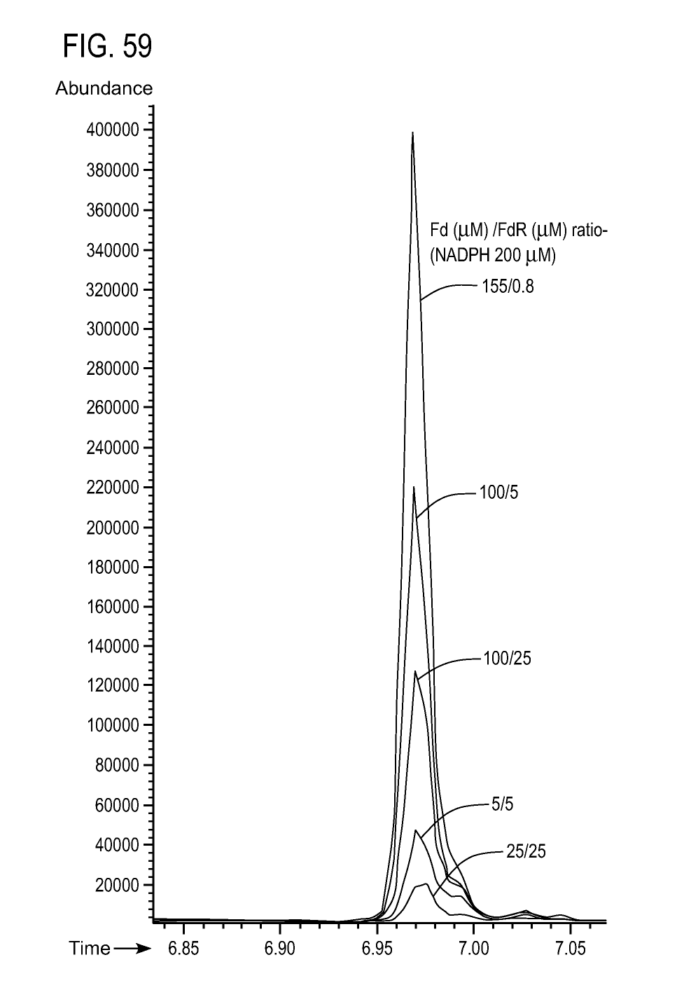

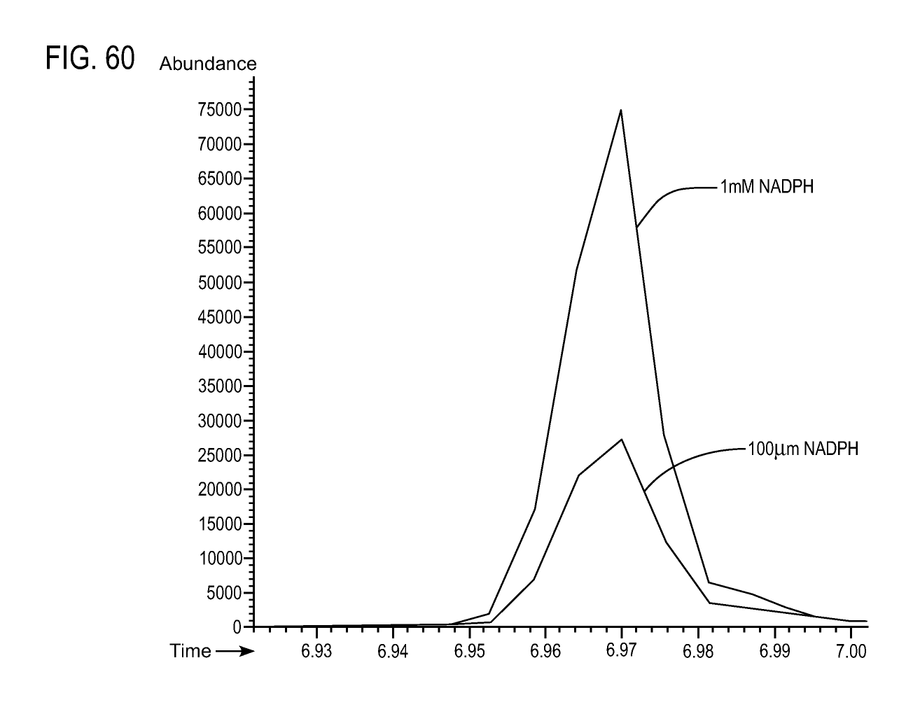

At various time points, for example, 20-hour, 40-hour, 60-hour, etc, an extraction was performed on 500 μl of fermentation broth using an equal volume of butyl acetate by vortexing for 15 minutes. The organic phase was analyzed using GC/MS as described herein. The concentration of alkanes, alkenes, and fatty alcohols produced at these different time points were plotted in FIG. 58.

After fermentation and induction of two fermentation runs, the resulting fermentation broths were pooled and centrifuged at 3,500 rpm for 15 min. The broth was then removed from the cell pellet and extracted with an equal volume of hexane and allowed to stir for 15 min. This mixture was then centrifuged again for 15 min at 3,500 rpm and the hexane layer was removed. This hexane layer was passed over sufficient amounts silica gel, pre-washed with hexane, to remove all polar contaminants. After this step the hexane dissolved alkanes were 99% pure based on GC/MS analysis. To remove the hexane, the mixture was vacuum distilled. After all of these steps, 14 g of alkanes were recovered.

### Example 37

This example demonstrates the production of a hydrocarbon by aldehyde decarbonylase (ADC) from Nostoc puntiforme PCC73102.

Aldehyde Decarbonylase (ADC), Ferredoxin (Fd), and Ferredoxin Reductase (FdR) from Nostoc puntiforme PCC73102 Npun02004178 were all purified via recombinant expression in E. coli. All proteins were N-terminally His tagged and expressed on plasmid pET15b, as previously described in Example 26. ADC at 100 uM in 100 mM pH 6.4 Bis Tris was reconstituted with ferrous sulfate, heptahydrate at a 2:1 ratio of Fe to ADC, under nitrogen. After reconstitution, no further care was taken to maintain anaerobiasis of ADC(Fe).

The following reactions were then performed as described below: ADC(Fe)+Fd+FdR+NADPH+C16:1 RHO+O₂→C15:1 RH+CO

For a solution with a total volume of 200 ul, 25 uM ADC(Fe), 0.8-25 uM FdR, 5-155 uM Fd, 0.1-1 mM NADPH, 200 uM C16:1 RHO in 0.1% Triton X, 100 mM Bis Tris pH 6.4 buffer were combined. The solution was vortexed gently and run for 15 minutes at room temperature, and then quenched and extracted with 100 ul n-butyl acetate. The organic layer was extracted and run on GC-MS. See FIGS. 62 and 63.

### Example 38

This example demonstrates the saturation mutagenesis of aldehyde decarbonylase (ADC) MBIC 11017.

The following sixteen different amino acid residues of aldehyde decarbonylase MBIC 11017 were mutated: E32, A35, Y39, M59, E60, H63, F67, C70, Q110, E115, A118, Y122, V140, D143, E144, and H147. All of these residues, with the exception of C70, are within 6 Å of the diiron centers of the ADC. The proximity of all of the residues, with the exception of the cysteine located at position 70, to the diiron centers of the ADC increases the likelihood of their involvement in in vivo activity of the enzyme. In addition, the cysteine residue at position 70 appeared to play a role in enzymatic function. These mutations were generated as described below.

Aldehyde decarbonylase MBIC 11017 was cloned into E. coli expression plasmid pLS9-193. PCR enzyme Phusion (New England Biolabs, Inc., Ipswich, Mass.) was used to amplify from before the start of the gene (using primer LB343: GCACTCGACCGGAATTATCG (SEQ ID NO: 116)) to just before the intended site of saturation. Phusion was also used to amplify from the saturation site forward through the second part of the gene to a sequence after the end of the gene (using primer LB295: TGGTCGACGGCGCTATTCAG (SEQ ID NO: 117)). The pLS9-193 plasmid was also used as a template for a PCR reaction using the Phusion enzyme in which the reverse sequences of LB343 (CGATAATTCCGGTCGAGTGC (SEQ ID NO: 118)) and LB295 (CTGAATAGCGCCGTCGACCA (SEQ ID NO: 119)) were used to amplify the vector. The primers and sequences used to perform the amplifications are listed below in Table 10.

TABLE 10

| Primer | Mutation | Sequence |
|---|---|---|
| oNH170 | ADC sat | CGATGGTATCGTGATCGAGGGTNNSCAAGAGgc |
| | E32 | gCATGAGAACtacATTCG |
| | | (SEQ ID NO: 120) |
| oNH171 | ADC E32 | ACCCTCGATCACGATACCATCG |
| | RC | (SEQ ID NO: 121) |
| oNH172 | ADC sat | GTGATCGAGGGTgagCAAGAGNNSCATGAGAACt |
| | A35 | acATTCGTCTGGGTG |
| | | (SEQ ID NO: 122) |
| oNHI73 | ADC A35 | CTCTTGctcACCCTCGATCAC |
| | RC | (SEQ ID NO: 123) |
| oNH174 | ADC sat | gagCAAGAGgcgCATGAGAACNNKATTCGTCTGG |
| | Y39 | GTGAAATGTTGCC |
| | | (SEQ ID NO: 124) |
| oNH175 | ADC Y39 | GTTCTCATGcgcCTCTTGctc |
| | RC | (SEQ ID NO: 125) |
| oNH176 | ADC sat | GACGACTTTATCCGTTTGAGCAAGNNSgagGCCC |
| | M59 | GTcacAAGAAGG |
| | | (SEQ ID NO: 126) |
| oNH177 | ADC M59 | CTTGCTCAAACGGATAAAGTCGTC |
| | RC | (SEQ ID NO: 127) |
| oNH178 | ADC sat | GACGACTTTATCCGTTTGAGCAAGatgNNSGCCC |
| | E60 | GTcacAAGAAGGGCtttG |
| | | (SEQ ID NO: 128) |
| | ADC E60 | used oNH177 |
| | RC | |
| oNH179 | ADC sat | GAGCAAGatggagGCCCGTNNKAAGAAGGGCtttG |
| | H63 | AGGCTTGTG |
| | | (SEQ ID NO: 129) |
| oNH180 | ADC H63 | ACGGGCctccatCTTGCTC |
| | RC | (SEQ ID NO: 130) |
| oNH181 | ADC sat | GCCCGTcacAAGAAGGGCNNSGAGGCTTGTGGT |
| | F67 | CGTAACTTGAAG |
| | | (SEQ ID NO: 131) |
| oNH182 | ADC F67 | GCCCTTCTTgtgACGGGC |
| | RC | (SEQ ID NO: 132) |
| oNH183 | ADC sat | GATAAAGTTCCGACCTGCTTGGTTATTNNSTCC |
| | Q110 | CTGATCATCgaaTGCTTCgc |
| | | (SEQ ID NO: 133) |
| oNH184 | ADC Q110 | AATAACCAAGCAGGTCGGAACTTTATC |
| | RC | (SEQ ID NO: 134) |
| oNH185 | ADC sat | GCTTGGTTATTcagTCCCTGATCATCNNSTGCTTC |
| | E115 | gcgATTGCAGCG |
| | | (SEQ ID NO: 135) |
| oNH186 | ADC E115 | GATGATCAGGGActgAATAACCAAGC |
| | RC | (SEQ ID NO: 136) |
| oNH187 | ADC sat | cagTCCCTGATCATCgaaTGCTTCNNSATTGCAGC |
| | A118 | GtatAACATTTACATCCCG |
| | | (SEQ ID NO: 137) |
| oNH188 | ADC A118 | GAAGCAttcGATGATCAGGGActg |
| | RC | (SEQ ID NO: 138) |
| oNH189 | ADC sat | TGCTTCgcgATTGCAGCGNNSAACATTTACATCC |
| | Y122 | CGGTTGCCG |
| | | (SEQ ID NO: 139) |
| oNH190 | ADC Y122 | CGCTGCAATcgcGAAGCA |
| | RC | (SEQ ID NO: 140) |
| oNH191 | ADC sat | CGCTCGTAAGATTACCGAGAGCNNKGTCAAGga |
| | V140 | cgaaTACCAGcatCTG |
| | | (SEQ ID NO: 141) |
| oNH192 | ADC V140 | GCTCTCGGTAATCTTACGAGCG |
| | RC | (SEQ ID NO: 142) |
| oNH193 | ADC sat | GTAAGATTACCGAGAGCgtcGTCAAGNNKgaaTA |
| | D143 | CCAGcatCTGAACTATGGCG |
| | | (SEQ ID NO: 143) |
| oNH194 | ADC D143 | CTTGACgacGCTCTCGGTAATCTTAC |
| | RC | (SEQ ID NO: 144) |
| oNH195 | ADC sat | GTAAGATTACCGAGAGCgtcGTCAAGgacNNSTAC |
| | E144 | CAGcatCTGAACTATGGCG |
| | | (SEQ ID NO: 145) |
| | ADC E144 | used oNH194 |
| | RC | |
| oNH196 | ADC sat | CgtcGTCAAGgacgaaTACCAGNNSCTGAACTATGG |
| | H147 | CGAGGAGTGG |
| | | (SEQ ID NO: 146) |
| oNH197 | ADC H147 | CTGGTAttcgtcCTTGACgacG |
| | RC | (SEQ ID NO: 147) |
| oNH218 | ADCsat | cacAAGAAGGGCtttGAGGCTNNSGGTCGTAACTT |
| | C70 fwd | GAAGGTGACTTGC |
| | | (SEQ ID NO: 148) |
| oNH219 | ADCsat | AGCCTCaaaGCCCTTCTTgtg |
| | C70 RC | (SEQ ID NO: 149) |

All PCR reactions described above were run on an agarose gel (1% agarose in TAE, Biorad, Hercules, Calif.) and were purified using a Gel DNA Recovery Kit (Zymo Research, Orange, Calif.) according to the instructions of the manufacturer. The DNA fragments that resulted from these reactions were paired up and stitched together using Phusion polymerase and the outside primers LB343 and LB295. Each of these 16 splicing by overlap extension “SOEing” reactions resulted in a DNA fragment that was run on an agarose gel and purified as above. Each contained the decarbonylase gene with one specifically chosen codon (three consecutive nucleotides) replaced by randomized nucleotides. In other words, each of the 16 DNA fragments that was purified from the agarose gel was actually a pool of DNA fragments that were identical to each other, except at the randomized position.

These 16 DNA fragments, with ends that correspond to the sequences of LB343 and LB295, were ligated to the vector (generated with LB343-reverse and LB295-reverse as described above) using a CloneEZ kit (Genscript, Piscataway, N.J.). Each of these 16 ligations was performed and 3 uL of each reaction, was transformed into NEB Turbo cells (New England Biolabs, Ipswich, Mass.), and plated on LB-agar plates with Carbenicillin antibiotic and incubated at 37° C. overnight. In order to verify the quality of the library, 8 colonies of the first reaction were PCR'd using GreenTaq (New England Biolabs, Ipswich, Mass.) and LB343 and LB295. These DNA fragments were sequenced and each of the 8 contained a sequence unique from all the others and from the wild-type sequence at the saturated position, indicating the high quality of the library. These 16 plates were scraped and miniprepped using a NucleoSpin Plasmid kit (Macherey-Nagel GmbH, Duren, Germany). Electrocompetent cells were prepared from the strain DV2.pLS9-185 and the 16 libraries were transformed into this strain via electroporation. Cells were recovered in SOC for 1 hour and plated at various dilutions on LB-agar plates with spectinomycin and carbenicillin antibiotics at 37° C. overnight.

Deep-well fermentation plates were prepared with 400 uL/well liquid LB plus spectinomycin and carbenicillin. Three wells on each plate were used to pick colonies from a plate of control colonies containing the wild-type decarbonylase (namely, DV2.pLS9-185.pLS9-193 wt). The other 93 wells on the plate were used to culture colonies picked from the saturation mutagenesis library. These LB culture plates were incubated in a 37° C. shaker at 200 rpm for 7 hours. 20 uL of this culture was used to inoculate a new 400 uL culture of 2NBT media. These 16 plates were grown overnight at 37° C. shaking at 200 rpm. The following morning 16 plates were prepared with 5NBT, 400 uL per well. Into each well, 20 uL of the overnight 2NBT culture was used as inoculum. After two hours the cultures were induced by the addition to each well of 50 uL of IPTG (10× concentration at 10 mM). Extraction was performed as previously described in, e.g., Example 3. The cells were then analyzed by GC-MS as described in Example 26.

The data were grouped such that a “low” colony had titers less than 10% of the average of the controls on its plate. A “high” colony had titers greater than 90% of the average of the controls on its plate. Colonies between 10-90% were termed “middle.” For positions E32, E60, H63, C70, E115, E144, H147, all clones that were greater than 10% of the on-plate controls were PCR'd with GreenTaq (NEB) and oNH253 (CATATGCCGCAAACGCAAGC (SEQ ID NO: 150)) and LB295 and sent for DNA sequencing using the primer LB295. Every one of these clones was found to encode the wild-type gene. For positions A35, Y39, M59, F67, Q110, A118, Y122, all clones that were greater than 10% of the on-plate controls were picked and rearrayed into 96-well plates with LB and spectinomycin and carbenicillin antibiotics. These plates were used to inoculate deep well plates which were incubated at 37° C. shaking at 200 rpm to begin another fermentation using the same protocol described as above. Identical extraction and GC protocols were used to generate the data shown below. In addition, all of these clones were PCR'd with GreenTaq (NEB) and oNH253 and LB295 and sent for DNA sequencing using the primer LB295. These data were combined to identify the in vivo activity of the mutant enzymes relative to the wild-type decarbonylase enzyme and is shown below in Table 11.

TABLE 11

| | Average Alkane |
|---|---|
| | Production (Fraction of |
| Mutation | control) |
| A35G | 0.64 |
| A35R | 0.33 |
| A35S | 0.15 |
| Y39C | 0.36 |
| Y39F | 1.09 |
| Y39H | 0.35 |
| Y39L | 0.62 |
| Y39M | 0.55 |
| Y39R | 0.19 |
| Y39V | 0.22 |
| Y39W | 0.41 |
| M59A | 0.54 |
| M59C | 0.62 |
| M59D | 0.10 |
| M59F | 0.86 |
| M59G | 0.47 |
| M59H | 0.31 |
| M59I | 1.03 |
| M59K | 0.82 |
| M59L | 1.00 |
| M59N | 0.36 |
| M59Q | 0.34 |
| M59R | 0.60 |
| M59S | 0.60 |
| M59T | 0.52 |
| M59V | 0.73 |
| M59W | 0.71 |
| M59Y | 0.98 |
| F67L | 0.31 |
| F67M | 0.39 |
| F67Q | 0.14 |
| F67V | 0.15 |
| F67W | 0.26 |
| F67Y | 0.63 |
| L90V | 0.53 |
| Q110A | 0.53 |
| Q110C | 0.42 |
| Q110E | 0.29 |
| Q110F | 0.12 |
| Q110G | 0.27 |
| Q110H | 0.28 |
| Q110I | 0.14 |
| Q110M | 0.46 |
| Q110N | 0.08 |
| Q110P | 0.12 |
| Q110R | 0.16 |
| Q110S | 0.46 |
| Q110T | 0.32 |
| Q110V | 0.40 |
| A118G | 0.28 |
| A118I | 0.65 |
| A118P | 0.33 |
| A118R | 0.41 |
| A118S | 0.14 |
| A118V | 0.20 |
| Y122C | 0.10 |
| Y122F | 1.20 |
| Y122I | 0.30 |
| Y122L | 0.70 |
| Y122M | 0.36 |
| Y122P | 0.14 |
| Y122Q | 0.13 |

As is apparent from the data set forth above, several of the mutants had similar or improved activity when compared with the wild type enzyme, which suggests possible targets for additional mutatgenesis experiments. Based on the experiments described in Examples 31 and 39, a polypeptide motif, SEQ ID NO: 115, was created as a model of the residues necessary to constitute a functional decarbonylase. As described in Example 31, homology models indicate that decarbonylases share the same six amino acid residues within about 6 Å of the metallic center of the enzyme. Based on this information, SEQ ID NO: 105-108 were developed. The role of these residues in enzymatic stability and function was further investigated in Example 39, where it was discovered that certain residues could be substituted for others, resulting in a decarbonylase having the same or better activity than wild type decarbonylase. Four additional sequence motifs (SEQ ID NO: 151-154) that are likely to exhibit decarbonylase activity similar to or better than that of wild type decarbonylases were developed.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated polypeptide comprising the amino acid sequence of SEQ ID NO: 37 or SEQ ID NO: 115, wherein the polypeptide is not a member of the group consisting of SEQ ID NO: 2, 4, 6, 8, 10, 12, 14, 16, 18, 20, 22, 24, 26, 28, 30, 32, 34, and 36.

2. The isolated polypeptide of claim 1, wherein the polypeptide has decarbonylase activity.

3. The isolated polypeptide of claim 1, wherein the polypeptide is selected from the group consisting of SEQ ID NO: 105, 106, 107, 108, 151, 152, 153, and 154.

4. The isolated polypeptide of claim 1, wherein one or more amino acid residues of the polypeptide is bound to one or more metal ions.

5. The isolated polypeptide of claim 4, wherein at least one of the metal ions is iron.

6. A genetically engineered microorganism comprising a nucleic acid sequence encoding the amino acid sequence of SEQ ID NO: 37 or SEQ ID NO: 115, wherein the nucleic acid sequence is expressed in the microorganism to produce a polypeptide comprising the amino acid sequence of SEQ ID NO: 37 or SEQ ID NO: 115.

7. The genetically engineered microorganism of claim 6, wherein the polypeptide has decarbonylase activity.

8. The genetically engineered microorganism of claim 6, wherein the polypeptide is selected from the group consisting of SEQ ID NO: 105, 106, 107, 108, 151, 152, 153, and 154.

9. The genetically engineered microorganism of claim 6, wherein one or more amino acid residues of the polypeptide is bound to one or more metal ions.

10. The genetically engineered microorganism of claim 9, wherein at least one of the metal ions is iron.

11. The genetically engineered microorganism of claim 6, wherein the polypeptide is not a member of the group consisting of SEQ ID NO: 2, 4, 6, 8, 10, 12, 14, 16, 18, 20, 22, 24, 26, 28, 30, 32, 34, and 36.

12. The genetically engineered microorganism of claim 6, wherein an exogenous control sequence is stably incorporated into the genomic DNA of the microorganism upstream of the nucleic acid sequence.

13. The genetically engineered microorganism of claim 6, wherein the microorganism produces an increased level of a hydrocarbon relative to a wild-type microorganism.

14. The genetically engineered microorganism of claim 6, further comprising a substrate for the polypeptide.

15. The genetically engineered microorganism of claim 14, wherein the substrate is a fatty acid derivative.

16. A method of producing a hydrocarbon, the method comprising
(a) expressing in a host cell a nucleic acid sequence encoding a polypeptide comprising the amino acid sequence of SEQ ID NO: 37 or SEQ ID NO: 115,
(b) providing the polypeptide, wherein the polypeptide interacts with the host cell to produce a hydrocarbon, and
(c) isolating the hydrocarbon from the host cell.

17. The method of claim 16, wherein the polypeptide is selected from the group consisting of SEQ ID NO: 105, 106, 107, 108, 151, 152, 153, and 154.

18. The method of claim 16, wherein the host cell is selected from the group consisting of a mammalian cell, plant cell, insect cell, yeast cell, fungus cell, filamentous fungi cell, and bacterial cell.

19. The method of claim 16, wherein the hydrocarbon is secreted by the host cell.

20. The method of claim 16, wherein the hydrocarbon comprises a C₁₃-C₂₁ alkane.

21. The method of claim 16, wherein the hydro comprises a C₁₃-C₂₁ alkene.

22. The method of claim 16, further comprising culturing the host cell in the presence of at least one substrate for the polypeptide.

23. The method of claim 22, wherein the polypeptide interacts with the substrate to form the hydrocarbon.

24. The method of claim 23, wherein the substrate is a fatty acid derivative.

25. The method of claim 16, wherein the polypeptide has decarbonylase activity.

26. The method of claim 16, wherein one or more amino acid residues of the polypeptide is bound to one or more metal ions.

27. The method of claim 26, wherein at least one of the metal ions is iron.

28. The method of claim 16, wherein the polypeptide is not a member of the group consisting of SEQ ID NO: 2, 4, 6, 8, 10, 12, 14, 16, 18, 20, 22, 24, 26, 28, 30, 32, 34, and 36.
